SEQUENCE LISTING (159 sequences)

SEQ ID NO: 1 - protein, 221 aa, synthetic construct
  REGION 1..221: note = Human PR3 mature sequence (WT) or PR3_1
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLR

SEQ ID NO: 2 - protein, 221 aa, synthetic construct
  REGION 1..221: note = PR3_2 (S176A)
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDAGGPLICDGIIQGIDSFVIWGCATRLFPDFFTRVALYVDWIRSTLR

SEQ ID NO: 3 - protein, 229 aa, synthetic construct
  REGION 1..229: note = PR3_3 (C-terminal addition)
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDAGGPLICDGIIQGIDSFVIWGCATRLFPDFFTRVALYVDWIRSTLRRVEAKGRP

SEQ ID NO: 4 - protein, 221 aa, synthetic construct
  REGION 1..221: note = PR3_4 (D91N)
IVGGHEAQPHSRPYMASLQMRGNPGSHFCGGTLIHPSFVLTAAHCLRDIPQRLVNVVLGAHNVRTQEPTQQHFSVAQVFLNNYDAENKLNNVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLR

SEQ ID NO: 5 - protein, 223 aa, synthetic construct
  REGION 1..223: note = PR3_5 (Unprocessed N-terminus)
AEIVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLR

SEQ ID NO: 6 - protein, 220 aa, synthetic construct
  REGION 1..220: note = PR3_6 (Ile Deletion)
VGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLR

SEQ ID NO: 7 - protein, 221 aa, synthetic construct
  REGION 1..221: note = PR3_7 (D175N)
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGNSGGPLICDGIIQGIDSFVIWGCATRLFPDFFTRVALYVDWIRSTLR

SEQ ID NO: 8 - protein, 221 aa, synthetic construct
  REGION 1..221: note = PR3_8 (S176C)
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDCGGPLICDGIIQGIDSFVIWGCATRLFPDFFTRVALYVDWIRSTLR

SEQ ID NO: 9 - protein, 221 aa, synthetic construct
  REGION 1..221: note = PR3_9 (G4P)
IVGPHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLR

SEQ ID NO: 10 - protein, 221 aa, synthetic construct
  REGION 1..221: note = PR3_10 (H44A)
IVGGHEAQPHSRPYMASLQMRGNPGSHFCGGTLIHPSFVLTAAACLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLR

SEQ ID NO: 11 - DNA, 663 bp, synthetic construct
  misc_feature 1..663: note = Human PR3 mature sequence (WT) or PR3_1
  source 1..663: mol_type = other DNA
atcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgaga

SEQ ID NO: 12 - DNA, 663 bp, synthetic construct
  misc_feature 1..663: note = PR3_2 S176A
  source 1..663: mol_type = other DNA
atcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgctttggagatgctggcggccctctgatctgcgacggaatcatccagggcatcgacagcttcgtgatctggggctgtgccaccagactgttccccgacttctttacccgggtggccctctacgtggactggatccggtctacactgaga

SEQ ID NO: 13 - DNA, 687 bp, synthetic construct
  misc_feature 1..687: note = PR3_3 (C-terminal addition)
  source 1..687: mol_type = other DNA
atcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgctttggagatgctggcggccctctgatctgcgacggaatcatccagggcatcgacagcttcgtgatctggggctgtgccaccagactgttccccgacttctttacccgcgtggccctctacgtggactggatcagatctaccctgcggagagtggaagccaagggcagacct

SEQ ID NO: 14 - DNA, 663 bp, synthetic construct
  misc_feature 1..663: note = PR3_4 (D91N)
  source 1..663: mol_type = other DNA
atcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacaacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgaga

SEQ ID NO: 15 - DNA, 669 bp, synthetic construct
  misc_feature 1..669: note = PR3_5 ((Unprocessed N-terminus)
  source 1..669: mol_type = other DNA
gccgagatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgaga

SEQ ID NO: 16 - DNA, 660 bp, synthetic construct
  misc_feature 1..660: note = PR3_6 (Ile Deletion)
  source 1..660: mol_type = other DNA
gttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgaga

SEQ ID NO: 17 - DNA, 663 bp, synthetic construct
  misc_feature 1..663: note = PR3_7 (D175N)
  source 1..663: mol_type = other DNA
atcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggaaactctggcggccctctgatctgcgacggaatcatccagggcatcgacagcttcgtgatctggggctgtgccaccagactgttccccgacttctttacccgggtggccctctacgtggactggatccggtctacactgaga

SEQ ID NO: 18 - DNA, 663 bp, synthetic construct
  misc_feature 1..663: note = PR3_8 (S176C)
  source 1..663: mol_type = other DNA
atcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattgcggcggccctctgatctgcgacggaatcatccagggcatcgacagcttcgtgatctggggctgtgccaccagactgttccccgacttctttacccgggtggccctctacgtggactggatccggtctacactgaga

SEQ ID NO: 19 - DNA, 663 bp, synthetic construct
  misc_feature 1..663: note = PR3_9 (G4P)
  source 1..663: mol_type = other DNA
atcgttggaccccatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgaga

SEQ ID NO: 20 - DNA, 663 bp, synthetic construct
  misc_feature 1..663: note = PR3_10 (H44A)
  source 1..663: mol_type = other DNA
atcgttggaggacatgaagcccagcctcacagcagaccttacatggccagcctgcagatgagaggcaatcccggctctcacttttgtggcggcaccctgattcacccctccttcgttctgaccgccgctgcctgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgaga

SEQ ID NO: 21 - protein, 16 aa, synthetic construct
  REGION 1..16: note = CD33 signal peptide
MPLLLLLPLLWAGALA

SEQ ID NO: 22 - protein, 13 aa, synthetic construct
  REGION 1..13: note = IgG4 hinge spacer (aa) Short spacer (SS)
ESKYGPPCPPCPM

SEQ ID NO: 23 - protein, 12 aa, synthetic construct
  REGION 1..12: note = IgG4 hinge spacer (aa) Short spacer (SS)
ESKYGPPCPPCP

SEQ ID NO: 24 - protein, 120 aa, synthetic construct
  REGION 1..120: note = IgG4 Hinge-CH3 spacer (aa) Medium spacer (MS)
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 25 - protein, 229 aa, synthetic construct
  REGION 1..229: note = IgG4 hinge- IgG2/IgG4 CH2- IgG4 CH3 spacer (aa) Long spacer (LS)
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 26 - protein, 27 aa, synthetic construct
  REGION 1..27: note = CD28 transmembrane domain (aa)
FWVLVVVGGVLACYSLLVTVAFIIFWV

SEQ ID NO: 27 - protein, 42 aa, synthetic construct
  REGION 1..42: note = 4-1BB-derived intracellular co-signaling sequence (aa)
KRGRKKLLYIFKQPFMRPVQTTQEEDGCSCRFPEEEEGGCEL

SEQ ID NO: 28 - protein, 112 aa, synthetic construct
  REGION 1..112: note = CD3-zeta derived intracellular signaling domain (aa)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 415 aa, synthetic construct
  REGION 1..415: note = Human PR3 mature sequence (WT) or PR3_1 SS CAAR
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 522 aa, synthetic construct
  REGION 1..522: note = Human PR3 mature sequence (WT) or PR3_1 MS CAAR
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 631 aa, synthetic construct
  REGION 1..631: note = Human PR3 mature sequence (WT) or PR3_1 LS CAAR
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 415 aa, synthetic construct
  REGION 1..415: note = PR3_2 (S176A) SS CAAR
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDAGGPLICDGIIQGIDSFVIWGCATRLFPDFFTRVALYVDWIRSTLRESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 522 aa, synthetic construct
  REGION 1..522: note = PR3_2 (S176A) MS CAAR PR3_2 (S176A) MS CAAR
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDAGGPLICDGIIQGIDSFVIWGCATRLFPDFFTRVALYVDWIRSTLRESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 631 aa, synthetic construct
  REGION 1..631: note = PR3_2 (S176A) LS CAAR
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDAGGPLICDGIIQGIDSFVIWGCATRLFPDFFTRVALYVDWIRSTLRESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 423 aa, synthetic construct
  REGION 1..423: note = PR3_3 (C-terminal addition) SS CAAR
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDAGGPLICDGIIQGIDSFVIWGCATRLFPDFFTRVALYVDWIRSTLRRVEAKGRPESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 530 aa, synthetic construct
  REGION 1..530: note = PR3_3 (C-terminal addition) MS CAAR
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDAGGPLICDGIIQGIDSFVIWGCATRLFPDFFTRVALYVDWIRSTLRRVEAKGRPESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 639 aa, synthetic construct
  REGION 1..639: note = PR3_3 (C-terminal addition) LS CAAR
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDAGGPLICDGIIQGIDSFVIWGCATRLFPDFFTRVALYVDWIRSTLRRVEAKGRPESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 415 aa, synthetic construct
  REGION 1..415: note = PR3_4 (D91N) SS CAAR
IVGGHEAQPHSRPYMASLQMRGNPGSHFCGGTLIHPSFVLTAAHCLRDIPQRLVNVVLGAHNVRTQEPTQQHFSVAQVFLNNYDAENKLNNVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 522 aa, synthetic construct
  REGION 1..522: note = PR3_4 (D91N) MS CAAR
IVGGHEAQPHSRPYMASLQMRGNPGSHFCGGTLIHPSFVLTAAHCLRDIPQRLVNVVLGAHNVRTQEPTQQHFSVAQVFLNNYDAENKLNNVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 631 aa, synthetic construct
  REGION 1..631: note = PR3_4 (D91N) LS CAAR
IVGGHEAQPHSRPYMASLQMRGNPGSHFCGGTLIHPSFVLTAAHCLRDIPQRLVNVVLGAHNVRTQEPTQQHFSVAQVFLNNYDAENKLNNVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 417 aa, synthetic construct
  REGION 1..417: note = PR3_5 (Unprocessed N-terminus) SS CAAR
AEIVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 524 aa, synthetic construct
  REGION 1..524: note = PR3_5 (Unprocessed N-terminus) MS CAAR
AEIVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 633 aa, synthetic construct
  REGION 1..633: note = PR3_5 (Unprocessed N-terminus) LS CAAR
AEIVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 414 aa, synthetic construct
  REGION 1..414: note = PR3_6 (Ile Deletion) SS CAAR
VGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 521 aa, synthetic construct
  REGION 1..521: note = PR3_6 (Ile Deletion) MS CAAR
VGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 630 aa, synthetic construct
  REGION 1..630: note = PR3_6 (Ile Deletion) LS CAAR
VGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 415 aa, synthetic construct
  REGION 1..415: note = PR3_7 (D175N) SS CAAR
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGNSGGPLICDGIIQGIDSFVIWGCATRLFPDFFTRVALYVDWIRSTLRESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 522 aa, synthetic construct
  REGION 1..522: note = PR3_7 (D175N) MS CAAR
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGNSGGPLICDGIIQGIDSFVIWGCATRLFPDFFTRVALYVDWIRSTLRESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 631 aa, synthetic construct
  REGION 1..631: note = PR3_7 (D175N) LS CAAR
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGNSGGPLICDGIIQGIDSFVIWGCATRLFPDFFTRVALYVDWIRSTLRESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 415 aa, synthetic construct
  REGION 1..415: note = PR3_8 (S176C) SS CAAR
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDCGGPLICDGIIQGIDSFVIWGCATRLFPDFFTRVALYVDWIRSTLRESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 522 aa, synthetic construct
  REGION 1..522: note = PR3_8 (S176C) MS CAAR 42
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDCGGPLICDGIIQGIDSFVIWGCATRLFPDFFTRVALYVDWIRSTLRESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 631 aa, synthetic construct
  REGION 1..631: note = PR3_8 (S176C) LS CAAR
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDCGGPLICDGIIQGIDSFVIWGCATRLFPDFFTRVALYVDWIRSTLRESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 415 aa, synthetic construct
  REGION 1..415: note = PR3_9 (G4P) SS CAAR
IVGPHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 522 aa, synthetic construct
  REGION 1..522: note = PR3_9 (G4P) MS CAAR
IVGPHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 631 aa, synthetic construct
  REGION 1..631: note = PR3_9 (G4P) LS CAAR
IVGPHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 415 aa, synthetic construct
  REGION 1..415: note = PR3_10 (H44A) SS CAAR
IVGGHEAQPHSRPYMASLQMRGNPGSHFCGGTLIHPSFVLTAAACLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 522 aa, synthetic construct
  REGION 1..522: note = PR3_10 (H44A) MS CAAR
IVGGHEAQPHSRPYMASLQMRGNPGSHFCGGTLIHPSFVLTAAACLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 631 aa, synthetic construct
  REGION 1..631: note = PR3_10 (H44A) LS CAAR
IVGGHEAQPHSRPYMASLQMRGNPGSHFCGGTLIHPSFVLTAAACLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - DNA, 1293 bp, synthetic construct
  misc_feature 1..1293: note = Human PR3 sequence (WT) or PR3_1 SS CAAR
  source 1..1293: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 60 - DNA, 1614 bp, synthetic construct
  misc_feature 1..1614: note = Human PR3 sequence (WT) or PR3_1 MS CAAR
  source 1..1614: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 61 - DNA, 1941 bp, synthetic construct
  misc_feature 1..1941: note = Human PR3 sequence (WT) or PR3_1 LS CAAR
  source 1..1941: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 62 - DNA, 1293 bp, synthetic construct
  misc_feature 1..1293: note = PR3_2 (S176A) SS CAAR
  source 1..1293: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgctttggagatgctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 63 - DNA, 1614 bp, synthetic construct
  misc_feature 1..1614: note = PR3_2 (S176A) MS CAAR
  source 1..1614: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgctttggagatgctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 64 - DNA, 1941 bp, synthetic construct
  misc_feature 1..1941: note = PR3_2 (S176A) LS CAAR
  source 1..1941: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgctttggagatgctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 65 - DNA, 1317 bp, synthetic construct
  misc_feature 1..1317: note = PR3_3 (C-terminal addition) SS CAAR
  source 1..1317: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgctttggagatgctggcggccctctgatctgcgacggaatcatccagggcatcgacagcttcgtgatctggggctgtgccaccagactgttccccgacttctttacccgcgtggccctctacgtggactggatcagatctaccctgcggagagtggaagccaagggcagacctgagtctaaatacggaccgccttgtcctccttgtcccatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 66 - DNA, 1638 bp, synthetic construct
  misc_feature 1..1638: note = PR3_3 (C-terminal addition) MS CAAR
  source 1..1638: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgctttggagatgctggcggccctctgatctgcgacggaatcatccagggcatcgacagcttcgtgatctggggctgtgccaccagactgttccccgacttctttacccgcgtggccctctacgtggactggatcagatctaccctgcggagagtggaagccaagggcagacctgagtctaaatacggaccgccttgtcctccttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 67 - DNA, 1965 bp, synthetic construct
  misc_feature 1..1965: note = PR3_3 (C-terminal addition) LS CAAR
  source 1..1965: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgctttggagatgctggcggccctctgatctgcgacggaatcatccagggcatcgacagcttcgtgatctggggctgtgccaccagactgttccccgacttctttacccgcgtggccctctacgtggactggatcagatctaccctgcggagagtggaagccaagggcagacctgagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 68 - DNA, 1293 bp, synthetic construct
  misc_feature 1..1293: note = PR3_4 (D91N) SS CAAR
  source 1..1293: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacaacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 69 - DNA, 1614 bp, synthetic construct
  misc_feature 1..1614: note = PR3_4 (D91N) MS CAAR
  source 1..1614: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacaacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 70 - DNA, 1941 bp, synthetic construct
  misc_feature 1..1941: note = PR3_4 (D91N) LS CAAR
  source 1..1941: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacaacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 71 - DNA, 1299 bp, synthetic construct
  misc_feature 1..1299: note = PR3_5 (Unprocessed N-terminus) SS CAAR
  source 1..1299: mol_type = other DNA
atgcctttgttgctgctgctgcctcttctgtgggctggcgctctggctgccgagatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 72 - DNA, 1620 bp, synthetic construct
  misc_feature 1..1620: note = PR3_5 (Unprocessed N-terminus) MS CAAR
  source 1..1620: mol_type = other DNA
atgcctttgttgctgctgctgcctcttctgtgggctggcgctctggctgccgagatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 73 - DNA, 1947 bp, synthetic construct
  misc_feature 1..1947: note = PR3_5 (Unprocessed N-terminus) LS CAAR
  source 1..1947: mol_type = other DNA
atgcctttgttgctgctgctgcctcttctgtgggctggcgctctggctgccgagatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 74 - DNA, 1290 bp, synthetic construct
  misc_feature 1..1290: note = PR3_6 (Ile Deletion) SS CAAR
  source 1..1290: mol_type = other DNA
atgcctttgttgctgctgctgcctcttctgtgggctggcgctctggctgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 75 - DNA, 1611 bp, synthetic construct
  misc_feature 1..1611: note = PR3_6 (Ile Deletion) MS CAAR
  source 1..1611: mol_type = other DNA
atgcctttgttgctgctgctgcctcttctgtgggctggcgctctggctgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 76 - DNA, 1938 bp, synthetic construct
  misc_feature 1..1938: note = PR3_6 (Ile Deletion) LS CAAR
  source 1..1938: mol_type = other DNA
atgcctttgttgctgctgctgcctcttctgtgggctggcgctctggctgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 77 - DNA, 1293 bp, synthetic construct
  misc_feature 1..1293: note = PR3_7 (D175N) SS CAAR
  source 1..1293: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggaaactctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 78 - DNA, 1614 bp, synthetic construct
  misc_feature 1..1614: note = PR3_7 (D175N) MS CAAR
  source 1..1614: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggaaactctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 79 - DNA, 1941 bp, synthetic construct
  misc_feature 1..1941: note = PR3_7 (D175N) LS CAAR
  source 1..1941: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggaaactctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 80 - DNA, 1293 bp, synthetic construct
  misc_feature 1..1293: note = PR3_8 (S176C) SS CAAR
  source 1..1293: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattgcggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 81 - DNA, 1614 bp, synthetic construct
  misc_feature 1..1614: note = PR3_8 (S176C) MS CAAR PR3_8 (S176C) MS CAAR 42
  source 1..1614: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattgcggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 82 - DNA, 1941 bp, synthetic construct
  misc_feature 1..1941: note = PR3_8 (S176C) LS CAAR
  source 1..1941: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattgcggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 83 - DNA, 1293 bp, synthetic construct
  misc_feature 1..1293: note = PR3_9 (G4P) SS CAAR
  source 1..1293: mol_type = other DNA
atgcctttgttgctgctgctgcctcttctgtgggctggcgctctggctatcgttggaccccatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 84 - DNA, 1614 bp, synthetic construct
  misc_feature 1..1614: note = PR3_9 (G4P) MS CAAR
  source 1..1614: mol_type = other DNA
atgcctttgttgctgctgctgcctcttctgtgggctggcgctctggctatcgttggaccccatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 85 - DNA, 1941 bp, synthetic construct
  misc_feature 1..1941: note = PR3_9 (G4P) LS CAAR
  source 1..1941: mol_type = other DNA
atgcctttgttgctgctgctgcctcttctgtgggctggcgctctggctatcgttggaccccatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 86 - DNA, 1293 bp, synthetic construct
  misc_feature 1..1293: note = PR3_10 (H44A) SS CAAR
  source 1..1293: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctgcctgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 87 - DNA, 1614 bp, synthetic construct
  misc_feature 1..1614: note = PR3_10 (H44A) MS CAAR
  source 1..1614: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctgcctgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 88 - DNA, 1941 bp, synthetic construct
  misc_feature 1..1941: note = PR3_10 (H44A) LS CAAR
  source 1..1941: mol_type = other DNA
atgcctttgttgctgctgctgcctcttctgtgggctggcgctctggctatcgttggaggacatgaagcccagcctcacagcagaccttacatggccagcctgcagatgagaggcaatcccggctctcacttttgtggcggcaccctgattcacccctccttcgttctgaccgccgctgcctgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 89 - protein, 10 aa, synthetic construct
  REGION 1..10: note = ANCA1 epitope
MAHRPPSPAL

SEQ ID NO: 90 - protein, 11 aa, synthetic construct
  REGION 1..11: note = ANCA2 epitope
AQPHSRPYMAS

SEQ ID NO: 91 - protein, 8 aa, synthetic construct
  REGION 1..8: note = ANCA3 epitope
PGSHFCGG

SEQ ID NO: 92 - protein, 11 aa, synthetic construct
  REGION 1..11: note = ANCA4 epitope
VVLGAHNVRTQ

SEQ ID NO: 93 - protein, 8 aa, synthetic construct
  REGION 1..8: note = ANCA5 epitope
FLNNYDAE

SEQ ID NO: 94 - protein, 8 aa, synthetic construct
  REGION 1..8: note = ANCA6 epitope
PVPHGTQC

SEQ ID NO: 95 - protein, 8 aa, synthetic construct
  REGION 1..8: note = ANCA7 eptitope
CFGDSGGP

SEQ ID NO: 96 - protein, 119 aa, synthetic construct
  REGION 1..119: note = IgG4 Hinge-CH3 spacer (aa) Medium spacer (MS)
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 97 - protein, 228 aa, synthetic construct
  REGION 1..228: note = IgG4 hinge- IgG2/IgG4 CH2- IgG4 CH3 spacer (aa) Long spacer (LS)
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 98 - protein, 21 aa, synthetic construct
  REGION 1..21: note = CD8a transmembrane domain (aa)
IYIWAPLAGTCGVLLLSLVIT

SEQ ID NO: 99 - protein, 5 aa, synthetic construct
  REGION 1..5: note = G4s spacer (aa)
GGGGS

SEQ ID NO: 100 - protein, 15 aa, synthetic construct
  REGION 1..15: note = (G4S)3 spacer (aa)
GGGGSGGGGSGGGGS

SEQ ID NO: 101 - protein, 47 aa, synthetic construct
  REGION 1..47: note = CD8a spacer (aa)
KPTTTPAPRPPTPAPTIASQPLSLRPEACRPAAGGAVHTRGLDFACD

SEQ ID NO: 102 - protein, 5 aa, synthetic construct
  REGION 1..5: note = EAAAK spacer (aa)
EAAAK

SEQ ID NO: 103 - protein, 411 aa, synthetic construct
  REGION 1..411: note = PR3_9 (G4P)/(G4S)3/CD8aTM CAAR
IVGPHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRGGGGSGGGGSGGGGSIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - protein, 417 aa, synthetic construct
  REGION 1..417: note = PR3_9 (G4P)/(G4S)3/CD28TM CAAR
IVGPHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRGGGGSGGGGSGGGGSFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - protein, 401 aa, synthetic construct
  REGION 1..401: note = PR3_9 (G4P)/(G4S)/CD8aTM CAAR
IVGPHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRGGGGSIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 407 aa, synthetic construct
  REGION 1..407: note = PR3_9 (G4P)/(G4S)/CD28TM CAAR
IVGPHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRGGGGSFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - protein, 443 aa, synthetic construct
  REGION 1..443: note = PR3_9 (G4P)/CD8a/CD8aTM CAAR
IVGPHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - protein, 401 aa, synthetic construct
  REGION 1..401: note = PR3_9 (G4P)/EAAAK/CD8aTM CAAR
IVGPHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLREAAAKIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 109 - protein, 516 aa, synthetic construct
  REGION 1..516: note = PR3_9 (G4P)/MS/CD8aTM CAAR
IVGPHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 110 - protein, 409 aa, synthetic construct
  REGION 1..409: note = PR3_9 (G4P)/SS/CD8aTM CAAR
IVGPHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPMIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - protein, 411 aa, synthetic construct
  REGION 1..411: note = PR3_10 (H44A)/(G4S)3/CD8aTM CAAR
IVGGHEAQPHSRPYMASLQMRGNPGSHFCGGTLIHPSFVLTAAACLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRGGGGSGGGGSGGGGSIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - protein, 417 aa, synthetic construct
  REGION 1..417: note = PR3_10 (H44A)/(G4S)3/CD28TM CAAR
IVGGHEAQPHSRPYMASLQMRGNPGSHFCGGTLIHPSFVLTAAACLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRGGGGSGGGGSGGGGSFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 113 - protein, 401 aa, synthetic construct
  REGION 1..401: note = PR3_10 (H44A)/(G4S)/CD8aTM CAAR
IVGGHEAQPHSRPYMASLQMRGNPGSHFCGGTLIHPSFVLTAAACLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRGGGGSIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 114 - protein, 407 aa, synthetic construct
  REGION 1..407: note = PR3_10 (H44A)/(G4S)/CD28TM CAAR
IVGGHEAQPHSRPYMASLQMRGNPGSHFCGGTLIHPSFVLTAAACLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRGGGGSFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - protein, 443 aa, synthetic construct
  REGION 1..443: note = PR3_10 (H44A)/CD8a/CD8aTM CAAR
IVGGHEAQPHSRPYMASLQMRGNPGSHFCGGTLIHPSFVLTAAACLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - protein, 401 aa, synthetic construct
  REGION 1..401: note = PR3_10 (H44A)/EAAAK/CD8aTM CAAR
IVGGHEAQPHSRPYMASLQMRGNPGSHFCGGTLIHPSFVLTAAACLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLREAAAKIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - protein, 516 aa, synthetic construct
  REGION 1..516: note = PR3_10 (H44A)/MS/CD8aTM CAAR
IVGGHEAQPHSRPYMASLQMRGNPGSHFCGGTLIHPSFVLTAAACLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - protein, 409 aa, synthetic construct
  REGION 1..408: note = PR3_10 (H44A)/SS/CD8aTM CAAR
IVGGHEAQPHSRPYMASLQMRGNPGSHFCGGTLIHPSFVLTAAACLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRESKYGPPCPPCPMIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - protein, 413 aa, synthetic construct
  REGION 1..413: note = PR3_10 (H44A)/(G4S)3/CD8aTM CAAR
AEIVGGHEAQPHSRPYMASLQMRGNPGSHFCGGTLIHPSFVLTAAACLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRGGGGSGGGGSGGGGSIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 419 aa, synthetic construct
  REGION 1..419: note = PR3_10 (H44A)/(G4S)3/CD28TM CAAR
AEIVGGHEAQPHSRPYMASLQMRGNPGSHFCGGTLIHPSFVLTAAACLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRGGGGSGGGGSGGGGSFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 403 aa, synthetic construct
  REGION 1..403: note = PR3_10 (H44A)/(G4S)/CD8aTM CAAR
AEIVGGHEAQPHSRPYMASLQMRGNPGSHFCGGTLIHPSFVLTAAACLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRGGGGSIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - protein, 409 aa, synthetic construct
  REGION 1..409: note = PR3_10 (H44A)/(G4S)/CD28TM CAAR
AEIVGGHEAQPHSRPYMASLQMRGNPGSHFCGGTLIHPSFVLTAAACLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRGGGGSFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 25 aa, synthetic construct
  REGION 1..25: note = PR3 Signal Peptide
MAHRPPSPALASVLLALLLSGAARA

SEQ ID NO: 124 - skipped

SEQ ID NO: 125 - protein, 15 aa, synthetic construct
  REGION 1..15: note = Hinge
EPKSCDKTHTCPPCP

SEQ ID NO: 126 - protein, 12 aa, synthetic construct
  REGION 1..12: note = Hinge
ERKCCVECPPCP

SEQ ID NO: 127 - protein, 61 aa, synthetic construct
  REGION 1..61: note = Hinge
ELKTPLGDTHTCPRCPEPKSCDTPPPCPRCPEPKSCDTPPPCPRCPEPKSCDTPPPCPRCP

SEQ ID NO: 128 - protein, 12 aa, synthetic construct
  REGION 1..12: note = Hinge
ESKYGPPCPSCP

SEQ ID NO: 129 - protein, 9 aa, synthetic construct
  REGION 1..9: note = Hinge
YGPPCPPCP

SEQ ID NO: 130 - protein, 10 aa, synthetic construct
  REGION 1..10: note = Hinge
KYGPPCPPCP

SEQ ID NO: 131 - protein, 14 aa, synthetic construct
  REGION 1..14: note = Hinge
EVVVKYGPPCPPCP

SEQ ID NO: 132 - protein, 326 aa, synthetic construct
  REGION 1..326: note = Human IgG2 Fc (Uniprot P01859)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 133 - protein, 327 aa, synthetic construct
  REGION 1..327: note = Human IgG4 Fc (Uniprot P01861)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 134 - protein, 28 aa, synthetic construct
  REGION 1..28: note = CD28 transmembrane domain (aa)
MFWVLVVVGGVLACYSLLVTVAFIIFWV

SEQ ID NO: 135 - protein, 22 aa, synthetic construct
  REGION 1..22: note = CD8a transmembrane domain (aa)
MIYIWAPLAGTCGVLLLSLVIT

SEQ ID NO: 136 - protein, 112 aa, synthetic construct
  REGION 1..112: note = CD3-zeta derived intracellular signaling domain (aa)
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 112 aa, synthetic construct
  REGION 1..112: note = CD3-zeta derived intracellular signaling domain (aa)
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 41 aa, synthetic construct
  REGION 1..41: note = CD28 (amino acids 180-220 of P10747)
RSKRSRLLHSDYMNMTPRRPGPTRKHYQPYAPPRDFAAYRS

SEQ ID NO: 139 - DNA, 123 bp, synthetic construct
  misc_feature 1..123: note = CD28 endodomain (nt)
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 140 - protein, 41 aa, synthetic construct
  REGION 1..41: note = CD28 (LL to GG)
RSKRSRGGHSDYMNMTPRRPGPTRKHYQPYAPPRDFAAYRS

SEQ ID NO: 141 - protein, 357 aa, synthetic construct
  REGION 1..357: note = tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 142 - protein, 335 aa, synthetic construct
  REGION 1..335: note = tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 143 - protein, 357 aa, synthetic construct; identical to SEQ ID NO: 141
  REGION 1..357: note = truncated EGFR (tEGFR) sequence (aa)

SEQ ID NO: 144 - DNA, 1233 bp, synthetic construct
  source 1..1233: mol_type = other DNA
  unsure 1..1233: note = PR3_9 (G4P)/(G4S)3/CD8aTM CAAR (PR3_9_103)
atcgttggaccccatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagaggcggtggcggttctggtggcggtggctccggcggtggaggttcaatctatatctgggcacctctggccggaacatgtggcgtgctgctgctgtctctggtcatcaccaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 145 - DNA, 1251 bp, synthetic construct
  source 1..1251: mol_type = other DNA
  unsure 1..1251: note = PR3_9 (G4P)/(G4S)3/CD28TM CAAR (PR3_9_104)
atcgttggaccccatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagaggcggtggcggttctggtggcggtggctccggcggtggaggttcattctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 146 - DNA, 1203 bp, synthetic construct
  source 1..1203: mol_type = other DNA
  unsure 1..1203: note = PR3_9 (G4P)/(G4S)/CD8aTM CAAR (PR3_9_105)
atcgttggaccccatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagaggcggtggaggttcaatctatatctgggcacctctggccggaacatgtggcgtgctgctgctgtctctggtcatcaccaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 147 - DNA, 1221 bp, synthetic construct
  source 1..1221: mol_type = other DNA
  unsure 1..1221: note = PR3_9 (G4P)/(G4S)/CD28TM CAAR (PR3_9_106)
atcgttggaccccatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagaggcggtggaggttcattctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 148 - DNA, 1329 bp, synthetic construct
  source 1..1329: mol_type = other DNA
  unsure 1..1329: note = PR3_9 (G4P)/CD8a/CD8aTM CAAR (PR3_9_107)
atcgttggaccccatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagaaagcctaccacaaccccagcacctagaccccctacaccagcaccaaccatcgcctctcagccactgagcctgcggcccgaggcctgtaggccagcagcaggaggagccgtgcacaccaggggcctggactttgcctgcgatatctatatctgggcacctctggccggaacatgtggcgtgctgctgctgtctctggtcatcaccaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 149 - DNA, 1203 bp, synthetic construct
  source 1..1203: mol_type = other DNA
  unsure 1..1203: note = PR3_9 (G4P)/EAAAK/CD8aTM CAAR (PR3_9_108)
atcgttggaccccatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagaagccgccgctaaaatctatatctgggcacctctggccggaacatgtggcgtgctgctgctgtctctggtcatcaccaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 150 - DNA, 1548 bp, synthetic construct
  source 1..1548: mol_type = other DNA
  unsure 1..1548: note = PR3_9 (G4P)/MS/CD8aTM CAAR (PR3_9_109)
atcgttggaccccatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgatctatatctgggcacctctggccggaacatgtggcgtgctgctgctgtctctggtcatcaccaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 151 - DNA, 1227 bp, synthetic construct
  source 1..1227: mol_type = other DNA
  unsure 1..1227: note = PR3_9 (G4P)/SS/CD8aTM CAAR (PR3_9_110)
atcgttggaccccatgaagcccagcctcacagcagaccttacatggccagcctgcagatgagaggcaatcccggctctcacttttgtggcggcaccctgattcacccctccttcgttctgaccgccgctcactgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccatgatctatatctgggcacctctggccggaacatgtggcgtgctgctgctgtctctggtcatcaccaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 152 - DNA, 1233 bp, synthetic construct
  source 1..1233: mol_type = other DNA
  unsure 1..1233: note = PR3_10 (H44A)/(G4S)3/CD8aTM CAAR (PR3_10_111)
atcgttggaggacatgaagcccagcctcacagcagaccttacatggccagcctgcagatgagaggcaatcccggctctcacttttgtggcggcaccctgattcacccctccttcgttctgaccgccgctgcctgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagaggcggtggcggttctggtggcggtggctccggcggtggaggttcaatctatatctgggcacctctggccggaacatgtggcgtgctgctgctgtctctggtcatcaccaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 153 - DNA, 1251 bp, synthetic construct
  source 1..1251: mol_type = other DNA
  unsure 1..1251: note = PR3_10 (H44A)/(G4S)3/CD28TM CAAR (PR3_10_112)
atcgttggaggacatgaagcccagcctcacagcagaccttacatggccagcctgcagatgagaggcaatcccggctctcacttttgtggcggcaccctgattcacccctccttcgttctgaccgccgctgcctgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagaggcggtggcggttctggtggcggtggctccggcggtggaggttcattctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 154 - DNA, 1203 bp, synthetic construct
  source 1..1203: mol_type = other DNA
  unsure 1..1203: note = PR3_10 (H44A)/(G4S)/CD8aTM CAAR (PR3_10_113)
atcgttggaggacatgaagcccagcctcacagcagaccttacatggccagcctgcagatgagaggcaatcccggctctcacttttgtggcggcaccctgattcacccctccttcgttctgaccgccgctgcctgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagaggcggtggaggttcaatctatatctgggcacctctggccggaacatgtggcgtgctgctgctgtctctggtcatcaccaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 155 - DNA, 1221 bp, synthetic construct
  source 1..1221: mol_type = other DNA
  unsure 1..1221: note = PR3_10 (H44A)/(G4S)/CD28TM CAAR (PR3_10_114)
atcgttggaggacatgaagcccagcctcacagcagaccttacatggccagcctgcagatgagaggcaatcccggctctcacttttgtggcggcaccctgattcacccctccttcgttctgaccgccgctgcctgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagaggcggtggaggttcattctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 156 - DNA, 1329 bp, synthetic construct
  source 1..1329: mol_type = other DNA
  unsure 1..1329: note = PR3_10 (H44A)/CD8a/CD8aTM CAAR (PR3_10_115)
atcgttggaggacatgaagcccagcctcacagcagaccttacatggccagcctgcagatgagaggcaatcccggctctcacttttgtggcggcaccctgattcacccctccttcgttctgaccgccgctgcctgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagaaagcctaccacaaccccagcacctagaccccctacaccagcaccaaccatcgcctctcagccactgagcctgcggcccgaggcctgtaggccagcagcaggaggagccgtgcacaccaggggcctggactttgcctgcgatatctatatctgggcacctctggccggaacatgtggcgtgctgctgctgtctctggtcatcaccaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 157 - DNA, 1203 bp, synthetic construct
  source 1..1203: mol_type = other DNA
  unsure 1..1203: note = PR3_10 (H44A)/EAAAK/CD8aTM CAAR (PR3_10_116)
atcgttggaggacatgaagcccagcctcacagcagaccttacatggccagcctgcagatgagaggcaatcccggctctcacttttgtggcggcaccctgattcacccctccttcgttctgaccgccgctgcctgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagaagccgccgctaaaatctatatctgggcacctctggccggaacatgtggcgtgctgctgctgtctctggtcatcaccaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 158 - DNA, 1548 bp, synthetic construct
  source 1..1548: mol_type = other DNA
  unsure 1..1548: note = PR3_10 (H44A)/MS/CD8aTM CAAR (PR3_10_117)
atcgttggaggacatgaagcccagcctcacagcagaccttacatggccagcctgcagatgagaggcaatcccggctctcacttttgtggcggcaccctgattcacccctccttcgttctgaccgccgctgcctgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgatctatatctgggcacctctggccggaacatgtggcgtgctgctgctgtctctggtcatcaccaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 159 - DNA, 1227 bp, synthetic construct
  source 1..1227: mol_type = other DNA
  unsure 1..1227: note = PR3_10 (H44A)/SS/CD8aTM CAAR (PR3_10_118)
atcgttggaggacatgaagcccagcctcacagcagaccttacatggccagcctgcagatgagaggcaatcccggctctcacttttgtggcggcaccctgattcacccctccttcgttctgaccgccgctgcctgcctgagagacatccctcagagactggtcaacgtggtgctgggagcccacaacgtgcggacacaagaacctacacagcagcacttcagcgtggcccaggtgttcctgaacaactacgacgccgagaacaagctgaacgacgtgctgctgatccagctgagcagccctgctaatctgtctgcctctgtggccactgtgcagctgcctcagcaagatcagcctgtgcctcacggcacacagtgtctggctatgggatggggaagagtgggcgctcatgatcctccagctcaagtgctgcaagagctgaacgtgaccgtcgtgaccttcttctgccggcctcacaacatctgcaccttcgtgcccagaagaaaggccggcatctgcttcggagattctggcggccctctgatctgcgacggaatcatccagggcatcgacagcttcgtgatctggggctgtgccaccagactgttccccgacttctttacccgggtggccctctacgtggactggatccggtctacactgagagagtctaaatacggaccgccttgtcctccttgtcccatgatctatatctgggcacctctggccggaacatgtggcgtgctgctgctgtctctggtcatcaccaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

(19) United States
(12) Patent Application Publication
(10) Pub. No.: US 2025/0161359 A1
(43) Pub. Date: May 22, 2025

(54) PROTEINASE 3 (PR3) CHIMERIC AUTOANTIBODY RECEPTOR T CELLS AND RELATED METHODS AND USES

(71) Applicant: Juno Therapeutics, Inc., Seattle, WA (US)

(72) Inventors: Kofi Agyare MENSAH, Cambridge, MA (US); Dennis Michael ZALLER, Lexington, MA (US); Steven Anthony SAENZ, Cambridge, MA (US); Xi JIN, Needham, MA (US); William Andrew JOHNSEN, Seattle, WA (US); Ginger Chao RAKESTRAW, Cambridge, MA (US); Joseph Rene PAQUETTE, Cambridge, MA (US); Robert PLENGE, Cambridge, MA (US); Stephen Jacob GOLDFLESS, Newton Highlands, MA (US); Jeffrey Lee DANTZLER, Seattle, WA (US); Collin William HAUSKINS, Seattle, WA (US); Cyr Clovis Chua DE IMUS, Kenmore, WA (US); Sanjib DUTTA, Seattle, WA (US); Kristoff Theodor HOMAN, Seattle, WA (US)

(73) Assignee: Juno Therapeutics, Inc., Seattle, WA (US)

(21) Appl. No.: 18/840,482

(22) PCT Filed: Feb. 21, 2023
(86) PCT No.: PCT/US2023/062965

§ 371 Date: Aug. 21, 2024

Related U.S. Application Data
(60) Provisional application No. 63/312,809, filed on Feb. 22, 2022.

(51) Int. Cl. A61K 35/17; A61K 40/11; A61K 40/31; A61K 40/42; A61P 35/00; C07K 14/705; C07K 14/725; C07K 16/40; C12N 5/0783; C12N 15/86

(52) U.S. Cl. CPC A61K 35/17; A61K 40/11; A61K 40/31; A61K 40/4244; A61P 35/00; C07K 14/7051; C07K 14/70517; C07K 14/70521; C07K 14/70596; C07K 16/40; C12N 5/0636; C12N 15/86; A61K 2239/13; A61K 2239/17; A61K 2239/21; A61K 2239/22; C07K 2317/524; C07K 2317/526; C07K 2319/02; C07K 2319/03; C12N 2510/00; C12N 2740/15043

(57) ABSTRACT

Provided herein are cell and protein therapeutics that comprise a Proteinase 3 (PR3) antibody binding domain, such as a wild type PR3 protein or mutant PR3 protein, and methods of use thereof. Also provided herein arc chimeric autoantibody receptors (CAARs) comprising an extracellular PR3 antibody binding domain. Among the provided CAARs are those in which the extracellular PR3 antibody binding domain ia a wild type or mutated PR3 protein or fragment thereof that are able to be specifically bound by an anti-PR3 antineutrophil cytoplasmic antibody (ANCA). Also provided herein are polynucleotides that encode the provided CAARs, genetically engineered cells such as T cells containing the provided CAARs, and to related methods and uses thereof in adoptive cell therapy.

### CROSS-REFERENCE TO RELATED APPLICATIONS

This application is a U.S. national stage application under 35 U.S.C. § 371 of International Application No. PCT/US2023/062965, filed internationally on Feb. 21, 2023, which claims priority from U.S. provisional application No. 63/312,809 filed Feb. 22, 2022, entitled “Proteinase 3 (PR3) Chimeric Autoantibody Receptor T Cells and Related Methods and Uses,” the contents of which is incorporated by reference in its entirety.

### INCORPORATION BY REFERENCE OF SEQUENCE LISTING

The present application is being filed along with a Sequence Listing in electronic format. The Sequence Listing is provided as a file entitled 735042022000SeqList.xml, created Aug. 15, 2024, which is 304,200 bytes in size. The information in the electronic format of the Sequence Listing is incorporated by reference in its entirety.

### FIELD

The present disclosure provides a cell or protein therapeutic that comprises a proteinase 3 (PR3) antibody binding domain, such as a wild type PR3 or a mutant PR3 protein. The present disclosure also provides chimeric autoantibody receptors (CAARs) comprising an extracellular PR3 antibody binding domain. Among the provided CAARs of the present disclosure are those in which the extracellular PR3 antibody binding domain is a wild type or mutated PR3 protein or fragment thereof that is able to be specifically bound by an anti-PR3 antineutrophil cytoplasmic antibody (ANCA). The present disclosure also provides polynucleotides that encode the provided CAARs, genetically engineered cells such as T cells containing the provided CAARs, and related methods and uses thereof in adoptive cell therapy.

### BACKGROUND

Anti-neutrophil cytoplasmic antibody-associated vasculitis (AAV) is a rare autoimmune disease characterized by inflammation and damage to small blood vessels. The neutrophil serine protease proteinase 3 (PR3), found in the primary granules of neutrophils, is a main autoantigen in AAV. Autoantibodies to PR3 (ANCA) produced by autoreactive B cells, are crucial in the development of AAV by activating the primed neutrophils which damages the vessels. Improved methods to target and kill disease-causing B cells are needed. Provided are embodiments that meet such needs.

### SUMMARY

Provided herein is a chimeric autoantibody receptor (CAAR) comprising: (a) an extracellular proteinase 3 (PR3) antibody binding domain; (b) a transmembrane region; and (c) an intracellular signaling region. In some embodiments, the CAAR further comprises a spacer between the extracellular PR3 antibody binding domain and the transmembrane domain. In some of any embodiments, the PR3 antibody binding domain is from a human PR3 protein or is a mutant thereof. In some of any embodiments, the PR3 antibody binding domain is a mature PR3 protein. In some of any embodiments, the PR3 antibody binding domain comprises the sequence of amino acids set forth in SEQ ID NO:1 or a sequence of amino acids that exhibits at least 90% sequence identity to SEQ ID NO:1. In some of any embodiments, the PR3 antibody binding domain is a wild-type human PR3. In some of any embodiments, the PR3 antibody binding domain is set forth in SEQ ID NO:1.

In some of any embodiments, the PR3 antibody binding domain is a mutant PR3 protein comprising an amino acid substitution at a position selected from the group consisting of G4, H44, D91, D175 and S176 with reference to positions set forth in SEQ ID NO:1. In some embodiments, the amino acid substitution is selected from the group consisting of G4P, H44A, D91N, D175N, S176A and S176C, or is a conservative substitution thereof. In some embodiments, the amino acid substitution is selected from the group consisting of G4P, H44A, D91N, D175N, S176A and S176C. In some embodiments, the amino acid substitution is a conservative amino acid substitution of an amino acid substitution selected from the group consisting of G4P, H44A, D91N, D175N, S176A and S176C.

In some of any embodiments, the PR3 antibody binding domain is a mutant PR3 protein that has reduced enzymatic activity compared with a wild type PR3 protein (e.g., the PR3 protein set forth in SEQ ID NO:1).

Among provided embodiments is a chimeric autoantibody receptor (CAAR) comprising: (a) an extracellular proteinase 3 (PR3) antibody binding domain, wherein the PR3 antibody binding domain is a mutant PR3 protein that has reduced enzymatic activity compared with a wild type PR3 protein having the sequence set forth in SEQ ID NO:1; (b) a transmembrane region; and (c) an intracellular signaling region.

In some embodiments, the mutant PR3 protein comprises an amino acid substitution is in or near the catalytic triad. In some embodiments, the amino acid substitution is in an active site residue selected from the group consisting of H44, D91 and S176, with reference to numbering set forth in SEQ ID NO:1. In some embodiments the amino acid substitution is selected from the group consisting of H44A, D91N, S176A and S176C. In some of any embodiments, the mutant PR3 protein comprises an amino acid substitution at position H44, with reference to the numbering of positions in SEQ ID NO:1.

In some embodiments, the mutant PR3 protein comprises an amino acid substitution at a position that interferes with the formation of the substrate binding pocket. In some embodiments, the position is G4. In some of any embodiments, the mutant PR3 protein comprises and amino acid substitution at position G4, with reference to the numbering of positions in SEQ ID NO:1. In some of any embodiments, the mutant PR3 protein comprises 1, 2, 3, 4 or 5 amino acid substitutions compared to wild-type PR3 set forth in SEQ ID NO:1. In some of any embodiments, the mutant PR3 protein comprises a single amino acid substitution compared to SEQ ID NO:1. In some of any embodiments, the mutant PR3 protein comprises the amino acid substitution G4P.

Provided herein is a CAAR comprising: (a) a PR3 antibody binding domain; (b) a transmembrane region; and (c) an intracellular signaling region, wherein the PR3 antibody binding domain is a mutant PR3 protein comprising the amino acid substitution G4P. In some of any embodiments, the PR3 antibody binding domain comprises the sequence of amino acids set forth in SEQ ID NO:9. In some embodiments, the PR3 antibody binding domain comprises a sequence of amino acids that exhibits at least 90% sequence identity to SEQ ID NO:9. In some embodiments, the PR3 antibody binding domain is set forth in SEQ ID NO:9. In some of any embodiments, the mutant PR3 protein comprises the amino acid substitution H44A.

Provided herein is a CAAR comprising: (a) a PR3 antibody binding domain; (b) a transmembrane region; and (c) an intracellular signaling region, wherein the PR3 antibody binding domain is a mutant PR3 protein comprising the amino acid substitution H44A. In some of any embodiments, the PR3 antibody binding domain comprises the sequence of amino acids set forth in SEQ ID NO:10. In some of any embodiments, the PR3 antibody binding domain comprises a sequence of amino acids that exhibits at least 90% sequence identity to SEQ ID NO:10. In some of any embodiments, the PR3 antibody binding domain comprises the sequence of amino acids set forth in SEQ ID NO:10. In some embodiments, the amino acid sequence of the PR3 antibody binding domain consists of the sequence set forth in SEQ ID NO:10. In some of any embodiments, the mutant PR3 protein comprises the amino acid substitution D91N. In some of any embodiments, the antibody binding domain comprises the sequence of amino acids set forth in SEQ ID NO:4 or a sequence of amino acids that exhibits at least 90% sequence identity to SEQ ID NO:4. In some embodiments, the PR3 antibody binding domain is set forth in SEQ ID NO:4.

In some of any embodiments, the mutant PR3 protein comprises the amino acid substitution D175N. In some of any embodiments, the PR3 antibody binding domain comprises the sequence of amino acids set forth in SEQ ID NO:7 or a sequence of amino acids that exhibits at least 90% sequence identity to SEQ ID NO:7. In some embodiments, the PR3 antibody binding domain is set forth in SEQ ID NO:7. In some of any embodiments, the mutant PR3 protein comprises the amino acid substitution S176A or S176C. In some of any embodiments, the PR3 antibody binding domain comprises the sequence of amino acids set forth in SEQ ID NO:2 or a sequence of amino acids that exhibits at least 90% sequence identity to SEQ ID NO:2. In some embodiments, the PR3 antibody binding domain is set forth in SEQ ID NO:2. In some of any embodiments, the PR3 antibody binding domain comprises the sequence of amino acids set forth in SEQ ID NO:8 or a sequence of amino acids that exhibits at least 90% sequence identity to SEQ ID NO:8. In some embodiments, the PR3 antibody binding domain is set forth in SEQ ID NO:8.

In some of any embodiments, the transmembrane region is or comprises a transmembrane domain from CD4, CD28, or CD8. In some of any embodiments, the transmembrane region is or comprises a transmembrane domain from CD28. In some embodiments, the transmembrane region comprises a human CD28. In some of any embodiments, the transmembrane region is or comprises SEQ ID NO: 26 or an amino acid sequence having at least at or about 90%, 91%, 92%, 93%, 94%, 95%, 96%, 97%, 98% or 99% sequence identity to SEQ ID NO:26. In some of any embodiments, the transmembrane region is set forth in SEQ ID NO:26. In some of any embodiments, the transmembrane region is or comprises a transmembrane domain from CD8a. In some of any embodiments, the transmembrane domain is a human CD8a. In some of any embodiments, the transmembrane region is or comprises SEQ ID NO: 98 or an amino acid sequence having at least at or about 90%, 91%, 92%, 93%, 94%, 95%, 96%, 97%, 98% or 99% sequence identity to SEQ ID NO:98. In some of any embodiments, the transmembrane region is set forth in SEQ ID NO:98.

In some of any embodiments, the intracellular signaling region comprises an intracellular signaling domain capable of inducing a primary activation signal in a T cell. In some embodiment, the intracellular signaling domain is a domain from a T cell receptor (TCR) component and/or comprises an immunoreceptor tyrosine-based activation motif (ITAM). In some of any embodiments, the intracellular signaling domain is a cytoplasmic signaling domain of a CD3-zeta (CD3ζ) chain. In some embodiments, the intracellular signaling domain is a human CD3ζ chain. In some of any embodiments, the intracellular signaling domain comprises the sequence set forth in SEQ ID NO:28, or an amino acid sequence having at least at or about 90%, 91%, 92%, 93%, 94%, 95%, 96%, 97%, 98% or 99% sequence identity to SEQ ID NO:28. In some of any embodiments, the intracellular signaling domain is set forth in SEQ ID NO:28.

In some of any embodiments, the intracellular signaling region further comprises a costimulatory signaling region. In some embodiment, the costimulatory signaling region is between the transmembrane region and the intracellular signaling domain. In some of any embodiments, the costimulatory signaling region comprises an intracellular signaling domain of a T cell costimulatory molecule or a signaling portion thereof. In some of any embodiments, the costimulatory signaling region comprises an intracellular signaling domain of CD28, 4-1BB, or ICOS. In some of any embodiments, the costimulatory signaling region comprises an intracellular signaling domain of 4-1BB. In some embodiments, the costimulatory signaling region comprises a human 4-1BB. In some of any embodiments, the costimulatory signaling region comprises the sequence set forth in SEQ ID NO:27 or an amino acid sequence having at least at or about 90%, 91%, 92%, 93%, 94%, 95%, 96%, 97%, 98% or 99% sequence identity to SEQ ID NO: 27. In some of any embodiments, the costimulatory signaling region is set forth in SEQ ID NO:27.

In some of any embodiments, the spacer comprises at least a portion of an immunoglobulin or a variant thereof. In some of any embodiments, the spacer comprises a hinge region of an immunoglobulin or a variant thereof. In some embodiment, the hinge region of an immunoglobulin is an IgG4 hinge region. In some embodiments, the hinge region is a human IgG4 hinge region, or a variant thereof. In some of any embodiments, the spacer comprises a variant IgG4 hinge region comprising substitution of amino acids CPSC to CPPC compared to the wild-type IgG4 hinge region. In some of any embodiments, the spacer is less than at or about 15 amino acids in length. In some of any embodiments, the spacer is between 12 and 15 amino acids in length. In some of any embodiments, the spacer comprises the sequence set forth in SEQ ID NO: 22 or SEQ ID NO:23. In some of any embodiments, the spacer is set forth in SEQ ID NO:22.

In some of any embodiments, the spacer is between 100 and 150 amino acids in length. In some embodiments, the spacer is between 110 and 130 amino acids in length. In some of any embodiments, the spacer comprises a hinge region of an immunoglobulin and a CH3 region of an immunoglobulin. In some embodiment, the spacer comprises an IgG4 hinge region or a variant thereof and IgG4 CH3 region. In some of any embodiments, the spacer comprises the sequence set forth in SEQ ID NO: 24 or SEQ ID NO:96. In some of any embodiments, the spacer is set forth in SEQ ID NO:24.

In some of any embodiments, the spacer is between 200 and 250 amino acids in length. In some embodiments, the spacer is between 220 and 240 amino acids in length. In some of any embodiments, the spacer comprises a hinge region of an immunoglobulin, a CH2 region of an immunoglobulin or a chimeric CH2 region of two different immunoglobulins, and a CH3 region of an immunoglobulin. In some embodiment, the spacer comprises IgG4 hinge region or a variant thereof, a chimeric CH2 region comprising a portion of an IgG4 CH2 and a portion of an IgG2 CH2 (IgG2/4 CH2 region), and an IgG4 CH3 region. In some of any embodiments, the spacer comprises the sequence set forth in SEQ ID NO: 25 or SEQ ID NO:97. In some of any embodiments, the spacer is set forth in SEQ ID NO:25. In some of any embodiments, the spacer comprises amino acids GGGGS (SEQ ID NO:99). In some of any embodiments, the spacer is set forth in SEQ ID NO:99. In some of any embodiments, the spacer is set forth in SEQ ID NO:100.

In some of any embodiments, the CAAR comprises a sequence of amino acids that exhibits at least 85% sequence identity to the sequence set forth in any of SEQ ID NOS: 29, 30, 31, 38, 39, 40, 53, 54, 55, 56, 57, 58, 104, 110, or 111. In some embodiments, the CAAR comprises the sequence set forth in any one of SEQ ID NOS: 29, 30, 31, 38, 39, 40, 53, 54, 55, 56, 57, 58, 104, 110, or 111. Provided herein is a CAAR comprising the sequence of amino acids set forth in SEQ ID NO: 29 or a sequence of amino acids that exhibits at least 90% sequence identity to SEQ ID NO:29. Provided herein is a CAAR comprising the sequence of amino acids set forth in SEQ ID NO: 30 or a sequence of amino acids that exhibits at least 90% sequence identity to SEQ ID NO:30. Provided herein is a CAAR comprising the sequence of amino acids set forth in SEQ ID NO: 38 or a sequence of amino acids that exhibits at least 90% sequence identity to SEQ ID NO:38. Provided herein is a CAAR comprising the sequence of amino acids set forth in SEQ ID NO: 55 or a sequence of amino acids that exhibits at least 90% sequence identity to SEQ ID NO:55. Provided herein is a CAAR comprising the sequence of amino acids set forth in SEQ ID NO: 56 or a sequence of amino acids that exhibits at least 90% sequence identity to SEQ ID NO:56. Provided herein is a CAAR comprising the sequence of amino acids set forth in SEQ ID NO: 57 or a sequence of amino acids that exhibits at least 90% sequence identity to SEQ ID NO:57. Provided herein is a CAAR comprising the sequence of amino acids set forth in SEQ ID NO: 58 or a sequence of amino acids that exhibits at least 90% sequence identity to SEQ ID NO:58.

Provided herein is a CAAR comprising: (a) a PR3 antibody binding domain comprising the sequence set forth in SEQ ID NO:9; (b) a spacer comprising the sequence set forth in SEQ ID NO:25; (c) a transmembrane region comprising the sequence set forth in SEQ ID NO:26; (d) a costimulatory signaling region comprising the sequence set forth in SEQ ID NO:27; and (e) an intracellular signaling region comprising the sequence set forth in SEQ ID NO:28. Provided herein is a CAAR comprising a sequence of amino acids that exhibits at least 90% sequence identity to SEQ ID NO:55 wherein the sequence of amino acids comprises a G4P mutation with reference to the numbering of positions in SEQ ID NO:1. In some embodiments, the CAAR comprises the sequence of amino acids set forth in SEQ ID NO: 55.

Provided herein is a CAAR comprising: (a) a PR3 antibody binding domain comprising the sequence set forth in SEQ ID NO:10; (b) a spacer comprising the sequence set forth in SEQ ID NO:24; (c) a transmembrane region comprising the sequence set forth in SEQ ID NO:26; (d) a costimulatory signaling region comprising the sequence set forth in SEQ ID NO:27; and (e) an intracellular signaling region comprising the sequence set forth in SEQ ID NO:28. Provided herein is a CAAR comprising a sequence of amino acids that exhibits at least 90% sequence identity to SEQ ID NO:57 wherein the sequence of amino acids comprises a G4P mutation with reference to the numbering of positions in SEQ ID NO:1. In some embodiments, the CAAR comprises the sequence of amino acids set forth in SEQ ID NO: 57.

Provided herein is a CAAR comprising: (a) a PR3 antibody binding domain comprising the sequence set forth in SEQ ID NO:9; (b) a spacer comprising the sequence set forth in SEQ ID NO:100; (c) a transmembrane region comprising the sequence set forth in SEQ ID NO:26; (d) a costimulatory signaling region comprising the sequence set forth in SEQ ID NO:27; and (e) an intracellular signaling region comprising the sequence set forth in SEQ ID NO:28. Provided herein is a CAAR comprising a sequence of amino acids that exhibits at least 90% sequence identity to SEQ ID NO:104 wherein the sequence of amino acids comprises a G4P mutation with reference to the numbering of positions in SEQ ID NO:1. In some embodiments, the CAAR comprises the sequence of amino acids set forth in SEQ ID NO: 104.

Provided herein is a CAAR comprising: (a) a PR3 antibody binding domain comprising the sequence set forth in SEQ ID NO:9; (b) a spacer comprising the sequence set forth in SEQ ID NO:22; (c) a transmembrane region comprising the sequence set forth in SEQ ID NO:98; (d) a costimulatory signaling region comprising the sequence set forth in SEQ ID NO:27; and (e) an intracellular signaling region comprising the sequence set forth in SEQ ID NO:28. Provided herein is a CAAR comprising a sequence of amino acids that exhibits at least 90% sequence identity to SEQ ID NO:110 wherein the sequence of amino acids comprises a G4P mutation with reference to the numbering of positions in SEQ ID NO:1. In some embodiments, the CAAR comprises the sequence of amino acids set forth in SEQ ID NO: 110.

Provided herein is a CAAR comprising: (a) a PR3 antibody binding domain comprising the sequence set forth in SEQ ID NO:10; (b) a spacer comprising the sequence set forth in SEQ ID NO:100; (c) a transmembrane region comprising the sequence set forth in SEQ ID NO:98; (d) a costimulatory signaling region comprising the sequence set forth in SEQ ID NO:27; and (e) an intracellular signaling region comprising the sequence set forth in SEQ ID NO:28. Provided herein is a CAAR comprising a sequence of amino acids that exhibits at least 90% sequence identity to SEQ ID NO:111 or 119 wherein the sequence of amino acids comprises a H44A mutation with reference to the numbering of positions in in SEQ ID NO:1. In some embodiments, the CAAR comprises the sequence of amino acids set forth in SEQ ID NO: 111 or 119.

In some of any embodiments, the CAAR is able to be specifically bound by an anti-PR3 antineutrophil cytoplasmic antibody (PR3-ANCA). In some of any embodiments, the PR3 antibody binding domain comprises at least 2, 3, 4, 5, 6 or 7 epitopes recognized by an anti-PR3 antineutrophil cytoplasmic antibody (PR3-ANCA). In some embodiment, the epitopes are selected from the group consisting of ANCA2 (AQPHSRPYMAS, SEQ ID NO:90), ANCA3 (PGSHFCGG, SEQ ID NO:91), ANCA4 (VVLGAHNVRTQ, SEQ ID NO:92), ANCA5 (FLNNYDAE, SEQ ID NO:93), ANCA6 (PVPHGTQC, SEQ ID NO: 94) and ANCA7 (CFGDSGGP, SEQ ID NO:95).

In some of any embodiments, the CAAR is specific for PR3-reactive B cells. In some of any embodiments, Jurkat reporter cells which have Td-tomato knocked in at the endogenous Nur77 locus and that are transduced with the CAAR exhibit less than at or about 25%, at or about 20%, at or about 15%, at or about 10%, at or about 9%, at or about 8%, at or about 7%, at or about 5%, at or about 4%, at or about 3%, at or about 2% or at or about 1% tonic signalling, as measured by fluorescence of Td-Tomato by flow cytometry and determined as the total percentage of cells that express Td-Tomato in the transduced Jurkat reporter cells. In some of any embodiments, cells transduced with the CAAR show antigen-specific activation after exposure to an anti-human PR3 antibody. In some of any embodiments, the CAAR comprises a sequence of amino acids that exhibits at least 85% sequence identity to the sequence set forth in any of SEQ ID NOS: 55, 57, 104, 110, and 111. In some of any embodiments, the CAAR comprises the sequence set forth in any one of SEQ ID NOS: 55, 57, 104, 110, and 111.

In some of any embodiments, the CAAR is able to kill at least 65% of an anti-PR3 antibody expressing cell target. In some of any embodiments, the CAAR is able to kill at least 65% of each of at least three cell targets expressing different anti-PR3 antibodies, wherein each of the at least three different antibodies binds to a distinct PR3 epitope. In some of any embodiments, the CAAR comprises a sequence of amino acids that exhibits at least 85% sequence identity to the sequence set forth in any of SEQ ID NOS: 55, 57, 104, 110, and 111. In some of any embodiments, the CAAR comprises the sequence set forth in any one of SEQ ID NOS: 55, 57, 104, 110, and 111.

In some of any embodiments, a polynucleotide comprises a nucleic acid encoding the CAAR. In some embodiment, the polynucleotide is optimized by splice site elimination. In some of any embodiments, the polynucleotide is codon-optimized for expression in a human cell. In some of any embodiments, the polynucleotide comprises a nucleic acid sequence that exhibits at least 85% sequence identity to the sequence set forth in any of SEQ ID NOS: 59, 60, 61, 68, 69, 70, 83, 84, 85, 86, 87, 88, 144, 145, 146, 147, 148, 149, 150, 151, 152, 153, 154, 155, 156, 157, 158, or 159. In some embodiments, the polynucleotide comprises the sequence set forth in any one of SEQ ID NOS: 59, 60, 61, 68, 69, 70, 83, 84, 85, 86, 87, 88, 144, 145, 146, 147, 148, 149, 150, 151, 152, 153, 154, 155, 156, 157, 158, or 159. In some of any embodiments, the polynucleotide comprises a nucleic acid sequence that exhibits at least 90% sequence identity to the sequence set forth in any of SEQ ID NOS: 59, 60, 68, 85, 86, 87, 88, 145, 151, or 152. In some embodiments, the polynucleotide comprises the sequence set forth in any one of SEQ ID NOS: 59, 60, 68, 85, 86, 87 or 88.

In some of any embodiments, a vector comprises the polynucleotide. In some embodiment, the vector is a viral vector. In some embodiment, the viral vector is a retroviral vector (e.g., lentiviral vector). In some of any embodiments, a cell comprises the CAAR. In some of any embodiments, a cell comprises the polynucleotide or the vector. In some of any embodiments, the cell is a lymphocyte. In some of any embodiments, the cell is an NK cell or a T cell. In some of any embodiments, the cell is a T cell and the T cell is a CD4+ T cell or a CD8+ T cell. In some of any embodiments, the cell is a primary cell obtained from a subject. In some of any embodiments, the cells is an induced pluripotent stem cell. In some of any embodiments, the cell has been differentiated from an induced pluripotent stem cell. In some of any embodiments, the cell is an allogeneic cell. In some of any embodiments, the cell is engineered to be hypoimmune.

In some of any embodiments, the cell exhibits cytotoxic activity against PR3-reactive B cells. In some embodiment, the cytotoxic activity is against a plurality of PR3-reactive B cells, wherein the plurality of PR3-reactive B cells are specific for two or more different PR3 antineutrophil cytoplasmic antibody (PR3-ANCA) epitopes. In some embodiment, the plurality of PR3-reactive B cells are specific for at least 2, 3, 4, 5, 6 or 7 different PR3 ANCA epitopes. In some of any embodiments, the plurality of PR3-reactive B cells are specific for at least 4 different PR3 ANCA eptitopes. In some of any embodiments, the plurality of PR3-reactive B cells are specific for at least 5 different PR3 ANCA epitopes. In some of any embodiments, the plurality of PR3-reactive B cells are specific for at least 6 different PR3 ANCA epitopes. In some of any embodiments, the ANCA epitopes are selected from the group consisting of ANCA2 (AQPHSRPYMAS, SEQ ID NO:90), ANCA3 (PGSHFCGG, SEQ ID NO:91), ANCA4 (VVLGAHNVRTQ, SEQ ID NO:92), ANCA5 (FLNNYDAE, SEQ ID NO:93), ANCA6 (PVPHGTQC, SEQ ID NO: 94) and ANCA7 (CFGDSGGP, SEQ ID NO:95).

In some of any embodiments, a composition comprises the cell. In some embodiment, the composition further comprises a pharmaceutically acceptable excipient. In some of any embodiments, the composition comprises CD4+ and CD8+ T cells. In some of any embodiments, the ratio of CD4+ to CD8+ T cells is from at or about 1:3 to 3:1. In some embodiments, the ratio of CD4+ to CD8+ T cells is at or about 1:2 to 2:1. In some embodiments, the ratio of CD4+ to CD8+ is at or about 1:1. In some of any embodiments, greater than at or about 90%, greater than at or about 95% or greater than at or about 98% of cells in the composition are CD3+ T cells. In some of any embodiments, at least 40%, at least 50%, at least 60%, at least 70%, at least 80% or at least 90% of cells in the composition express the CAAR. In some of any embodiments, among a plurality of the cells in the composition expressing the CAAR, less than at or about 10%, at or about 9%, at or about 8%, at or about 7%, at or about 5%, at or about 4%, at or about 3%, at or about 2% or at or about 1% of the cells in the plurality exhibits tonic signaling and/or antigen independent activity or signaling. In some of any embodiments, a method of killing PR3-reactive B cells comprises contacting a PR3-reactive B cell with the cell or the composition. In some embodiment, the method is performed in vitro or ex vivo. In some embodiment, the method is performed in vivo in a subject.

In some of any embodiments, the PR3-reactive B cell comprises a plurality of PR3-reactive B cells, wherein the plurality of PR3-reactive B cells are specific for two or more different PR3 antineutrophil cytoplasmic antibody (PR3-ANCA) epitopes. In some embodiment, the plurality of PR3-reactive B cells are specific for 2, 3, 4, 5, 6 or 7 different PR3 ANCA epitopes. In some of any embodiments, the plurality of PR3-reactive B cells are specific for at least 4 different PR3 ANCA epitopes. In some of any embodiments, the plurality of PR3-reactive B cells are specific for at least 5 different PR3ANCA epitopes. In some of any embodiments, the plurality of PR3-reactive B cells are specific for at least 6 different PR3 ANCA epitopes. In some of any embodiments, the ANCA epitopes are selected from the group consisting of ANCA2 (AQPHSRPYMAS, SEQ ID NO:90), ANCA3 (PGSHFCGG, SEQ ID NO:91), ANCA4 (VVLGAHNVRTQ, SEQ ID NO:92), ANCA5 (FLNNYDAE, SEQ ID NO:93), ANCA6 (PVPHGTQC, SEQ ID NO: 94) and ANCA7 (CFGDSGGP, SEQ ID NO:95).

In some of any embodiments, among a plurality of PR3-reactive B cells, the method results in killing of greater than at or about 80%, greater than at or about 90%, greater than at or about 95%, PR3-reactive B cells. In some embodiments, the PR3-reactive B cells are IgG+. In some of any embodiments, the method comprises administering the cell or the composition to a subject in need of treatment thereof. In some embodiment, the disease or disorder is an autoimmune condition. In some of any embodiments, the disease or disorder is antineutrophil cytoplasmic antibody (ANCA)-associated vasculitis (AAV). In some embodiments, the AAV is PR3-AAV. In some of any embodiments, the disease or disorder is selected from the group consisting of granulomatosis with polyangiitis (GPA), microscopic polyangiitis (MPA), or eosinophilic granulomatosis with polyangiitis (EGPA). In some of any embodiments, the subject is positive for PR3-ANCA. In some of any embodiments, the method comprises testing the subject for PR3-ANCA. In some embodiment, the method comprises verifying that the subject has tested positive for PR3-ANCA.

In some of any embodiments, the subject has been treated with a prior therapy (e.g., one or more prior therapies) for AAV. In some embodiment, the subject has failed to achieve remission or has relapsed following treatment with the prior therapy. In some of any embodiments, the prior therapy comprises one or more of a glucocorticoid, cyclophosphamide (CYC), rituximab (RTX), plasma exchange, Avacopan, methotrexate (MTX), mycophenolate mofetil (MMF), azathioprine (AZA), leflunomide (LEF), belimumab, mepolizumab, and omalizumab. In some embodiment, the glucocorticoid is one or more of prednisolone, methylprednisolone, prednisone, dexamethasone.

In some of any embodiments, the prior therapy comprises a remission induction therapy (e.g., a remission induction agent). In some embodiment, the remission induction therapy comprises one or more of rituximab, cyclophosphamide, and a glucocorticoid. In some embodiment, the glucocorticoid is one or more of prednisolone, methylprednisolone, prednisone, and dexamethasone. In some embodiment, the glucocorticoid is prednisolone.

In some of any embodiments, the prior therapy comprises a remission maintenance therapy. In some embodiment, the remission maintenance therapy comprises one or more of rituximab, methotrexate, azathioprine, mycophenolate mofetil, leflunomide, mepolizumab, and omalizumab. In some of any embodiments, a method of treating a subject having PR3-ANCA vasculitis comprises (i) administering a remission induction therapy to the subject and (ii) administering the cell or the composition to the subject. In some embodiment, the remission induction therapy comprises cyclophosphamide. In some embodiment, cyclophosphamide is administered to the subject before the cell or composition is administered to the subject. In some embodiment, a course of cyclophosphamide is administered to the subject at least 1 week, 2 weeks, 3 weeks, or 4 weeks before the cell or composition is administered to the subject. In some of any embodiments, the remission induction therapy further comprises a glucocorticoid. In some of any embodiments, the glucocorticoid is prednisolone.

In some of any embodiments, a method of treating a subject having PR3-ANCA vasculitis comprises (i) administering cyclophosphamide to the subject and (ii) administering the cell or the composition to the subject. In some of any embodiments, the method comprises administering cyclophosphamide to the subject before administering the cell or the composition to the subject. In some embodiment, a course of cyclophosphamide is administered to the subject at least 1 week, 2 weeks, 3 weeks, or 4 weeks before the cell or composition is administered to the subject. In some of any embodiments, the course of cyclophosphamide is administered according to a dosing schedule that has been determined to be sufficient to reduce ANCA titers in a patient having PR3-ANCA vasculitis. In some of any embodiments, cyclophosphamide is administered at an oral dose of 2 mg/kg/day for at least 1 week, 2 weeks, 3 weeks, or 4 weeks before the cell or composition is administered to the subject. In some of any embodiments, cyclophosphamide is administered at an IV dose of 15 mg/kg every 2 weeks for up to 3 doses prior to administering the cell or composition to the subject. In some of any embodiments, after the cell or composition is administered to the subject, the subject is not treated with cyclophosphamide.

In some of any embodiments, the method comprises administering a glucocorticoid to the subject before administering the cell or composition to the subject. In some of any embodiments, the method comprises administering a glucocorticoid to the subject after administering the cell or composition to the subject. In some of any embodiments, a method of treating a subject having PR3-ANCA vasculitis comprises (i) administering to the subject a preconditioning therapy that has been shown to be effective to deplete ANCAs and subsequently (ii) administering the cell or the composition to the subject. In some embodiment, the preconditioning therapy comprises cyclophosphamide.

### BRIEF DESCRIPTION OF THE DRAWINGS

FIG. 1 depicts a schematic of a PR3 CAAR construct showing components including a signal peptide, an extracellular anti-PR3 antibody binding domain (PR3), a spacer, a transmembrane domain (TM), a costimulatory (Co-Stim) domain and a signaling domain.

FIGS. 2A-2B depict the differential tonic signaling and activation observed across various PR3-CAAR expressing Nur77-TdTomato reporter Jurkat T cells as described in Example 1, after a 24 hour incubation in the absence of target (PBS control condition, top row) and in the presence of target α-PR3 (anti-PR3) antibody (activation condition, bottom row).

FIG. 3 depicts reported PR3 ANCA epitopes on the PR3 crystal structure (left) and epitope binning (right) of PR3-reactive antibodies that were generated through sub-cutaneous immunization of rodents with native, active human PR3 protein in comparison with reference antibodies. Each circle in the community plot represents an antibody in the pairwise competition experiment, with lines indicating competition between the two antibodies connected by a line. The antibodies are grouped into epitope bins as indicated based on similar blocking profiles.

FIG. 4 depicts the results from an in vitro cytotoxicity assay performed to assess the impact of target to effector ratios, as described in Example 4, part A. After co-culture of IgG+ CHO-S target cells and primary T cells expressing one of four PR3 CAARs or no CAR (mock CAAR), killing efficiency was assessed by measuring the loss of viable target cells at 24 hours. As controls, cultures of mock CAR cells (T cells not expressing a CAR) with the target cells were assessed.

FIG. 5 depicts CAAR-T cell potency against targets expressing different anti-PR3 antibodies with a variety of epitope reactivities, as described in Example 4, part B.

FIG. 6 depicts the impact of the surface density of anti-PR3 antibodies on PR3-CAAR T cell potency. αPR3 antibody expression density on IgG⁺-CHO-S targets was assessed by mean fluorescence intensity (MFI) and CAAR T cell killing was assessed against each individual IgG⁺-CHO-S target. Killing efficiencies against high (H) and low (L) density targets as well as combined high and low density targets (pool) are shown.

FIG. 7 depicts CAAR T cell activation in the absence of target cells (CAARs alone) and following 24 h exposure to anti-PR3 expressing cellular targets (L=low density, H=high density, Pool=combined low and high density). Activation was measured by CD69 levels on CD8⁺ PR3-CAAR T cells.

FIG. 8 depicts the killing efficiencies of PR3 CAAR T cells in the presence of soluble anti-PR3 monoclonal antibodies (left graph) or soluble anti-neutrophil cytoplasmic autoantibodies (ANCA) derived from patients with ANCA-associated vasculitis (right graph).

FIGS. 9A-9C depict in vivo PR3-CAAR T cell targeting of immunization-elicited PR3-reactive B cell populations. FIG. 9A shows that PR3-immunized mice that received PR3-CAAR T cells showed statistically significant depletion of PR3-reactive splenic class-switched B2 cell populations. The FACS dot plot on the top left shows presence of PR3 reactive class switched B2 cells after treatment with mock CAAR T cells and the graph on the right shows depletion of such cells after treatment with PR3 CAAR T cells. The graph on the bottom shows significant reductions in the percentage of PR3-reactive splenic class-switched B2 cells following treatment with PR3 CAAR T cells compared with control conditions (mock CAAR T cells and no T cells). FIG. 9B shows that PR3-immunized mice that received PR3-CAAR T cells showed statistically significant depletion of PR3-reactive CD19-CD43+ cells in the bone marrow as indicted by the percentage (top graph) and the total counts (bottom graph) of such cells. FIG. 9C shows that PR3-immunized mice that received PR3-CAAR T cells showed statistically significant depletion of PR3-reactive class switched B2 cells in the mesenteric lymph nodes, as indicated by the percentage (top graph) and the total counts (bottom graph) of such cells.

FIG. 10 depicts minimal off-target killing of IgG^{neg} hybridomas by PR3-CAAR T cells with G4S linkers.

FIG. 11 depicts CAAR T cell activation in the absence of target cells (CARs alone) and following 24 h exposure to anti-PR3 expressing cellular targets. Activation was measured by CD69 levels on CD8+PR3-CAAR T cells.

FIG. 12 depicts in vivo PR3-CAAR T cell targeting of PR3-reactive K562 cells in NSG-xenograft model.

### DETAILED DESCRIPTION

Provided herein is a cell or biologic (e.g., protein) therapeutic that comprises a PR3 antibody binding domain, such as a wild type PR3 protein or a mutant PR3 protein. The term “PR3 antibody binding domain,” as used herein, is a protein or polypeptide that can be bound by one or more anti-PR3 antibodies (e.g., one more PR3 ANCA). The PR3 antibody binding domain can be a wild type PR3 protein or a mutant PR3 protein, or a portion of a wild type PR3 protein or a mutant PR3 protein. In some embodiments, the PR3 antibody binding domain comprises one or more ANCA epitopes. In some embodiments, the PR3 antibody binding domain comprises a plurality of ANCA epitopes. In some embodiments, the PR3 antibody binding domain comprises at least 2, 3, 4, 5, 6, or 7 ANCA epitopes. In some embodiments, the PR3 antibody binding domain comprises at least 1, 2, 3, 4, 5, 6, or 7 of the ANCA epitopes disclosed in Table 1. In some embodiments, the PR3 antibody binding domain comprises a plurality (e.g., at least, 2, 3, 4, 5, 6, or 7) of the ANCA epitopes disclosed in Table 1. In some embodiments, the PR3 antibody binding domain is one or more of the PR3 antibody binding domains described in Section IA.

In certain embodiments, the PR3 antibody binding domain, e.g., the mutant PR3 protein, posseses a mutation (e.g., a point mutation) compared to a wild type PR3 protein. In some embodiments, the mutation of the PR3 antibody binding domain reduces its enzymatic activity (compared with a corresponding binding domain that does not contain the mutation) and/or the mutation renders the PR3 antibody binding domain enzymatically inactive. In some embodiments, the corresponding antibody binding domain is the wild type PR3 protein (e.g., the wild type PR3 protein having a sequence of SEQ IN NO:1). Enzymatic activity of a PR3 antibody binding domain can be assessed using the substrate N-methoxysuccinyl-Ala-Ala-Pro-Val p-nitroanilide (MeAAPV) (MilliporeSigma) according to Silva et al. (2010) J Autoimmunity and Van Der Geld et al. (2002) Clin Exp Immunol. In some embodiments, the proteolysis of the synthetic substrate by a PR3 antibody binding domain is monitored via measurement of absorbance at 405 nm over time. In some embodiments, specific activity can be calculated by referencing a standard with p-nitroaniline (cleaved substrate). In certain embodiments, the PR3 antibody binding domain, e.g., the mutant PR3 protein, includes one or more epitopes recognized or bound by a PR3-ANCA.

In some embodiments, the biologic (e.g., protein) therapeutic comprises a soluble PR3 antibody binding domain (e.g., a wild type PR3 protein or mutant PR3 protein, or a portion of a wild type PR3 protein or a mutant PR3 protein). In some embodiments, the cell therapeutic comprises a cell and a PR3 antibody binding domain (e.g., a wild type PR3 protein or mutant PR3 protein) expressed on the surface of the cell. In some embodiments, the PR3 antibody binding domain is part of a recombinant receptor.

Also provided herein is a method of treating an autoimmune disease (e.g., AAV) comprising administering the cell or protein therapeutic.

In another aspect provided herein is a method of depleting antigen specific immune cells, the method comprising administering a cell or biologic therapeutic (e.g. protein) comprising a mutant PR3 protein that binds or is capable of binding at least 1, 2, 3, 4, 5, 6, or 7 epitopes on PR3 specific immune cells (e.g., at least 1, 2, 3, 4, 5, 6, or 7 of the ANCA epitopes disclosed in Table 1). In one embodiment, the therapeutic is a cell therapeutic. In another embodiment, the therapeutic is a protein therapeutic.

Among the provided embodiments are cell therapeutics containing a wild type PR3 protein or a mutant PR3 protein. In some embodiments, provided herein are chimeric autoantibody receptors (CAARs) comprising an extracellular proteinase 3 (PR3) antibody binding domain. In some embodiments, the extracellular domain of the provided CAARs consists of a PR3 antibody binding domain. In some embodiments, the PR3 antibody binding domain is able to be bound by anti-neutrophil cytoplasmic antibodies (ANCA), and therefore serves as a binding target for recognition by disease causing autoantibodies. The autoantibodies expressed on autoreactive cells thus serve as a specific target of the CAARs, and subsequently, the CAARs are able to destroy or kill the autoreactive cells. Also provided herein are methods wherein a CAAR targets autoantibodies and the B cells that produce them. In some embodiments, such methods include methods of treatment and uses for treating diseases or conditions such as autoimmune disease or conditions, such as anti-neutrophil cytoplasmic autoantibody (ANCA)-associated vasculitis (AAV), typically caused by ANCAs targeting PR3.

PR3, expressed in granulomatous lesions, is an autoantigen target for autoreactive B and T cells. Autoantigens can include a self-antigen or antigen from a normal tissue that is the target of a cell-mediated or an antibody-mediated immune response that may result in the development of an auto-immune disease. Autoantigen PR3 (neutrophil serine protease Proteinase 3) is able to be bound by anti-neutrophil cytoplasmic antibodies (ANCA) which induce neutrophils to produce superoxide and alter their apoptosis. Anti-PR3 autoantibodies (PR3-ANCA) target and bind PR3 and are associated with the development of vasculitis.

Certain therapeutic interventions for treatment of such autoantibody-mediated diseases target the immune cells, e.g., B cells, that produce and display the autoantibodies on their cell surfaces. An advantage of the approach disclosed herein is that it allows targeted killing of anti-PR3 autoantibody-expressing immune cells, such as anti-PR3 autoantibody-expressing B cells. Other immune cells (including other B cells) are not targeted by this approach, and accordingly, it is expected that they can continue producing antibodies that protect the subject from infection. Furthermore, among the provided CAARs are those in which the PR3 antibody binding domain is able to be recognized by a plurality of different ANCAs (e.g. 2, 3, 4, 5, 6 or more) to provide a breadth of killing activity against cells that express different reactive autoantibodies. Provided CAARs also exhibit antibody target-specific killing with low tonicity (low tonic activity, as exemplified herein).

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. PR3 CHIMERIC AUTOANTIBODY RECEPTOR

Provided in some aspects are CAARs that comprise a PR3 antibody binding domain. Typically, the PR3 antibody binding domain is capable of being specifically bound by an anti-PR3 antibody, e.g., one or more PR3 ANCAs. Also provided are polynucleotides that encode the PR3 CAARs. In some embodiments, the provided polynucleotides can be incorporated into DNA or RNA constructs, such as those that can be introduced into cells. Cells expressing the PR3 CAARs, compositions containing such cells, and uses thereof in adoptive cell therapy are also provided herein.

### A. PR3 Antibody Binding Domain

The CAARs disclosed herein generally include an extracellular PR3 antibody binding domain (e.g. PR3 autoantibody binding domain). The PR3 antibody binding domain may be a wild-type PR3 protein or a fragment or mutant thereof. In certain embodiments, the antibody binding domain comprises a PR3 protein or a fragment or mutant thereof that is able to be specifically bound by an anti-PR3 antineutrophil cytoplasmic antibody (PR3-ANCA). The extracellular antibody binding domain of the provided CAARs includes one or more eptitopes recognized or bound by a PR3-ANCA. In some embodiments, the extracellular antibody binding domain is a PR3 autoantigen.

The serine protease Proteinase 3 (PR3) is an enzyme released during neutrophil inflammation. PR3 is initially transcribed as an inactive precursor (zymogen) and then undergoes a two-stage posttranslational modification to become active. Firstly (via signal peptidase), there is N-terminal signal peptide cleavage, followed by cleavage of the N-terminal pro-di-peptide by the cysteine proteinase, cathepsin C which is essential for enzymatic activity. Secondly the propeptide form undergoes pro-peptide cleavage at the C terminus, which is crucial for granule packaging. This forms the catalytic triad of residues and the final conformation of mature PR3 (Crisford et al., Respiratory Research, volume 19, Article number: 180, 2018). Residues histidine-44, aspartic acid-91, and serine-176 are the triad which make up the active site of PR3.

In some embodiments, the PR3 antibody binding domain is a wild-type PR3 protein. In some embodiments, the PR3 protein is a human protein. In some embodiments, the PR3 antibody binding domain is a precursor PR3 protein, such as a PR3 with an N-terminal di-peptide and/or a C-terminal pro-peptide. In some embodiments, the PR3 antibody binding domain is a mature PR3 protein. The PR3 antibody binding domain also may be a variant of a human PR3, such as a mutant or isoform of a human PR3, including a precursor or mature PR3 thereof.

In some embodiments, the PR3 antibody binding domain is a precursor PR3 protein that has not been processed at the N-terminus and/or C-terminus. In some embodiments, the precursor PR3 protein is not catalytically active. In some embodiments, the PR3 protein contains an N-terminal di-peptide. In some embodiments, the PR3 antibody binding domain comprises the sequence of SEQ ID NO:5. In some embodiments, the PR3 protein contains an N-terminal di-peptide. In some embodiments, the PR3 antibody binding domain comprises the sequence of SEQ ID NO:3. The PR3 antibody binding domain also may be a variant of any of the foregoing, such as a mutant or isoform of any of the foregoing. In some embodiments, the PR3 antibody binding domain has a sequence that exhibits at least 85%, 86%, 87%, 88%, 89%, 90%, 91%, 92%, 93%, 94%, 95%, 96%, 97%, 98%, or 99% sequence identity to SEQ ID NO:3 or 5.

In some embodiments, the PR3 antibody binding domain is a mature wild-type PR3 protein. In some embodiments, the PR3 antibody binding domain comprises the sequence of SEQ ID NO:1. In some embodiments, the PR3 antibody binding domain is a mature PR3 protein having the sequence of SEQ ID NO:1. The PR3 antibody binding domain also may be a variant of any of the foregoing, such as a mutant or isoform of SEQ ID NO: 1. In some embodiments, the PR3 antibody binding domain has a sequence that exhibits at least 85%, 86%, 87%, 88%, 89%, 90%, 91%, 92%, 93%, 94%, 95%, 96%, 97%, 98%, or 99% sequence identity to SEQ ID NO:1.

In some embodiment, the PR3 antibody binding domain is a mutant PR3 protein. In certain embodiments, the PR3 antibody binding domain is a mutant PR3 protein that posseses a mutation (e.g., a point mutation) compared to wild type PR3, such as compared to PR3 set forth in SEQ ID NO:1. The point mutation can occur, for example, at a residue corresponding to residue 4, 44, 91, 175, or 176 of SEQ ID NO:1. In some embodiments, the point mutation can be to any other amino acid at the position. In some embodiments, the point mutation is to any other amino acid other than an amino acid with a bulky side chain. In some embodiments, the point mutation is to an alanine (A), valine (V), isoleucine (I), leucine (L), methionine (M), glycine (G), proline (P), cysteine (C), serine (S), thereonine (T), asparagine (N) or glutamine (Q). In some embodiments, the amino acid mutation is G4P, H44A, D91N, D175N, S176A or S176C.

In some embodiments, a point mutation of PR3 reduces enzymatic activity of PR3, such as renders the antibody binding domain enzymatically inactive. In some embodiments, the point mutation of PR3 reduces the enzymatic activity of the mutated PR3 compared with a wild-type PR3 (e.g., a wild-type PR3 comprising a sequence of SEQ ID NO:1). In some embodiments, the point mutation of PR3 reduces the enzymatic activity of the mutated PR3 by at least 1%, 5%, 10%, 15%, 20%, 25%, 30%, 35%, 40%, 45%, 50%, 55%, 60%, 65%, 70%, 75%, 80%, 85%, 90%, or 95% as compared to the enzymatic activity of wild type PR3 (e.g. set forth in SEQ ID NO:1). In certain embodiments, the point mutation of PR3 reduces the enzymatic activity of the mutated PR3 by at least 1%, at least 10%, at least 20%, at least 30%, at least 40%, at least 50%, at least 60%, at least 70%, at least 80%, at least 90% or more. In some embodiments, the point mutation of PR3 reduces the enzymatic activity by 1% to 11%, 10% to 21%, 20% to 31%, 30% to 41%, 40% to 51%, 50% to 61%, 60% to 71%, 70% to 81%, 80% to 91% or 90% to 100% as compared to the enzymatic activity of wild type PR3 (e.g. set forth in SEQ ID NO:1). In particular embodiments, the enzymatic activity is reduced by at least 50%, such as generally at least 60%, 70%, 80%, 90% or more compared to the enzymatic activity of wild type PR3 (e.g., set forth in SEQ ID NO:1).

The PR3 antibody binding domain of the CAARs provided herein is recognized and able to be bound by anti-PR3 antibodies, e.g., PR3 antineutrophil cytoplasmic antibodies (PR3-ANCA). ANCA are found in the sera of approximately 90% of all active and/or untreated Wegener's granulomatosis (WG) patient and approximately 90% of all cytoplasmic ANCA WG patients bind PR3 (Bruner et al. (2010) Clin and Experimental Immunol). In some embodiments, the PR3 antibody binding domain disclosed herein comprises one or more ANCA binding epitopes. In some embodiments, the PR3 antibody binding domain comprises 1, 2, 3, 4, 5, 6, or 7 of the ANCA epitopes listed in Table 1 below.

TABLE 1
ANCA epitopes on PR3 antibody binding domain

| Epitope | Sequence | SEQ ID NO: |
|---|---|---|
| ANCA1 epitope | MAHRPPSPAL | 89 |
| ANCA2 epitope | AQPHSRPYMAS | 90 |
| ANCA3 epitope | PGSHFCGG | 91 |
| ANCA4 epitope | VVLGAHNVRTQ | 92 |
| ANCA5 epitope | FLNNYDAE | 93 |
| ANCA6 epitope | PVPHGTQC | 94 |
| ANCA7 eptitope | CFGDSGGP | 95 |

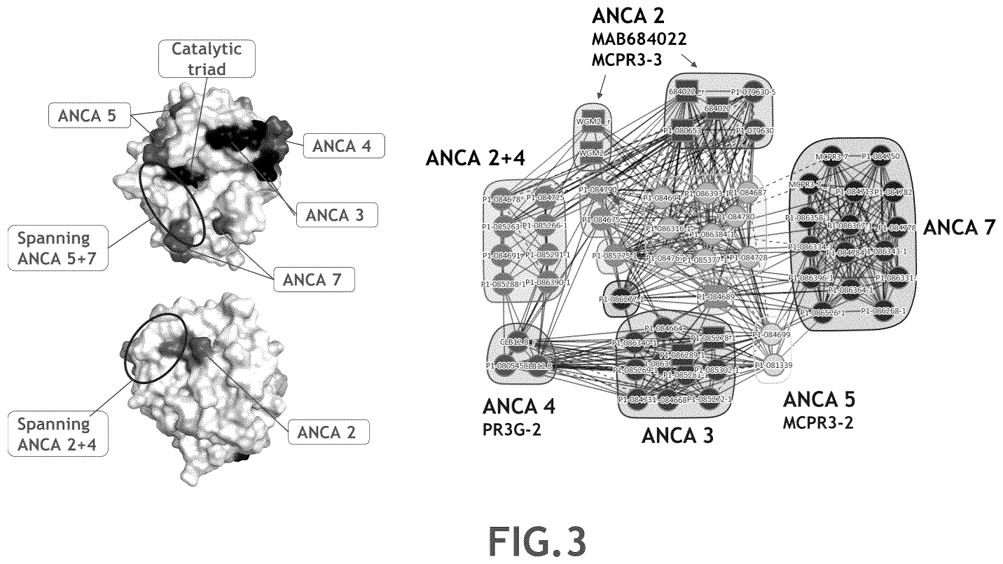

In some embodiments, the PR3 antibody binding domain contains one or more PR3 epitopes that are able to be bound by ANCA. FIG. 3 illustrates the certain ANCA epitopes that may be present on the PR3 antibody binding domain of a provided CAAR. In some embodiments, any one or more, such as a combination of two or more, of the ANCA epitopes in FIG. 3, may be present in a PR3 antibody binding domain of a provided CAAR. In some embodiments, the PR3 antibody binding domain comprises ANCA epitope 2. In some embodiments, the PR3 antibody binding domain comprises ANCA epitope 3. In some embodiments, the PR3 antibody binding domain comprises ANCA epitope 4. In some embodiments, the PR3 antibody binding domain comprises ANCA epitope 5. In some embodiments, the PR3 antibody binding domain comprises ANCA epitope 6. In some embodiments, the PR3 antibody binding domain comprises ANCA epitope 7. In some embodiments, the PR3 antibody binding domain comprises ANCA epitope 1. In some embodiments, the PR3 antibody binding domain comprises a combination of 2 ANCA epitopes, 3 ANCA epitopes, 4 ANCA epitopes, 5 ANCA epitopes, 6 ANCA epitopes, or 7 ANCA epitopes.

In some embodiments, the PR3 antibody binding domain comprises at least two ANCA epitopes. In some embodiments, the PR3 antibody binding domain comprises at least three ANCA epitopes. In some embodiments, the PR3 antibody binding domain comprises at least four ANCA epitopes. In some embodiments, the PR3 antibody binding domain comprises at least five ANCA epitopes. In some embodiments, the PR3 antibody binding domain comprises five ANCA epitopes. In some embodiments, the PR3 antibody binding domain comprises at least six ANCA epitopes. In some aspects of the foregoing embodiments, the ANCA epitopes include one or more of SEQ ID NO:90, SEQ ID NO:91, SEQ ID NO:92, SEQ ID NO:93, SEQ ID NO:94, and SEQ ID NO:95. In some aspects of the foregoing embodiments, the ANCA epitopes include one or more of SEQ ID NO: 89, SEQ ID NO:90, SEQ ID NO:91, SEQ ID NO:92, SEQ ID NO:93, SEQ ID NO:94, and SEQ ID NO:95. In some aspects of the foregoing embodiments, the ANCA epitopes are selected from SEQ ID NO:90, SEQ ID NO:91, SEQ ID NO:92, SEQ ID NO:93, SEQ ID NO:94, and SEQ ID NO:95. In some aspects of the foregoing embodiments, the ANCA epitopes are selected from SEQ ID NO: 89, SEQ ID NO:90, SEQ ID NO:91, SEQ ID NO:92, SEQ ID NO:93, SEQ ID NO:94, and SEQ ID NO:95.

In some embodiments, the PR3 antibody binding domain comprises at least two, three, four, five or six of SEQ ID NO:90, SEQ ID NO:91, SEQ ID NO:92, SEQ ID NO:93, SEQ ID NO:94, and SEQ ID NO:95. In some embodiments, the PR3 antibody binding domain comprises in its sequence at least two sequences of an epitope selected from the group consisting of SEQ ID NO:90, SEQ ID NO:91, SEQ ID NO:92, SEQ ID NO:93, SEQ ID NO:94, and SEQ ID NO:95. In some embodiments, the PR3 antibody binding domain comprises in its sequence at least three sequences of an epitope from the group consisting of SEQ ID NO:90, SEQ ID NO:91, SEQ ID NO:92, SEQ ID NO:93, SEQ ID NO:94, and SEQ ID NO:95. In some embodiments, the PR3 antibody binding domain comprises in its sequence at least four sequences of an epitope selected from the group consisting of SEQ ID NO:90, SEQ ID NO:91, SEQ ID NO:92, SEQ ID NO:93, SEQ ID NO:94, and SEQ ID NO:95. In some embodiments, the PR3 antibody binding domain comprises in its sequence at least five sequences of an epitope selected from the group consisting of SEQ ID NO:90, SEQ ID NO:91, SEQ ID NO:92, SEQ ID NO:93, SEQ ID NO:94, and SEQ ID NO:95. In some embodiments, the PR3 antibody binding domain comprises in its sequence the sequences of epitopes set forth as SEQ ID NO:90, SEQ ID NO:91, SEQ ID NO:92, SEQ ID NO:93, SEQ ID NO:94, and SEQ ID NO:95.

In certain embodiments, the human PR3 antibody binding domain has a length of 231 or fewer amino acids. In certain embodiments, the human PR3 antibody binding domain has length of 229 or fewer amino acids, 227 or fewer amino acids, 225 or fewer amino acids, 223 or fewer amino acids, 222 or fewer amino acids, 221 or fewer amino acids, 219 or fewer amino acids, or 217 or fewer amino acids. In some embodiments, the PR3 antibody binding domain has a length of 217 to 231 amino acids. In some embodiments, the PR3 antibody binding domain has a length of 217 to 222 amino acids. In some embodiments, the PR3 antibody binding domain has a length of 219 to 231 amino acids. In some embodiments, the PR3 antibody binding domain has a length of 221 to 231 amino acids. In some embodiments, the PR3 antibody binding domain has a length of 221 amino acids.

In some embodiments, the CAAR is specific for PR3-reactive immune cells (e.g., B cells). In some embodiments, “specific” with reference to binding is meant binding of a protein (e.g. a ligand) to a cognate binding partner protein present in a sample or expressed on a cell, but which protein does not substantially recognize or bind other molecules in the sample or expression on a cell. For instance, the extracellular PR3 antibody binding domain is specific for a PR3-ANCA expressed on one or more reactive immune cells, such as B cells. In some embodiments, the observation that a protein or other binding molecule binds to a particular PR3-ANCA or specifically binds to a PR3-ANCA protein does not necessarily mean that it binds to a different PR3-ANCA recognizing a different epitope.

In some embodiments, the extent of binding of a PR3 antibody binding domain, or CAAR containing the same, to an unrelated, non-PR3-ANCA protein, is less than at or about 10% of the binding of the PR3 antibody binding domain or the CAAR to a target PR3-ANCA as measured, e.g., by flow cytometry. A variety of assays are known for assessing binding or determining whether a protein (e.g., a ligand) specifically binds to cognate binding partner (e.g., PR3-ANCA). Suitable assays for measuring the binding of one protein to another include, for example, using surface plasmon resonance (SPR) analysis (see, e.g., Scatchard et al., Ann. N. Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent), immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy, flow cytometry, sequencing and other methods for detection of expressed polynucleotides or binding of proteins. In particular, binding can be assessed by flow cytometry.

### 1. Exemplary PR3 Antibody Binding Domain

The PR3 CAARs generally include an extracellular antibody binding domain (e.g., an extracellular autoantibody binding domain) that includes, is, or comprises, one or more of the PR3 antibody binding domains disclosed herein. In some of the provided embodiments, the PR3 antibody binding domain is or comprises the sequence set forth in any one of SEQ ID NOs: 1 to 10 or a sequence that exhibits at least 85%, 86%, 87%, 88%, 89%, 90%, 91%, 92%, 93%, 94%, 95%, 96%, 97%, 98%, or 99% sequence identity to the sequence set forth in any one of SEQ ID NOs: 1 to 10.

In some embodiments, the PR3 antibody binding domain is a mature human PR3 antibody binding domain, e.g. as set forth in SEQ ID NO: 1. In some embodiments, the PR3 antibody binding domain comprises the amino acid sequence set forth in SEQ ID NO:1, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:1. In some embodiments, the PR3 antibody binding domain consists of the sequence set forth in SEQ ID NO:1.

In some embodiments, the PR3 antibody binding domain is a mutant PR3 antibody binding domain containing a mutation at position 176 compared to a mature human PR3, e.g., mature human PR3 as set forth in SEQ ID NO: 1. In some embodiments, the mutation is S176A. In some embodiments, the PR3 antibody binding domain comprises the amino acid sequence set forth in SEQ ID NO:2, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:2 and that contains the mutation S176A. In some embodiments, the PR3 antibody binding domain consists of the sequence set forth in SEQ ID NO:2.

In some embodiments, the PR3 antibody binding domain is a mutant PR3 antibody binding domain containing an unprocessed C terminus compared to mature human PR3, e.g., mature human PR3 as set forth in SEQ ID NO: 1. In some embodiments, the PR3 antibody binding domain comprises the amino acid sequence set forth in SEQ ID NO:3, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:3 and that contains an unprocessed C terminus. In some embodiments, the PR3 antibody binding domain consists of the sequence set forth in SEQ ID NO:3.

In some embodiments, the PR3 antibody binding domain is a mutant PR3 antibody binding domain containing a mutation at position 91 compared to a mature human PR3, e.g., mature human PR3 as set forth in SEQ ID NO: 1. In some embodiments, the mutation is D91N. In some embodiments, the PR3 antibody binding domain comprises the amino acid sequence set forth in SEQ ID NO:4, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:4 and that contains the mutation D91N. In some embodiments, the PR3 antibody binding domain consists of the sequence set forth in SEQ ID NO:4

In some embodiments, the PR3 antibody binding domain is a mutant PR3 antibody binding domain containing an unprocessed N terminus compared to a mature human PR3, e.g., mature human PR3 as set forth in SEQ ID NO: 1. In some embodiments, the PR3 antibody binding domain comprises the amino acid sequence set forth in SEQ ID NO:5, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:5 and that contains an unprocessed N terminus. In some embodiments, the PR3 antibody binding domain consists of the sequence set forth in SEQ ID NO:5.

In some embodiments, the PR3 antibody binding domain is a mutant PR3 antibody binding domain containing a mutation at the N terminus compared to a mature human PR3, e.g., mature human PR3 set forth in SEQ ID NO: 1. In some embodiments, the mutation is an N terminus Ile deletion. In some embodiments, the PR3 antibody binding domain comprises the amino acid sequence set forth in SEQ ID NO:6, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:6 and that contains a N terminus Ile deletion. In some embodiments, the PR3 antibody binding domain consists of the sequence set forth in SEQ ID NO:6.

In some embodiments, the PR3 antibody binding domain is a mutant PR3 antibody binding domain containing a mutation at position 175 compared to a mature human PR3, e.g., mature human PR3 as set forth in SEQ ID NO: 1. In some embodiments, the mutation is D175N. In some embodiments, the PR3 antibody binding domain comprises the amino acid sequence set forth in SEQ ID NO:7, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:7 and that contains the mutation D175N. In some embodiments, the PR3 antibody binding domain consists of the sequence set forth in SEQ ID NO:7.

In some embodiments, the PR3 antibody binding domain is a mutant PR3 antibody binding domain containing a mutation at position 176 compared to a mature human PR3, e.g., mature human PR3 set forth in SEQ ID NO: 1. In some embodiments, the mutation is S176C. In some embodiments, the PR3 antibody binding domain comprises the amino acid sequence set forth in SEQ ID NO:8, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:8 and that contains the mutation S176C. In some embodiments, the PR3 antibody binding domain consists of the sequence set forth in SEQ ID NO:8.

In some embodiments, the PR3 antibody binding domain is a mutant PR3 antibody binding domain containing a mutation at position 4 compared to a mature human PR3, e.g., a mature human PR3 as set forth in SEQ ID NO: 1. In some embodiments, the mutation is G4P. In some embodiments, the PR3 antibody binding domain comprises the amino acid sequence set forth in SEQ ID NO:9, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:9 and that contains the mutation G4P. In some embodiments, the PR3 antibody binding domain consists of the sequence set forth in SEQ ID NO:9.

In some embodiments, the PR3 antibody binding domain is a mutant PR3 antibody binding domain containing a mutation at position 44 compared to a mature human PR3, e.g., a mature human PR3 as set forth in SEQ ID NO: 1. In some embodiments, the mutation is H44A. In some embodiments, the PR3 antibody binding domain comprises the amino acid sequence set forth in SEQ ID NO:10, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:10 and that contains the mutation H44A. In some embodiments, the PR3 antibody binding domain consists of the sequence set forth in SEQ ID NO:10.

In some of the provided embodiments, the PR3 antibody binding domain is encoded by the sequence set forth in any one of SEQ ID NOs: 11 to 20 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence set forth in any one of SEQ ID NOs: 11 to 20.

In some embodiments, the PR3 antibody binding domain is encoded by the nucleotide sequence set forth in SEQ ID NO:11, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:11. In some embodiments, the PR3 antibody binding domain is encoded by a sequence that comprises the sequence set forth in SEQ ID NO: 11. In some embodiments, the PR3 antibody binding domain is encoded by a sequence that consists of the sequence set forth in SEQ ID NO:11.

In some embodiments, the PR3 antibody binding domain is encoded by the nucleotide sequence set forth in SEQ ID NO:12, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:12. In some embodiments, the PR3 antibody binding domain is encoded by a sequence that comprises the sequence set forth in SEQ ID NO: 12. In some embodiments, the PR3 antibody binding domain is encoded by a sequence that consists of the sequence set forth in SEQ ID NO:12. In some embodiments, the encoded PR3 antibody binding domain contains the mutation S176A, with reference to numbering of residues of mature PR3 set forth in SEQ ID NO: 1.

In some embodiments, the PR3 antibody binding domain is encoded by the nucleotide sequence set forth in SEQ ID NO:13, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:13. In some embodiments, the PR3 antibody binding domain is encoded by a sequence that comprises the sequence set forth in SEQ ID NO: 13. In some embodiments, the PR3 antibody binding domain is encoded by a sequence that consists of the sequence set forth in SEQ ID NO:13.

In some embodiments, the PR3 antibody binding domain is encoded by the nucleotide sequence set forth in SEQ ID NO:14, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:14. In some embodiments, the PR3 antibody binding domain is encoded by a sequence that comprises the sequence set forth in SEQ ID NO: 14. In some embodiments, the PR3 antibody binding domain is encoded by a sequence that consists of the sequence set forth in SEQ ID NO:14. In some embodiments, the encoded PR3 antibody binding domain contains the mutation D91N, with reference to numbering of residues of mature PR3 set forth in SEQ ID NO: 1.

In some embodiments, the PR3 antibody binding domain is encoded by the nucleotide sequence set forth in SEQ ID NO:15, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:15. In some embodiments, the PR3 antibody binding domain is encoded by a sequence that comprises the sequence set forth in SEQ ID NO: 15. In some embodiments, the PR3 antibody binding domain is encoded by a sequence that consists of the sequence set forth in SEQ ID NO:15.

In some embodiments, the PR3 antibody binding domain is encoded by the nucleotide sequence set forth in SEQ ID NO:16, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:16. In some embodiments, the PR3 antibody binding domain is encoded by a sequence that comprises the sequence set forth in SEQ ID NO: 16. In some embodiments, the PR3 antibody binding domain is encoded by a sequence that consists of the sequence set forth in SEQ ID NO:16.

In some embodiments, the PR3 antibody binding domain is encoded by the nucleotide sequence set forth in SEQ ID NO:17, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:17. In some embodiments, the PR3 antibody binding domain is encoded by a sequence that comprises the sequence set forth in SEQ ID NO: 17. In some embodiments, the PR3 antibody binding domain is encoded by a sequence that consists of the sequence set forth in SEQ ID NO:17. In some embodiments, the encoded PR3 antibody binding domain contains the mutation D175N, with reference to numbering of residues of mature PR3 set forth in SEQ ID NO: 1.

In some embodiments, the PR3 antibody binding domain is encoded by the nucleotide sequence set forth in SEQ ID NO:18, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:18. In some embodiments, the PR3 antibody binding domain is encoded by a sequence that comprises the sequence set forth in SEQ ID NO: 18. In some embodiments, the PR3 antibody binding domain is encoded by a sequence that consists of the sequence set forth in SEQ ID NO:18. In some embodiments, the encoded PR3 antibody binding domain contains the mutation S176C, with reference to numbering of residues of mature PR3 set forth in SEQ ID NO: 1.

In some embodiments, the PR3 antibody binding domain is encoded by the nucleotide sequence set forth in SEQ ID NO:19, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:19. In some embodiments, the PR3 antibody binding domain is encoded by a sequence that comprises the sequence set forth in SEQ ID NO: 19. In some embodiments, the PR3 antibody binding domain is encoded by a sequence that consists of the sequence set forth in SEQ ID NO:19. In some embodiments, the encoded PR3 antibody binding domain contains the mutation G4P, with reference to numbering of residues of mature PR3 set forth in SEQ ID NO: 1.

In some embodiments, the PR3 antibody binding domain is encoded by the nucleotide sequence set forth in SEQ ID NO:20, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:20. In some embodiments, the PR3 antibody binding domain is encoded by a sequence that comprises the sequence set forth in SEQ ID NO:20. In some embodiments, the PR3 antibody binding domain is encoded by a sequence that consists of the sequence set forth in SEQ ID NO:20. In some embodiments, the encoded PR3 antibody binding domain contains the mutation H44A, with reference to numbering of residues of mature PR3 set forth in SEQ ID NO: 1.

In some embodiments, the PR3 antibody binding domain is or contains the sequence set forth in SEQ ID NO:1. In some embodiments, the PR3 antibody binding domain is or contains the amino acid sequence encoded by SEQ ID NO: 11 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 11. In some embodiments, the PR3 antibody binding domain is or contains the amino acid sequence encoded by SEQ ID NO: 11. In some embodiments, the PR3 CAAR is or contains the amino acid sequence encoded by SEQ ID NO: 11.

In some embodiments, the PR3 antibody binding domain is or contains the sequence set forth in SEQ ID NO:2. In some such embodiments, the PR3 antibody binding domain is or contains the amino acid sequence encoded by SEQ ID NO: 12 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 12. In some embodiments, the PR3 antibody binding domain is or contains the amino acid sequence encoded by SEQ ID NO: 12. In some such embodiments, the PR3 CAAR is or contains the amino acid sequence encoded by SEQ ID NO: 12.

In some such embodiments, the PR3 antibody binding domain is or contains the sequence set forth in SEQ ID NO:3. In some such embodiments, the PR3 antibody binding domain is or contains the amino acid sequence encoded by SEQ ID NO: 13 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 13. In some embodiments, the PR3 antibody binding domain is or contains the amino acid sequence encoded by SEQ ID NO: 13. In some such embodiments, the PR3 CAAR is or contains the amino acid sequence encoded by SEQ ID NO: 13.

In some embodiments, the PR3 antibody binding domain is or contains the sequence set forth in SEQ ID NO:4. In some embodiments, the PR3 antibody binding domain is or contains the amino acid sequence encoded by SEQ ID NO: 14 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 14. In some embodiments, the PR3 antibody binding domain is or contains the amino acid sequence encoded by SEQ ID NO: 14. In some embodiments, the PR3 CAAR is or contains the amino acid sequence encoded by SEQ ID NO: 14.

In some embodiments, the PR3 antibody binding domain is or contains the sequence set forth in SEQ ID NO:5. In some embodiments, the PR3 antibody binding domain is or contains the amino acid sequence encoded by SEQ ID NO: 15 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 15. In some embodiments, the PR3 antibody binding domain is or contains the amino acid sequence encoded by SEQ ID NO: 15. In some embodiments, the PR3 CAAR is or contains the amino acid sequence encoded by SEQ ID NO: 15.

In some embodiments, the PR3 antibody binding domain is or contains the sequence set forth in SEQ ID NO:6. In some embodiments, the PR3 antibody binding domain is or contains the amino acid sequence encoded by SEQ ID NO: 16 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 16. In some embodiments, the PR3 antibody binding domain is or contains the amino acid sequence encoded by SEQ ID NO: 16. In some embodiments, the PR3 CAAR is or contains the amino acid sequence encoded by SEQ ID NO: 16.

In some embodiments, the PR3 antibody binding domain is or contains the sequence set forth in SEQ ID NO:7. In some embodiments, the PR3 antibody binding domain is or contains the amino acid sequence encoded by SEQ ID NO: 17 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 17. In some embodiments, the PR3 antibody binding domain is or contains the amino acid sequence encoded by SEQ ID NO: 17. In some embodiments, the PR3 CAAR is or contains the amino acid sequence encoded by SEQ ID NO: 17.

In some embodiments, the PR3 antibody binding domain is or contains the sequence set forth in SEQ ID NO:8. In some embodiments, the PR3 antibody binding domain is or contains the amino acid sequence encoded by SEQ ID NO: 18 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 18. In some embodiments, the PR3 antibody binding domain is or contains the amino acid sequence encoded by SEQ ID NO: 18. In some embodiments, the PR3 CAAR is or contains the amino acid sequence encoded by SEQ ID NO: 18.

In some embodiments, the PR3 antibody binding domain is or contains the sequence set forth in SEQ ID NO:9. In some embodiments, the PR3 antibody binding domain is or contains the amino acid sequence encoded by SEQ ID NO: 19 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 19. In some embodiments, the PR3 antibody binding domain is or contains the amino acid sequence encoded by SEQ ID NO: 19. In some embodiments, the PR3 CAAR is or contains the amino acid sequence encoded by SEQ ID NO: 19.

In some embodiments, the PR3 antibody binding domain is or contains the sequence set forth in SEQ ID NO:10. In some embodiments, the PR3 antibody binding domain is or contains the amino acid sequence encoded by SEQ ID NO: 20 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 20. In some embodiments, the PR3 antibody binding domain is or contains the amino acid sequence encoded by SEQ ID NO: 20. In some such embodiments, the PR3 CAAR is or contains the amino acid sequence encoded by SEQ ID NO: 20.

Exemplary PR3 antibody binding domains (or autoantibody antibody binding domains) and the corresponding SEQ ID NOs are listed in Table 2 below.

TABLE 2
PR3 antibody binding domain

| | | Amino | |
|---|---|---|---|
| | | acid | Nucleotide |
| | Mutation compared | SEQ | SEQ |
| Construct | to WT sequence | ID NO: | ID NO: |
| PR3_1 | WT (native) | 1 | 11 |
| PR3_2 | S176A | 2 | 12 |
| PR3_3 | Unprocessed C | 3 | 13 |
| | terminus | | |
| PR3_4 | D91N | 4 | 14 |
| PR3_5 | Unprocessed N | 5 | 15 |
| | terminus | | |
| PR3_6 | N terminus Ile | 6 | 16 |
| | deletion | | |
| PR3_7 | D175N | 7 | 17 |
| PR3_8 | S176C | 8 | 18 |
| PR3_9 | G4P | 9 | 19 |
| PR3_10 | H44A | 10 | 20 |

### B. Spacer

In some embodiments, the CAAR disclosed herein further includes a spacer (in some cases also called a spacer region). In some embodiments, the spacer region is positioned between an extracellular domain that comprises or consists of an antibody binding or autoantibody-recognition component (e.g., the PR3 antibody binding domain), and the transmembrane domain (also referred to herein as a transmembrane region).

In some embodiments, the length of the spacer is adjusted to optimize the biophysical synapse distance between the CAAR-expressing cell and the target of the CAAR, (e.g., anti-PR3 antibodies, e.g., PR3 autoantibodies). In some embodiments, the CAAR is expressed by a T cell, and the length of the spacer is adjusted to a length that is compatible for T cell activation or to optimize CAAR T-cell performance.

In some embodiments, the spacer is selected such that the CAAR has tonic signalling of less than 20%, as assessed using the assay described in Example 2. In some embodiments, the tonic signaling is less than 15%, such as assessed using the assay described in Example 2. In some embodiments, the tonic signaling is less than 10%, such as assessed using the assay described in Example 2. In some embodiments, the spacer is selected such that the CAAR is able to kill at least 50%, 55%, 60%, 65%, 70%, 75%, or 80% of an anti-PR3 antibody expressing cell target. In some embodiments, the CAAR is able to kill at least 65% of an anti-PR3 antibody expressing cell target, such as assessed using the assay described in Example 3.

In some embodiments, the spacer can be of a length that provides for increased responsiveness of the cell following antigen binding, as compared to in the absence of the spacer or as compared to an alternative spacer of a different length (e.g. longer in length).

Exemplary spacers include those having at least at or about 10 to at or about 300 amino acids, at or about 10 to at or about 229 amino acids, at or about 10 to at or about 200 amino acids, at or about 10 to at or about 175 amino acids, at or about 10 to at or about 150 amino acids, at or about 10 to at or about 125 amino acids, at or about 10 to at or about 100 amino acids, at or about 10 to at or about 75 amino acids, at or about 10 to at or about 50 amino acids, at or about 10 to at or about 40 amino acids, at or about 10 to at or about 30 amino acids, at or about 10 to at or about 20 amino acids, or at or about 10 to at or about 15 amino acids in length, and including any integer between the endpoints of any of the listed ranges. Exemplary spacers include those having at least at or about at or about 50 to at or about 175 amino acids, at or about 50 to at or about 150 amino acids, at or about 10 to at or about 125 amino acids, at or about 50 to at or about 100 amino acids, at or about 100 to at or about 300 amino acids, at or about 100 to at or about 250 amino acids, at or about 125 to at or about 250 amino acids, or at or about 200 to at or about 250 amino acids, and including any integer between the endpoints of any of the listed ranges. In some embodiments, a spacer is at least at or about 12 amino acids, at least at or about 119 amino acids, at least at or about 125 amino acids, at least at or about 200 amino acids, or at least at or about 220 amino acids, or at least at or about 225 amino acids in length. In some embodiments, a spacer is at least at or about 13 amino acids, at least at or about 120 amino acids, at least at or about 125 amino acids, at least at or about 200 amino acids, or at least at or about 220 amino acids, or at least at or about 229 amino acids in length. In some embodiments, a spacer is at or about 11, 12, 13, 14, 15, 16, 17, 18, 19, 20, 21, 22, 23, 24, 25 amino acids or less in length. In some embodiments, the spacer is at least at or about 100 amino acids in length, such as at least at or about 110, 111, 112, 113, 114, 115, 116, 117, 118, 119, 120, 121, 122, 123, 124, 125, 130, 135, 140, 145, 150, 160, 170, 180, 190, 200, 210, 220, 230, 240, or 250 amino acids in length.

In some embodiments, the spacer is at least at or about 125 to at or about 300 amino acids, at or about 125 to at or about 250 amino acids, at or about 125 to at or about 230 amino acids, at or about 125 to at or about 200 amino acids, at or about 125 to at or about 180 amino acids, at or about 125 to at or about 150 amino acids, at or about 150 to at or about 300 amino acids, at or about 150 to at or about 250 amino acids, at or about 150 to at or about 230 amino acids, at or about 150 to at or about 200 amino acids, at or about 150 to at or about 180 amino acids, at or about 180 to at or about 300 amino acids, at or about 180 to at or about 250 amino acids, at or about 180 to at or about 230 amino acids, at or about 180 to at or about 200 amino acids, at or about 200 to at or about 300 amino acids, at or about 200 to at or about 250 amino acids, at or about 200 to at or about 230 amino acids, at or about 230 to at or about 300 amino acids, at or about 230 to at or about 250 amino acids in length or 250 to at or about 300 amino acids in length. In some embodiments, the spacer is at least at or about 129, 130, 140, 150, 160, 170, 180, 190, 200, 210, 220, 221, 222, 223, 224, 225, 226, 227, 228, 229 or 230 amino acids in length, or a length between any of the foregoing.

In some embodiments, the spacer may be or include at least a portion of an immunoglobulin constant region or variant or modified version thereof, such as a hinge region. In some embodiments, the constant region or portion is of a human IgG, such as IgG4, IgG2 or IgG1. In some embodiments, the portion of an immunoglobulin constant region includes a hinge region (e.g. IgG4 hinge region) and, optionally one or more additional domains of the constant region, such as a C_{H}1/C_{L}, C_{H}2 and/or C_{H}3 and/or Fc region. In some aspects, the portion of the constant region serves as a spacer between the autoantibody-recognition component, e.g., the PR3 antibody binding domain, and the transmembrane domain.

Exemplary spacers include an IgG hinge alone, an IgG hinge linked to one or more of a C_{H}2 and C_{H}3 domain, or IgG hinge linked to the C_{H}3 domain. In some embodiments, the spacer includes an IgG hinge alone. In some embodiments, the IgG hinge, C_{H}2 and/or C_{H}3 can be derived all or in part from IgG4 or IgG2, such as all or in part from human IgG4 or human IgG2. In some embodiments, the spacer can be a chimeric polypeptide containing one or more of a hinge, C_{H}2 and/or C_{H}3 sequence(s) derived from IgG4, IgG2, and/or IgG2 and IgG4. In some embodiments, the hinge region comprises all or a portion of an IgG4 hinge region. In some embodiments, the hinge region comprises all or a portion of an IgG4 hinge region and/or of an IgG2 hinge region, wherein the IgG4 hinge region is optionally a human IgG4 hinge region and the IgG2 hinge region is optionally a human IgG2 hinge region; the C_{H}2 region comprises all or a portion of an IgG4 C_{H}2 region and/or of an IgG2 C_{H}2 region, wherein the IgG4 C_{H}2 region is optionally a human IgG4 C_{H}2 region and the IgG2 C_{H}2 region is optionally a human IgG2 C_{H}2 region; and/or the C_{H}3 region comprises all or a portion of an IgG4 C_{H}3 region and/or of an IgG2 C_{H}3 region, wherein the IgG4 C_{H}3 region is optionally a human IgG4 C_{H}3 region and the IgG2 C_{H}3 region is optionally a human IgG2 C_{H}3 region. In some embodiments, the hinge, C_{H}2 and C_{H}3 comprises all or a portion of each of a hinge region, C_{H}2 and C_{H}3 from IgG4. In some embodiments, the hinge region is chimeric and comprises a hinge region from human IgG4 and human IgG2; the C_{H}2 region is chimeric and comprises a C_{H}2 region from human IgG4 and human IgG2; and/or the C_{H}3 region is chimeric and comprises a C_{H}3 region from human IgG4 and human IgG2. In some embodiments, the spacer comprises an IgG4/2 chimeric hinge or a modified IgG4 hinge comprising at least one amino acid replacement compared to human IgG4 hinge region; an human IgG2/4 chimeric C_{H}2 region; and a human IgG4 C_{H}3 region.

In some embodiments, the spacer and can contain one or more single amino acid mutations in one or more domains of the immunoglobulin, such as in one or more domains of the hinge, C_{H}2 or C_{H}3 region. In some examples, the amino acid modification is a substitution of a proline (P) for a serine (S) in the hinge region of an IgG4. In some embodiments, the spacer is or contains a IgG4 hinge that is a variant IgG4 hinge region comprising substitution of amino acids CPSC to CPPC compared to the wild-type IgG4 hinge region. In some embodiments, the spacer contains a C_{H}2 of an IgG4 that is a variant C_{H}2 in which amino acid modification is a substitution of a glutamine (Q) for an asparagine (N) to reduce glycosylation heterogeneity, such as an N177Q mutation at position 177, in the C_{H}2 region, of the full-length IgG4 Fc sequence set forth in SEQ ID NO: 132 or an N176Q at position 176, in the C_{H}2 region, of the full-length IgG2 Fc sequence set forth in SEQ ID NO:133.

In some examples, the spacer is at or about 12 amino acids in length or is no more than at or about 12 amino acids in length. In some examples, the spacer is at or about 15 amino acids in length or is no more than at or about 15 amino acids in length.

In some embodiments, the spacer comprises or consists of all or a portion of an immunoglobulin hinge or a modified version thereof. In some embodiments, the spacer is at or about 15 amino acids or less in length. In some embodiments, the spacer comprises or consists of all or a portion of an immunoglobulin hinge, optionally an IgG4 hinge, or a modified version thereof and/or comprises about 15 amino acids or less. In some embodiments, the spacer is at or about 13 amino acids in length and/or comprises or consists of all or a portion of an immunoglobulin hinge, optionally an IgG4, or a modified version thereof. In some embodiments, the spacer is at or about 12 amino acids in length and/or comprises or consists of all or a portion of an immunoglobulin hinge, optionally an IgG4, or a modified version thereof. In some embodiments, the spacer comprises the formula X₁PPX₂P (SEQ ID NO:124), where X₁ is glycine, cysteine or arginine and X₂ is cysteine or threonine. In some embodiments, the spacer does not comprise a CD28 extracellular region or a CD8 extracellular region. In certain cases, the spacer has a methionine residue at the C-terminus. In some embodiments, the spacer comprises or consists of the sequence of SEQ ID NO: 22, 23, 125, 126, 127, 128, 129, 130, or 131 or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the spacer is or contains the sequence set forth in SEQ ID NO:22. In some embodiments, the spacer is or contains the sequence set forth in SEQ ID NO:23.

In some embodiments, the spacer is or comprises IgG hinge linked to the C_{H}3 domain, e.g., of a human immunoglobulin, such as IgG4 and/or IgG2. In some aspects, the spacer is at or about 111, 112, 113, 114, 115, 116, 117, 118, 119, 120, 121, 122, 123, 124 or 125 amino acids in length, or has a length between any of the foregoing. In some aspects, the spacer is at or about 119 or 120 amino acids in length. In some embodiments, the spacer comprises or consists of the sequence of SEQ ID NO: 24 or 96, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the spacer is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 24 or 96. In some embodiments, the spacer is or contains the sequence set forth in SEQ ID NO: 24. In some embodiments, the spacer is or contains the sequence set forth in SEQ ID NO: 96. In some embodiments, the spacer is at or about 120 amino acids in length.

In some embodiments, the spacer can be from all or in part from IgG4 and/or IgG2 and can contain mutations, such as one or more single amino acid mutations in one or more domains. In some examples, the amino acid modification is a substitution of a proline (P) for a serine (S) in the hinge region of an IgG4. In some embodiments, the spacer is or contains a IgG4 hinge that is a variant IgG4 hinge region comprising substitution of amino acids CPSC to CPPC compared to the wild-type IgG4 hinge region. In some embodiments, the amino acid modification is a substitution of a glutamine (Q) for an asparagine (N) to reduce glycosylation heterogeneity, such as an N177Q mutation at position 177, in the C_{H}2 region, of the full-length IgG4 Fc sequence set forth in SEQ ID NO: 132 or an N176Q at position 176, in the C_{H}2 region, of the full-length IgG2 Fc sequence set forth in SEQ ID NO:133.

In some embodiments, the spacer is or comprises an IgG4/2 chimeric hinge or a modified IgG4 hinge; an IgG2/4 chimeric C_{H}2 region; and an IgG4 C_{H}3 region and optionally is about 228 or 229 amino acids in length; or a spacer set forth in SEQ ID NO: 25 or 97. In some embodiments, the spacer is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 25 or 97. In some embodiments, the spacer is or contains the sequence set forth in SEQ ID NO: 25 or 97.

Additional exemplary spacers include, but are not limited to, those described in Hudecek et al. (2013) Clin. Cancer Res., 19:3153, Hudecek et al. (2015) Cancer Immunol. Res., 3(2):125-135, or WO2014031687. In some embodiments, the nucleotide sequence of the spacer is optimized to reduce RNA heterogeneity upon expression. In some embodiments, the nucleotide sequence of the spacer is optimized to reduce cryptic splice sites or reduce the likelihood of a splice event at a splice site.

In some embodiments, the spacer comprises or consists of the amino acid sequence set forth in SEQ ID NO:23. In some embodiments, the spacer comprises or consists of the amino acid sequence set forth in SEQ ID NO:22. In some embodiments, the spacer comprises or consists of the amino acid sequence set forth in SEQ ID NO:96. In some embodiments, the spacer comprises or consists of the amino acid sequence set forth in SEQ ID NO:24. In some embodiments, the spacer comprises or consists of the amino acid sequence set forth in SEQ ID NO:97. In some embodiments, the spacer comprises or consists of the amino acid sequence set forth in SEQ ID NO:25.

In some embodiments, the spacer comprises or consists of an amino acid sequence that exhibits at least at or about 85%, 86%, 87%, 88%, 89%, 90%, 91%, 92%, 93%, 94%, 95%, 96%, 97%, 98% or 99% sequence identity to SEQ ID NO:23, 96, or 97. In some such embodiments, the spacer is encoded by a polynucleotide that has been optionally optimized for codon usage and/or to reduce RNA heterogeneity. In some embodiments, the spacer comprises or consists of an amino acid sequence that exhibits at least at or about 85%, 86%, 87%, 88%, 89%, 90%, 91%, 92%, 93%, 94%, 95%, 96%, 97%, 98% or 99% sequence identity to SEQ ID NO:22, 24, or 25. In some such embodiments, the spacer is encoded by a polynucleotide that has been optionally optimized for codon usage and/or to reduce RNA heterogeneity.

In some embodiments, the CAAR comprises a hinge domain derived from the CD8 polypeptide. In some embodiments, the spacer comprises or consists of the sequence of SEQ ID NO: 101, or a variant thereof having at least 85%, 86%, 87%, 88%, 89%, 90%, 91%, 92%, 93%, 94%, 95%, 96%, 97%, 98%, 99% or more sequence identity thereto. In some embodiments, the spacer is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 101. In some embodiments, the spacer is or contains the sequence set forth in SEQ ID NO: 101.

In some embodiments, the amino acid sequence of the spacer comprises or consists of (GGGGS) n. In some such embodiments, the spacer has between 5 and 30 amino acids. In some embodiments, n is between 1 and 6 (inclusive). In one embodiment, n=1 (SEQ ID NO:99). In another embodiment, n=2. In another embodiment, n=3 (SEQ ID NO: 100).

In some embodiments, the spacer comprises or consists of the sequence of SEQ ID NO: 99, or a variant thereof having at least 85%, 86%, 87%, 88%, 89%, 90%, 91%, 92%, 93%, 94%, 95%, 96%, 97%, 98%, 99% or more sequence identity thereto. In some embodiments, the spacer is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 99. In some embodiments, the spacer is or contains the sequence set forth in SEQ ID NO: 99.

In some embodiments, the spacer comprises or consists of the sequence of SEQ ID NO: 100, or a variant thereof having at least 85%, 86%, 87%, 88%, 89%, 90%, 91%, 92%, 93%, 94%, 95%, 96%, 97%, 98%, 99% or more sequence identity thereto. In some embodiments, the spacer is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 100. In some embodiments, the spacer is or contains the sequence set forth in SEQ ID NO: 100.

In some embodiments, the spacer is a flexible linker spacer. In some embodiments, the amino acid sequence of the spacer comprises or consists of EAAAK (SEQ ID NO: 102).

In some embodiments, the spacer comprises or consists of the sequence of SEQ ID NO: 102, or a variant thereof having at least 85%, 86%, 87%, 88%, 89%, 90%, 91%, 92%, 93%, 94%, 95%, 96%, 97%, 98%, 99% or more sequence identity thereto. In some embodiments, the spacer is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 102. In some embodiments, the spacer is or contains the sequence set forth in SEQ ID NO: 102.

### C. Transmembrane Domain

In some embodiments, the extracellular binding domain containing the PR3 antibody binding component is linked to one or more intracellular signaling components (e.g., signaling components that mimic activation through a TCR complex) through a transmembrane domain (also referred to herein as a transmembrane region). Thus, in some embodiments, a PR3 antibody binding domain and spacer is linked to one or more transmembrane domains such as those described herein and intracellular signaling domains comprising one or more intracellular components such as those described herein. In some embodiments, the transmembrane domain is fused to the extracellular domain. In one embodiment, a transmembrane domain that naturally is associated with one of the domains in the receptor is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane domains include those derived from (i.e. comprise at least the transmembrane domain(s) of) the alpha, beta or zeta chain of the T-cell receptor, CD3 epsilon, CD4, CD5, CD8, CD9, CD16, CD22, CD28, CD33, CD37, CD45, CD64, CD80, CD86, CD134, CD137, and/or CD154. For example, the transmembrane domain can be a CD28 transmembrane domain that comprises the sequence of amino acids set forth in SEQ ID NO: 26 or 134. In some embodiments, the transmembrane domain of the receptor is a transmembrane domain of human CD28 or variant thereof, e.g., a 27-amino acid transmembrane domain of a human CD28 (Accession No.: P10747.1), or a 28-amino acid sequence, or is a transmembrane domain that comprises the sequence of amino acids set forth in SEQ ID NO:26 or 134 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 26 or 134. In some embodiments, the transmembrane domain of the receptor is a transmembrane domain of human C8a or variant thereof, e.g., a 21-amino acid transmembrane domain of a human CD8a (Accession No.: P01732), or a 22-amino acid sequence, or is a transmembrane domain that comprises the sequence of amino acids set forth in SEQ ID NO: 98 or 135 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 98 or 135.

In some embodiments, the transmembrane domain is encoded by a polynucleotide that has been optionally optimized for codon usage and/or to reduce RNA heterogeneity, e.g., by removing cryptic splice sites. In certain cases, the transmembrane domain has a methionine residue at the N-terminus.

In some embodiments, the transmembrane domain is or contains SEQ ID NO: 26 or 134 or an amino acid sequence having at least at or about 90%, 91%, 92%, 93%, 94%, 95%, 96%, 97%, 98% or 99% sequence identity to SEQ ID NO: 26 or 134. In some embodiments, the transmembrane domain is or contains the sequence set forth in SEQ ID NO: 26 or 134. In some embodiments, the transmembrane domain is or contains SEQ ID NO: 98 or 135 or an amino acid sequence having at least at or about 90%, 91%, 92%, 93%, 94%, 95%, 96%, 97%, 98% or 99% sequence identity to SEQ ID NO: 98 or 135. In some embodiments, the transmembrane domain is or contains the sequence set forth in SEQ ID NO: 98 or 135.

Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

### D. Intracellular Signaling Domain

The CAAR generally includes an intracellular signaling domain comprising at least one intracellular signaling component or components. Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the intracellular signaling domain of the CAAR.

In some embodiments, the CAAR includes an intracellular component or signaling domain of a TCR complex, such as a TCR CD3 chain that mediates T-cell activation and cytotoxicity, e.g., CD3 zeta (CD3-ζ) chain. Thus, in some aspects, the PR3-antibody binding domain is linked to one or more cell signaling modules. In some embodiments, cell signaling modules include CD3 intracellular signaling domains and/or other CD transmembrane domains. In some embodiments, the CAAR further includes a portion of one or more additional molecules such as Fc receptor γ, CD8, CD4, CD25, or CD16.

In some embodiments, upon ligation of the CAAR, the cytoplasmic domain or intracellular signaling region of the CAR stimulates and/or activates at least one of the normal effector functions or responses of the immune cell, e.g., T cell engineered to express the CAR. For example, in some contexts, the CAAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAAR. In other embodiments, the CAAR does not include a component for generating a costimulatory signal. In some aspects, an additional CAAR is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAAR includes one or both of such classes of cytoplasmic signaling sequences.

In some aspects, the CAAR includes a primary cytoplasmic signaling sequence that regulates primary stimulation and/or activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, CD3 gamma, CD3 delta and CD3 epsilon. In some embodiments, the intracellular signaling region in the CAAR contain(s) a cytoplasmic signaling domain, portion thereof, or sequence derived from CD3 zeta. In some embodiments the CD3 zeta comprises the sequence of amino acids set forth in SEQ ID NO: 28, 136, or 137. In some embodiments, the CD3 zeta is encoded by a polynucleotide that has been optionally optimized for codon usage and/or to reduce RNA heterogeneity, e.g., by removing cryptic splice sites. In some embodiments, the intracellular signaling domain comprises a human CD3 zeta stimulatory signaling domain or functional variant thereof, such as an 112 AA cytoplasmic domain of isoform 3 of human CD3ζ (Accession No.: P20963.2) or a CD3 zeta signaling domain as described in U.S. Pat. No. 7,446,190 or U.S. Pat. No. 8,911,993. In some embodiments, the intracellular signaling domain comprises the sequence of amino acids set forth in SEQ ID NO: 28, 136, or 137 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 28, 136, or 137. In some embodiments, the CD3 zeta is or contains the sequence set forth in SEQ ID NO: 28.

In some embodiments, the CAAR includes a signaling domain (e.g., an intracellular or cytoplasmic signaling domain) and/or transmembrane portion of a costimulatory molecule, such as a T cell costimulatory molecule. Exemplary costimulatory molecules include CD28, 4-1BB, OX40, DAP10, and ICOS. For example, a costimulatory molecule can be derived from 4-1BB and can comprise the amino acid sequence set forth in SEQ ID NO: 27. In some embodiments, the costimulatory molecule from 4-1BB is encoded by a polynucleotide that has been optionally optimized for codon usage and/or to reduce RNA heterogeneity, e.g., by removing cryptic splice sites. In some embodiments, the costimulatory molecule from 4-1BB comprises the amino acid sequence set forth in SEQ ID NO:27. In some embodiments, the intracellular domain comprises an intracellular costimulatory signaling domain of 4-1BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 27 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 27. In some embodiments, a costimulatory molecule can be derived from CD28 and can comprise the amino acid sequence set forth in SEQ ID NO: 138, encoded by the nucleotide sequence set forth in SEQ ID NO: 139. In some embodiments, the intracellular signaling domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling domain can comprise the sequence of amino acids set forth in SEQ ID NO: 138 or 140 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 138 or 140. In some aspects, the same CAR includes both the stimulatory or activating components (e.g., cytoplasmic signaling sequence) and costimulatory components.

In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 4-1BB.

In some embodiments, the stimulatory or activating components are included within one CAAR, whereas the costimulatory component is provided by another CAAR recognizing another antigen. In some embodiments, the CAARs include activating or stimulatory CAARs, and costimulatory CAARs, both expressed on the same cell (see WO 2014/055668). In some aspects, the PR3-CAAR is the stimulatory or activating CAAR; in other aspects, it is the costimulatory CAAR. In some embodiments, the cells further include inhibitory CAARs (iCARs, see Fedorov et al., Sci. Transl. Medicine, 5(215) (December, 2013) whereby a stimulatory or an activating signal delivered through the PR3-CAAR is diminished or inhibited by binding of the inhibitory CAAR to its ligand, e.g., to reduce off-target effects.

In some embodiments, the two receptors induce, respectively, an activating and an inhibitory signal to the cell, such that ligation of one of the receptor to its antigen activates the cell or induces a response, but ligation of the second inhibitory receptor to its antigen induces a signal that suppresses or dampens that response. Examples are combinations of activating CAARs and inhibitory CAARs. Such a strategy may be used, for example, to reduce the likelihood of off-target effects in the context in which the activating CAAR binds an antigen expressed in a disease or condition but which is also expressed on normal cells, and the inhibitory receptor binds to a separate antigen which is expressed on the normal cells but not cells of the disease or condition.

In some aspects, the chimeric receptor is or includes an inhibitory CAAR and includes intracellular components that dampen or suppress an immune response, such as an ITAM- and/or co stimulatory-promoted response in the cell. Exemplary of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAAR including a signaling domain of or derived from such an inhibitory molecule, such that it serves to dampen the response of the cell, for example, that induced by an activating and/or costimulatory CAAR.

In certain embodiments, the intracellular signaling region comprises a CD28 transmembrane and signaling domain linked to a CD3 (e.g., CD3-zeta) intracellular domain. In some embodiments, the intracellular signaling domain comprises a chimeric CD28 and 4-1BB (CD137; TNFRSF9) co-stimulatory domains, linked to a CD3 zeta intracellular domain.

In some embodiments, the CAR encompasses one or more, e.g., two or more, costimulatory domains and a stimulatory or an activation domain, e.g., primary activation domain, in the cytoplasmic portion. Exemplary CARs include intracellular components of CD3-zeta and 4-1BB.

### E. Exemplary CAARs

Provided herein is a CAAR comprising any of the PR3 antibody binding domains such as any described herein, an IgG4 hinge spacer, a CD28 transmembrane domain, a 4-1BB costimulatory domain, and a CD3 zeta signaling domain. In certain embodiments, the CAAR comprises any of the PR3 antibody binding domains described herein; a spacer that is or contains the sequence set forth in SEQ ID NO: 22; a transmembrane domain that is or contains the sequence set forth in SEQ ID NO: 26; and an intracellular signaling domain comprising the 4-1BB signaling domain sequence set forth in SEQ ID NO: 27 and the CD3 zeta signaling domain sequence set forth in SEQ ID NO: 28. In some of any embodiments, the PR3 CAAR comprises the amino acid sequence set forth in SEQ ID NO:29, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:29. In some of any embodiments, the PR3 CAAR comprises the amino acid sequence set forth in SEQ ID NO:32, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:32 and that contains the mutation S176A. In some of any embodiments, the PR3 CAAR comprises the amino acid sequence set forth in SEQ ID NO:35, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:35 and that contains an unprocessed C terminus. In some of any embodiments, the PR3 CAAR comprises the amino acid sequence set forth in SEQ ID NO:38, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:38 and that contains the mutation D91N. In some of any embodiments, the PR3 CAAR comprises the amino acid sequence set forth in SEQ ID NO:41, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:41 and that contains an unprocessed N terminus. In some of any embodiments, the PR3 CAAR comprises the amino acid sequence set forth in SEQ ID NO:44, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:44 and that contains an N terminus Ile deletion. In some of any embodiments, the PR3 CAAR comprises the amino acid sequence set forth in SEQ ID NO:47, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:47 and that contains the mutation D175N. In some of any embodiments, the PR3 CAAR comprises the amino acid sequence set forth in SEQ ID NO:50, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:50 and that contains the mutation S176C. In some of any embodiments, the PR3 CAAR comprises the amino acid sequence set forth in SEQ ID NO:53, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:53 and that contains the mutation G4P. In some of any embodiments, the PR3 CAAR comprises the amino acid sequence set forth in SEQ ID NO:56, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:56 and that contains the mutation H44A.

In some embodiments, the antibody binding domain of the PR3 CAAR comprises a mutant PR3 antibody binding domain containing a mutation at position 91 compared to mature human PR3. In some embodiments, the mutation of the PR3 antibody binding domain is D91N. In some embodiments, the PR3 CAAR comprises an antibody binding domain with the amino acid sequence set forth in SEQ ID NO:4 and a spacer with the amino acid sequence set forth in SEQ ID NO:23. In some embodiments, the PR3 CAAR comprises an antibody binding domain with the amino acid sequence set forth in SEQ ID NO:4 and a spacer with the amino acid sequence set forth in SEQ ID NO:22. In some embodiments, the PR3 CAAR has the amino acid sequence set forth in SEQ ID NO:38.

In some embodiments, the antibody binding domain of the PR3 CAAR comprises a mutant PR3 antibody binding domain containing a mutation at position 44 compared to mature human PR3. In some embodiments, the mutation of the PR3 antibody binding domain is H44A. In some embodiments, the PR3 CAAR comprises an antibody binding domain with the amino acid sequence set forth in SEQ ID NO:10 and a spacer with the amino acid sequence set forth in SEQ ID NO:23. In some embodiments, the PR3 CAAR comprises an antibody binding domain with the amino acid sequence set forth in SEQ ID NO:10 and a spacer with the amino acid sequence set forth in SEQ ID NO:22. In some embodiments, the PR3 CAAR has the amino acid sequence set forth in SEQ ID NO: 56.

Provided herein is a CAAR comprising any of the PR3 antibody binding domains such as any described herein, an IgG4 hinge spacer, a CD28 transmembrane domain, a 4-1BB costimulatory domain, and a CD3 zeta signaling domain. In certain embodiments, the CAAR comprises any of the PR3 antibody binding domains described herein; a spacer that is or contains the sequence set forth in SEQ ID NO: 24; a transmembrane domain that is or contains the sequence set forth in SEQ ID NO: 26; and an intracellular signaling domain comprising the 4-1BB signaling domain sequence set forth in SEQ ID NO: 27 and the CD3 zeta signaling domain sequence set forth in SEQ ID NO: 28. In some of any embodiments, the PR3 CAAR comprises the amino acid sequence set forth in SEQ ID NO:30, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:30. In some of any embodiments, the PR3 CAAR comprises the amino acid sequence set forth in SEQ ID NO:33, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:33 and that contains the mutation S176A. In some of any embodiments, the PR3 CAAR comprises the amino acid sequence set forth in SEQ ID NO:36, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:36 and that contains an unprocessed C terminus. In some of any embodiments, the PR3 CAAR comprises the amino acid sequence set forth in SEQ ID NO:39, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:39 and that contains the mutation D91N. In some of any embodiments, the PR3 CAAR comprises the amino acid sequence set forth in SEQ ID NO:42, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:42 and that contains an unprocessed N terminus. In some of any embodiments, the PR3 CAAR comprises the amino acid sequence set forth in SEQ ID NO:45, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:45 and that contains an N terminus Ile deletion. In some of any embodiments, the PR3 CAAR comprises the amino acid sequence set forth in SEQ ID NO:48, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:48 and that contains the mutation D175N. In some of any embodiments, the PR3 CAAR comprises the amino acid sequence set forth in SEQ ID NO:51, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:51 and that contains the mutation S176C. In some of any embodiments, the PR3 CAAR comprises the amino acid sequence set forth in SEQ ID NO:54, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:54 and that contains the mutation G4P. In some of any embodiments, the PR3 CAAR comprises the amino acid sequence set forth in SEQ ID NO:57, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:57 and that contains the mutation H44A.

In some embodiments, the antibody binding domain of the PR3 CAAR comprises a mutant PR3 antibody binding domain containing a mutation at position 44 compared to mature human PR3. In some embodiments, the mutation of the PR3 antibody binding domain is H44A. In some embodiments, the PR3 CAAR comprises an antibody binding domain with the amino acid sequence set forth in SEQ ID NO:10 and a spacer with the amino acid sequence set forth in SEQ ID NO:96. In some embodiments, the PR3 CAAR comprises an antibody binding domain with the amino acid sequence set forth in SEQ ID NO:10 and a spacer with the amino acid sequence set forth in SEQ ID NO:24. In some embodiments, the PR3 CAAR has the amino acid sequence set forth in SEQ ID NO:57.

Provided herein is a CAAR comprising any of the PR3 antibody binding domains such as any described herein, an IgG4 hinge spacer, a CD28 transmembrane domain, a 4-1BB costimulatory domain, and a CD3 zeta signaling domain. In certain embodiments, the CAAR comprises any of the PR3 antibody binding domains described herein; a spacer that is or contains the sequence set forth in SEQ ID NO: 25; a transmembrane domain that is or contains the sequence set forth in SEQ ID NO: 26; and an intracellular signaling domain comprising the 4-1BB signaling domain sequence set forth in SEQ ID NO: 27 and the CD3 zeta signaling domain sequence set forth in SEQ ID NO: 28. In some of any embodiments, the PR3 CAAR comprises the amino acid sequence set forth in SEQ ID NO:31, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:31. In some of any embodiments, the PR3 CAAR comprises the amino acid sequence set forth in SEQ ID NO:34, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:34 and that contains the mutation S176A. In some of any embodiments, the PR3 CAAR comprises the amino acid sequence set forth in SEQ ID NO:37, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:37 and that contains an unprocessed C terminus. In some of any embodiments, the PR3 CAAR comprises the amino acid sequence set forth in SEQ ID NO:40, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:40 and that contains the mutation D91N. In some of any embodiments, the PR3 CAAR comprises the amino acid sequence set forth in SEQ ID NO:43, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:43 and that contains an unprocessed N terminus. In some of any embodiments, the PR3 CAAR comprises the amino acid sequence set forth in SEQ ID NO:46, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:46 and that contains an N terminus Ile deletion. In some of any embodiments, the PR3 CAAR comprises the amino acid sequence set forth in SEQ ID NO:49, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:49 and that contains the mutation D175N. In some of any embodiments, the PR3 CAAR comprises the amino acid sequence set forth in SEQ ID NO:52, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:52 and that contains the mutation S176C. In some of any embodiments, the PR3 CAAR comprises the amino acid sequence set forth in SEQ ID NO:55, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:55 and that contains the mutation G4P. In some of any embodiments, the PR3 CAAR comprises the amino acid sequence set forth in SEQ ID NO:58, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:58 and that contains the mutation H44A.

In some embodiments, the antibody binding domain of the PR3 CAAR comprises a mutant PR3 antibody binding domain containing a mutation at position 4 compared to mature human PR3. In some embodiments, the mutation of the PR3 antibody binding domain is G4P. In some embodiments, the PR3 CAAR comprises an antibody binding domain with the amino acid sequence set forth in SEQ ID NO:9 and a spacer with the amino acid sequence set forth in SEQ ID NO:97. In some embodiments, the PR3 CAAR comprises an antibody binding domain with the amino acid sequence set forth in SEQ ID NO:9 and a spacer with the amino acid sequence set forth in SEQ ID NO:25. In some embodiments, the PR3 CAAR has the amino acid sequence set forth in SEQ ID NO:55.

In some embodiments, the antibody binding domain of the PR3 CAAR comprises a mutant PR3 antibody binding domain containing a mutation at position 44 compared to mature human PR3. In some embodiments, the mutation of the PR3 antibody binding domain is H44A. In some embodiments, the PR3 CAAR comprises an antibody binding domain with the amino acid sequence set forth in SEQ ID NO:10 and a spacer with the amino acid sequence set forth in SEQ ID NO:97. In some embodiments, the PR3 CAAR comprises an antibody binding domain with the amino acid sequence set forth in SEQ ID NO:10 and a spacer with the amino acid sequence set forth in SEQ ID NO:25. In some embodiments, the PR3 CAAR has the amino acid sequence set forth in SEQ ID NO:56.

Provided herein is a CAAR comprising any of the PR3 antibody binding domains such as any described herein, an IgG4 hinge spacer, a CD8a transmembrane domain, a 4-1BB costimulatory domain, and a CD3 zeta signaling domain. In certain embodiments, the CAAR comprises any of the PR3 antibody binding domains described herein; a spacer that is or contains the sequence set forth in SEQ ID NO: 22; a transmembrane domain that is or contains the sequence set forth in SEQ ID NO: 98; and an intracellular signaling domain comprising the 4-1BB signaling domain sequence set forth in SEQ ID NO: 27 and the CD3 zeta signaling domain sequence set forth in SEQ ID NO: 28. In some of any embodiments, the PR3 CAAR comprises the amino acid sequence set forth in SEQ ID NO:110, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:110 and that contains the mutation G4P. In some of any embodiments, the PR3 CAAR comprises the amino acid sequence set forth in SEQ ID NO:118, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:118 and that contains the mutation H44A.

Provided herein is a CAAR comprising any of the PR3 antibody binding domains such as any described herein, an IgG4 hinge spacer, a CD8a transmembrane domain, a 4-1BB costimulatory domain, and a CD3 zeta signaling domain. In certain embodiments, the CAAR comprises any of the PR3 antibody binding domains described herein; a spacer that is or contains the sequence set forth in SEQ ID NO: 24; a transmembrane domain that is or contains the sequence set forth in SEQ ID NO: 98; and an intracellular signaling domain comprising the 4-1BB signaling domain sequence set forth in SEQ ID NO: 27 and the CD3 zeta signaling domain sequence set forth in SEQ ID NO: 28. In some of any embodiments, the PR3 CAAR comprises the amino acid sequence set forth in SEQ ID NO:109, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:109 and that contains the mutation G4P. In some of any embodiments, the PR3 CAAR comprises the amino acid sequence set forth in SEQ ID NO:117, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:117 and that contains the mutation H44A.

Provided herein is a CAAR comprising any of the PR3 antibody binding domains such as any described herein, an IgG4 hinge spacer, a CD8a transmembrane domain, a 4-1BB costimulatory domain, and a CD3 zeta signaling domain. In certain embodiments, the CAAR comprises any of the PR3 antibody binding domains described herein; a spacer that is or contains the sequence set forth in SEQ ID NO: 101; a transmembrane domain that is or contains the sequence set forth in SEQ ID NO: 98; and an intracellular signaling domain comprising the 4-1BB signaling domain sequence set forth in SEQ ID NO: 27 and the CD3 zeta signaling domain sequence set forth in SEQ ID NO: 28. In some of any embodiments, the PR3 CAAR comprises the amino acid sequence set forth in SEQ ID NO:107, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:107 and that contains the mutation G4P. In some of any embodiments, the PR3 CAAR comprises the amino acid sequence set forth in SEQ ID NO:115, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:115 and that contains the mutation H44A.

Provided herein is a CAAR comprising any of the PR3 antibody binding domains such as any described herein, a spacer, a CD8a transmembrane domain, a 4-1BB costimulatory domain, and a CD3 zeta signaling domain. In certain embodiments, the CAAR comprises any of the PR3 antibody binding domains described herein; a spacer that is or contains the sequence set forth in SEQ ID NO: 102; a transmembrane domain that is or contains the sequence set forth in SEQ ID NO: 98; and an intracellular signaling domain comprising the 4-1BB signaling domain sequence set forth in SEQ ID NO: 27 and the CD3 zeta signaling domain sequence set forth in SEQ ID NO: 28. In some of any embodiments, the PR3 CAAR comprises the amino acid sequence set forth in SEQ ID NO:108, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:108 and that contains the mutation G4P. In some of any embodiments, the PR3 CAAR comprises the amino acid sequence set forth in SEQ ID NO:116, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:116 and that contains the mutation H44A.

Provided herein is a CAAR comprising any of the PR3 antibody binding domains such as any described herein, a spacer, a CD8a transmembrane domain, a 4-1BB costimulatory domain, and a CD3 zeta signaling domain. In certain embodiments, the CAAR comprises any of the PR3 antibody binding domains described herein; a spacer that is or contains the sequence set forth in SEQ ID NO: 100; a transmembrane domain that is or contains the sequence set forth in SEQ ID NO: 98; and an intracellular signaling domain comprising the 4-1BB signaling domain sequence set forth in SEQ ID NO: 27 and the CD3 zeta signaling domain sequence set forth in SEQ ID NO: 28. In some of any embodiments, the PR3 CAAR comprises the amino acid sequence set forth in SEQ ID NO:103, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:103 and that contains the mutation G4P. In some of any embodiments, the PR3 CAAR comprises the amino acid sequence set forth in SEQ ID NO:111, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:111 and that contains the mutation H44A.

Provided herein is a CAAR comprising any of the PR3 antibody binding domains such as any described herein, a spacer, a CD28 transmembrane domain, a 4-1BB costimulatory domain, and a CD3 zeta signaling domain. In certain embodiments, the CAAR comprises any of the PR3 antibody binding domains described herein; a spacer that is or contains the sequence set forth in SEQ ID NO: 100; a transmembrane domain that is or contains the sequence set forth in SEQ ID NO: 26; and an intracellular signaling domain comprising the 4-1BB signaling domain sequence set forth in SEQ ID NO: 27 and the CD3 zeta signaling domain sequence set forth in SEQ ID NO: 28. In some of any embodiments, the PR3 CAAR comprises the amino acid sequence set forth in SEQ ID NO:104, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:104 and that contains the mutation G4P. In some of any embodiments, the PR3 CAAR comprises the amino acid sequence set forth in SEQ ID NO:112, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 112 and that contains the mutation H44A.

Provided herein is a CAAR comprising any of the PR3 antibody binding domains such as any described herein, a spacer, a CD8a transmembrane domain, a 4-1BB costimulatory domain, and a CD3 zeta signaling domain. In certain embodiments, the CAAR comprises any of the PR3 antibody binding domains described herein; a spacer that is or contains the sequence set forth in SEQ ID NO: 99; a transmembrane domain that is or contains the sequence set forth in SEQ ID NO: 98; and an intracellular signaling domain comprising the 4-1BB signaling domain sequence set forth in SEQ ID NO: 27 and the CD3 zeta signaling domain sequence set forth in SEQ ID NO: 28. In some of any embodiments, the PR3 CAAR comprises the amino acid sequence set forth in SEQ ID NO:105, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:105 and that contains the mutation G4P. In some of any embodiments, the PR3 CAAR comprises the amino acid sequence set forth in SEQ ID NO:113, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:113 and that contains the mutation H44A.

Provided herein is a CAAR comprising any of the PR3 antibody binding domains such as any described herein, a spacer, a CD28 transmembrane domain, a 4-1BB costimulatory domain, and a CD3 zeta signaling domain. In certain embodiments, the CAAR comprises any of the PR3 antibody binding domains described herein; a spacer that is or contains the sequence set forth in SEQ ID NO: 99; a transmembrane domain that is or contains the sequence set forth in SEQ ID NO: 26; and an intracellular signaling domain comprising the 4-1BB signaling domain sequence set forth in SEQ ID NO: 27 and the CD3 zeta signaling domain sequence set forth in SEQ ID NO: 28. In some of any embodiments, the PR3 CAAR comprises the amino acid sequence set forth in SEQ ID NO:106, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:106 and that contains the mutation G4P. In some of any embodiments, the PR3 CAAR comprises the amino acid sequence set forth in SEQ ID NO:114, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:114 and that contains the mutation H44A.

In some of any embodiments, the PR3 CAAR comprises the amino acid sequence set forth in SEQ ID NO:119, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:119 and that contains the mutation H44A. In some of any embodiments, the PR3 CAAR comprises the amino acid sequence set forth in SEQ ID NO: 120, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:120 and that contains the mutation H44A. In some of any embodiments, the PR3 CAAR comprises the amino acid sequence set forth in SEQ ID NO:121, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:121 and that contains the mutation H44A. In some of any embodiments, the PR3 CAAR comprises the amino acid sequence set forth in SEQ ID NO:122, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:122 and that contains the mutation H44A.

In some of any of the provided embodiments, the PR3 CAAR is encoded by the sequence set forth in SEQ ID NOs: 59-88 and 144-159 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NOs: 59-88 and 144-159. In some of any embodiments, the PR3 CAAR is encoded by the nucleotide sequence set forth in SEQ ID NO:59, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:59. In some of any embodiments, the PR3 CAAR is encoded by the nucleotide sequence set forth in SEQ ID NO:60, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:60. In some of any embodiments, the PR3 CAAR is encoded by the nucleotide sequence set forth in SEQ ID NO:61, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:61.

In some of any embodiments, the PR3 CAAR is encoded by the nucleotide sequence set forth in SEQ ID NO:62, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:62. In some of any embodiments, the PR3 CAAR is encoded by the nucleotide sequence set forth in SEQ ID NO:63, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:63. In some of any embodiments, the PR3 CAAR is encoded by the nucleotide sequence set forth in SEQ ID NO:64, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:64.

In some of any embodiments, the PR3 CAAR is encoded by the nucleotide sequence set forth in SEQ ID NO:65, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:65. In some of any embodiments, the PR3 CAAR is encoded by the nucleotide sequence set forth in SEQ ID NO:66, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:66. In some of any embodiments, the PR3 CAAR is encoded by the nucleotide sequence set forth in SEQ ID NO:67, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:67.

In some of any embodiments, the PR3 CAAR is encoded by the nucleotide sequence set forth in SEQ ID NO:68, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:68. In some of any embodiments, the PR3 CAAR is encoded by the nucleotide sequence set forth in SEQ ID NO:69, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:69. In some of any embodiments, the PR3 CAAR is encoded by the nucleotide sequence set forth in SEQ ID NO:70, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:70.

In some of any embodiments, the PR3 CAAR is encoded by the nucleotide sequence set forth in SEQ ID NO:71, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:71. In some of any embodiments, the PR3 CAAR is encoded by the nucleotide sequence set forth in SEQ ID NO:72, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:72. In some of any embodiments, the PR3 CAAR is encoded by the nucleotide sequence set forth in SEQ ID NO:73, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:73.

In some of any embodiments, the PR3 CAAR is encoded by the nucleotide sequence set forth in SEQ ID NO:74, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:74. In some of any embodiments, the PR3 CAAR is encoded by the nucleotide sequence set forth in SEQ ID NO:75, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:75. In some of any embodiments, the PR3 CAAR is encoded by the nucleotide sequence set forth in SEQ ID NO:76, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:76.

In some of any embodiments, the PR3 CAAR is encoded by the nucleotide sequence set forth in SEQ ID NO:77, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:77. In some of any embodiments, the PR3 CAAR is encoded by the nucleotide sequence set forth in SEQ ID NO:78, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:78. In some of any embodiments, the PR3 CAAR is encoded by the nucleotide sequence set forth in SEQ ID NO:79, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:79.

In some of any embodiments, the PR3 CAAR is encoded by the nucleotide sequence set forth in SEQ ID NO:80, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:80. In some of any embodiments, the PR3 CAAR is encoded by the nucleotide sequence set forth in SEQ ID NO:81, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:81. In some of any embodiments, the PR3 CAAR is encoded by the nucleotide sequence set forth in SEQ ID NO:82, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:82.

In some of any embodiments, the PR3 CAAR is encoded by the nucleotide sequence set forth in SEQ ID NO:83, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:83. In some of any embodiments, the PR3 CAAR is encoded by the nucleotide sequence set forth in SEQ ID NO:84, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:84. In some of any embodiments, the PR3 CAAR is encoded by the nucleotide sequence set forth in SEQ ID NO:85, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:85.

In some of any embodiments, the PR3 CAAR is encoded by the nucleotide sequence set forth in SEQ ID NO:86, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:86. In some of any embodiments, the PR3 CAAR is encoded by the nucleotide sequence set forth in SEQ ID NO:87, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:87. In some of any embodiments, the PR3 CAAR is encoded by the nucleotide sequence set forth in SEQ ID NO:88, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:88.

In some of any embodiments, the PR3 CAAR is encoded by the nucleotide sequence set forth in SEQ ID NO:144, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:144. In some of any embodiments, the PR3 CAAR is encoded by the nucleotide sequence set forth in SEQ ID NO:145, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:145. In some of any embodiments, the PR3 CAAR is encoded by the nucleotide sequence set forth in SEQ ID NO:146, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO: 146.

In some of any embodiments, the PR3 CAAR is encoded by the nucleotide sequence set forth in SEQ ID NO:147, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:147. In some of any embodiments, the PR3 CAAR is encoded by the nucleotide sequence set forth in SEQ ID NO:148, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:148. In some of any embodiments, the PR3 CAAR is encoded by the nucleotide sequence set forth in SEQ ID NO:149, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:149.

In some of any embodiments, the PR3 CAAR is encoded by the nucleotide sequence set forth in SEQ ID NO:150, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:150. In some of any embodiments, the PR3 CAAR is encoded by the nucleotide sequence set forth in SEQ ID NO:151, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:151. In some of any embodiments, the PR3 CAAR is encoded by the nucleotide sequence set forth in SEQ ID NO:152, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO: 152.

In some of any embodiments, the PR3 CAAR is encoded by the nucleotide sequence set forth in SEQ ID NO:153, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:153. In some of any embodiments, the PR3 CAAR is encoded by the nucleotide sequence set forth in SEQ ID NO:154, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:154. In some of any embodiments, the PR3 CAAR is encoded by the nucleotide sequence set forth in SEQ ID NO:155, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:155.

In some of any embodiments, the PR3 CAAR is encoded by the nucleotide sequence set forth in SEQ ID NO:156, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:156. In some of any embodiments, the PR3 CAAR is encoded by the nucleotide sequence set forth in SEQ ID NO:157, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:157. In some of any embodiments, the PR3 CAAR is encoded by the nucleotide sequence set forth in SEQ ID NO:158, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:158.

In some of any embodiments, the PR3 CAAR is encoded by the nucleotide sequence set forth in SEQ ID NO:159, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:159.

In some of any such embodiments, the PR3 CAAR is or contains the sequence set forth in SEQ ID NO:29. In some of any such embodiments, the PR3 CAAR is or contains the amino acid sequence encoded by SEQ ID NO: 59 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 59. In some of any such embodiments, the PR3 CAAR is or contains the amino acid sequence encoded by SEQ ID NO: 59.

In some of any such embodiments, the PR3 CAAR is or contains the sequence set forth in SEQ ID NO:30. In some of any such embodiments, the PR3 CAAR is or contains the amino acid sequence encoded by SEQ ID NO: 60 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 60. In some of any such embodiments, the PR3 CAAR is or contains the amino acid sequence encoded by SEQ ID NO: 60.

In some of any such embodiments, the PR3 CAAR is or contains the sequence set forth in SEQ ID NO:31. In some of any such embodiments, the PR3 CAAR is or contains the amino acid sequence encoded by SEQ ID NO: 61 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 61. In some of any such embodiments, the PR3 CAAR is or contains the amino acid sequence encoded by SEQ ID NO: 61.

In some of any such embodiments, the PR3 CAAR is or contains the sequence set forth in SEQ ID NO:32. In some of any such embodiments, the PR3 CAAR is or contains the amino acid sequence encoded by SEQ ID NO: 62 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 62 and that contains the mutation S176A. In some of any such embodiments, the PR3 CAAR is or contains the amino acid sequence encoded by SEQ ID NO: 62.

In some of any such embodiments, the PR3 CAAR is or contains the sequence set forth in SEQ ID NO:33. In some of any such embodiments, the PR3 CAAR is or contains the amino acid sequence encoded by SEQ ID NO: 63 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 63 and that contains the mutation S176A. In some of any such embodiments, the PR3 CAAR is or contains the amino acid sequence encoded by SEQ ID NO: 63.

In some of any such embodiments, the PR3 CAAR is or contains the sequence set forth in SEQ ID NO:34. In some of any such embodiments, the PR3 CAAR is or contains the amino acid sequence encoded by SEQ ID NO: 64 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 64 and that contains the mutation S176A. In some of any such embodiments, the PR3 CAAR is or contains the amino acid sequence encoded by SEQ ID NO: 64.

In some of any such embodiments, the PR3 CAAR is or contains the sequence set forth in SEQ ID NO:35. In some of any such embodiments, the PR3 CAAR is or contains the amino acid sequence encoded by SEQ ID NO: 65 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 65 and that contains an unprocessed C terminus. In some of any such embodiments, the PR3 CAAR is or contains the amino acid sequence encoded by SEQ ID NO: 65.

In some of any such embodiments, the PR3 CAAR is or contains the sequence set forth in SEQ ID NO:36. In some of any such embodiments, the PR3 CAAR is or contains the amino acid sequence encoded by SEQ ID NO: 66 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 66 and that contains an unprocessed C terminus. In some of any such embodiments, the PR3 CAAR is or contains the amino acid sequence encoded by SEQ ID NO: 66.

In some of any such embodiments, the PR3 CAAR is or contains the sequence set forth in SEQ ID NO:37. In some of any such embodiments, the PR3 CAAR is or contains the amino acid sequence encoded by SEQ ID NO: 67 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 67 and that contains an unprocessed C terminus. In some of any such embodiments, the PR3 CAAR is or contains the amino acid sequence encoded by SEQ ID NO: 67.

In some of any such embodiments, the PR3 CAAR is or contains the sequence set forth in SEQ ID NO:38. In some of any such embodiments, the PR3 CAAR is or contains the amino acid sequence encoded by SEQ ID NO: 68 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 68 and that contains the mutation D91N. In some of any such embodiments, the PR3 CAAR is or contains the amino acid sequence encoded by SEQ ID NO: 68.

In some of any such embodiments, the PR3 CAAR is or contains the sequence set forth in SEQ ID NO:39. In some of any such embodiments, the PR3 CAAR is or contains the amino acid sequence encoded by SEQ ID NO: 69 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 69 and that contains the mutation D91N. In some of any such embodiments, the PR3 CAAR is or contains the amino acid sequence encoded by SEQ ID NO: 69.

In some of any such embodiments, the PR3 CAAR is or contains the sequence set forth in SEQ ID NO:40. In some of any such embodiments, the PR3 CAAR is or contains the amino acid sequence encoded by SEQ ID NO: 70 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 70 and that contains the mutation D91N. In some of any such embodiments, the PR3 CAAR is or contains the amino acid sequence encoded by SEQ ID NO: 70.

In some of any such embodiments, the PR3 CAAR is or contains the sequence set forth in SEQ ID NO:41. In some of any such embodiments, the PR3 CAAR is or contains the amino acid sequence encoded by SEQ ID NO: 71 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 71 and that contains an unprocessed N terminus. In some of any such embodiments, the PR3 CAAR is or contains the amino acid sequence encoded by SEQ ID NO: 71.

In some of any such embodiments, the PR3 CAAR is or contains the sequence set forth in SEQ ID NO:42. In some of any such embodiments, the PR3 CAAR is or contains the amino acid sequence encoded by SEQ ID NO: 72 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 72 and that contains an unprocessed N terminus. In some of any such embodiments, the PR3 CAAR is or contains the amino acid sequence encoded by SEQ ID NO: 72.

In some of any such embodiments, the PR3 CAAR is or contains the sequence set forth in SEQ ID NO:43. In some of any such embodiments, the PR3 CAAR is or contains the amino acid sequence encoded by SEQ ID NO: 73 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 73 and that contains an unprocessed N terminus. In some of any such embodiments, the PR3 CAAR is or contains the amino acid sequence encoded by SEQ ID NO: 73.

In some of any such embodiments, the PR3 CAAR is or contains the sequence set forth in SEQ ID NO:44. In some of any such embodiments, the PR3 CAAR is or contains the amino acid sequence encoded by SEQ ID NO: 74 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 74 and that contains the N terminus Ile deletion. In some of any such embodiments, the PR3 CAAR is or contains the amino acid sequence encoded by SEQ ID NO: 74.

In some of any such embodiments, the PR3 CAAR is or contains the sequence set forth in SEQ ID NO:45. In some of any such embodiments, the PR3 CAAR is or contains the amino acid sequence encoded by SEQ ID NO: 75 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 75 and that contains the N terminus Ile deletion. In some of any such embodiments, the PR3 CAAR is or contains the amino acid sequence encoded by SEQ ID NO: 75.

In some of any such embodiments, the PR3 CAAR is or contains the sequence set forth in SEQ ID NO:46. In some of any such embodiments, the PR3 CAAR is or contains the amino acid sequence encoded by SEQ ID NO: 76 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 76 and that contains the N terminus Ile deletion. In some of any such embodiments, the PR3 CAAR is or contains the amino acid sequence encoded by SEQ ID NO: 76.

In some of any such embodiments, the PR3 CAAR is or contains the sequence set forth in SEQ ID NO:47. In some of any such embodiments, the PR3 CAAR is or contains the amino acid sequence encoded by SEQ ID NO: 77 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 77 and that contains the mutation D175N. In some of any such embodiments, the PR3 CAAR is or contains the amino acid sequence encoded by SEQ ID NO: 77.

In some of any such embodiments, the PR3 CAAR is or contains the sequence set forth in SEQ ID NO:48. In some of any such embodiments, the PR3 CAAR is or contains the amino acid sequence encoded by SEQ ID NO: 78 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 78 and that contains the mutation D175N. In some of any such embodiments, the PR3 CAAR is or contains the amino acid sequence encoded by SEQ ID NO: 78.

In some of any such embodiments, the PR3 CAAR is or contains the sequence set forth in SEQ ID NO:49. In some of any such embodiments, the PR3 CAAR is or contains the amino acid sequence encoded by SEQ ID NO: 79 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 79 and that contains the mutation D175N. In some of any such embodiments, the PR3 CAAR is or contains the amino acid sequence encoded by SEQ ID NO: 79.

In some of any such embodiments, the PR3 CAAR is or contains the sequence set forth in SEQ ID NO:50. In some of any such embodiments, the PR3 CAAR is or contains the amino acid sequence encoded by SEQ ID NO: 80 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 80 and that contains the mutation S176C. In some of any such embodiments, the PR3 CAAR is or contains the amino acid sequence encoded by SEQ ID NO: 80.

In some of any such embodiments, the PR3 CAAR is or contains the sequence set forth in SEQ ID NO:51. In some of any such embodiments, the PR3 CAAR is or contains the amino acid sequence encoded by SEQ ID NO: 81 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 81 and that contains the mutation S176C. In some of any such embodiments, the PR3 CAAR is or contains the amino acid sequence encoded by SEQ ID NO: 81.

In some of any such embodiments, the PR3 CAAR is or contains the sequence set forth in SEQ ID NO:52. In some of any such embodiments, the PR3 CAAR is or contains the amino acid sequence encoded by SEQ ID NO: 82 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO:82 and that contains the mutation S176C. In some of any such embodiments, the PR3 CAAR is or contains the amino acid sequence encoded by SEQ ID NO: 82.

In some of any such embodiments, the PR3 CAAR is or contains the sequence set forth in SEQ ID NO:53. In some of any such embodiments, the PR3 CAAR is or contains the amino acid sequence encoded by SEQ ID NO: 83 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 83 and that contains the mutation G4P. In some of any such embodiments, the PR3 CAAR is or contains the amino acid sequence encoded by SEQ ID NO: 83.

In some of any such embodiments, the PR3 CAAR is or contains the sequence set forth in SEQ ID NO:54. In some of any such embodiments, the PR3 CAAR is or contains the amino acid sequence encoded by SEQ ID NO: 84 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 84 and that contains the mutation G4P. In some of any such embodiments, the PR3 CAAR is or contains the amino acid sequence encoded by SEQ ID NO: 84.

In some of any such embodiments, the PR3 CAAR is or contains the sequence set forth in SEQ ID NO:55. In some embodiments, the PR3 CAAR comprises the amino acid sequence that is at least at or about 90% sequence identity to the amino acid sequence set forth in SEQ ID NO: 55 and that contains the mutation G4P. In some embodiments, the PR3 CAAR comprises the amino acid sequence that is at least at or about 95% sequence identity to the amino acid sequence set forth in SEQ ID NO: 55 and that contains the mutation G4P.

In some embodiments, the PR3 CAAR comprises the amino acid sequence that is at least at or about 97% sequence identity to the amino acid sequence set forth in SEQ ID NO: 55 and that contains the mutation G4P. In some embodiments, the PR3 CAAR comprises the amino acid sequence that is at least at or about 99% sequence identity to the amino acid sequence set forth in SEQ ID NO: 55 and that contains the mutation G4P. In some of any such embodiments, the PR3 CAAR is or contains the amino acid sequence encoded by SEQ ID NO: 85 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 85 and that contains the mutation G4P. In some of any such embodiments, the PR3 CAAR is or contains the amino acid sequence encoded by SEQ ID NO: 85.

In some of any such embodiments, the PR3 CAAR is or contains the sequence set forth in SEQ ID NO:56. In some of any such embodiments, the PR3 CAAR is or contains the amino acid sequence encoded by SEQ ID NO: 86 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 86 and that contains the mutation H44A. In some of any such embodiments, the PR3 CAAR is or contains the amino acid sequence encoded by SEQ ID NO: 86.

In some of any such embodiments, the PR3 CAAR is or contains the sequence set forth in SEQ ID NO:57. In some embodiments, the PR3 CAAR comprises the amino acid sequence that is at least at or about 90% sequence identity to the amino acid sequence set forth in SEQ ID NO: 57 and that contains the mutation H44A. In some embodiments, the PR3 CAAR comprises the amino acid sequence that is at least at or about 95% sequence identity to the amino acid sequence set forth in SEQ ID NO: 57 and that contains the mutation H44A. In some embodiments, the PR3 CAAR comprises the amino acid sequence that is at least at or about 97% sequence identity to the amino acid sequence set forth in SEQ ID NO: 57 and that contains the mutation H44A. In some embodiments, the PR3 CAAR comprises the amino acid sequence that is at least at or about 99% sequence identity to the amino acid sequence set forth in SEQ ID NO: 57 and that contains the mutation H44A. In some of any such embodiments, the PR3 CAAR is or contains the amino acid sequence encoded by SEQ ID NO: 87 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 87 and that contains the mutation H44A. In some of any such embodiments, the PR3 CAAR is or contains the amino acid sequence encoded by SEQ ID NO: 87.

In some of any such embodiments, the PR3 CAAR is or contains the sequence set forth in SEQ ID NO:58. In some of any such embodiments, the PR3 CAAR is or contains the amino acid sequence encoded by SEQ ID NO: 88 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 88 and that contains the mutation H44A. In some of any such embodiments, the PR3 CAAR is or contains the amino acid sequence encoded by SEQ ID NO: 88.

In some of any such embodiments, the PR3 CAAR is or contains the sequence set forth in SEQ ID NO:103. In some of any such embodiments, the PR3 CAAR is or contains the amino acid sequence encoded by SEQ ID NO: 144 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 144 and that contains the mutation G4P. In some of any such embodiments, the PR3 CAAR is or contains the amino acid sequence encoded by SEQ ID NO: 144.

In some of any such embodiments, the PR3 CAAR is or contains the sequence set forth in SEQ ID NO:104. In some embodiments, the PR3 CAAR comprises the amino acid sequence that is at least at or about 90% sequence identity to the amino acid sequence set forth in SEQ ID NO: 104 and that contains the mutation G4P. In some embodiments, the PR3 CAAR comprises the amino acid sequence that is at least at or about 95% sequence identity to the amino acid sequence set forth in SEQ ID NO: 104 and that contains the mutation G4P. In some embodiments, the PR3 CAAR comprises the amino acid sequence that is at least at or about 97% sequence identity to the amino acid sequence set forth in SEQ ID NO: 104 and that contains the mutation G4P. In some embodiments, the PR3 CAAR comprises the amino acid sequence that is at least at or about 99% sequence identity to the amino acid sequence set forth in SEQ ID NO: 104 and that contains the mutation G4P. In some of any such embodiments, the PR3 CAAR is or contains the amino acid sequence encoded by SEQ ID NO: 145 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 145 and that contains the mutation G4P. In some of any such embodiments, the PR3 CAAR is or contains the amino acid sequence encoded by SEQ ID NO: 145.

In some of any such embodiments, the PR3 CAAR is or contains the sequence set forth in SEQ ID NO:105. In some of any such embodiments, the PR3 CAAR is or contains the amino acid sequence encoded by SEQ ID NO: 146 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 146 and that contains the mutation G4P. In some of any such embodiments, the PR3 CAAR is or contains the amino acid sequence encoded by SEQ ID NO: 146.

In some of any such embodiments, the PR3 CAAR is or contains the sequence set forth in SEQ ID NO:106. In some of any such embodiments, the PR3 CAAR is or contains the amino acid sequence encoded by SEQ ID NO: 147 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 147 and that contains the mutation G4P. In some of any such embodiments, the PR3 CAAR is or contains the amino acid sequence encoded by SEQ ID NO: 147.

In some of any such embodiments, the PR3 CAAR is or contains the sequence set forth in SEQ ID NO:107. In some of any such embodiments, the PR3 CAAR is or contains the amino acid sequence encoded by SEQ ID NO: 148 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 148 and that contains the mutation G4P. In some of any such embodiments, the PR3 CAAR is or contains the amino acid sequence encoded by SEQ ID NO: 148.

In some of any such embodiments, the PR3 CAAR is or contains the sequence set forth in SEQ ID NO:108. In some of any such embodiments, the PR3 CAAR is or contains the amino acid sequence encoded by SEQ ID NO: 149 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 149 and that contains the mutation G4P. In some of any such embodiments, the PR3 CAAR is or contains the amino acid sequence encoded by SEQ ID NO: 149.

In some of any such embodiments, the PR3 CAAR is or contains the sequence set forth in SEQ ID NO:109. In some of any such embodiments, the PR3 CAAR is or contains the amino acid sequence encoded by SEQ ID NO: 150 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 150 and that contains the mutation G4P. In some of any such embodiments, the PR3 CAAR is or contains the amino acid sequence encoded by SEQ ID NO: 150.

In some of any such embodiments, the PR3 CAAR is or contains the sequence set forth in SEQ ID NO:110. In some embodiments, the PR3 CAAR comprises the amino acid sequence that is at least at or about 90% sequence identity to the amino acid sequence set forth in SEQ ID NO: 110 and that contains the mutation G4P. In some embodiments, the PR3 CAAR comprises the amino acid sequence that is at least at or about 95% sequence identity to the amino acid sequence set forth in SEQ ID NO: 110 and that contains the mutation G4P. In some embodiments, the PR3 CAAR comprises the amino acid sequence that is at least at or about 97% sequence identity to the amino acid sequence set forth in SEQ ID NO: 110 and that contains the mutation G4P. In some embodiments, the PR3 CAAR comprises the amino acid sequence that is at least at or about 99% sequence identity to the amino acid sequence set forth in SEQ ID NO: 110 and that contains the mutation G4P. In some of any such embodiments, the PR3 CAAR is or contains the amino acid sequence encoded by SEQ ID NO: 151 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 151 and that contains the mutation G4P. In some of any such embodiments, the PR3 CAAR is or contains the amino acid sequence encoded by SEQ ID NO: 151.

In some of any such embodiments, the PR3 CAAR is or contains the sequence set forth in SEQ ID NO:111. In some embodiments, the PR3 CAAR comprises the amino acid sequence that is at least at or about 90% sequence identity to the amino acid sequence set forth in SEQ ID NO: 111 and that contains the mutation H44A. In some embodiments, the PR3 CAAR comprises the amino acid sequence that is at least at or about 95% sequence identity to the amino acid sequence set forth in SEQ ID NO: 111 and that contains the mutation H44A. In some embodiments, the PR3 CAAR comprises the amino acid sequence that is at least at or about 97% sequence identity to the amino acid sequence set forth in SEQ ID NO: 111 and that contains the mutation H44A. In some embodiments, the PR3 CAAR comprises the amino acid sequence that is at least at or about 99% sequence identity to the amino acid sequence set forth in SEQ ID NO: 111 and that contains the mutation H44A. In some of any such embodiments, the PR3 CAAR is or contains the amino acid sequence encoded by SEQ ID NO: 152 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 152 and that contains the mutation H44A. In some of any such embodiments, the PR3 CAAR is or contains the amino acid sequence encoded by SEQ ID NO: 152.

In some of any such embodiments, the PR3 CAAR is or contains the sequence set forth in SEQ ID NO:112. In some of any such embodiments, the PR3 CAAR is or contains the amino acid sequence encoded by SEQ ID NO: 153 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 153 and that contains the mutation H44A. In some of any such embodiments, the PR3 CAAR is or contains the amino acid sequence encoded by SEQ ID NO: 153.

In some of any such embodiments, the PR3 CAAR is or contains the sequence set forth in SEQ ID NO:113. In some of any such embodiments, the PR3 CAAR is or contains the amino acid sequence encoded by SEQ ID NO: 154 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 154 and that contains the mutation H44A. In some of any such embodiments, the PR3 CAAR is or contains the amino acid sequence encoded by SEQ ID NO: 154.

In some of any such embodiments, the PR3 CAAR is or contains the sequence set forth in SEQ ID NO:114. In some of any such embodiments, the PR3 CAAR is or contains the amino acid sequence encoded by SEQ ID NO: 155 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 155 and that contains the mutation H44A. In some of any such embodiments, the PR3 CAAR is or contains the amino acid sequence encoded by SEQ ID NO: 155.

In some of any such embodiments, the PR3 CAAR is or contains the sequence set forth in SEQ ID NO:115. In some of any such embodiments, the PR3 CAAR is or contains the amino acid sequence encoded by SEQ ID NO: 156 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 156 and that contains the mutation H44A. In some of any such embodiments, the PR3 CAAR is or contains the amino acid sequence encoded by SEQ ID NO: 156.

In some of any such embodiments, the PR3 CAAR is or contains the sequence set forth in SEQ ID NO:116. In some of any such embodiments, the PR3 CAAR is or contains the amino acid sequence encoded by SEQ ID NO: 157 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 157 and that contains the mutation H44A. In some of any such embodiments, the PR3 CAAR is or contains the amino acid sequence encoded by SEQ ID NO: 157.

In some of any such embodiments, the PR3 CAAR is or contains the sequence set forth in SEQ ID NO:117. In some of any such embodiments, the PR3 CAAR is or contains the amino acid sequence encoded by SEQ ID NO: 158 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 158 and that contains the mutation H44A. In some of any such embodiments, the PR3 CAAR is or contains the amino acid sequence encoded by SEQ ID NO: 158.

In some of any such embodiments, the PR3 CAAR is or contains the sequence set forth in SEQ ID NO:118. In some of any such embodiments, the PR3 CAAR is or contains the amino acid sequence encoded by SEQ ID NO: 159 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 159 and that contains the mutation H44A. In some of any such embodiments, the PR3 CAAR is or contains the amino acid sequence encoded by SEQ ID NO: 159.

In some of any such embodiments, the PR3 CAAR is or contains the sequence set forth in SEQ ID NO:119. In some embodiments, the PR3 CAAR comprises the amino acid sequence that is at least at or about 90% sequence identity to the amino acid sequence set forth in SEQ ID NO: 119 and that contains the mutation H44A. In some embodiments, the PR3 CAAR comprises the amino acid sequence that is at least at or about 95% sequence identity to the amino acid sequence set forth in SEQ ID NO: 119 and that contains the mutation H44A. In some embodiments, the PR3 CAAR comprises the amino acid sequence that is at least at or about 97% sequence identity to the amino acid sequence set forth in SEQ ID NO: 119 and that contains the mutation H44A. In some embodiments, the PR3 CAAR comprises the amino acid sequence that is at least at or about 99% sequence identity to the amino acid sequence set forth in SEQ ID NO: 119 and that contains the mutation H44A.

In some of any such embodiments, the PR3 CAAR is or contains the sequence set forth in SEQ ID NO:120. In some of any such embodiments, the PR3 CAAR is or contains the sequence set forth in SEQ ID NO:121. In some of any such embodiments, the PR3 CAAR is or contains the sequence set forth in SEQ ID NO:122.

In some embodiments, the provided CAAR-expressing cells exhibit biological activity or function, including cytotoxic activity, cytokine production, and ability to proliferate.

In some embodiments, biological activity or functional activity of a chimeric receptor, such as cytotoxic activity, can be measured using any of a number of known methods. The activity can be assessed or determined either in vitro or in vivo. In some embodiments, activity can be assessed once the cells are administered to the subject (e.g., human). Parameters to assess include specific binding of an engineered or natural T cell or other immune cell to target binding partner (e.g. PR3-ANCA), e.g., in vivo, e.g., by imaging, or ex vivo, e.g., by ELISA or flow cytometry. In certain embodiments, the ability of the engineered cells to destroy target cells can be measured using any suitable method, such as cytotoxicity assays described herein in the Examples. In some embodiments, the provided CAARs are able to kill at least 50%, 55%, 60%, 65%, 70%, 75%, or 80% of an anti-PR3 antibody expressing cell target. In some embodiments, the CAAR is able to kill at least 65% of an anti-PR3 antibody expressing cell target, such as assessed using the assay described in Example 3. Other cytotoxicity assays are known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as interlekukin-2 (IL-2), interferon-gamma (IFNγ), interleukin-4 (IL-4), TNF-alpha (TNFα), interleukin-6 (IL-6), interleukin-10 (IL-10), interleukin-12 (IL-12), granulocyte-macrophage colony-stimulating factor (GM-CSF), CD107a, and/or TGF-beta (TGFβ). Assays to measure cytokines are well known in the art, and include but are not limited to, ELISA, intracellular cytokine staining, cytometric bead array, RT-PCR, ELISPOT, flow cytometry and bio-assays in which cells responsive to the relevant cytokine are tested for responsiveness (e.g. proliferation) in the presence of a test sample. In some aspects, the biological activity is measured by assessing clinical outcome.

In some embodiments, among the provided CAARs are CAARs that exhibit target-dependent activity or signaling, i.e. signaling activity that is measurably absent or at background levels in the absence of target, e.g. PR3-ANCA. Thus, in some aspects, provided CAARs do not exhibit, or exhibit no more than background or a tolerable or low level of, tonic signaling. Tonic signaling refers to CAAR activity (e.g. cytotoxicity activity or other functional activity such as production of cytokines) that may occur and be target-independent activity or signaling in the absence of target, e.g. PR3-ANCA, being present. In some embodiments, among a among a plurality of the cells in the composition expressing the CAAR, less than at or about 10%, at or about 9%, at or about 8%, at or about 7%, at or about 5%, at or about 4%, at or about 3%, at or about 2% or at or about 1% of the cells in the plurality exhibits tonic signaling and/or antigen independent activity or signaling. In some embodiments, antigen independent activity or signaling is represent by off-target killing as described in Example 8.

In some aspects, a reporter cell line can be employed to monitor antigen-independent activity and/or tonic signaling through CAAR-expressing cells. In some embodiments, a T cell line, such as a Jurkat cell line, contains a reporter molecule, such as a fluorescent protein or other detectable molecule, such as a red fluorescent protein, expressed under the control of the endogenous Nur77 transcriptional regulatory elements. In some embodiments, the Nur77 reporter expression is cell intrinsic and dependent upon signaling through a recombinant reporter containing a primary activation signal in a T cell, a signaling domain of a T cell receptor (TCR) component, and/or a signaling domain comprising an immunoreceptor tyrosine-based activation motif (ITAM), such as a CD3 chain. Nur77 expression is generally not affected by other signaling pathways such as cytokine signaling or toll-like receptor (TLR) signaling, which may act in a cell extrinsic manner and may not depend on signaling through the recombinant receptor. Thus, only cells that express the exogenous CAAR containing the appropriate signaling regions is capable of expressing Nur77 upon stimulation (e.g., binding of the specific antigen). In some cases, Nur77 expression also can show a dose-dependent response to the amount of stimulation (e.g., antigen).

An exemplary reporter assay to assess tonic signaling is described in Example 2. In some embodiments, the cells transduced with the CAAR show antigen-specific activation after exposure to an anti-human PR3 antibody. In some embodiments, the anti-human PR3 antibody is MAB684022.

### F. Polynucleotides

Provided are polynucleotides encoding a therapeutic protein containing a PR3 antibody binding domain as provided herein. Also provided are polynucleotides encoding the CAARs described herein. The polynucleotides may include those encompassing natural and/or non-naturally occurring nucleotides and bases, e.g., including those with backbone modifications. The terms “nucleic acid molecule”, “nucleic acid”, “sequence of nucleotides”, and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

Provided are polynucleotides that contain a nucleic acid encoding any of the PR3 antibody binding domain constructs described herein. Also provided are polynucleotides that contain a nucleic acid encoding any of the PR3 CAARs described herein. In some embodiments, the PR3 CAARs contain multiple domains, and all of the CAAR is encoded in more than one polynucleotide, such as two or more polynucleotides. In some embodiments, the polynucleotides are comprised in a vector.

In some cases, the polynucleotide encoding the PR3 CAARs contains a signal sequence that encodes a signal peptide, in some cases encoded upstream of the nucleic acid sequences encoding the PR3 CAAR, or joined at the 5′ terminus of the nucleic acid sequences encoding the antigen-binding domain. In some cases, the polynucleotide containing nucleic acid sequences encoding the PR3 CAAR contains a signal sequence that encodes a signal peptide. In some aspects, the signal sequence may encode a signal peptide derived from a native polypeptide. In other aspects, the signal sequence may encode a heterologous or non-native signal peptide. In some aspects, non-limiting exemplary signal peptide include a signal peptide of a CD33 signal peptide set forth in SEQ ID NO:21. In some aspects, non-limiting exemplary signal peptide include a signal peptide of a PR3 signal peptide set forth in SEQ ID NO:123. In some cases, the polynucleotide encoding the PR3 CAARs can contain nucleic acid sequence encoding additional molecules, such as a surrogate marker or other markers, or can contain additional components, such as promoters, regulatory elements and/or multicistronic elements. In some embodiments, the nucleic acid sequence encoding the PR3 CAARs can be operably linked to any of the additional components.

In some embodiments, the polynucleotides contains a nucleic acid sequence encoding any of the PR3 CAARs described in Section I.F. In some embodiments, the nucleic acid sequences include any as described in Section I.F.

In some embodiments, provided are polynucleotides containing nucleic acid sequences encoding a PR3 antibody binding domain construct or a PR3 CAAR containing the PR3 antibody binding domain construct. In some of any embodiments, said polynucleotide contains a nucleic acid encoding the PR3 antibody binding domain construct comprising the sequence set forth in any one of SEQ ID NOs: 11-20 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to any one of SEQ ID NOs: 11-20.

In some of any embodiments, said polynucleotide contains a nucleic acid encoding the PR3 antibody binding domain comprising the sequence set forth in SEQ ID NO: 11 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 11. In some embodiments, said polynucleotide contains a nucleic acid encoding the PR3 antibody binding domain comprising the sequence set forth in SEQ ID NO: 12, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 12. In some of any embodiments, said polynucleotide contains a nucleic acid encoding the PR3 antibody binding domain comprising the sequence set forth in SEQ ID NO: 13 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 13. In some embodiments, said polynucleotide contains a nucleic acid encoding the PR3 antibody binding domain comprising the sequence set forth in SEQ ID NO: 14, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 14. In some embodiments, said polynucleotide contains a nucleic acid encoding the PR3 antibody binding domain comprising the sequence set forth in SEQ ID NO: 15, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 15.

In some of any embodiments, said polynucleotide contains a nucleic acid encoding the PR3 antibody binding domain comprising the sequence set forth in SEQ ID NO: 16 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 16. In some embodiments, said polynucleotide contains a nucleic acid encoding the PR3 antibody binding domain comprising the sequence set forth in SEQ ID NO: 17, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 17. In some of any embodiments, said polynucleotide contains a nucleic acid encoding the PR3 antibody binding domain comprising the sequence set forth in SEQ ID NO: 18 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 18. In some embodiments, said polynucleotide contains a nucleic acid encoding the PR3 antibody binding domain comprising the sequence set forth in SEQ ID NO: 19, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 19. In some embodiments, said polynucleotide contains a nucleic acid encoding the PR3 antibody binding domain comprising the sequence set forth in SEQ ID NO: 20, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 20.

Also provided are polynucleotides that have been optimized for codon usage and/or to eliminate splice sites, such as cryptic splice sites. In some embodiments, the polynucleotides are modified to optimize codon usage. In some embodiments, the polynucleotides are codon optimized for expression in a human cell such as a human T cell such as a primary human T cell. In some embodiments, the polynucleotides, such as those encoding any of the PR3 constructs or the PR3 CAARs provided herein, are or have been modified to reduce heterogeneity or contain one or more nucleic acid sequences observed herein (such as by the optimization methods) to result in improved features of the polypeptides, such as the CAARs, as compared to those containing distinct, reference, sequences or that have not been optimized. In some embodiments, the polynucleotide is optimized by splice site elimination. Among such features include improvements in RNA heterogeneity, such as that resulting from the presence of one or more splice sites, such as one or more cryptic splice sites, and/or improved expression and/or surface expression of the encoded protein, such as increased levels, uniformity, or consistency of expression among cells or different therapeutic cell compositions engineered to express the polypeptides. In some embodiments, the polynucleotides can be codon optimized for expression in human cells.

Genomic nucleic acid sequences generally, in nature, in a mammalian cell, undergo processing co-transcriptionally or immediately following transcription, wherein a nascent precursor messenger ribonucleic acid (pre-mRNA), transcribed from a genomic deoxyribonucleic acid (DNA) sequence, is in some cases edited by way of splicing, to remove introns, followed by ligation of the exons in eukaryotic cells. Consensus sequences for splice sites are known, but in some aspects, specific nucleotide information defining a splice site may be complex and may not be readily apparent based on available methods. Cryptic splice sites are splice sites that are not predicted based on the standard consensus sequences and are variably activated. Hence, variable splicing of pre-mRNA at cryptic splice sites leads to heterogeneity in the transcribed mRNA products upon expression in eukaryotic cells.

Polynucleotides generated for the expression of transgenes are typically constructed from nucleic acid sequences, such as complementary DNA (cDNA), or portions thereof, that do not contain introns. Thus, splicing of such sequences is not expected to occur. However, the presence of cryptic splice sites within the cDNA sequence can lead to unintended or undesired splicing reactions and heterogeneity in the transcribed mRNA. Such heterogeneity results in translation of unintended protein products, such as truncated protein products with variable amino acid sequences that exhibit modified expression and/or activity.

In some embodiments, eliminating splice sites, such as cryptic splice sites, can improve or optimize expression of a transgene product, such as a polypeptide translated from the transgene, such as a PR3 CAAR polypeptide. Splicing at cryptic splice sites of an encoded transgene, such as an encoded PR3 CAAR molecule, can lead to reduced protein expression, e.g., expression on cell surfaces, and/or reduced function, e.g., reduced intracellular signaling. In some embodiments, the polynucleotides encoding the PR3 CAAR proteins have been optimized to reduce or eliminate cryptic splice sites. In some embodiments, the polynucleotides encoding the PR3 CAAR proteins have been optimized for codon expression and/or in which one or more sequence, such as one identified by the methods or observations herein regarding splice sites, is present, and/or in which an identified splice site, such as any of the identified splice sites herein, is not present.

RNA heterogeneity can be determined by any of a number of methods provided herein or described or known. In some embodiments, RNA heterogeneity of a transcribed nucleic acid is determined by amplifying the transcribed nucleic acid, such as by reverse transcriptase polymerase chain reaction (RT-PCR) followed by detecting one or more differences, such as differences in size, in the one or more amplified products. In some embodiments, the RNA heterogeneity is determined based on the number of differently sized amplified products, or the proportion of various differently sized amplified products. In some embodiments, RNA, such as total RNA or cytoplasmic polyadenylated RNA, is harvested from cells, expressing the transgene to be optimized, and amplified by reverse transcriptase polymerase chain reaction (RT-PCR) using a primer specific to the 5′ untranslated region (5′ UTR), in some cases corresponding to a portion of the promoter sequence in the expression vector, located upstream of the transgene in the transcribed RNA, and a primer specific to the 3′ untranslated region (3′ UTR), located downstream of the expressed transgene in the transcribed RNA sequence or a primer specific to a sequence within the transgene. In particular embodiments, at least one primer complementary to a sequence in the 5′ untranslated region (UTR) and at least one primer complementary to a sequence in the 3′ untranslated region (UTR) are employed to amplify the transgene. One can resolve RNA, such as messenger RNA, and analyze the heterogeneity thereof by several methods. Non-limiting, exemplary methods include agarose gel electrophoresis, chip-based capillary electrophoresis, analytical centrifugation, field flow fractionation, and chromatography, such as size exclusion chromatography or liquid chromatography.

In some embodiments, a provided polynucleotide encoding a PR3 CAAR provided herein, or PR3 construct provided herein, includes modifications to remove one or more splice donor and/or acceptor site that may contribute to splice events and/or reduced expression and/or increased RNA heterogeneity. In some embodiments, provided polynucleotides are modified in one or more polynucleotides in the spacer region to eliminate or reduce splice events.

Provided in are exemplary PR3 CAARs and polynucleotides containing nucleic acid sequences encoding all or a portion, fragment, or domain of any of the exemplary CAARs described herein. Also provided herein are polynucleotides encoding the PR3 CAARs. In some embodiments, the CAAR can be encoded by more than one different polynucleotides, such as two or more polynucleotides. In some of any such embodiments, two or more polynucleotides can each contain nucleic acids encoding a portion, fragment, or domain of the CAAR. In some cases, the polynucleotide also includes the CD33 signal sequence or a PR3 signal sequence. Polynucleotide sequences of exemplary PR3 CAARs are set forth in SEQ ID NOs: 59-88 and 144-159, encoding the amino acid sequences set forth in SEQ ID NOs: 29-58 and 103-118.

In some of any embodiments, said polynucleotide contains the sequence set forth in SEQ ID NOs: 59-88 and 144-159 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NOs: 59-88 and 144-159. In some of any embodiments, said polynucleotide contains the sequence set forth in SEQ ID NO: 59 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 59. In some of any embodiments, said polynucleotide contains the sequence set forth in SEQ ID NO: 59. In some of any embodiments, said polynucleotide contains the sequence set forth in SEQ ID NO: 60 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 60. In some of any embodiments, said polynucleotide contains the sequence set forth in SEQ ID NO: 60. In some of any embodiments, said polynucleotide contains the sequence set forth in SEQ ID NO: 61 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 61. In some of any embodiments, said polynucleotide contains the sequence set forth in SEQ ID NO: 61.

In some of any embodiments, said polynucleotide contains the sequence set forth in SEQ ID NO: 68 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 68. In some of any embodiments, said polynucleotide contains the sequence set forth in SEQ ID NO: 68. In some of any embodiments, said polynucleotide contains the sequence set forth in SEQ ID NO: 69 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 69. In some of any embodiments, said polynucleotide contains the sequence set forth in SEQ ID NO: 69. In some of any embodiments, said polynucleotide contains the sequence set forth in SEQ ID NO: 70 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 70. In some of any embodiments, said polynucleotide contains the sequence set forth in SEQ ID NO: 70.

In some of any embodiments, said polynucleotide contains the sequence set forth in SEQ ID NO: 83 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 83. In some of any embodiments, said polynucleotide contains the sequence set forth in SEQ ID NO: 83. In some of any embodiments, said polynucleotide contains the sequence set forth in SEQ ID NO: 84 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 84. In some of any embodiments, said polynucleotide contains the sequence set forth in SEQ ID NO: 84. In some of any embodiments, said polynucleotide contains the sequence set forth in SEQ ID NO: 85 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 85. In some of any embodiments, said polynucleotide contains the sequence set forth in SEQ ID NO: 85.

In some of any embodiments, said polynucleotide contains the sequence set forth in SEQ ID NO: 86 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 86 In some of any embodiments, said polynucleotide contains the sequence set forth in SEQ ID NO: 86. In some of any embodiments, said polynucleotide contains the sequence set forth in SEQ ID NO: 87 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 87. In some of any embodiments, said polynucleotide contains the sequence set forth in SEQ ID NO: 87. In some of any embodiments, said polynucleotide contains the sequence set forth in SEQ ID NO: 88 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 88. In some of any embodiments, said polynucleotide contains the sequence set forth in SEQ ID NO: 88.

In some of any embodiments, said polynucleotide contains the sequence set forth in SEQ ID NO: 144 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 144 In some of any embodiments, said polynucleotide contains the sequence set forth in SEQ ID NO: 144. In some of any embodiments, said polynucleotide contains the sequence set forth in SEQ ID NO: 145 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 145. In some of any embodiments, said polynucleotide contains the sequence set forth in SEQ ID NO: 145. In some of any embodiments, said polynucleotide contains the sequence set forth in SEQ ID NO: 146 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 146. In some of any embodiments, said polynucleotide contains the sequence set forth in SEQ ID NO: 146.

In some of any embodiments, said polynucleotide contains the sequence set forth in SEQ ID NO: 147 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 147 In some of any embodiments, said polynucleotide contains the sequence set forth in SEQ ID NO: 147. In some of any embodiments, said polynucleotide contains the sequence set forth in SEQ ID NO: 148 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 148. In some of any embodiments, said polynucleotide contains the sequence set forth in SEQ ID NO: 148. In some of any embodiments, said polynucleotide contains the sequence set forth in SEQ ID NO: 149 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 149. In some of any embodiments, said polynucleotide contains the sequence set forth in SEQ ID NO: 149.

In some of any embodiments, said polynucleotide contains the sequence set forth in SEQ ID NO: 150 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 150 In some of any embodiments, said polynucleotide contains the sequence set forth in SEQ ID NO: 150. In some of any embodiments, said polynucleotide contains the sequence set forth in SEQ ID NO: 151 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 151. In some of any embodiments, said polynucleotide contains the sequence set forth in SEQ ID NO: 151. In some of any embodiments, said polynucleotide contains the sequence set forth in SEQ ID NO: 152 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 152. In some of any embodiments, said polynucleotide contains the sequence set forth in SEQ ID NO: 152.

In some of any embodiments, said polynucleotide contains the sequence set forth in SEQ ID NO: 153 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 153 In some of any embodiments, said polynucleotide contains the sequence set forth in SEQ ID NO: 153. In some of any embodiments, said polynucleotide contains the sequence set forth in SEQ ID NO: 154 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 154. In some of any embodiments, said polynucleotide contains the sequence set forth in SEQ ID NO: 154. In some of any embodiments, said polynucleotide contains the sequence set forth in SEQ ID NO: 155 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 155. In some of any embodiments, said polynucleotide contains the sequence set forth in SEQ ID NO: 155.

In some of any embodiments, said polynucleotide contains the sequence set forth in SEQ ID NO: 156 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 156 In some of any embodiments, said polynucleotide contains the sequence set forth in SEQ ID NO: 156. In some of any embodiments, said polynucleotide contains the sequence set forth in SEQ ID NO: 157 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 157. In some of any embodiments, said polynucleotide contains the sequence set forth in SEQ ID NO: 157. In some of any embodiments, said polynucleotide contains the sequence set forth in SEQ ID NO: 158 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 158. In some of any embodiments, said polynucleotide contains the sequence set forth in SEQ ID NO: 158.

In some of any embodiments, said polynucleotide contains the sequence set forth in SEQ ID NO: 159 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 159. In some of any embodiments, said polynucleotide contains the sequence set forth in SEQ ID NO: 159.

In some of any embodiments, said polynucleotide contains a nucleic acid encoding the sequence set forth in SEQ ID NO: 29-58 or 103-122 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 29-58 or 103-122. In some of any embodiments, said polynucleotide contains a nucleic acid encoding the sequence set forth in SEQ ID NO: 29 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 29. In some of any embodiments, said polynucleotide contains a nucleic acid encoding the sequence set forth in SEQ ID NO: 29. In some of any embodiments, said polynucleotide contains a nucleic acid encoding the sequence set forth in SEQ ID NO: 30 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 30. In some of any embodiments, said polynucleotide contains a nucleic acid encoding the sequence set forth in SEQ ID NO: 30. In some of any embodiments, said polynucleotide contains a nucleic acid encoding the sequence set forth in SEQ ID NO: 31 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 31. In some of any embodiments, said polynucleotide contains a nucleic acid encoding the sequence set forth in SEQ ID NO: 31.

In some of any embodiments, said polynucleotide contains a nucleic acid encoding the sequence set forth in SEQ ID NO: 38 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 38. In some of any embodiments, said polynucleotide contains a nucleic acid encoding the sequence set forth in SEQ ID NO: 38. In some of any embodiments, said polynucleotide contains a nucleic acid encoding the sequence set forth in SEQ ID NO: 39 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 39. In some of any embodiments, said polynucleotide contains a nucleic acid encoding the sequence set forth in SEQ ID NO: 39. In some of any embodiments, said polynucleotide contains a nucleic acid encoding the sequence set forth in SEQ ID NO: 40 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 40. In some of any embodiments, said polynucleotide contains a nucleic acid encoding the sequence set forth in SEQ ID NO: 40.

In some of any embodiments, said polynucleotide contains a nucleic acid encoding the sequence set forth in SEQ ID NO: 53 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 53. In some of any embodiments, said polynucleotide contains a nucleic acid encoding the sequence set forth in SEQ ID NO: 53. In some of any embodiments, said polynucleotide contains a nucleic acid encoding the sequence set forth in SEQ ID NO: 54 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 54. In some of any embodiments, said polynucleotide contains a nucleic acid encoding the sequence set forth in SEQ ID NO: 54. In some of any embodiments, said polynucleotide contains a nucleic acid encoding the sequence set forth in SEQ ID NO: 55 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 55. In some of any embodiments, said polynucleotide contains a nucleic acid encoding the sequence set forth in SEQ ID NO: 55.

In some of any embodiments, said polynucleotide contains a nucleic acid encoding the sequence set forth in SEQ ID NO: 56 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 56. In some of any embodiments, said polynucleotide contains a nucleic acid encoding the sequence set forth in SEQ ID NO: 56. In some of any embodiments, said polynucleotide contains a nucleic acid encoding the sequence set forth in SEQ ID NO: 57 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 57. In some of any embodiments, said polynucleotide contains a nucleic acid encoding the sequence set forth in SEQ ID NO: 57. In some of any embodiments, said polynucleotide contains a nucleic acid encoding the sequence set forth in SEQ ID NO: 58 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 58. In some of any embodiments, said polynucleotide contains a nucleic acid encoding the sequence set forth in SEQ ID NO: 58.

In some of any embodiments, said polynucleotide contains a nucleic acid encoding the sequence set forth in SEQ ID NO: 103 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 103. In some of any embodiments, said polynucleotide contains a nucleic acid encoding the sequence set forth in SEQ ID NO: 103. In some of any embodiments, said polynucleotide contains a nucleic acid encoding the sequence set forth in SEQ ID NO: 104 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 104. In some of any embodiments, said polynucleotide contains a nucleic acid encoding the sequence set forth in SEQ ID NO: 104. In some of any embodiments, said polynucleotide contains a nucleic acid encoding the sequence set forth in SEQ ID NO: 105 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 105. In some of any embodiments, said polynucleotide contains a nucleic acid encoding the sequence set forth in SEQ ID NO: 105. In some of any embodiments, said polynucleotide contains a nucleic acid encoding the sequence set forth in SEQ ID NO: 106 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 106. In some of any embodiments, said polynucleotide contains a nucleic acid encoding the sequence set forth in SEQ ID NO: 106.

In some of any embodiments, said polynucleotide contains a nucleic acid encoding the sequence set forth in SEQ ID NO: 107 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 107. In some of any embodiments, said polynucleotide contains a nucleic acid encoding the sequence set forth in SEQ ID NO: 107. In some of any embodiments, said polynucleotide contains a nucleic acid encoding the sequence set forth in SEQ ID NO: 108 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 108. In some of any embodiments, said polynucleotide contains a nucleic acid encoding the sequence set forth in SEQ ID NO: 108. In some of any embodiments, said polynucleotide contains a nucleic acid encoding the sequence set forth in SEQ ID NO: 109 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 109. In some of any embodiments, said polynucleotide contains a nucleic acid encoding the sequence set forth in SEQ ID NO: 109. In some of any embodiments, said polynucleotide contains a nucleic acid encoding the sequence set forth in SEQ ID NO: 110 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 110. In some of any embodiments, said polynucleotide contains a nucleic acid encoding the sequence set forth in SEQ ID NO: 110.

In some of any embodiments, said polynucleotide contains a nucleic acid encoding the sequence set forth in SEQ ID NO: 111 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 111. In some of any embodiments, said polynucleotide contains a nucleic acid encoding the sequence set forth in SEQ ID NO: 111. In some of any embodiments, said polynucleotide contains a nucleic acid encoding the sequence set forth in SEQ ID NO: 112 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 112. In some of any embodiments, said polynucleotide contains a nucleic acid encoding the sequence set forth in SEQ ID NO: 112. In some of any embodiments, said polynucleotide contains a nucleic acid encoding the sequence set forth in SEQ ID NO: 113 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 113. In some of any embodiments, said polynucleotide contains a nucleic acid encoding the sequence set forth in SEQ ID NO: 113. In some of any embodiments, said polynucleotide contains a nucleic acid encoding the sequence set forth in SEQ ID NO: 114 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 114. In some of any embodiments, said polynucleotide contains a nucleic acid encoding the sequence set forth in SEQ ID NO: 114.

In some of any embodiments, said polynucleotide contains a nucleic acid encoding the sequence set forth in SEQ ID NO: 115 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 115. In some of any embodiments, said polynucleotide contains a nucleic acid encoding the sequence set forth in SEQ ID NO: 115. In some of any embodiments, said polynucleotide contains a nucleic acid encoding the sequence set forth in SEQ ID NO: 116 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 116. In some of any embodiments, said polynucleotide contains a nucleic acid encoding the sequence set forth in SEQ ID NO: 116. In some of any embodiments, said polynucleotide contains a nucleic acid encoding the sequence set forth in SEQ ID NO: 117 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 117. In some of any embodiments, said polynucleotide contains a nucleic acid encoding the sequence set forth in SEQ ID NO: 117. In some of any embodiments, said polynucleotide contains a nucleic acid encoding the sequence set forth in SEQ ID NO: 118 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 118. In some of any embodiments, said polynucleotide contains a nucleic acid encoding the sequence set forth in SEQ ID NO: 118.

In some of any embodiments, said polynucleotide contains a nucleic acid encoding the sequence set forth in SEQ ID NO: 119 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 119. In some of any embodiments, said polynucleotide contains a nucleic acid encoding the sequence set forth in SEQ ID NO: 119. In some of any embodiments, said polynucleotide contains a nucleic acid encoding the sequence set forth in SEQ ID NO: 120 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 120. In some of any embodiments, said polynucleotide contains a nucleic acid encoding the sequence set forth in SEQ ID NO: 120. In some of any embodiments, said polynucleotide contains a nucleic acid encoding the sequence set forth in SEQ ID NO: 121 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 121. In some of any embodiments, said polynucleotide contains a nucleic acid encoding the sequence set forth in SEQ ID NO: 121. In some of any embodiments, said polynucleotide contains a nucleic acid encoding the sequence set forth in SEQ ID NO: 122 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 122. In some of any embodiments, said polynucleotide contains a nucleic acid encoding the sequence set forth in SEQ ID NO: 122.

### II. ENGINEERED CELLS

Also provided are cells such as engineered cells that contain a CAAR such as one that contains an extracellular domain including a PR3 antibody binding domain construct as described herein. Also provided are populations of such cells, compositions containing such cells and/or enriched for such cells, such as in which cells expressing the PR3 CAAR make up at least 50, 60, 70, 80, 90, 91, 92, 93, 94, 95, 96, 97, 98, 99, or more percent of the total cells in the composition or cells of a certain type such as T cells or CD8+ or CD4+ cells. Among the compositions are pharmaceutical compositions and formulations for administration, such as for adoptive cell therapy. Also provided are therapeutic methods for administering the cells and compositions to subjects, e.g., patients.

Also provided are genetically engineered cells expressing the PR3 CAARs. The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen.

In certain embodiments, the cell expressing the PR3 CAAR is an induced pluripotent stem cell (iPsC). In some embodiments, the cell is an iPSC derivative cell that has been differentiated from an iPSC.

In some embodiments, IPCs are differentiated into T cells or NK cells. In some embodiments, methods of differentiating iPSCs into T cells or NK cells are described, for example, in published PCT application Nos. WO2022120334, WO2022216514 and WO2022216624. In certain embodiments, the iPSC can be differentiated by any method known in the art. Exemplary methods are described in U.S. Pat. Nos. 8,846,395, 8,945,922, 8,318,491, WO2010/099539, WO2012/109208, WO2017/070333, WO2017/179720, WO2016/010148, WO2018/048828 and WO2019/157597, each of which are herein incorporated by reference in its entirety. In some embodiments, the differentiated T cell is a gamma-delta T cell. In some embodiments, the differentiated T cell is an alpha-beta T cell. The differentiation protocol may use feeder cells or may be feeder-free. As used herein, “feeder cells” or “feeders” are terms describing cells of one type that are co-cultured with cells of a second type to provide an environment in which the cells of the second type can grow, expand, or differentiate, as the feeder cells provide stimulation, growth factors and nutrients for the support of the second cell type.

In some embodiments, the iPSC derivative cells are NK cells which are prepared by a method of differentiating an iPSC cell into an NK cell by subjecting the cells to a differentiation protocol including the addition of recombinant human IL-12p70 for the final 24 hours of culture.

In some embodiments, the iPSC is modified by genomic engineering. Use of iPSCs enables cellular engineering to produce a controlled cell bank of modified cells that can be expanded and differentiated into desired immune effector cells, supplying large amounts of homogeneous allogeneic therapeutic products. In some embodiments, the genomic engineering comprises targeted editing which can include, but are not limited to, but are not limited to, deletion, insertion, or in/del carried out by CRISPR, ZFN, TALEN, homing nuclease, homology recombination, or any other functional variation of these methods. In some embodiments, the genomic engineering comprises one or more exogenous polynucleotides integrated at one or more loci on the chromosome of an iPSC. In some embodiments, iPSCs or cells differentiated therefrom are engineered with genomic modifications that enhance the therapeutic properties of the derivative cells. In some embodiments, the resulted differentiated cells derived from engineered iPSCs contain the modifications that render the cells hypoimmune for use of the cells as an allogeneic cell product. Exemplary genomic modifications are described, for example, in published PCT application Nos. WO2022120334, WO2022216514 and WO2022216624.

In some embodiments, the iPSC or iPSC derivative cell (e.g. T cell or NK cell) is modified by one or more gene edits (e.g., knockouts) or insertion of one or more exogenous genes (e.g., knockins) that render the cell hypoimmune. In some embodiments, the cell is modified by deletion of one or more target genes or insertion of one or more exogenous genes to render the cell hypoimmune or to modulate its activity.

In some embodiments, the cell is an allogeneic cell. In some embodiments, the cell is engineered to be hypoimmune.

In some embodiments, the cell is an autologous cell.

In some embodiments, the cells are T cells. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, provided herein is a T cell comprising a provided CAAR. In some embodiments the T cell is a CD4+ T cell. In some embodiments, the T cell is a CD8+ T cell.

With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4⁺ and/or of CD8⁺ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

Also provided herein are populations of cells in which cells of the population comprise a provided CAAR. In some embodiments, the population of cells are cells from the peripheral blood of a subject, such as from a subject to be treated (e.g. by autologous adoptive cell therapy). In some embodiments, the population of cells are obtained from by apheresis. In some embodiments, the population of cells are obtained by leukapheresis. In some embodiments, the population of cells are peripheral blood mononuclear cells (PBMCs). In some embodiments, among the cells in the population, greater than at or about 30%, greater than at or about 40%, greater than at or about 50%, greater than at or about 60%, greater than at or about 70%, or more of the cells are T cells.

Also provided herein are populations of T cells in which T cells of the population comprise a provided CAAR. In some embodiments, the population of T cells are enriched, such as selected, from a biological sample (e.g. blood, leukapheresis or apheresis sample) from a subject. In some embodiments, the population of T cells is enriched in CD3+ cells. In some embodiments, the population of T cells contains greater than or greater than about 75% CD3+ T cells, such as greater than or greater than about 80% CD3+ T cells, greater than or greater than about 85% CD3+ T cells, greater than or greater than about 90% CD3+ T cells or greater than or greater than about 95% CD3+ T cells. In some embodiments, the population of T cells contains greater than or greater than about 95% CD3+ T cells. In some embodiments, the population of T cells contains CD4+ and CD8+ T cells. In some embodiments, the CD4+ and CD8+ T cells are present at a ratio of at our about 1:3 to 3:1, such as 1:2 to 2:1, such as at or about 1:1. In some embodiments, the population of T cells contains greater than or greater than about 75% CD4+ and CD8+ T cells, such as greater than or greater than about 80% CD4+ and CD8+ T cells, greater than or greater than about 85% CD4+ and CD8+ T cells, greater than or greater than about 90% CD4+ and CD8+ T cells or greater than or greater than about 95% CD4+ and CD8+ T cells. In some embodiments, the population of T cells contains greater than or greater than about 95% CD4+ and CD8+ T cells.

In some embodiments, at least 25% of the cells in the population express the CAAR. In some embodiments, at least or about 30%, at least or about 40%, at least or about 50%, at least or about 50%, at least or about 60%, at least or about 70%, at least or about 80% or at least or about 90% or more of the cells in the population express the CAAR. In some embodiments, the expression refers to positive surface expression of the CAAR on the cells surface, for example as determined by flow cytometry.

In some embodiments, the cells include one or more polynucleotides introduced via genetic engineering, and thereby express recombinant or genetically engineered products of such polynucleotides. In some embodiments, the polynucleotides are heterologous, i.e., normally not present in a cell or sample obtained from the cell, such as one obtained from another organism or cell, which for example, is not ordinarily found in the cell being engineered and/or an organism from which such cell is derived. In some embodiments, the polynucleotides are not naturally occurring, such as a polynucleotide not found in nature, including one comprising chimeric combinations of polynucleotides encoding various domains from multiple different cell types. In some embodiments, the cells (e.g., engineered cells) comprise a vector (e.g., a viral vector, expression vector, etc.) as described herein such as a vector comprising a nucleic acid encoding a PR3 CAAR described herein.

### A. Vectors and Methods for Genetic Engineering

Also provided are methods, nucleic acids, compositions, and kits, for expressing the PR3 CAARs and for producing the genetically engineered cells expressing said CAARs. In some embodiments, one or more CAARs can be genetically engineered into cells or plurality of cells. The genetic engineering generally involves introduction of a nucleic acid encoding the recombinant or engineered component into the cell, such as, for example, by retroviral transduction, transfection, or transformation.

The nucleic acids may include those encompassing natural and/or non-naturally occurring nucleotides and bases, e.g., including those with backbone modifications. The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide. In some aspects, the nucleic acid sequence encoding at least a portion of the antibody or antigen-binding fragment thereof, such as an scFv, conjugates, receptors (e.g., CAARs) provided herein can be optimized, for example, codon-optimized for expression in a human cell and/or optimized to reduce or eliminate cryptic splice sites.

In some embodiments, the polynucleotides also include one or more additional sequences, such as those encoding one or more additional molecules, such as a marker, or promoters, regulatory elements and/or multicistronic elements. In some embodiments, the provided polynucleotides include any of the polynucleotides described herein.

Also provided are vectors containing the nucleic acids, e.g., polynucleotides, and engineered cells containing the vectors, e.g., engineered immune cells expressing the PR3 CAAR. Also provided are methods for engineering cells, such as immune cells, to express the PR3 CAAR. The nucleic acid may encode an amino acid sequence comprising the wild-type PR3 antibody binding domain or a mutant PR3 antibody binding domain as the extracellular antigen-binding domain; a transmembrane domain and intracellular domains, such as a CD3 zeta and a costimulatory signaling domain.

In a further embodiment, one or more vectors (e.g., expression vectors) comprising such polynucleotides are provided. In a further embodiment, a host cell comprising such polynucleotides is provided. In one such embodiment, a host cell comprises (e.g., has been transformed with) a vector comprising a nucleic acid that encodes an amino acid sequence comprising a PR3 CAAR. In some embodiments, one or more such host cells are provided. In some embodiments, a composition containing one or more such host cells are provided. In some embodiments, the one or more host cells can express different PR3 CAARs, or the same PR3 CAARs. In some embodiments, each of the host cells can express more than one PR3 CAAR.

Also provided are methods of making the PR3 CAARs. For recombinant production of the CAARs, a nucleic acid sequence encoding a CAAR, e.g., as described herein, may be isolated and inserted into one or more vectors for further cloning and/or expression in a host cell. Such nucleic acid sequences may be readily isolated and sequenced using conventional procedures. In some embodiments, a method of making the PR3 CAAR is provided, wherein the method comprises culturing a host cell comprising a nucleic acid sequence encoding the antibody, as provided above, under conditions suitable for expression of the receptor.

The provided embodiments further include vectors and host cells and other expression systems for expressing and producing the PR3 CAAR, including eukaryotic and prokaryotic host cells, including bacteria, filamentous fungi, and yeast, as well as mammalian cells such as human cells, as well as cell-free expression systems.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 2:223, 1977) which confers ganciclovir sensitivity; the cellular hypoxanthine phosphoribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some aspects, the cells further are engineered to promote expression of cytokines or other factors. Various methods for the introduction of genetically engineered components, e.g., PR3 CAARs, are well known and may be used with the provided methods and compositions. Exemplary methods include those for transfer of polynucleotides encoding the receptors, including via viral, e.g., retroviral or lentiviral, transduction, transposons, and electroporation.

In some embodiments, recombinant polynucleotides are transferred into cells using recombinant infectious virus particles, such as, e.g., vectors derived from simian virus 40 (SV40), adenoviruses, or adeno-associated virus. In some embodiments, recombinant polynucleotides are transferred into T cells using recombinant lentiviral vectors, such as HIV-1 lentivirus-based vectors (lentivectors; see, e.g., Amado et al., Science. 1999 Jul. 30; 285(5428):674-676), or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 November 29(11): 550-557).

In some embodiments, the retroviral vector or lentiviral vector has a long terminal repeat sequence (LTR). In some embodiments the vector is derived from the Moloney murine leukemia virus (MoMLV), myeloproliferative sarcoma virus (MPSV), murine embryonic stem cell virus (MESV), murine stem cell virus (MSCV), spleen focus forming virus (SFFV), human immunodeficiency virus type 1 (HIV-1) or human immunodeficiency virus type 2 (HIV-2/SIV). In some embodiments, the vectors are self-inactivating (SIN). In some embodiments, the vectors are conditionally replicating (mobilizable) vectors. Most lentiviral vectors are derived from human, feline or simian lentiviruses. Most retroviral vectors are derived from murine retroviruses. In some embodiments, the lentiviruses or retroviruses include those derived from any avian or mammalian cell source. The lentiviruses or retroviruses typically are amphotropic, meaning that they are capable of infecting host cells of several species, including humans. In one embodiment, the gene to be expressed replaces the retroviral gag, pol and/or env sequences. Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505. A number of illustrative retroviral systems have also been described (e.g., Amado et al., (1999) Science 285(5428):674-676, U.S. Pat. Nos. 5,219,740; 6,207,453; 5,219,740; Miller and Rosman (1989) BioTechniques 7:980-990; Miller (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109).

In some embodiments, recombinant polynucleotides are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant polynucleotides are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston (1990) Nature 346: 776-777); and strontium phosphate DNA co-precipitation (Brash et al., (1987) Mol. Cell Biol. 7: 2031-2034). Other approaches and vectors for transfer of the polynucleotides encoding the recombinant products are those described, e.g., in WO2014055668, and U.S. Pat. No. 7,446,190.

Among additional polynucleotides, e.g., genes for introduction are those to improve the outcome of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

In some embodiments the vector or construct can contain a promoter and/or enhancer or regulatory elements to regulate expression of the encoded recombinant receptor. In some examples the promoter and/or enhancer or regulatory elements can be condition-dependent promoters, enhancers, and/or regulatory elements. In some examples these elements drive expression of the transgene. In some examples, the CAR transgene can be operatively linked to a promoter, such as an EF1alpha promoter with an HTLV1 enhancer (SEQ ID NO:164). In some examples, the CAR transgene is operatively linked to a Woodchuck Hepatitis Virus (WHP) Posttranscriptional Regulatory Element (WPRE; SEQ ID NO: 165), located downstream of the transgene.

In some embodiments, the vector or construct can contain a single promoter that drives the expression of one or more nucleic acid molecules. In some embodiments, such nucleic acid molecules, e.g., transcripts, can be multicistronic (bicistronic or tricistronic, see e.g., U.S. Pat. No. 6,060,273). For example, in some embodiments, transcription units can be engineered as a bicistronic unit containing an IRES (internal ribosome entry site), which allows coexpression of gene products (e.g., encoding a first and second chimeric receptor) by a message from a single promoter. For example, in some embodiments, the vector or construct can contain a nucleic acid encoding an anti-ROR1 receptor (e.g., an anti-ROR1 CAR) provided herein and a nucleic acid encoding a different molecule, separated by an IRES, under the regulation of a single promoter.

Alternatively, in some cases, a single promoter may direct expression of an RNA that contains, in a single open reading frame (ORF), two or three genes (e.g. encoding a first and second binding molecules, e.g., antibody recombinant receptor) separated from one another by sequences encoding a self-cleavage peptide (e.g., 2A cleavage sequences) or a protease recognition site (e.g., furin). The ORF thus encodes a single polypeptide, which, either during (in the case of T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and polynucleotides disclosed herein, without limitation, 2A sequences from the foot-and-mouth disease virus (F2A, e.g., SEQ ID NO:21 or 168), equine rhinitis A virus (E2A, e.g., SEQ ID NO: 20 or 167), Thosea asigna virus (T2A, e.g., SEQ ID NO: 6, 17 or 166), and porcine teschovirus-1 (P2A, e.g., SEQ ID NO: 18 or 19) as described in U.S. Patent Publication No. 20070116690. In some embodiments, the one or more different or separate promoters drive the expression of one or more nucleic acid molecules encoding the one or more binding molecules, e.g., recombinant receptors.

Any of the PR3 CAARs provided herein can be encoded by polynucleotides containing one or more nucleic acid molecules encoding the receptors, in any combinations or arrangements. For example, one, two, three or more polynucleotides can encode one, two, three or more different receptors or domains. In some embodiments, one vector or construct contains nucleic acid molecules encoding one or more PR3 CAARs, and a separate vector or construct contains nucleic acid molecules encoding an additional PR3 CAAR.

In some embodiments, the nucleic acid molecules can also encode one or more surrogate marker(s), such as fluorescent protein (e.g., green fluorescent protein (GFP)) or a cell surface marker (e.g., a truncated surface marker such as truncated EGFR (tEGFR), which may be used to confirm transduction or engineering of the cell to express the receptor. For example, in some aspects, extrinsic marker genes are utilized in connection with engineered cell therapies to permit detection or selection of cells and, in some cases, also to promote cell suicide by ADCC. Exemplary marker genes include truncated epidermal growth factor receptor (EGFRt), which can be co-expressed with a transgene of interest (e.g., a CAAR or TCR) in transduced cells (see, e.g., U.S. Pat. No. 8,802,374). EGFRt contains an epitope recognized by the antibody cetuximab (Erbitux®). For this reason, Erbitux® can be used to identify or select cells that have been engineered with the EGFRt construct, including in cells also co-engineered with another recombinant receptor, such as a chimeric antigen receptor (CAR).

In some embodiments, the nucleic acid encoding the binding molecules further contain contains a nucleic acid sequence encoding one or more marker(s). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the polynucleotide, e.g., a polynucleotide encoding the PR3 CAAR. In some embodiments, the transduction marker can indicate or confirm modification of a cell. In some embodiments, the surrogate marker is a protein that is made to be co-expressed on the cell surface with the PR3 CAAR. In particular embodiments, such a surrogate marker is a surface protein that has been modified to have little or no activity. In certain embodiments, the surrogate marker is encoded on the same polynucleotide that encodes the PR3 CAAR. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO: 141-143) or a prostate-specific membrane antigen (PSMA) or modified form thereof tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR).

In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, the nucleic acid encoding the marker is operably linked to a polynucleotide encoding for a linker sequence, such as a cleavable linker sequence, e.g., a T2A. For example, a marker, and optionally a linker sequence, can be any as disclosed in PCT Pub. No. WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 141-143 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 141-143.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

Also provided are compositions containing one or more of the nucleic acid molecules, vectors or constructs, such as any described above. In some embodiments, the nucleic acid molecules, vectors, constructs or compositions can be used to engineer cells, such as T cells, to express any of the binding molecules, e.g., antibody or recombinant receptor, and/or the additional binding molecules.

### B. Preparation of Cells for Engineering

In some embodiments, preparation of the engineered cells includes one or more culture and/or preparation steps. The cells for introduction of the CAAR may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contain cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques.

For example, CD3+, CD28+ T cells can be positively selected using anti-CD3/anti-CD28 conjugated magnetic beads (e.g., DYNABEADS® M-450 CD3/CD28 T Cell Expander, MACSiBeads™, etc.).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase certain features, such as to improve long-term survival, expansion, and/or engraftment following administration, which in some aspects is particularly robust in such sub-populations (see Terakura et al. (2012) Blood. 1:72-82; Wang et al. (2012) J Immunother. 35(9):689-701). In some embodiments, combining TCM-enriched CD8+ T cells and CD4+ T cells further enhances response.

In embodiments, memory T cells are present in both CD62L+ and CD62L-subsets of CD8+ peripheral blood lymphocytes. PBMC can be enriched for or depleted of CD62L-CD8+ and/or CD62L+CD8+ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA, and positive selection based on a marker characteristic of central memory T cells, such as CD62L or CCR7, where the positive and negative selections are carried out in either order.

CD4+ T helper cells are sorted into naïve, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO−.

In one example, to enrich for CD4+ cells by negative selection, a monoclonal antibody cocktail typically includes antibodies to CD14, CD20, CD11 b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In vitro and In vivo, p 17-25 Edited by: S. A. Brooks and U. Schumacher © Humana Press Inc., Totowa, NJ).

In some aspects, the sample or composition of cells to be separated is incubated with small, magnetizable or magnetically responsive material, such as magnetically responsive particles or microparticles, such as paramagnetic beads (e.g., such as Dynabeads® or MACS® beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084, are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS®) (Miltenyi Biotec, Auburn, CA). Magnetic Activated Cell Sorting (MACS®) systems are capable of high-purity selection of cells having magnetized particles attached thereto. In certain embodiments, MACS® operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in WO2009/072003 or US 20110003380.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS® system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS® system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy® system (Miltenyi Biotec). The CliniMACS Prodigy® system in some aspects is equipped with a cell processing unity that permits automated washing and fractionation of cells by centrifugation. The CliniMACS Prodigy® system can also include an onboard camera and image recognition software that determines the optimal cell fractionation endpoint by discerning the macroscopic layers of the source cell product. For example, peripheral blood may be automatically separated into erythrocytes, white blood cells and plasma layers. The CliniMACS Prodigy® system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope (see, e.g., Klebanoff et al. (2012) J Immunother. 35(9): 651-660, Terakura et al. (2012) Blood. 1:72-82, and Wang et al. (2012) J Immunother. 35(9):689-701).

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° C. per minute and stored in the vapor phase of a liquid nitrogen storage tank.

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of stimulating or activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti-CD28 antibody to the culture medium (e.g., at a concentration of at least about 0.5 ng/mL). In some embodiments, the stimulating agents include IL-2 and/or IL-15, for example, an IL-2 concentration of at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

### III. PHARMACEUTICAL COMPOSITIONS

Also provided are compositions including the PR3 CAARs and engineered cells, including pharmaceutical compositions and formulations. Also provided are compositions comprising engineered cells that express the PR3 CAARs provided herein, including pharmaceutical compositions and formulations.

Provided are pharmaceutical formulations comprising a PR3 CAAR, engineered cells expressing said CAAR, a plurality of engineered cells expressing said CAAR and/or additional agents for combination treatment or therapy. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell, binding molecule, and/or antibody, and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being treated with the binding molecules or cells, preferably those with activities complementary to the binding molecule or cell, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the cells or antibodies are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

Active ingredients may be entrapped in microcapsules, in colloidal drug delivery systems (for example, liposomes, albumin microspheres, microemulsions, nano-particles and nanocapsules) or in macroemulsions. In certain embodiments, the pharmaceutical composition is formulated as an inclusion complex, such as cyclodextrin inclusion complex, or as a liposome. Liposomes can serve to target the host cells (e.g., T-cells or NK cells) to a particular tissue. Many methods are available for preparing liposomes, such as those described in, for example, Szoka et al., Ann. Rev. Biophys. Bioeng., 9: 467 (1980), and U.S. Pat. Nos. 4,235,871, 4,501,728, 4,837,028, and U.S. Pat. No. 5,019,369.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

The pharmaceutical composition in some embodiments contains the binding molecules and/or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In certain embodiments, in the context of genetically engineered cells containing the CAAR, a subject is administered the range of at or about one million to at or about 100 billion cells, such as, e.g., at or about 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), such as at or about 10 million to at or about 100 billion cells (e.g., at or about 20 million cells, at or about 30 million cells, at or about 40 million cells, at or about 60 million cells, at or about 70 million cells, at or about 80 million cells, at or about 90 million cells, at or about 10 billion cells, at or about 25 billion cells, at or about 50 billion cells, at or about 75 billion cells, at or about 90 billion cells, or a range defined by any two of the foregoing values), and in some cases at or about 100 million cells to at or about 50 billion cells (e.g., at or about 120 million cells, at or about 250 million cells, at or about 350 million cells, at or about 450 million cells, at or about 650 million cells, at or about 800 million cells, at or about 900 million cells, at or about 3 billion cells, at or about 30 billion cells, at or about 45 billion cells) or any value in between these ranges, and/or such a number of cells per kilogram of body weight of the subject.

The cells may be administered using standard administration techniques, formulations, and/or devices. Provided are formulations and devices, such as syringes and vials, for storage and administration of the compositions. Administration of the cells can be autologous or heterologous. For example, immune cells, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immune cell such as a T cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, intracranial, intrathoracic, and intraperitoneal administration. In some embodiments, the cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the binding molecule in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

Also provided are pharmaceutical compositions for combination therapy. Any of the additional agents for combination therapy described herein can be prepared and administered as one or more pharmaceutical compositions with the PR3 CAAR and/or engineered cells expressing said CAAR described herein. The combination therapy can be administered in one or more pharmaceutical compositions, e.g., where the CAARs and/or cells are in the same pharmaceutical composition as the additional agent, or in separate pharmaceutical compositions. For example, in some embodiments, the additional agent is an additional engineered cell, e.g., cell engineered to express a different CAAR that targets a different epitope on PR3, and is administered in the same composition or in a separate composition. In some embodiments, each of the pharmaceutical composition is formulated in a suitable formulation according to the particular binding molecule, recombinant receptor, cell, e.g., engineered cell, and/or additional agent, and the particular dosage regimen and/or method of delivery.

### IV. METHODS AND USES

Also provided herein are methods, such as methods of treatment, of using and uses of the PR3 CAAR-engineered cells, and pharmaceutical compositions and formulations thereof, such as in the treatment of diseases, conditions, and disorders in which antibodies to PR3 are expressed, and/or detection, diagnostic, and prognostic methods. Also provided are methods of combination therapy and/or treatment.

In some embodiments, the methods and uses involve administering PR3 CAARs-engineered cells expressing the PR3 CAAR (also referred to as PR3 CAAR cells), such as a plurality of engineered cells expressing the CAAR, and/or compositions comprising the same, to a subject in need thereof. Such methods and uses include therapeutic methods and uses, for example, involving administration of the PR3 CAAR cells (e.g., engineered cells), or compositions containing the same, to a subject having a disease, condition, or disorder associated with anti-PR3 ANCA such as a disease, condition, or disorder associated with autoreactive immune cells (e.g., autoreactive B cells). In some embodiments, the cellor composition thereof is administered in an effective amount to effect treatment of the disease or disorder. Provided herein are uses of the PR3 CAARs cells (e.g., engineered cells) in such methods and treatments, and in the preparation of a medicament in order to carry out such therapeutic methods. Uses include uses of the CAARs-engineered cells in such methods and treatments, and in the preparation of a medicament in order to carry out such therapeutic methods. In some embodiments, the methods are carried out by administering the PR3 CAAR-engineered cells, or compositions comprising the same, to the subject having, having had, or suspected of having the disease or condition. In some embodiments, the methods thereby treat the disease or condition or disorder in the subject. Also provided herein are of use of any of the compositions, such as pharmaceutical compositions provided herein, for the treatment of a disease or disorder associated with anti-PR3 ANCA.

Provided herein is a cell comprising the PR3 CAAR as described for use in any of the provided methods or uses. In some embodiments, the cell is a lymphocyte. In some embodiments, the cell is an NK cell or a T cell. In some embodiments, the PR3 CAARs or cells or compositions exhibit cytotoxic activity against target cells that express a plurality of anti-PR3 antibodies, wherein the plurality of anti-PR3 antibodies includes antibodies that specifically bind two or more different epitopes on PR3. The target cells can be, for example, CHO-S or Sp2a hybridomas engineered to express the anti-PR3 antibodies. The cytotoxic activity of a target cell can be shown as described in Example 4 part B. In some embodiments, the anti-PR3 antibodies are antibodies disclosed herein. The antibodies can include, for instance, one or more commercially available antibodies, e.g., one or more of MAB684022, MCPR3-3, PR3G2, MCPR3-2, and WGM2.

In some such embodiments, the PR3 CAARs cells or compositions exhibit cytotoxic activity against target cells that express a plurality of anti-PR3 antibodies, wherein the plurality of anti-PR3 antibodies includes antibodies that specifically bind three or more different epitopes on PR3. In some such embodiments, the PR3 CAARs cells or compositions exhibit cytotoxic activity against target cells that express a plurality of anti-PR3 antibodies, wherein the plurality of anti-PR3 antibodies includes antibodies that specifically bind four or more different epitopes on PR3. In some such embodiments, the PR3 CAARs cells or compositions exhibit cytotoxic activity against target cells that express a plurality of anti-PR3 antibodies, wherein the plurality of anti-PR3 antibodies includes antibodies that specifically bind five or more different epitopes on PR3. In some such embodiments, the PR3 CAARs cells or compositions exhibit cytotoxic activity against target cells that express a plurality of anti-PR3 antibodies, wherein the plurality of anti-PR3 antibodies includes antibodies that specifically bind six or more different epitopes on PR3.

In some instances of each of the foregoing embodiments, the PR3 CAARs cells or compositions exhibit at least 70% killing efficiency against the target cells that express each of the plurality of anti-PR3 expressing antibodies. In some instances of each of the foregoing embodiments, the cell exhibits at least 75% killing efficiency against the target cells that express each of the plurality of anti-PR3 expressing antibodies. In some instances of each of the foregoing embodiments, the PR3 CAARs cells or compositions exhibit at least 80% killing efficiency against the target cells that express each of the plurality of anti-PR3 expressing antibodies. In some instances of each of the foregoing embodiments, the PR3 CAARs cells or compositions exhibit at least 85% killing efficiency against the target cells that express each of the plurality of anti-PR3 expressing antibodies. In some instances of each of the foregoing embodiments, the PR3 CAARs cells or compositions exhibit at least 90% killing efficiency against the target cells that express each of the plurality of anti-PR3 expressing antibodies. In some instances of each of the foregoing embodiments, the PR3 CAARs cells or compositions exhibit at least 95% killing efficiency against the target cells that express each of the plurality of anti-PR3 expressing antibodies.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “active disease” means new, persistent, or worsening clinical signs and/or symptoms attributed to granulomatosis with polyangiitis (GPA; formerly known as Wegener's granulomatosis), microscopic polyangiitis (MPA), or eosinophilic granulomatosis with polyangiitis (EGPA; previously known as Churg-Strauss syndrome) and not related to prior damage.

As used herein, “severe disease” means vasculitis with life- or organ-threatening manifestations (e.g., alveolar hemorrhage, glomerulonephritis, central nervous system vasculitis, mononeuritis multiplex, cardiac involvement, mesenteric ischemia, limb/digit ischemia).

As used herein, “nonsevere disease” means vasculitis without life- or organ-threatening manifestations (e.g., rhinosinusitis, asthma, mild systemic symptoms, uncomplicated cutaneous disease, mild inflammatory arthritis).

As used herein, “refractory disease” means persistent active disease despite an appropriate course of immunosuppressive therapy.

As used herein, “relapse” means a recurrence of active disease following a period of remission.

As used herein, “remission” means absence of clinical signs or symptoms attributed to granulomatosis with GPA, MPA, or EGPA, on or off immunosuppressive therapy.

As used herein, “delaying development of a disease” means to defer, hinder, slow, retard, stabilize, suppress and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. “Preventing,” as used herein, includes providing prophylaxis with respect to the occurrence or recurrence of a disease in a subject that may be predisposed to the disease but has not yet been diagnosed with the disease. As used herein, to “suppress” a function or activity is to reduce the function or activity when compared to otherwise same conditions except for a condition or parameter of interest, or alternatively, as compared to another condition.

An “effective amount” of an agent, e.g., a pharmaceutical formulation, PR3 CAAR cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation, PR3 CAAR cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the molecules, cells, and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

As used herein, a “subject” is a mammal, such as a human or other animal, and typically is human.

Among the diseases to be treated by the PR3 CAAR cell or composition provided herein are any ANCA-associated disease or condition or anti-PR3 antibody-associated disease or condition. Typically, the disease or condition is associated with PR3 ANCA. Among the ANCA-associated diseases or conditions that can be treated include, but are not limited to, ANCA-associated vasculitis including granulomatosis with polyangiitis (GPA; formerly known as Wegener's granulomatosis), microscopic polyangiitis (MPA), and eosinophilic granulomatosis with polyangiitis (EGPA; previously known as Churg-Strauss syndrome). In some embodiments, the disease or condition to be treated is GPA. In some embodiments, the disease or condition to be treated is MPA. In some embodiments, the disease or condition to be treated is EGPA.

In some embodiments, the disease or condition is renal-limited vasculitis.

In some embodiments, the disease or condition is glomerulonephritis (e.g., renal-limited rapidly progressive glomerulonephritis).

In some embodiments, the disease or condition is ulcerative colitis. In some embodiments, the disease or condition is ulcerative colitis. In some embodiments, the disease or condition is primary sclerosing cholangitis.

In some embodiments, the diseases or condition to be treated can be active, severe, nonsevere, or refractory. In some embodiments, the subject has undergone prior treatment for the disease or condition. In some embodiments, the prior therapy can include, but is not limited to, administration of one or more of a glucocorticoid, cyclophosphamide (CYC), rituximab (RTX), plasma exchange, Avacopan, methotrexate (MTX), mycophenolate mofetil (MMF), azathioprine (AZA), leflunomide (LEF), belimumab, mepolizumab, and omalizumab. In some embodiments, the glucocorticoid administered during a prior therapy can include, but is not limited to, prednisolone, methylprednisolone, prednisone, or dexamethasone.

In certain embodiments, the prior therapy comprises a remission induction therapy (e.g., a remission induction agent) and/or a remission maintenance therapy. In certain embodiments, methods for treating a subject with any ANCA-associated disease or condition or anti-PR3 antibody-associated disease or condition (e.g. AAV) comprises administering to the subject a PR3 CAAR cell as provided herein and a remission induction therapy. In certain embodiments, the remission induction therapy can include, but is not limited to, cyclophosphamide.

In some embodiments, the additional therapy is a lymphodepleting therapy. In some embodiments, lymphodepletion is performed on a subject, e.g., prior to administering engineered cells, e.g., CAAR-expressing cells. In some embodiments, the lymphodepletion comprises administering one or more of melphalan, cyclophosphamide (e.g., Cytoxan), and fludarabine. In some embodiments, a lymphodepleting chemotherapy is administered to the subject prior to, concurrently with, or after administration (e.g., infusion) of engineered cells, e.g., CAAR-expressing cells. In an example, the lymphodepleting chemotherapy is administered to the subject prior to administration of engineered cells, e.g., CAAR-expressing cells.

In some embodiments, a course of cyclophosphamide is administered to the subject at least 1 week before the PR3 CAAR cell or composition is administered to the subject. In some embodiments, a course of cyclophosphamide is administered to the subject at least 2 weeks before the PR3 CAAR cell or composition is administered to the subject. In some embodiments, a course of cyclophosphamide is administered to the subject at least 3 weeks before the PR3 CAAR cell or composition is administered to the subject. In some embodiments, a course of cyclophosphamide is administered to the subject at least 4 weeks before the PR3 CAAR cell or composition is administered to the subject. In some embodiments, a course of cyclophosphamide is administered to the subject at least 5 weeks before the PR3 CAAR cell or composition is administered to the subject. In some embodiments, a course of cyclophosphamide is administered to the subject at least 6 weeks before the PR3 CAAR cell or composition is administered to the subject.

In certain embodiments, the cyclophosphamide is administered to the subject daily or intermittently. In certain embodiments, the cyclophosphamide is administered to the subject by any appropriate route of administration. In certain embodiments, the cyclophosphamide is administered orally. In certain embodiment, the cyclophosphamide is administered intravenously. In certain embodiments, the cyclophosphamide is administered at a range between 0.5 mg/kg/day to 5.0 mg/kg/day. In certain embodiments, the cyclophosphamide is administered at a range between 5 mg/kg to 25 mg/kg every week. In certain embodiments, the cyclophosphamide is administered at a range between 5 mg/kg to 25 mg/kg every two weeks. In certain embodiments, the cyclophosphamide is administered at a range between 5 mg/kg to 25 mg/kg every three weeks.

In certain embodiments, the remission induction therapy further comprises a glucocorticoid. In certain embodiments, the glucocorticoid is administered to the subject before PR3 CAAR cell treatment. In certain embodiments, the glucocorticoid is administered to the subject after PR3 CAAR cell treatment. In certain embodiments, the glucocorticoid is administered to the subject both before and after the PR3 CAAR cell treatment. In certain embodiments, the dose of glucocorticoids is tapered after the PR3 CAAR cell treatment. In certain embodiments, the dose of glucocorticoids is not tapered after the PR3 CAAR cell treatment. In some embodiments, a glucocorticoid is administered to the subject at least 1 week before the PR3 CAAR cell or composition is administered to the subject. In some embodiments, a glucocorticoid is administered to the subject at least 2 weeks before the PR3 CAAR cell or composition is administered to the subject. In some embodiments, a glucocorticoid is administered to the subject at least 3 weeks before the PR3 CAAR cell or composition is administered to the subject. In some embodiments, a glucocorticoid is administered to the subject at least 4 weeks before the PR3 CAAR cell or composition is administered to the subject. In some embodiments, a glucocorticoid is administered to the subject at least 5 weeks before the PR3 CAAR cell or composition is administered to the subject. In some embodiments, a glucocorticoid is administered to the subject at least 6 weeks before the PR3 CAAR cell or composition is administered to the subject.

In some embodiments, a glucocorticoid is administered to the subject after the administration of the PR3 CAAR cell or composition is administered to the subject. In some embodiments, a glucocorticoid is administered to the subject for up to 1 week after the PR3 CAAR cell or composition is administered to the subject. In some embodiments, a glucocorticoid is administered to the subject for up to 2 weeks after the PR3 CAAR cell or composition is administered to the subject. In some embodiments, a glucocorticoid is administered to the subject for up to 3 weeks after the PR3 CAAR cell or composition is administered to the subject. In some embodiments, a glucocorticoid is administered to the subject for up to 4 weeks after the PR3 CAAR cell or composition is administered to the subject. In some embodiments, a glucocorticoid is administered to the subject for up to 5 weeks after the PR3 CAAR cell or composition is administered to the subject. In some embodiments, a glucocorticoid is administered to the subject for up to 6 weeks after the PR3 CAAR cell or composition is administered to the subject. In certain embodiments, the glucocorticoids are administered at a dose range between 0.1 mg/kg/day and 3 mg/kg/day.

In certain embodiments, the remission maintenance therapy can include one or more of rituximab, methotrexate, azathioprine, mycophenolate mofetil, leflunomide, mepolizumab, and omalizumab. In certain embodiments, provided herein are methods for treating a subject with any ANCA-associated disease or condition or anti-PR3 antibody-associated disease or condition (e.g. AAV) comprises administering to the subject a PR3 CAAR in combination with a remission induction therapy and a remission maintenance therapy.

### Dosing and Administration

In some embodiments, the methods include adoptive cell therapy, whereby genetically engineered cells expressing the provided PR3 CAARs cells are administered to subjects. Such administration can promote activation of the cells (e.g., T cell activation) in a ANCA-targeting manner, such that the cells of the disease or disorder are targeted for destruction.

Thus, the provided methods and uses include methods and uses for adoptive cell therapy. In some embodiments, the methods include administration of the cells or a composition containing the cells to a subject, tissue, or cell, such as one having, at risk for, or suspected of having the disease, condition or disorder. In some embodiments, the cells, populations, and compositions are administered to a subject having the particular disease or condition to be treated, e.g., via adoptive cell therapy, such as adoptive T cell therapy. In some embodiments, the cells or compositions are administered to the subject, such as a subject having or at risk for the disease or condition. In some aspects, the methods thereby treat, e.g., ameliorate one or more symptom of the disease or condition, such as by targeting and killing ANCA-producing immune cells (e.g., ANCA-producing B cells).

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent. In some examples, the patient or subject is a validated animal model for disease, adoptive cell therapy, and/or for assessing toxic outcomes such as cytokine release syndrome (CRS).

The PR3 CAARcells expressing the CAAR can be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, intracranial, intrathoracic, or subcutaneous administration. Dosing and administration may depend in part on whether the administration is brief or chronic. Various dosing schedules include but are not limited to single or multiple administrations over various time-points, bolus administration, and pulse infusion.

For the prevention or treatment of disease, the appropriate dosage of the binding molecule or cell may depend on the type of disease to be treated, the type of binding molecule, the severity and course of the disease, whether the binding molecule is administered for preventive or therapeutic purposes, previous therapy, the patient's clinical history and response to the binding molecule, and the discretion of the attending physician. The compositions and molecules and cells are in some embodiments suitably administered to the patient at one time or over a series of treatments.

Depending on the type and severity of the disease, dosages of antibodies may include at or about 1 μg/kg to at or about 15 mg/kg (e.g. at or about 0.1 mg/kg-at or about 10 mg/kg), at or about 1 μg/kg to at or about 100 mg/kg or more, at or about 0.05 mg/kg to at or about 10 mg/kg, at or about 0.5 mg/kg, at or about 2.0 mg/kg, at or about 4.0 mg/kg or at or about 10 mg/kg. Multiple doses may be administered intermittently, e.g. every week or every three weeks. An initial higher loading dose, followed by one or more lower doses may be administered.

In certain embodiments, the cells, or individual populations of sub-types of cells, are administered to the subject at a range of at or about 0.1 million to at or about 100 billion cells and/or that amount of cells per kilogram of body weight of the subject, such as, e.g., at or about 0.1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), at or about 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), such as at or about 10 million to at or about 100 billion cells (e.g., at or about 20 million cells, at or about 30 million cells, at or about 40 million cells, at or about 60 million cells, at or about 70 million cells, at or about 80 million cells, at or about 90 million cells, at or about 10 billion cells, at or about 25 billion cells, at or about 50 billion cells, at or about 75 billion cells, at or about 90 billion cells, or a range defined by any two of the foregoing values), and in some cases at or about 100 million cells to at or about 50 billion cells (e.g., at or about 120 million cells, at or about 250 million cells, at or about 350 million cells, at or about 650 million cells, at or about 800 million cells, at or about 900 million cells, at or about 3 billion cells, at or about 30 billion cells, at or about 45 billion cells) or any value in between these ranges and/or per kilogram of body weight of the subject. Dosages may vary depending on attributes particular to the disease or disorder and/or patient and/or other treatments. In some embodiments, such values refer to numbers of recombinant receptor-expressing cells; in other embodiments, they refer to number of T cells or PBMCs or total cells administered.

In some embodiments, for example, where the subject is a human, the dose includes fewer than about 5×10⁸ total recombinant receptor (e.g., CAAR)-expressing cells, T cells, or peripheral blood mononuclear cells (PBMCs), e.g., in the range of at or about 1×10⁶ to at or about 5×10⁸ such cells, such as at or about 2×10⁶, 5×10⁶, 1×10⁷, 5×10⁷, 1×10⁸, 1.5×10⁸, or 5×10⁸ total such cells, or the range between any two of the foregoing values. In some embodiments, for example, where the subject is a human, the dose includes more than at or about 1×10⁶ total recombinant receptor (e.g., CAAR)-expressing cells, T cells, or peripheral blood mononuclear cells (PBMCs) and fewer than at or about 2×10⁹ total recombinant receptor (e.g., CAAR)-expressing cells, T cells, or peripheral blood mononuclear cells (PBMCs), e.g., in the range of at or about 2.5×10⁷ to at or about 1.2×10⁹ such cells, such as at or about 2.5×10⁷, 5×10⁷, 1×10⁸, 1.5×10⁸, 8×10⁸, or 1.2×10⁹ total such cells, or the range between any two of the foregoing values.

In some embodiments, the dose of genetically engineered cells comprises from at least or at least about or is 1×10⁵ to at or about 5×10⁸ total CAAR-expressing (CAAR⁺) T cells, from at or about 1×10⁵ to at or about 2.5×10⁸ total CAAR⁺ T cells, from at or about 1×10⁵ to at or about 1×10⁸ total CAAR⁺ T cells, from at or about 1×10⁵ to at or about 5×10⁷ total CAAR⁺ T cells, from at or about 1×10⁵ to at or about 2.5×10⁷ total CAAR⁺ T cells, from at or about 1×10⁵ to at or about 1×10⁷ total CAAR⁺ T cells, from at or about 1×10⁵ to at or about 5×10⁶ total CAAR⁺ T cells, from at or about 1×10⁵ to at or about 2.5×10⁶ total CAAR⁺ T cells, from at or about 1×10⁵ to at or about 1×10⁶ total CAAR⁺ T cells, from at or about 1×10⁶ to at or about 5×10⁸ total CAAR⁺ T cells, from at or about 1×10⁶ to at or about 2.5×10⁸ total CAAR⁺ T cells, from at or about 1×10⁶ to at or about 1×10⁸ total CAAR⁺ T cells, from at or about 1×10⁶ to at or about 5×10⁷ total CAAR⁺ T cells, from at or about 1×10⁶ to at or about 2.5×10⁷ total CAAR⁺ T cells, from at or about 1×10⁶ to at or about 1×10⁷ total CAAR⁺ T cells, from at or about 1×10⁶ to at or about 5×10⁶ total CAAR⁺ T cells, from at or about 1×10⁶ to at or about 2.5×10⁶ total CAAR⁺ T cells, from at or about 2.5×10⁶ to at or about 5×10⁸ total CAAR⁺ T cells, from at or about 2.5×10⁶ to at or about 2.5×10⁸ total CAAR⁺ T cells, from at or about 2.5×10⁶ to at or about 1×10⁸ total CAAR⁺ T cells, from at or about 2.5×10⁶ to at or about 5×10⁷ total CAAR⁺ T cells, from at or about 2.5×10⁶ to at or about 2.5×10⁷ total CAAR⁺ T cells, from at or about 2.5×10⁶ to at or about 1×10⁷ total CAAR⁺ T cells, from at or about 2.5×10⁶ to at or about 5×10⁶ total CAAR⁺ T cells, from at or about 5×10⁶ to at or about 5×10⁸ total CAAR⁺ T cells, from at or about 5×10⁶ to at or about 2.5×10⁸ total CAAR⁺ T cells, from at or about 5×10⁶ to at or about 1×10⁸ total CAAR⁺ T cells, from at or about 5×10⁶ to at or about 5×10⁷ total CAAR⁺ T cells, from at or about 5×10⁶ to at or about 2.5×10⁷ total CAAR⁺ T cells, from at or about 5×10⁶ to at or about 1×10⁷ total CAAR⁺ T cells, from at or about 1×10⁷ to at or about 5×10⁸ total CAAR⁺ T cells, from at or about 1×10⁷ to at or about 2.5×10⁸ total CAAR⁺ T cells, from at or about 1×10⁷ to at or about 1×10⁸ total CAAR⁺ T cells, from at or about 1×10⁷ to at or about 5×10⁷ total CAAR⁺ T cells, from at or about 1×10⁷ to at or about 2.5×10⁷ total CAAR⁺ T cells, from at or about 2.5×10⁷ to at or about 5×10⁸ total CAAR⁺ T cells, from at or about 2.5×10⁷ to at or about 2.5×10⁸ total CAAR⁺ T cells, from at or about 2.5×10⁷ to at or about 1×10⁸ total CAAR⁺ T cells, from at or about 2.5×10⁷ to at or about 5×10⁷ total CAAR⁺ T cells, from at or about 5×10⁷ to at or about 5×10⁸ total CAAR⁺ T cells, from at or about 5×10⁷ to at or about 2.5×10⁸ total CAAR⁺ T cells, from at or about 5×10⁷ to at or about 1×10⁸ total CAAR⁺ T cells, from at or about 1×10⁸ to at or about 5×10⁸ total CAAR⁺ T cells, from at or about 1×10⁸ to at or about 2.5×10⁸ total CAAR⁺ T cells, from at or about or 2.5×10⁸ to at or about 5×10⁸ total CAAR⁺ T cells. In some embodiments, the dose of genetically engineered cells comprises from or from about 2.5×10⁷ to at or about 1.5×10⁸ total CAAR⁺ T cells, such as from or from about 5×10⁷ to or to about 1×10⁸ total CAAR⁺ T cells.

In some embodiments, the dose of genetically engineered cells comprises at least at or about 1×10⁵ CAAR⁺ cells, at least at or about 2.5×10⁵ CAAR⁺ cells, at least at or about 5×10⁵ CAAR⁺ cells, at least at or about 1×10⁶ CAAR⁺ cells, at least at or about 2.5×10⁶ CAAR⁺ cells, at least at or about 5×10⁶ CAAR⁺ cells, at least at or about 1×10⁷ CAAR⁺ cells, at least at or about 2.5×10⁷ CAAR⁺ cells, at least at or about 5×10⁷ CAAR⁺ cells, at least at or about 1×10⁸ CAAR⁺ cells, at least at or about 1.5×10⁸ CAAR⁺ cells, at least at or about 2.5×10⁸ CAAR⁺ cells, or at least at or about 5×10⁸ CAAR⁺ cells.

In some embodiments, the cell therapy comprises administration of a dose comprising a number of cell from or from about 1×10⁵ to or to about 5×10⁸ total CAAR-expressing cells, total T cells, or total peripheral blood mononuclear cells (PBMCs), from or from about 5×10⁵ to or to about 1×10⁷ total CAAR-expressing cells, total T cells, or total peripheral blood mononuclear cells (PBMCs) or from or from about 1×10⁶ to or to about 1×10⁷ total CAAR-expressing cells, total T cells, or total peripheral blood mononuclear cells (PBMCs), each inclusive. In some embodiments, the cell therapy comprises administration of a dose of cells comprising a number of cells at least or at least about 1×10⁵ total CAAR-expressing cells, total T cells, or total peripheral blood mononuclear cells (PBMCs), such at least or at least 1×10⁶, at least or at least about 1×10⁷, at least or at least about 1×10⁸ of such cells. In some embodiments, the number is with reference to the total number of CD3⁺ or CD8⁺, in some cases also CAAR-expressing cells. In some embodiments, the cell therapy comprises administration of a dose comprising a number of cells from or from about 1×10⁵ to or to about 5×10⁸ CD3⁺ or CD8⁺ total T cells or CD3⁺ or CD8⁺ recombinant receptor-expressing cells, from or from about 5×10⁵ to or to about 1×10⁷ CD3⁺ or CD8⁺ total T cells or CD3⁺ or CD8⁺ CAAR-expressing cells, or from or from about 1×10⁶ to or to about 1×10⁷ CD3⁺ or CD8⁺ total T cells or CD3⁺ or CD8⁺ CAAR-expressing cells, each inclusive. In some embodiments, the cell therapy comprises administration of a dose comprising a number of cell from or from about 1×10⁵ to or to about 5×10⁸ total CD3⁺/CAAR⁺ or CD8⁺/CAAR⁺ cells, from or from about 5×10⁵ to or to about 1×10⁷ total CD3⁺/CAAR⁺ or CD8⁺/CAAR⁺ cells, or from or from about 1×10⁶ to or to about 1×10⁷ total CD3⁺/CAAR⁺ or CD8⁺/CAAR⁺ cells, each inclusive.

In some embodiments, the T cells of the dose include CD4+ T cells, CD8+ T cells or CD4+ and CD8+ T cells.

In some embodiments, for example, where the subject is human, the CD8⁺ T cells of the dose, including in a dose including CD4⁺ and CD8⁺ T cells, includes between at or about 1×10⁶ and at or about 5×10⁸ total CAAR-expressing CD8⁺ cells, e.g., in the range of from at or about 5×10⁶ to at or about 1×10⁸ such cells, such as 1×10⁷, 2.5×10⁷, 5×10⁷, 7.5×10⁷, 1×10⁸, 1.5×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to or to about 0.75×10⁸ total recombinant receptor-expressing CD8⁺ T cells, from or from about 1×10⁷ to or to about 5×10⁷ total recombinant receptor-expressing CD8⁺ T cells, from or from about 1×10⁷ to or to about 0.25×10⁸ total recombinant receptor-expressing CD8⁺ T cells, each inclusive. In some embodiments, the dose of cells comprises the administration of at or about 1×10⁷, 2.5×10⁷, 5×10⁷, 7.5×10, 1×10⁸, 1.5×10⁸, 2.5×10⁸, or 5×10⁸ total recombinant receptor-expressing CD8⁺ T cells.

In some embodiments, the dose of cells, e.g., CAAR-expressing T cells, is administered to the subject as a single dose or is administered only one time within a period of two weeks, one month, three months, six months, 1 year or more.

In some embodiments, the cells or antibodies are administered as part of a combination treatment, such as simultaneously with or sequentially with, in any order, another therapeutic intervention, such as another antibody or engineered cell or receptor or agent, such as a cytotoxic or therapeutic agent.

The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells or antibodies are administered prior to the one or more additional therapeutic agents. In some embodiments, the cells are administered after to the one or more additional therapeutic agents.

Once the cells are administered to a mammal (e.g., a human), the biological activity of the engineered cell populations is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD 107a, IFNγ, IL-2, and TNF. In some aspects the biological activity is measured by assessing clinical outcome.

In certain embodiments, engineered cells are modified in any number of ways, such that their therapeutic or prophylactic efficacy is increased. For example, the engineered CAAR expressed by the population in some embodiments are conjugated either directly or indirectly through a linker to a targeting moiety. The practice of conjugating compounds, e.g., the CAAR to targeting moieties is known. See, for instance, Wadwa et al., J. Drug Targeting 3: 1 1 1 (1995), and U.S. Pat. No. 5,087,616.

### V. ARTICLES OF MANUFACTURE OR KITS

Also provided are articles of manufacture or kit containing the provided PR3 CAARs cells, such as those genetically engineered to express the CAAR, and/or compositions comprising the same. The articles of manufacture may include a container and a label or package insert on or associated with the container. Suitable containers include, for example, bottles, vials, syringes, test tubes, IV solution bags, etc. The containers may be formed from a variety of materials such as glass or plastic. In some embodiments, the container has a sterile access port. Exemplary containers include an intravenous solution bags, vials, including those with stoppers pierceable by a needle for injection. The article of manufacture or kit may further include a package insert indicating that the compositions can be used to treat a particular condition such as a condition described herein (e.g., vasculitis). Alternatively, or additionally, the article of manufacture or kit may further include another or the same container comprising a pharmaceutically-acceptable buffer. It may further include other materials such as other buffers, diluents, filters, needles, and/or syringes.

The label or package insert may indicate that the composition is used for treating an autoimmune disease, disorder or condition in an individual (e.g., ANCA-associated vasculitis, e.g., PR3 ANCA associated vasculitis). The label or a package insert, which is on or associated with the container, may indicate directions for reconstitution and/or use of the formulation. The label or package insert may further indicate that the formulation is useful or intended for subcutaneous, intravenous, or other modes of administration for treating or preventing an autoimmune disease, disorder or condition in an individual. In some aspects, the label or package insert can include instructions for use, for example instructions for administering the CAAR, the cell, or the composition, in some aspects in accord with any of the methods or uses described herein.

The container in some embodiments holds a composition which is by itself or combined with another composition effective for treating, preventing and/or diagnosing the condition. The article of manufacture or kit may include (a) a first container with a composition contained therein (i.e., first medicament), wherein the composition includes the CAAR; and (b) a second container with a composition contained therein (i.e., second medicament), wherein the composition includes a further agent, such as a cytotoxic or otherwise therapeutic agent, and which article or kit further comprises instructions on the label or package insert for treating the subject with the second medicament, in an effective amount.

### VI. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polypeptide” and “protein” are used interchangeably to refer to a polymer of amino acid residues, and are not limited to a minimum length. Polypeptides, including the antibodies and antibody chains and other peptides, e.g., linkers, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, “percent (%) amino acid sequence identity” and “percent identity” and “sequence identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acid substitutions may be introduced into a binding molecule, e.g., antibody, of interest and the products screened for a desired activity, e.g., retained/improved antigen binding, or decreased immunogenicity.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative amino acid substitutions will involve exchanging a member of one of these classes for another class.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

As used herein, the singular forms “a,” “an,” and “the” include plural referents unless the context clearly dictates otherwise. For example, “a” or “an” means “at least one” or “one or more.” It is understood that aspects, embodiments, and variations described herein include “comprising,” “consisting,” and/or “consisting essentially of” aspects, embodiments and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “composition” refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

### VII. EXEMPLARY EMBODIMENTS

### Among the Provided Embodiments are:

1. A chimeric autoantibody receptor (CAAR) comprising: (a) an extracellular proteinase 3 (PR3) antibody binding domain; (b) a transmembrane region; and (c) an intracellular signaling region.

2. The CAAR of embodiment 1, further comprising a spacer between the extracellular PR3 antibody binding domain and the transmembrane domain.

3. The CAAR of embodiment 1 or embodiment 2, wherein the PR3 antibody binding domain is from a human PR3 protein or is a mutant thereof.

4. The CAAR of any of embodiments 1-3, wherein the PR3 antibody binding domain is a mature PR3 protein.

5. The CAAR of any of embodiments 1-4, wherein the PR3 antibody binding domain comprises the sequence of amino acids set forth in SEQ ID NO:1 or a sequence of amino acids that exhibits at least 90% sequence identity to SEQ ID NO:1.

6. The CAAR of any of embodiments 1-5, wherein the PR3 antibody binding domain is a wild-type human PR3.

7. The CAAR of any of embodiments 1-6, wherein the PR3 antibody binding domain is set forth in SEQ ID NO:1.

8. The CAAR of any of embodiments 1-6, wherein the PR3 antibody binding domain is a mutant PR3 protein comprising an amino acid substitution at a position selected from the group consisting of G4, H44, D91, D175 and S176 with reference to positions set forth in SEQ ID NO:1.

9. The CAAR of embodiment 8, wherein the amino acid substitution is selected from the group consisting of G4P, H44A, D91N, D175N, S176A and S176C, or is a conservative substitution thereof.

10. The CAAR of any of embodiments 1-9, wherein the PR3 antibody binding domain is a mutant PR3 protein that has reduced enzymatic activity compared with a wild type PR3 protein (e.g., the PR3 protein set forth in SEQ ID NO:1).

11. The CAAR of embodiment 10, wherein the mutant PR3 protein comprises an amino acid substitution is in or near the catalytic triad, optionally wherein the amino acid substitution is in an active site residue selected from the group consisting of H44, D91 and S176, with reference to numbering set forth in SEQ ID NO:1, optionally wherein the amino acid substitution is selected from the group consisting of H44A, D91N, S176A and S176C.

12. The CAAR of embodiment 10, wherein the mutant PR3 protein comprises an amino acid substitution in at a position that interferes with the formation of the substrate binding pocket, optionally wherein the position is G4.

13. The CAAR of any of embodiments 8-12, wherein the mutant PR3 protein comprises 1, 2, 3, 4 or 5 amino acid substitutions compared to wild-type PR3 set forth in SEQ ID NO:1.

14. The CAAR of any of embodiments 8-10, 12 and 13, wherein the mutant PR3 protein comprises the amino acid substitution G4P.

15. The CAAR of any of embodiments 1-10, 12, 13 and 14, wherein the PR3 antibody binding domain comprises the sequence of amino acids set forth in SEQ ID NO:9 or a sequence of amino acids that exhibits at least 90% sequence identity to SEQ ID NO:9, optionally wherein the PR3 antibody binding domain is set forth in SEQ ID NO:9.

16. The CAAR of any of embodiments 8-11 and 13, wherein the mutant PR3 protein comprises the amino acid substitution H44A.

17. The CAAR of any of embodiments 1-11, 13 and 16, wherein the PR3 antibody binding domain comprises the sequence of amino acids set forth in SEQ ID NO:10 or a sequence of amino acids that exhibits at least 90% sequence identity to SEQ ID NO:10, optionally wherein the PR3 antibody binding domain is set forth in SEQ ID NO:10.

18. The CAAR of any of embodiments 8-11 and 13, wherein the mutant PR3 protein comprises the amino acid substitution D91N.

19. The CAAR of any of embodiments 1-11, 13 and 18, wherein the antibody binding domain comprises the sequence of amino acids set forth in SEQ ID NO:4 or a sequence of amino acids that exhibits at least 90% sequence identity to SEQ ID NO:4, optionally wherein the PR3 antibody binding domain is set forth in SEQ ID NO:4.

20. The CAAR of any of embodiments 8-11 and 13, wherein the mutant PR3 protein comprises the amino acid substitution D175N.

21. The CAAR of any of embodiments 1-11, 13 and 20, wherein the PR3 antibody binding domain comprises the sequence of amino acids set forth in SEQ ID NO:7 or a sequence of amino acids that exhibits at least 90% sequence identity to SEQ ID NO:7, optionally wherein the PR3 antibody binding domain is set forth in SEQ ID NO:7.

22. The CAAR of any of embodiments 8-11 and 13, wherein the mutant PR3 protein comprises the amino acid substitution S176A or S176C.

23. The CAAR of any of embodiments 1-11, 13 and 22, wherein the PR3 antibody binding domain comprises the sequence of amino acids set forth in SEQ ID NO:2 or a sequence of amino acids that exhibits at least 90% sequence identity to SEQ ID NO:2, optionally wherein the PR3 antibody binding domain is set forth in SEQ ID NO:2.

24. The CAAR of any of embodiments 1-11, 13 and 22, wherein the PR3 antibody binding domain comprises the sequence of amino acids set forth in SEQ ID NO:8 or a sequence of amino acids that exhibits at least 90% sequence identity to SEQ ID NO:8, optionally wherein the PR3 antibody binding domain is set forth in SEQ ID NO:8.

25. The CAAR of any of embodiments 1-24, wherein the transmembrane region is or comprises a transmembrane domain from CD4, CD28, or CD8.

26. The CAAR of any of embodiments 1-25, wherein the transmembrane region is or comprises a transmembrane domain from CD28, optionally a human CD28.

27. The CAAR of any of embodiments 1-26, wherein the transmembrane region is or comprises SEQ ID NO: 26 or an amino acid sequence having at least at or about 90%, 91%, 92%, 93%, 94%, 95%, 96%, 97%, 98% or 99% sequence identity to SEQ ID NO:26.

28. The CAAR of any of embodiments 1-27, wherein the transmembrane region is set forth in SEQ ID NO:26.

29. The CAAR of any of embodiments 1-28, wherein the intracellular signaling region comprises an intracellular signaling domain capable of inducing a primary activation signal in a T cell.

30. The CAAR of embodiments 29, wherein the intracellular signaling domain is a domain from a T cell receptor (TCR) component and/or comprises an immunoreceptor tyrosine-based activation motif (ITAM).

31. The CAAR of embodiment 29 or embodiment 30, wherein the intracellular signaling domain is a cytoplasmic signaling domain of a CD3-zeta (CD3) chain, optionally a human CD3 chain.

32. The CAAR of any of embodiments 29-31, wherein the intracellular signaling domain comprises the sequence set forth in SEQ ID NO:28, or an amino acid sequence having at least at or about 90%, 91%, 92%, 93%, 94%, 95%, 96%, 97%, 98% or 99% sequence identity to SEQ ID NO:28.

33. The CAAR of any of embodiments 29-32, wherein the intracellular signaling domain is set forth in SEQ ID NO:28.

34. The CAAR of any of embodiments 29-33, wherein the intracellular signaling region further comprises a costimulatory signaling region.

35. The CAAR of embodiment 34, wherein the costimulatory signaling region is between the transmembrane region and the intracellular signaling domain.

36. The CAAR of embodiment 34 or embodiment 35, wherein the costimulatory signaling region comprises an intracellular signaling domain of a T cell costimulatory molecule or a signaling portion thereof.

37. The CAAR of any of embodiments 34-36, wherein the costimulatory signaling region comprises an intracellular signaling domain of CD28, 4-1BB, or ICOS.

38. The CAAR of any of embodiments 34-37, wherein the costimulatory signaling region comprises an intracellular signaling domain of 4-1BB, optionally a human 4-1BB.

39. The CAAR of any of embodiments 34-38, wherein the costimulatory signaling region comprises the sequence set forth in SEQ ID NO:27 or an amino acid sequence having at least at or about 90%, 91%, 92%, 93%, 94%, 95%, 96%, 97%, 98% or 99% sequence identity to SEQ ID NO:27.

40. The CAAR of any of embodiments 34-39, wherein the costimulatory signaling region is set forth in SEQ ID NO:27.

41. The CAAR of any of embodiments 2-40, wherein the spacer comprises at least a portion of an immunoglobulin or a variant thereof.

42. The CAAR of any of embodiments 2-41, wherein the spacer comprises a hinge region of an immunoglobulin or a variant thereof.

43. The CAAR of embodiment 42, wherein the hinge region of an immunoglobulin is an IgG4 hinge region, optionally a human IgG4 hinge region, or a variant thereof.

44. The CAAR of any of embodiments 41-43, wherein the spacer comprises a variant IgG4 hinge region comprising substitution of amino acids CPSC to CPPC compared to the wild-type IgG4 hinge region.

45. The CAAR of any of embodiments 2-44, wherein the spacer is less than at or about 15 amino acids in length.

46. The CAAR of any of embodiments 2-44, wherein the spacer is between 12 and 15 amino acids in length.

47. The CAAR of any of embodiments 1-46, wherein the spacer comprises the sequence set forth in SEQ ID NO: 22 or SEQ ID NO:23.

48. The CAAR of any of embodiments 1-47, wherein the spacer is set forth in SEQ ID NO:22.

49. The CAAR of any of embodiments 2-44 and 47, wherein the spacer is between 100 and 150 amino acids in length, optionally between 110 and 130 amino acids in length.

50. The CAAR of any of embodiments 2-44, 47 and 49, wherein the spacer comprises a hinge region of an immunoglobulin and a CH3 region of an immunoglobulin.

51. The CAAR of embodiment 50, wherein the spacer comprises an IgG4 hinge region or a variant thereof and IgG4 CH3 region.

52. The CAAR of any of embodiments 2-44, 47 and 49-51, wherein the spacer comprises the sequence set forth in SEQ ID NO: 24 or SEQ ID NO:96.

53. The CAAR of any of embodiments 2-44, 47 and 49-52, wherein the spacer is set forth in SEQ ID NO:24.

54. The CAAR of any of embodiments 2-44 and 47, wherein the spacer is between 200 and 250 amino acids in length, optionally between 220 and 240 amino acids in length.

55. The CAAR of any of embodiments 2-44, 47 and 54 wherein the spacer comprises a hinge region of an immunoglobulin, a CH2 region of an immunoglobulin or a chimeric CH2 region of two different immunoglobulins, and a CH3 region of an immunoglobulin.

56. The CAAR of embodiment 55, wherein the spacer comprises IgG4 hinge region or a variant thereof, a chimeric CH2 region comprising a portion of an IgG4 CH2 and a portion of an IgG2 CH2 (IgG2/4 CH2 region), and an IgG4 CH3 region.

57. The CAAR of any of embodiments 2-44, 47 and 54-56, wherein the spacer comprises the sequence set forth in SEQ ID NO: 25 or SEQ ID NO:97.

58. The CAAR of any of embodiments 2-44, 47 and 54-57, wherein the spacer is set forth in SEQ ID NO:25.

59. The CAAR of any of embodiments 1-58, wherein the CAAR comprises a sequence of amino acids that exhibits at least 85% sequence identity to the sequence set forth in any of SEQ ID NOS: 29, 30, 31, 38, 39, 40, 53, 54, 55, 56, 57 or 58, optionally wherein the CARR comprises the sequence set forth in any one of SEQ ID NOS: 29, 30, 31, 38, 39, 40, 54, 55, 56, 57, 58 or 59.

60. A CAAR comprising the sequence of amino acids set forth in SEQ ID NO: 29 or a sequence of amino acids that exhibits at least 90% sequence identity to SEQ ID NO:29.

61. A CAAR comprising the sequence of amino acids set forth in SEQ ID NO: 30 or a sequence of amino acids that exhibits at least 90% sequence identity to SEQ ID NO:30.

62. A CAAR comprising the sequence of amino acids set forth in SEQ ID NO: 38 or a sequence of amino acids that exhibits at least 90% sequence identity to SEQ ID NO:38.

63. A CAAR comprising the sequence of amino acids set forth in SEQ ID NO: 55 or a sequence of amino acids that exhibits at least 90% sequence identity to SEQ ID NO:55.

64. A CAAR comprising the sequence of amino acids set forth in SEQ ID NO: 56 or a sequence of amino acids that exhibits at least 90% sequence identity to SEQ ID NO:56.

65. A CAAR comprising the sequence of amino acids set forth in SEQ ID NO: 57 or a sequence of amino acids that exhibits at least 90% sequence identity to SEQ ID NO:57.

66. A CAAR comprising the sequence of amino acids set forth in SEQ ID NO: 58 or a sequence of amino acids that exhibits at least 90% sequence identity to SEQ ID NO:58.

67. The CAAR of any of embodiments 1-66, wherein the CAAR is able to be specifically bound by an anti-PR3 antineutrophil cytoplasmic antibody (PR3-ANCA).

68. The CAAR of any of embodiments 1-67, wherein the PR3 antibody binding domain comprises at least 2, 3, 4, 5, 6 or 7 epitopes recognized by an anti-PR3 antineutrophil cytoplasmic antibody (PR3-ANCA).

69. The CAAR of embodiment 68, wherein the epitopes are selected from the group consisting of ANCA2 (AQPHSRPYMAS, SEQ ID NO:90), ANCA3 (PGSHFCGG, SEQ ID NO:91), ANCA4 (VVLGAHNVRTQ, SEQ ID NO:92), ANCA5 (FLNNYDAE, SEQ ID NO:93), ANCA6 (PVPHGTQC, SEQ ID NO: 94) and ANCA7 (CFGDSGGP, SEQ ID NO:95).

70. The CAAR of any of embodiments 1-69, wherein the CAAR is specific for PR3-reactive B cells.

71. A polynucleotide comprising a nucleic acid encoding the CAAR of any of embodiments 1-70.

72. The polynucleotide of embodiment 71, wherein the polynucleotide is optimized by splice site elimination.

73. The polynucleotide of embodiment 71 or embodiment 72, wherein the polynucleotide is codon-optimized for expression in a human cell.

74. The polynucleotide of any of embodiments 71-73, wherein the polynucleotide comprises a nucleic acid sequence that exhibits at least 85% sequence identity to the sequence set forth in any of SEQ ID NOS: 59, 60, 61, 68, 69, 70, 83, 84, 85, 86, 87 or 88, optionally wherein the polynucleotide comprises the sequence set forth in any one of SEQ ID NOS: 59, 60, 61, 68, 69, 70, 83, 84, 85, 86, 87 or 88.

75. The polynucleotide of any of embodiments 71-74, wherein the polynucleotide comprises a nucleic acid sequence that exhibits at least 90% sequence identity to the sequence set forth in any of SEQ ID NOS: 59, 60, 68, 85, 86, 87 or 88, optionally wherein the polynucleotide comprises the sequence set forth in any one of SEQ ID NOS: 59, 60, 68, 85, 86, 87 or 88.

76. A vector, comprising the polynucleotide of any of embodiments 71-75.

77. The vector of embodiment 76, wherein the vector is a viral vector.

78. The vector of embodiment 77, wherein the viral vector is a retroviral vector (e.g., lentiviral vector).

79. A cell comprising the CAAR of any of embodiments 1-70.

80. A cell comprising the polynucleotide of any of embodiments 71-75 or the vector of any of embodiments 76-78.

81. The cell of embodiment 79 or embodiment 80, that is a lymphocyte.

82. The cell of any of embodiments 79-81, that is an NK cell or a T cell.

83. The cell of any of embodiments 79-82, wherein the cell is a T cell and the T cell is a CD4+ T cell or a CD8+ T cell.

84. The cell of any of embodiments 79-83, wherein the cell is a primary cell obtained from a subject.

85. The cell of any of embodiments 79-84, wherein the cell exhibits cytotoxic activity against PR3-reactive B cells.

86. The cell of embodiment 85, wherein the cytotoxic activity is against a plurality of PR3-reactive B cells, wherein the plurality of PR3-reactive B cells are specific for two or more different PR3 antineutrophil cytoplasmic antibody (PR3-ANCA) epitopes.

87. The cell of embodiment 86, wherein the plurality of PR3-reactive B cells are specific for at least 2, 3, 4, 5, 6 or 7 different PR3 ANCA epitopes.

88. The cell of embodiment 86 or embodiment 87, wherein the plurality of PR3-reactive B cells are specific for at least 4 different PR3 ANCA eptitopes.

89. The cell of any of embodiments 86-88, wherein the plurality of PR3-reactive B cells are specific for at least 5 different PR3 ANCA epitopes.

90. The cell of any of embodiments 86-89, wherein the plurality of PR3-reactive B cells are specific for at least 6 different PR3 ANCA epitopes.

91. The cell of any of embodiments 86-90, wherein the ANCA epitopes are selected from the group consisting of ANCA2 (AQPHSRPYMAS, SEQ ID NO:90), ANCA3 (PGSHFCGG, SEQ ID NO:91), ANCA4 (VVLGAHNVRTQ, SEQ ID NO:92), ANCA5 (FLNNYDAE, SEQ ID NO:93), ANCA6 (PVPHGTQC, SEQ ID NO: 94) and ANCA7 (CFGDSGGP, SEQ ID NO:95).

92. A composition comprising the cell of any of embodiments 79-91.

93. The composition of embodiment 92, further comprising a pharmaceutically acceptable excipient.

94. The composition of embodiment 92 or embodiment 93, wherein the composition comprises CD4+ and CD8+ T cells.

95. The composition of embodiment 94, wherein the ratio of CD4+ to CD8+ T cells is from at or about 1:3 to 3:1, optionally at or about 1:2 to 2:1, optionally at or about 1:1.

96. The composition of any of embodiments 92-95, wherein greater than at or about 90%, greater than at or about 95% or greater than at or about 98% of cells in the composition are CD3+ T cells.

97. The composition of any of embodiments 92-96, wherein at least 40%, at least 50%, at least 60%, at least 70%, at least 80% or at least 90% of cells in the composition express the CAAR.

98. The composition of any of embodiments 92-97, wherein, among a plurality of the cells in the composition expressing the CAAR, less than at or about 10%, at or about 9%, at or about 8%, at or about 7%, at or about 5%, at or about 4%, at or about 3%, at or about 2% or at or about 1% of the cells in the plurality exhibits tonic signaling and/or antigen independent activity or signaling.

99. A method of killing PR3-reactive B cells, the method comprising contacting a PR3-reactive B cell with the cell of any of embodiments 79-91 or the composition of any of embodiments 92-98.

100. The method of embodiment 99 that is performed in vitro or ex vivo.

101. The method of embodiment 99 that is performed in vivo in a subject.

102. The method of any of embodiments 99-101, wherein the PR3-reactive B cell comprises a plurality of PR3-reactive B cells, wherein the plurality of PR3-reactive B cells are specific for two or more different PR3 antineutrophil cytoplasmic antibody (PR3-ANCA) epitopes.

103. The method of embodiment 102, wherein the plurality of PR3-reactive B cells are specific for 2, 3, 4, 5, 6 or 7 different PR3 ANCA epitopes.

104. The method of embodiment 102 or embodiment 103, wherein the plurality of PR3-reactive B cells are specific for at least 4 different PR3 ANCA eptitopes.

105. The method of any of embodiments 102-104, wherein the plurality of PR3-reactive B cells are specific for at least 5 different PR3ANCA epitopes.

106. The method of any of embodiments 102-105, wherein the plurality of PR3-reactive B cells are specific for at least 6 different PR3 ANCA epitopes.

107. The method of any of embodiments 102-106, wherein the ANCA epitopes are selected from the group consisting of ANCA2 (AQPHSRPYMAS, SEQ ID NO:90), ANCA3 (PGSHFCGG, SEQ ID NO:91), ANCA4 (VVLGAHNVRTQ, SEQ ID NO:92), ANCA5 (FLNNYDAE, SEQ ID NO:93), ANCA6 (PVPHGTQC, SEQ ID NO: 94) and ANCA7 (CFGDSGGP, SEQ ID NO:95).

108. The method of any of embodiment 99-107, wherein, among a plurality of PR3-reactive B cells, the method results in killing of greater than at or about 80%, greater than at or about 90%, greater than at or about 95%, PR3-reactive B cells, optionally wherein the PR3-reactive B cells are IgG+.

109. A method of treating a disease or disorder in a subject, the method comprising administering the cell of any of embodiments 79-90 or the composition of any of embodiments 91-98 to a subject in need of treatment thereof.

110. The method of embodiment 109, wherein the disease or disorder is an autoimmune condition.

111. The method of embodiment 106 and embodiment 107, wherein the disease or disorder is antineutrophil cytoplasmic antibody (ANCA)-associated vasculitis (AAV), optionally wherein the AAV is PR3-AAV.

112. The method of any of embodiments 109-111, wherein the disease or disorder is selected from the group consisting of granulomatosis with polyangiitis (GPA), microscopic polyangiitis (MPA), or eosinophilic granulomatosis with polyangiitis (EGPA).

113. The method of embodiment 111 or 112, wherein the subject is positive for PR3-ANCA.

114. The method of any one of embodiments 111 to 113, wherein the method comprises testing the subject for PR3-ANCA.

115. The method of embodiment 114, wherein the method comprises verifying that the subject has tested positive for PR3-ANCA.

116. The method of any of embodiments 111 to 115, wherein the subject has been treated with a prior therapy (e.g., one or more prior therapies) for AAV.

117. The method of embodiment 116, wherein the subject has failed to achieve remission or has relapsed following treatment with the prior therapy.

118. The method of embodiment 116 or 117, wherein the prior therapy comprises one or more of a glucocorticoid, cyclophosphamide (CYC), rituximab (RTX), plasma exchange, Avacopan, methotrexate (MTX), mycophenolate mofetil (MMF), azathioprine (AZA), leflunomide (LEF), belimumab, mepolizumab, and omalizumab.

119. The method of embodiment 118, wherein the glucocorticoid is one or more of prednisolone, methylprednisolone, prednisone, dexamethasone.

120. The method of any one of embodiments 116 to 119, wherein the prior therapy comprises a remission induction therapy (e.g., a remission induction agent).

121. The method of embodiment 119, wherein the remission induction therapy comprises one or more of rituximab, cyclophosphamide, and a glucocorticoid.

122. The method of embodiment 121, wherein the glucocorticoid is one or more of prednisolone, methylprednisolone, prednisone, and dexamethasone.

123. The method of embodiment 122, wherein the glucocorticoid is prednisolone.

124. The method of any one of embodiments 116 to 123, wherein the prior therapy comprises a remission maintenance therapy.

125. The method of embodiment 124, wherein the remission maintenance therapy comprises one or more of rituximab, methotrexate, azathioprine, mycophenolate mofetil, leflunomide, mepolizumab, and omalizumab.

126. A method of treating a subject having PR3-ANCA vasculitis, the method comprising (i) administering a remission induction therapy to the subject and (ii) administering the cell of any of embodiments 79-90 or the composition of any of embodiments 91-98 to the subject.

127. The method of embodiment 126, wherein the remission induction therapy comprises cyclophosphamide.

128. The method of embodiment 127, wherein cyclophosphamide is administered to the subject before the cell or composition is administered to the subject.

129. The method of embodiment 127, wherein a course of cyclophosphamide is administered to the subject at least 1 week, 2 weeks, 3 weeks, or 4 weeks before the cell or composition is administered to the subject.

130. The method of any one of embodiments 126 to 129, wherein the remission induction therapy further comprises a glucocorticoid.

131. The method of embodiment 130, wherein the glucocorticoid is prednisolone.

132. A method of treating a subject having PR3-ANCA vasculitis, the method comprising (i) administering cyclophosphamide to the subject and (ii) administering the cell of any of embodiments 79-90 or the composition of any of embodiments 91-98 to the subject.

133. The method of embodiment 132, comprising administering cyclophosphamide to the subject before administering the cell of any of embodiments 79-90 or the composition of any of embodiments 91-98 to the subject.

134. The method of embodiment 133, wherein a course of cyclophosphamide is administered to the subject at least 1 week, 2 weeks, 3 weeks, or 4 weeks before the cell or composition is administered to the subject.

135. The method of embodiment 132 or 133, wherein the course of cyclophosphamide is administered according to a dosing schedule that has been determined to be sufficient to reduce ANCA titers in a patient having PR3-ANCA vasculitis.

136. The method of any one of embodiments 131 to 135, wherein cyclophosphamide is administered at an oral dose of 2 mg/kg/day for at least 1 week, 2 weeks, 3 weeks, or 4 weeks before the cell or composition is administered to the subject.

137. The method of any one of embodiments 131 to 135, wherein cyclophosphamide is administered at an IV dose of 15 mg/kg every 2 weeks for up to 3 doses prior to administering the cell or composition to the subject.

138. The method of any one of embodiments 131 to 137, wherein after the cell or composition is administered to the subject, the subject is not treated with cyclophosphamide.

139. The method of any one of embodiments 131 to 138, comprising administering a glucocorticoid to the subject before administering the cell or composition to the subject.

140. The method of any one of embodiments 131 to 139, comprising administering a glucocorticoid to the subject after administering the cell or composition to the subject.

141. A method of treating a subject having PR3-ANCA vasculitis, the method comprising (i) administering to the subject a preconditioning therapy that has been shown to be effective to deplete ANCAs and subsequently (ii) administering the cell of any of embodiments 79-90 or the composition of any of embodiments 91-98 to the subject.

142. The method of embodiment 141, wherein the preconditioning therapy comprises cyclophosphamide.

### VIII. EXAMPLES

The following examples are included for illustrative purposes only and are not intended to limit the scope of the invention.

### Example 1: Chimeric Autoantibody Receptor Design and Sequences

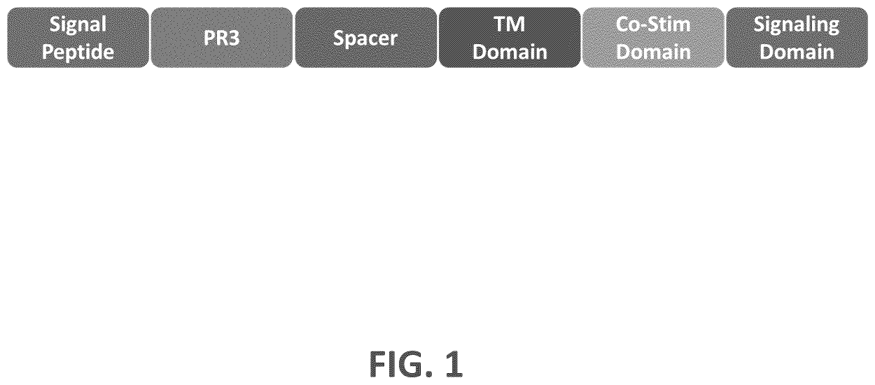

Chimeric autoantibody receptors (CAARs) were designed with components as shown in FIG. 1: signal peptide, PR3 protein (wild type or variant), a spacer, a transmembrane domain (TM), a costimulatory domain, and a signalling domain. Table E1 provides sequence ID numbers for amino acid (aa) and nucleotide (nt) sequences of CAARs and components of the CAARs. The CAARs were designed with the following components:
(1) an extracellular domain from one of ten human PR3 domains (either native PR3 protein (wild-type, ‘WT’) or PR3 protein with a designated modification (S176A, unprocessed C-terminal pro-peptide, D91N, unprocessed N-terminal pro-peptide, N-terminal Ile (I) deletion, D175N, S176C, G4P or H44A);
(2) a spacer chosen among an IgG4-derived spacer (a short spacer (“SS”) that is a modified IgG4 hinge, a medium spacer (“MS”) that is a modified IgG4 hinge plus IgG4-CH3 domain, or a long spacer (“LS”) that is a modified IgG4 hinge+IgG4-CH2 domain+IgG4-CH3 domain), a CD8a, or a peptide linker ((G4S)₃, G4S or EAAAK), and
(3) a transmembrane domain from a CD8a transmembrane domain or a CD28 transmembrane domain.

The CAAR constructs listed in Table E1 were generated and tested as described herein in Examples 2 to 10; the CAARs were generated on lentiviral backbones with sequential protein domains as follows: CD33 or PR3 signal peptide, human PR3 protein (wild type or variant), a spacer, a CD28 or CD8a transmembrane domain, a 4-1BB costimulatory signaling domain and a CD3ζ intracellular signaling domain.

TABLE E1
Components of PR3 CAAR constructs

| | | SEQ ID NO: | | | | | | | | |
|---|---|---|---|---|---|---|---|---|---|---|
| | | signal | | | | | Intracellular | | | |
| | CAAR | peptide | PR3 | | | | region | | CAAR | |
| Descriptor | Construct | aa | aa | nt | spacer | TM | 4-1BB | CD3ζ | aa | nt |
| PR3_1_29 | PR3_1_(WT) | 21 | 1 | 11 | 22 | 26 | 27 | 28 | 29 | 59 |
| | CD33SP SS | | | | | | | | | |
| | CD28TM | | | | | | | | | |
| PR3_1_30 | PR3_1_(WT) | 21 | 1 | 11 | 24 | 26 | 27 | 28 | 30 | 60 |
| | CD33SP MS | | | | | | | | | |
| | CD28TM | | | | | | | | | |
| PR3_1_31 | PR3_1_(WT) | 21 | 1 | 11 | 25 | 26 | 27 | 28 | 31 | 61 |
| | CD33SP LS | | | | | | | | | |
| | CD28TM | | | | | | | | | |
| PR3_2_32 | PR3_2 (176A) | 21 | 2 | 12 | 22 | 26 | 27 | 28 | 32 | 62 |
| | CD33SP SS | | | | | | | | | |
| | CD28TM | | | | | | | | | |
| PR3_2_33 | PR3_2 (176A) | 21 | 2 | 12 | 24 | 26 | 27 | 28 | 33 | 63 |
| | CD33SP MS | | | | | | | | | |
| | CD28TM | | | | | | | | | |
| PR3_2_34 | PR3_2 (176A) | 21 | 2 | 12 | 25 | 26 | 27 | 28 | 34 | 64 |
| | CD33SP LS | | | | | | | | | |
| | CD28TM | | | | | | | | | |
| PR3_3_35 | PR3_3 (C-terminal | 21 | 3 | 13 | 22 | 26 | 27 | 28 | 35 | 65 |
| | addition) CD33SP | | | | | | | | | |
| | SS CD28TM | | | | | | | | | |
| PR3_3_36 | PR3_3 (C-terminal | 21 | 3 | 13 | 24 | 26 | 27 | 28 | 36 | 66 |
| | addition) CD33SP | | | | | | | | | |
| | MS CD28TM | | | | | | | | | |
| PR3_3_37 | PR3_3 (C-terminal | 21 | 3 | 13 | 25 | 26 | 27 | 28 | 37 | 67 |
| | addition) CD33SP | | | | | | | | | |
| | LS CD28TM | | | | | | | | | |
| PR3_4_38 | PR3_4 (D91N) | 21 | 4 | 14 | 22 | 26 | 27 | 28 | 38 | 68 |
| | CD33SP SS | | | | | | | | | |
| | CD28TM | | | | | | | | | |
| PR3_4_39 | PR3_4 (D91N) | 21 | 4 | 14 | 24 | 26 | 27 | 28 | 39 | 69 |
| | CD33SP MS | | | | | | | | | |
| | CD28TM | | | | | | | | | |
| PR3_4_40 | PR3_4 (D91N) | 21 | 4 | 14 | 25 | 26 | 27 | 28 | 40 | 70 |
| | CD33SP LS | | | | | | | | | |
| | CD28TM | | | | | | | | | |
| PR3_5_41 | PR3 5 | 21 | 5 | 15 | 22 | 26 | 27 | 28 | 41 | 71 |
| | (Unprocessed N- | | | | | | | | | |
| | terminus) CD33SP | | | | | | | | | |
| | SS CD28TM | | | | | | | | | |
| PR3_5_42 | PR3 5 | 21 | 5 | 15 | 24 | 26 | 27 | 28 | 42 | 72 |
| | (Unprocessed N- | | | | | | | | | |
| | terminus) CD33SP | | | | | | | | | |
| | MS CD28TM | | | | | | | | | |
| PR3_5_43 | PR3 5 | 21 | 5 | 15 | 25 | 26 | 27 | 28 | 43 | 73 |
| | (Unprocessed N- | | | | | | | | | |
| | terminus) CD33SP | | | | | | | | | |
| | LS CD28TM | | | | | | | | | |
| PR3_6_44 | PR3_6 (Ile | 21 | 6 | 16 | 22 | 26 | 27 | 28 | 44 | 74 |
| | Deletion) CD33SP | | | | | | | | | |
| | SS CD28TM | | | | | | | | | |
| PR3_6_45 | PR3_6 (Ile | 21 | 6 | 16 | 24 | 26 | 27 | 28 | 45 | 75 |
| | Deletion) CD33SP | | | | | | | | | |
| | MS CD28TM | | | | | | | | | |
| PR3_6_46 | PR3_6 (Ile | 21 | 6 | 16 | 25 | 26 | 27 | 28 | 46 | 76 |
| | Deletion) CD33SP | | | | | | | | | |
| | LS CD28TM | | | | | | | | | |
| PR3_7_47 | PR3_7 (D175N) | 21 | 7 | 17 | 22 | 26 | 27 | 28 | 47 | 77 |
| | CD33SP SS | | | | | | | | | |
| | CD28TM | | | | | | | | | |
| PR3_7_48 | PR3_7 (D175N) | 21 | 7 | 17 | 24 | 26 | 27 | 28 | 48 | 78 |
| | CD33SP MS | | | | | | | | | |
| | CD28TM | | | | | | | | | |
| PR3_7_49 | PR3_7 (D175N) | 21 | 7 | 17 | 25 | 26 | 27 | 28 | 49 | 79 |
| | CD33SP LS | | | | | | | | | |
| | CD28TM | | | | | | | | | |
| PR3_8_50 | PR3_8 (S176C) | 21 | 8 | 18 | 22 | 26 | 27 | 28 | 50 | 80 |
| | CD33SP SS | | | | | | | | | |
| | CD28TM | | | | | | | | | |
| PR3_8_51 | PR3_8(S176C) | 21 | 8 | 18 | 24 | 26 | 27 | 28 | 51 | 81 |
| | CD33SP MS | | | | | | | | | |
| | CD28TM | | | | | | | | | |
| PR3_8_52 | PR3_8 (S176C) | 21 | 8 | 18 | 25 | 26 | 27 | 28 | 52 | 82 |
| | CD33SP LS | | | | | | | | | |
| | CD28TM | | | | | | | | | |
| PR3_9_53 | PR3_9 (G4P) | 21 | 9 | 19 | 22 | 26 | 27 | 28 | 53 | 83 |
| | CD33SP SS | | | | | | | | | |
| | CD28TM | | | | | | | | | |
| PR3_9_54 | PR3_9 (G4P) | 21 | 9 | 19 | 24 | 26 | 27 | 28 | 54 | 84 |
| | CD33SP MS | | | | | | | | | |
| | CD28TM | | | | | | | | | |
| PR3_9_55 | PR3_9 (G4P) | 21 | 9 | 19 | 25 | 26 | 27 | 28 | 55 | 85 |
| | CD33SP LS | | | | | | | | | |
| | CD28TM | | | | | | | | | |
| PR3_10_56 | PR3_10 (H44A) | 21 | 10 | 20 | 22 | 26 | 27 | 28 | 56 | 86 |
| | CD33SP SS | | | | | | | | | |
| | CD28TM | | | | | | | | | |
| PR3_10_57 | PR3_10 (H44A) | 21 | 10 | 20 | 24 | 26 | 27 | 28 | 57 | 87 |
| | CD33SP MS | | | | | | | | | |
| | CD28TM | | | | | | | | | |
| PR3_10_58 | PR3_10 (H44A) | 21 | 10 | 20 | 25 | 26 | 27 | 28 | 58 | 88 |
| | CD33SP LS | | | | | | | | | |
| | CD28TM | | | | | | | | | |
| PR3_9_103 | PR3_9 (G4P) | 21 | 9 | 19 | 100 | 98 | 27 | 28 | 103 | 144 |
| | CD33SP (G4S)3 | | | | | | | | | |
| | CD8aTM | | | | | | | | | |
| PR3_9_104 | PR3_9 (G4P) | 21 | 9 | 19 | 100 | 26 | 27 | 28 | 104 | 145 |
| | CD33SP (G4S)3 | | | | | | | | | |
| | CD28TM | | | | | | | | | |
| PR3_9_105 | PR3_9 (G4P) | 21 | 9 | 19 | 99 | 98 | 27 | 28 | 105 | 146 |
| | CD33SP (G4S) | | | | | | | | | |
| | CD8aTM | | | | | | | | | |
| PR3_9_106 | PR3_9 (G4P) | 21 | 9 | 19 | 99 | 26 | 27 | 28 | 106 | 147 |
| | CD33SP (G4S) | | | | | | | | | |
| | CD28TM | | | | | | | | | |
| PR3_9_107 | PR3_9 (G4P) | 21 | 9 | 19 | 101 | 98 | 27 | 28 | 107 | 148 |
| | CD33SP CD8a | | | | | | | | | |
| | CD8aTM | | | | | | | | | |
| PR3_9_108 | PR3_9 (G4P) | 21 | 9 | 19 | 102 | 98 | 27 | 28 | 108 | 149 |
| | CD33SP EAAAK | | | | | | | | | |
| | CD8aTM | | | | | | | | | |
| PR3_9_109 | PR3_9 (G4P) | 21 | 9 | 19 | 24 | 98 | 27 | 28 | 109 | 150 |
| | CD33SP MS | | | | | | | | | |
| | CD8aTM | | | | | | | | | |
| PR3_9_110 | PR3_9 (G4P) | 21 | 9 | 19 | 22 | 98 | 27 | 28 | 110 | 151 |
| | CD33SP SS | | | | | | | | | |
| | CD8aTM | | | | | | | | | |
| PR3_10_111 | PR3_10 (H44A) | 21 | 10 | 20 | 100 | 98 | 27 | 28 | 111 | 152 |
| | CD33SP (G4S)3 | | | | | | | | | |
| | CD8aTM | | | | | | | | | |
| PR3_10_112 | PR3_10 (H44A) | 21 | 10 | 20 | 100 | 26 | 27 | 28 | 112 | 153 |
| | CD33SP (G4S)3 | | | | | | | | | |
| | CD28TM | | | | | | | | | |
| PR3_10_113 | PR3_10 (H44A) | 21 | 10 | 20 | 99 | 98 | 27 | 28 | 113 | 154 |
| | CD33SP (G4S) | | | | | | | | | |
| | CD8aTM | | | | | | | | | |
| PR3_10_114 | PR3_10 (H44A) | 21 | 10 | 20 | 99 | 26 | 27 | 28 | 114 | 155 |
| | CD33SP (G4S) | | | | | | | | | |
| | CD28TM | | | | | | | | | |
| PR3_10_115 | PR3_10 (H44A) | 21 | 10 | 20 | 101 | 98 | 27 | 28 | 115 | 156 |
| | CD33SP CD8a | | | | | | | | | |
| | CD8aTM | | | | | | | | | |
| PR3_10_116 | PR3_10 (H44A) | 21 | 10 | 20 | 102 | 98 | 27 | 28 | 116 | 157 |
| | CD33SP EAAAK | | | | | | | | | |
| | CD8aTM | | | | | | | | | |
| PR3_10_117 | PR3_10 (H44A) | 21 | 10 | 20 | 24 | 98 | 27 | 28 | 117 | 158 |
| | CD33SP MS | | | | | | | | | |
| | CD8aTM | | | | | | | | | |
| PR3_10_118 | PR3_10 (H44A) | 21 | 10 | 20 | 22 | 98 | 27 | 28 | 118 | 159 |
| | CD33SP SS | | | | | | | | | |
| | CD8aTM | | | | | | | | | |
| PR3_10_111 | PR3_10 (H44A) | 123 | 10 | 20 | 100 | 98 | 27 | 28 | 119, | — |
| PR3SP | PR3SP (G4S)3 | | | | | | | | 111* | |
| | CD8aTM | | | | | | | | | |
| PR3_10_112 | PR3_10 (H44A) | 123 | 10 | 20 | 100 | 26 | 27 | 28 | 120, | — |
| PR3SP | PR3SP (G4S)3 | | | | | | | | 112* | |
| | CD28TM | | | | | | | | | |
| PR3_10_113 | PR3_10 (H44A) | 123 | 10 | 20 | 99 | 98 | 27 | 28 | 121, | — |
| PR3SP | PR3SP (G4S) | | | | | | | | 113* | |
| | CD8aTM | | | | | | | | | |
| PR3_10_114 | PR3_10 (H44A) | 123 | 10 | 20 | 99 | 26 | 27 | 28 | 122, | — |
| PR3SP | PR3SP (G4S) | | | | | | | | 114* | |
| | CD28TM | | | | | | | | | |

*This is without the N-terminal activation dipeptide

### Example 2: Differential Tonic Signaling and Activation Observed for Human PR3-CAAR Constructs

Single PR3-CAAR constructs were generated and stably transduced into a human Jurkat T cell line containing a Nur77 knock-in reporter (see e.g. WO 2019/089982). The Nur77 knock-in cell line contained nucleic acid sequences encoding a reporter molecule (a red fluorescent protein, Td-Tomato) knocked-in at the endogenous Nur77 locus, which is an immediate-early response gene induced by stimulation of signal from the T cell receptor and/or via molecules containing immunoreceptor tyrosine-based activation motif (ITAM).

Jurkat reporter cells stable transduced with the single CAAR constructs were cultured in RPMI culture media containing 10% heat-inactivated fetal bovine serum (FBS) for 24 hours. Tonic signaling (signaling in the absence of target) via the CAARs was measured by fluorescence of Td-Tomato by flow cytometry and plotted as the total percentage of cells that express Td-Tomato in the transduced Jurkat cell lines. As seen in Table E2 below, many of the constructs demonstrated low tonic signaling of 20% or less. For instance, CAAR constructs PR31_SS (PR3_129), PR3_1_MS (PR3_130), PR3_1LS (PR3_1_31), PR3_4_SS (PR3_4_38), PR3_7SS (PR3_7_47), PR3_9_LS (PR39_55) exhibited lower levels of tonic signaling ranging from 4 to 20% and CAAR constructs PR3_9_104, PR3_9_110, PR3_10_111, and PR3_10_111 PR3SP exhibiting tonic signaling levels around or below 1%.

TABLE E2
Differential tonic signaling observed across
PR3-CAAR+ Nur77-TdTomato reporter
Jurkat cells

| Construct | Mutation |
|---|---|
| PR3_1 | WT (native) |
| PR3_2 | S176A |
| PR3_3 | Unprocessed C |
| | terminus |
| PR3_4 | D91N |
| PR3_5 | Unprocessed N |
| | terminus |
| PR3_6 | N terminus Ile |
| | deletion |
| PR3_7 | D175N |
| PR3_8 | S176C |
| PR3_9 | G4P |
| PR3_10 | H44A |
| Construct | Tonic Signaling |
| Descriptor | (%) |
| PR3_1_29 | 3% |
| PR3_1_30 | 2% |
| PR3_1_31 | 11% |
| PR3_2_32 | 15% |
| PR3_2_33 | 30% |
| PR3_2_34 | 39% |
| PR3_3_35 | 58% |
| PR3_3_36 | 40% |
| PR3_3_37 | 43% |
| PR3_4_38 | 0.03% |
| PR3_4_39 | 27% |
| PR3_4_40 | 41% |
| PR3_5_41 | 59% |
| PR3_5_42 | 40% |
| PR3_5_43 | 43% |
| PR3_6_44 | 16% |
| PR3_6_45 | 52% |
| PR3_6_46 | 64% |
| PR3_7_47 | 11% |
| PR3_7_48 | 34% |
| PR3_7_49 | 52% |
| PR3_8_50 | 17% |
| PR3_8_51 | 36% |
| PR3_8_52 | 35% |
| PR3_9_53 | 5% |
| PR3_9_54 | 30% |
| PR3_9_55 | 22% |
| PR3_10_56 | 3% |
| PR3_10_57 | 12% |
| PR3_10_58 | 60% |
| PR3_9_103 | 0.3% |
| PR3_9_104 | 1.1% |
| PR3_9_105 | 0.03% |
| PR3_9_106 | 0.03% |
| PR3_9_107 | 0.3% |
| PR3_9_108 | 0% |
| PR3_9_109 | 0% |
| PR3_9_110 | 0.04% |
| PR3_10_111 | 0.02% |
| PR3_10_112 | 0.45% |
| PR3_10_113 | 0% |
| PR3_10_114 | 0% |
| PR3_10_115 | 0.06% |
| PR3_10_116 | 0% |
| PR3_10_117 | 0% |
| PR3_10_118 | 0.01% |
| PR3_10_111 PR3SP | 0.2% |
| PR3_10_112 PR3SP | 1.1% |
| PR3_10_113 PR3SP | 0% |
| PR3_10_114 PR3SP | 0% |

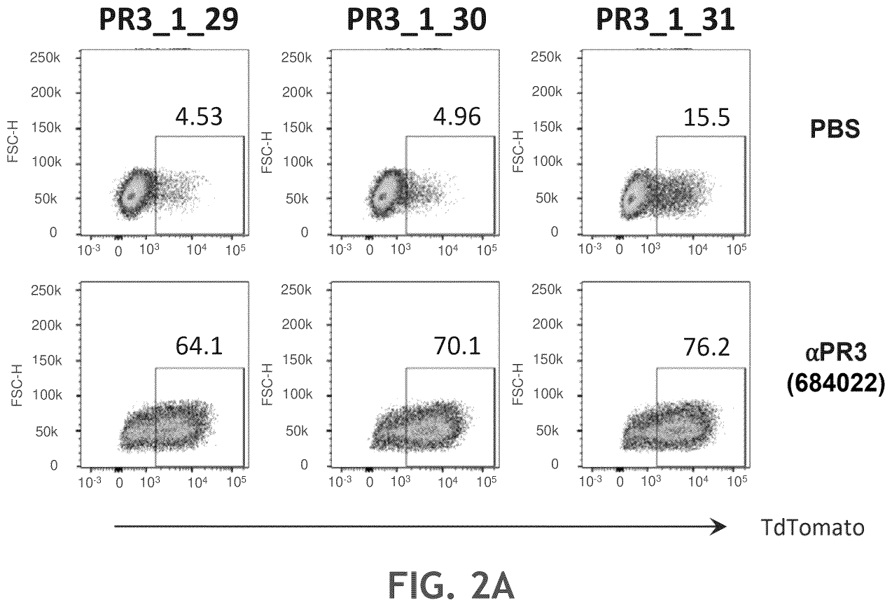

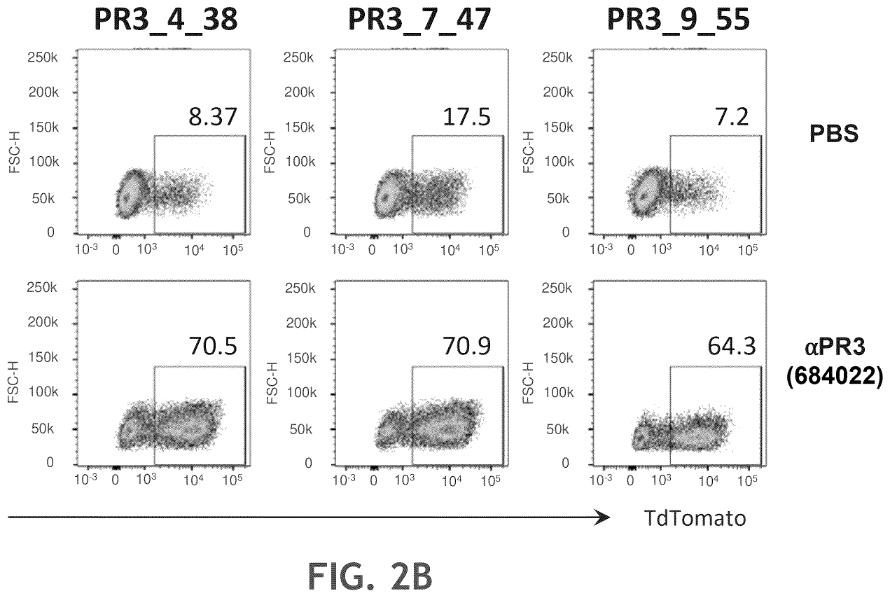

Antigen specific activation of the CAARs was also assessed by comparing signaling when target anti-PR3 antibody was present versus when it was absent. Single CAAR constructs were stably transduced into human Td-Tomato reporter Jurkat cells as above, and then were cultured for 24 hours at 4° C. without stimulation (phosphate buffer saline, ‘PBS’ condition) or on plate-bound anti-human PR3 antibody (R&D Systems, clone MAB684022, catalog #MAB61341). The plates for use in these experimental conditions were prepared by overnight incubation at 4° C. with either PBS alone (unstimulated condition) or with anti-human PR3 antibody resuspended in PBS. Prior to cell culture of CAARs, PBS solutions were removed. The cells were then evaluated for the expression of red fluorescent protein after the incubation and the results were expressed as the percentage of Td-Tomato+-Jurkat cells. Compared to the unactivated condition (PBS), which resulted in tonic signaling from between 4.5% and 17.5% among the tested CAARs, culture with anti-human PR3 antibody resulted in increased percentage of Td-Tomato+-Jurkat cells, ranging from 64% to 76% (FIGS. 2A-B). The level of activation achieved was comparable across the CAAR constructs tested. These results demonstrate antigen-specific stimulation and activation of the CAAR in the presence of anti-PR3 target antigen.

### Example 3: Epitope Reactivity of PR3-Specific Antibodies Mapped Via Competition Binding Assay

A panel of anti-human PR3-antibodies was generated through an in vivo de novo immunization campaign in rodents via sub-cutaneous immunization with native, active human PR3 protein. Resultant PR3-specific antibodies were screened, alongside known reference PR3-specific antibodies (R&D Systems, clone MAB684022, catalog #MAB61341; EMD Millipore, clone MCPR3-3, catalog #MABF973; Abcam, clone PR3G2, catalog #ab240507; Sigma Aldrich, clone MCPR3-2, catalog #mabt340; Abcam, clone WGM2, catalog #ab91181), in a competition binding assay to determine epitope reactivity and overlaid relative to reported ANCA epitopes from AAV patients (epitopes as reported in Bruner et al. (2010) Clin and Experimental Immunol; see also Silva et al. (2010) J Autoimmunity). Using an LSA array-based SPR (surface plasmon resonance) instrument (Carterra), the ability of each antibody to block another antibody's binding to their antigen was determined in a pairwise fashion. The blocking profile for each antibody relative to the others in the panel was used to group antibodies with similar blocking patterns into epitope bins. Epitope binning of these PR3-reactive antibodies revealed reactivities to numerous different epitopes on the PR3 protein including epitopes that overlap with five (5) previously reported ANCA epitopes (FIG. 3). Eight (8) antibodies representing six (6) distinct epitope reactivities were expressed in cellular systems for CAAR killing assays that were used to assess potency.

### Example 4: Determination of PR3 CAAR-T Cell Potency at Various Effector to Target Ratios (E:T Ratios) and Determination of PR3 CAAR-T Cell Potency Against and Selectivity for Targets Expressing Anti-PR3 Antibodies with Multiple Distinct Epitopes

### A. PR3 CAAR T Cells Showed Potency at Varying Effector (PR3 CAAR-T Cell) to Target (Anti-PR3 Antibody Expressing Cell) Ratios

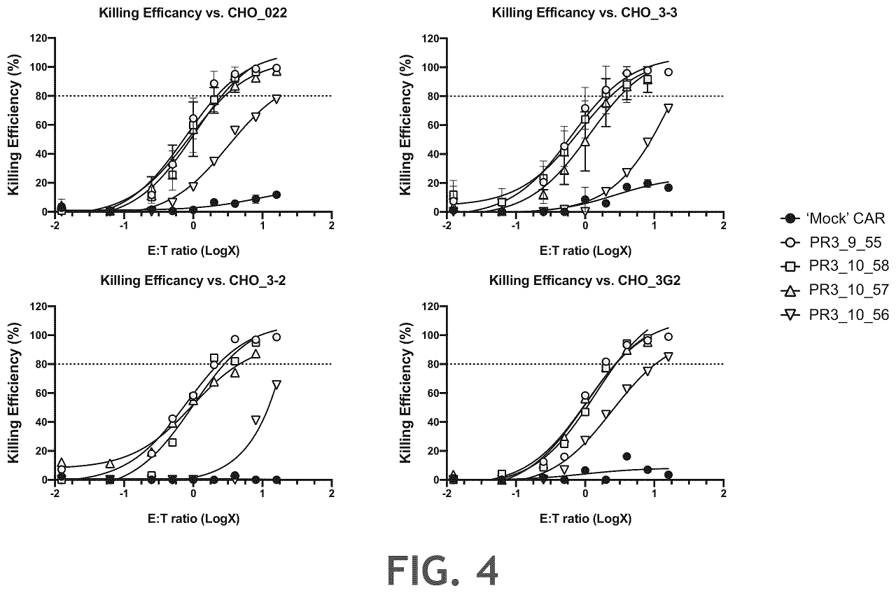

The function of selected anti-PR3-CAARs, described in Examples 1-3, were assessed in primary CD3+ T cells isolated from peripheral blood mononuclear cells (PBMCs) from normal healthy volunteers (NHVs). PR3-CAAR constructs (PR3_9_LS (PR39_55), PR3_10_SS (PR3_9_56), PR3_10_MS (PR3_9_57), PR3_10_LS (PR3_9_58)) were stably transfected into CD3⁺ T cells (1:1 ratio of CD4⁺ and CD8⁺ cells) isolated from peripheral blood mononuclear cells (PBMCs) derived from normal healthy volunteers. Mock T cells (T cells not transduced with the PR3-CAR constructs but otherwise obtained and treated like PR3-CAAR T cells) or PR3 CAAR-T cells (effector cells) were cultured at various E:T ratios (1:16, 1:8, 1:4, 1:2, 1:1, 2:1, 4:1, 8:1 and 16:1) with CHO-S cells engineered to express a single αPR3-monoclonal IgG antibody (target cells), either CHO_022, CHO_3-3, CHO_3-2 or CHO_3G2. Following a 24 hour culture, CAAR-T cell killing efficiencies of IgG⁺-CHO-S targets (on-target killing) were assessed (FIG. 4). IgG+ CHO-S cells (targets) were labeled with CFSE and IgG^{neg} CHO-P cells (parentals, non-target control cells) were labeled with VioletTrace fluorescent dye. Cytolytic activity was assessed by flow cytometry by measuring loss of viable target cells (IgG+ CFSE+ cells) at 24 hour post-culture. Compared to Mock T cells that did not express a CAAR, the PR3-CAAR T cells tested exhibited a high killing potency, with most of the tested CAARs exhibiting >80% killing efficiency at E:T ratios of 2:1, 4:1, 8:1 and 16:1.

B. PR3 CAAR T Cells Showed Potency Against and Selectivity for Targets Expressing Anti-PR3 Antibodies with Multiple Distinct Epitopes

Next, CAAR-T cells (effector cells, made as described above) were cultured with target cells (either CHO-S or Sp2a hybridomas) engineered to express αPR3-monoclonal IgG antibodies and IgG^{neg} control cells. CAAR T cells and IgG⁺-targets and IgG^{neg} control cells were plated at a ratio of 2:1:1, respectively. So that a consistent number of CAAR positive cells was used in these experiments, the transduction efficiency was determined for each experimental run, and samples were diluted with mock T cells to the extent needed so that all samples had the same CAAR expression percentage as the sample with the lowest transduction efficiency.

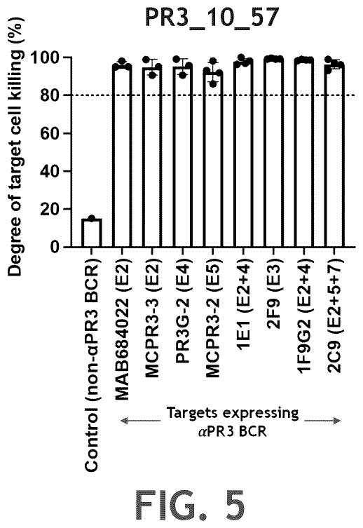

Eight (8) IgG⁺-targets (or a subset of three of these, in the case of experiments for which results are shown in Table E3B) were used to determine CAAR potency across a range of epitope reactivities. The eight (8) IgG⁺-targets, each expressing a single αPR3 antibody clone, represented six (6) distinct epitope reactivities. Following 24 hour culture, CAAR-T cell killing efficiencies of IgG⁺-targets (on-target killing) versus IgG^{neg}-control cells (off-target killing) was assessed. Representative data for PR3_10_MS (PR3_10_57) CAAR T cells shows the selectivity of this CAAR, as minimal killing is observed on IgG^{neg}-control cells, whereas robust potency (>80% killing) is achieved across all IgG⁺-targets tested (FIG. 5).

Table E3A and Table E3B summarize potency data for the tested PR3-CAARs. The percentages provided in columns 2 to 9 of Table E3A and in columns 2 to 4 of Table E3B indicate the percentage of IgG⁺ cells that were killed, calculated as a percentage of the ratio of CFSE+ target cell counts to Violet+ control cell counts in the hybridoma-only condition minus the ratio of CFSE+ target cell counts to Violet+ control cell counts in the CAAR-T condition relative to this ratio in the hybridoma-only condition. Percentages in Table E3A and Table E3B are presented as mean±standard deviation (SD) and are representative of 3-5 donors per group.

$$\frac{\begin{matrix}\{ \frac{Cell count ( {CFSE}^{+} Target cells )}{Cell count ( {Violet}^{+} Control cells )} ( Hybridoma only ) \} - \\ \{ \frac{Cell count ( {CFSE}^{+} Target cells )}{Cell count ( {Violet}^{+} Control cells )} ( CAR - T ) \}\end{matrix}}{\{ \frac{Cell count ( {CFSE}^{+} Target cells )}{Cell count ( {Violet}^{+} Control cells )} ( Hybridoma only ) \}} \times 100$$

In contrast to ‘Mock’ CAAR conditions (mock CAARs were T cells not transfected with the PR3-CAAR constructs but otherwise obtained and treated like the CAAR T cells), most PR3-CAAR T cells tested had high potencies against αPR3 (anti-PR3) antibody expressing cell targets (boldfaced values in Table E3A and Table E3B indicate that the assessed potency was 80% or greater, or 80% fell within 1 standard deviation of the mean of the assessed potency).

The breadth or scope of killing was determined by the CAAR killing efficiencies, for a single PR3-CAAR construct, across all of the αPR3 antibody-expressing cell targets; in other words, the breadth of killing was calculated as the percentage of the total number of the eight tested anti-PR3 antibody expressing targets for which a particular PR3 CAAR-T effector achieved high killing potency according to the rule indicated above for boldfacing. See column 10 of Table E3A. PR3_4 (D91N)_SS (PR3_4_38); PR3_9 (G4P)_LS (PR3_9_55); PR3_10 (H44A)_MS (PR3_10_57); and PR3_10 (H44A)_LS (PR3_10_58) achieved particularly broad scope (more than 80% breadth) of killing.

Off-target selectivity was assessed by measurement of killing efficiencies against IgG^{neg} control cells. No PR3-CAAR T cells were observed to target and kill IgG^{neg} CHO-P cells, whose levels, when cultured with PR3-CAAR T cells, remained comparable to CHO alone conditions and non-specific ‘mock’ transduced T cells. Accordingly, all of the PR3-CAAR T cells selectively killed anti-PR3 antibody expressing target cells.

TABLE E3A
Killing potencies of PR3-CAAR T cells against anti-PR3-IgG expressing cellular targets

| | Percent target killing or potency; mean (SD) | | | | | | | | |
|---|---|---|---|---|---|---|---|---|---|
| | MAB684022 | MCPR3-3 | PR3G-2 | MCPR3-2 | 1E1 | 1F9G2 | 2F9 | 2C9 | |
| | Epitope | Epitope | Epitope | Epitope | Epitope | Epitope | Epitope | Epitope | Breadth of |
| | 2 | 2 | 4 | 5 | 2 + 4 | 2 + 4 | 3 | 2 + 5 + 7 | killing |
| Mock | 5.0 (9.6) | 3.0 (5.0) | 4.5 (7.9) | 0.0 (0.0) | 4.6 (1.7) | 13.8 (7.5) | 2.1 | 3.2 | 0% |
| PR3_1_30 | 83.4 (2.6) | 72.5 (13.3) | 66.5 (17.3) | 79.0 (4.8) | 98.4 (9.5) | 98 (0.5) | 90.4 (7.6) | 82.4 (9.5) | 100% |
| PR3_1_29 | 87.7 (5.2) | 78.9 (14.5) | 79.9 (1.6) | 68.2 (2.5) | 98.3 (11.3) | 94.1 (6.1) | 81.9 (18.3) | 87.1 (12.2) | 88% |
| PR3_4_38 | 92.2 (1.4) | 89.6 (7.7) | 90.5 (1.1) | 75.2 (4.9) | 95.8 (12.1) | 97.9 (0.5) | 94.1 (4.9) | 98.3 (0.3) | 100% |
| PR3_9_55 | 98.5 (1.8) | 96.7 (4.1) | 97.7 (2.0) | 98.8 (1.6) | 99.5 (4.9) | 97.4 (1.8) | 83.2 (12.5) | 85.2 (1.0) | 100% |
| PR3_10_56 | 92.2 (4.1) | 91.5 (5.8) | 92.5 (5.1) | 42.2 (23.6) | 100 (12.0) | 95 (2.1) | 54.7 (9.5) | 92.3 (3.9) | 75% |
| PR3_10_57 | 96.1 (1.8) | 94.9 (4.2) | 95.2 (4.2) | 92.3 (5.0) | 100 (6.5) | 98.7 (0.3) | 99.3 (0.3) | 96.4 (2.5) | 100% |
| PR3_10_58 | 97.0 (1.2) | 94.6 (3.4) | 96.9 (2.1) | 96.1 (2.6) | 94.1 (17.8) | 98.7 (0.3) | 96.7 (3.2) | 84.3 (11.1) | 100% |

TABLE E3B
Killing potencies of PR3-CAAR T cells against
anti-PR3-IgG expressing Spa2 hybridoma cells

| | Target killing (%) (mean (SD)) | | |
|---|---|---|---|
| | Antibody clone name | | |
| | 1E1 | 2F9 | 2C9 |
| | CAAR Construct Descriptor | | |
| | Epitope | Epitope | Epitope |
| | 2 + 4 | 3 | 2 + 5 + 7 |
| PR3_9_103 | 76.8 (16.6) | 72.9 (12.0) | 43.8 (18.1) |
| PR3_9_104 | 90.1 (5.7) | 73.3 (11.5) | 73.0 (16.2) |
| PR3_9_107 | 55.0 (17.3) | 44.7 (11.1) | 4.2 (1.0) |
| PR3_9_109 | 89.6 (8.8) | 73.3 (27.7) | 36.9 (32.5) |
| PR3_9_110 | 84.5 (6.6) | 66.3 (16.3) | 84.5 (21.2) |
| PR3_10_111 | 88.1 (5.7) | 69.4 (17.6) | 66.5 (29.2) |
| PR3_10_112 | 91.7 (4.2) | 55.6 (18.2) | 89.6 (6.3) |
| PR3_10_115 | 47.0 (19.6) | 53.9 (22.9) | 3.7 (6.2) |
| PR3_10_118 | 88.0 (6.4) | 18.3 (14.0) | 76.9 (13.9) |
| PR3_10_111 PR3SP | 88.3 (10.6) | 72.4 (14.7) | 68.9 (25.5) |
| PR3_10_112 PR3SP | 85.3 (10.1) | 41.8 (18.4) | 79.1 (12.9) |

Table E3B shows percent target killing (potency) of additional PR3-CAAR T cells against Spa2 hybridoma cell lines expressing 3 distinct PR3-reactive antibodies. Killing assays were established as described above. Briefly, PR3-CAAR T cells were cultured with IgG^{neg} hybridomas and PR3-reactive IgG⁺ hybridomas in a ratio of 2:1:1.

Following 24 hour culture, the CAAR-T cell killing of IgG⁺-targets (on-target killing) versus IgG^{neg}-control cells (off-target killing) was assessed by flow cytometry.

Table E3B summarizes potency data for the tested PR3-CAARs, presented as mean±standard deviation (SD) and are representative of 3-5 donors per group. Most PR3-CAAR T cells tested achieved high killing potency against αPR3 (anti-PR3) antibody expressing cell targets, with PR3-CAARs PR3_9_104, PR3_9_110, PR3_10_111, and PR3_10_111 PR3SP demonstrating particularly broad scope killing (i.e., high killing potency against all of the Spa2-hybridoma targets tested).

### Example 5: PR3-CAAR T Cells were Able to Target Both High & Low Density αPR3 B Cell Receptor-Expressing CHO Cells and PR3-CAAR T Mediated Killing Increased CAAR T Cell Activation

PR3-CAAR T cells were assessed for killing of αR3 B cell receptor-expressing cells and for activation.

### A. PR3-CAAR T Cells Effectively Targeted Both High & Low Density αPR3 BCR-Expressing CHO Cells

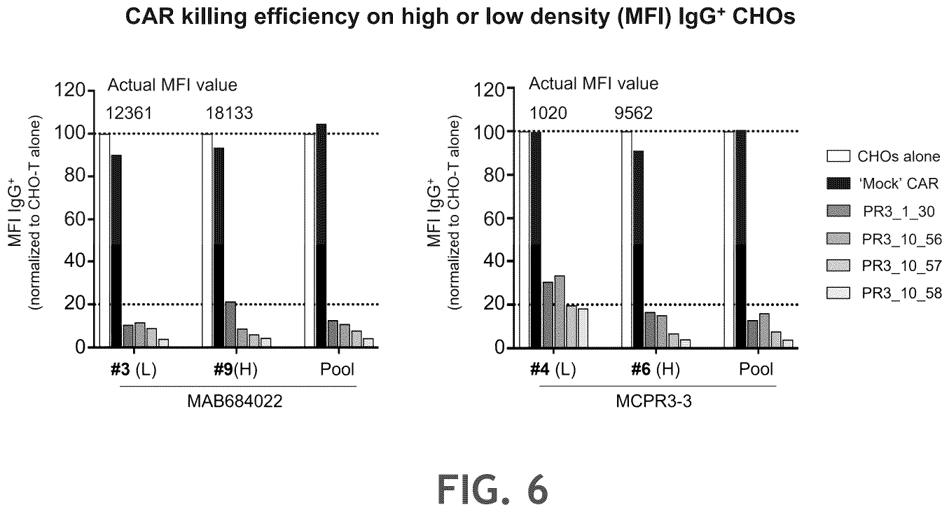

Multiple B cell lineages are reported to express PR3-reactive antibodies in AAV patients and these different B cell populations express varying amounts (or densities) of PR3-reactive antibodies on the cell surface. This density can be measured by flow cytometry and expressed as mean fluorescence intensity (MFI). To assess whether potency of PR3-CAAR T cells are impacted due to different relative surface expression of PR3-reactive antibodies, multiple αPR3 expressing IgG⁺-CHO-S cell targets were generated which expressed varying densities of αPR3 antibodies. αPR3 antibody expression density on IgG⁺-CHO-S targets was assessed by mean fluorescence intensity (MFI) and PR3 CAAR T cell killing was assessed against each individual IgG⁺-CHO-S target. PR3-CAAR T cells were able to achieve at least 80% killing of both high and low density targets. (FIG. 6). This result indicates that PR3-CAAR T cells are expected to be capable of targeting B cell populations with high surface IgG densities (such as, e.g., mature, transitional & naïve B cell populations) as well as B cell populations with low surface IgG densities (such as, e.g., plasmablasts and plasma cells). PR3_1_MS (PR3_130) and PR3_10_SS (PR3_10_56) CAAR killing efficiencies were slightly lower (˜65%) against the lowest density IgG⁺-CHO-S target (MCPR3-3 #4 (L)).

B. PR3-CAAR T Mediated Killing was Associated with CAAR T Cell Activation

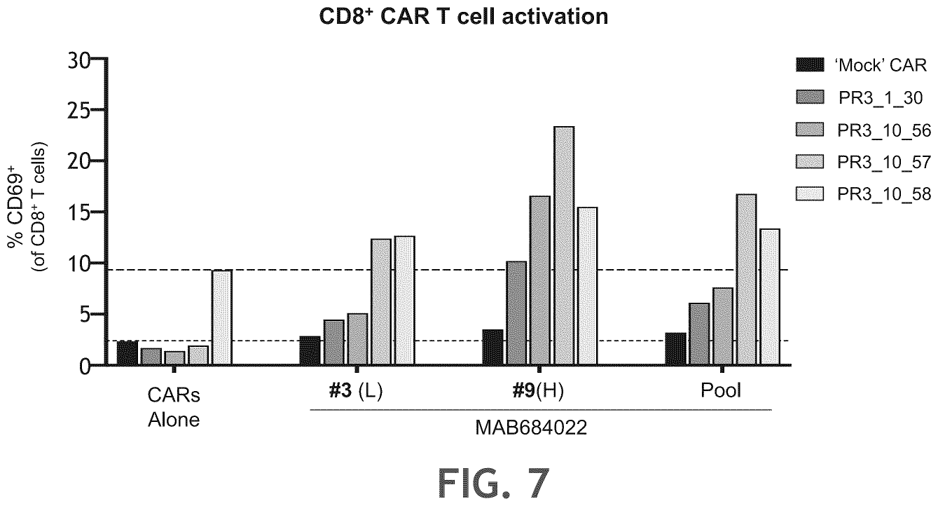

CAAR T cell activation was measured by CD69 levels on CD8⁺ PR3-CAAR T cells (FIG. 7). In unactivated conditions (‘CAARs alone’), CAAR T cells expressed <2% CD69, except that PR3_10_58 (PR3_10_58) CAAR T cells exhibited elevated basal activation, compared to other PR3-CAAR T cells, as evidenced by the heightened level of CD69 expression (˜10% on CD8⁺ PR3-CAAR T cells). Following culture with IgG⁺ CHO-S target cells for 24 hours, which resulted in >80% target cell killing, increased expression of CD69 was observed on all PR3-CAAR T cells ranging from ˜5% to 25%. Higher antigen density also drove increased activation of PR3-CAAR T cells, as evidenced by elevated CD69 expression, across all PR3-CAARs compared to low density targets. No CAAR T activation was observed greater than 25% and despite heightened basal activation (˜5-fold), PR3_10_LS (PR3_10_58) did not maintain this 5-fold magnitude difference following activation. Similar results were observed for CD25 expression and for CD69 and CD25 levels on CD4⁺ PR3-CAAR T cells (data not shown).

### Example 6: Soluble ANCAs Minimally Impacted PR3-CAAR T Cell-Mediated Killing Efficiencies

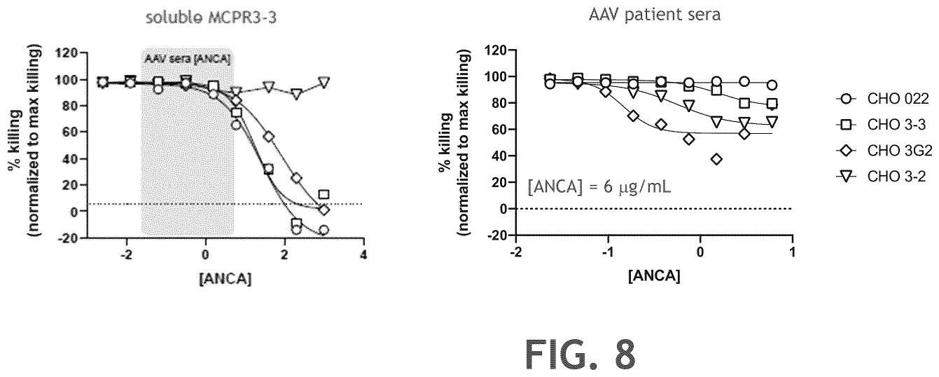

CAAR T cell killing efficiencies were assessed in the presence of soluble anti-PR3 monoclonal antibodies or in the presence of ANCAs, derived from AAV patient serum samples (FIG. 8). PR3-reactive monoclonal antibodies or AAV patient serum, with known concentrations of total PR3-reactive ANCAs, which was isolated from whole blood, were titrated into cultures containing PR3-CAAR T cells and polyclonal mixtures of 4 anti-PR3 IgG-expressing CHO-S cell lines. The top concentration used for the serum titration reflects 100% serum and therefore represents actual (absolute) ANCA concentration in patient blood. Cell killing of individual CHO targets was assessed following 24 hour culture and data normalized to % max killing to reflect an inhibition or interference of maximum killing. Results shown are for an exemplary PR3 CAAR T construct (PR3_10_57). Soluble anti-PR3 monoclonal antibody resulted in complete or near-complete inhibition of CAAR killing efficiencies at 1000 μg/mL for certain IgG⁺ CHO-S target cells and this interference was attenuated at lower concentrations (200 μg/mL or 40 μg/mL). Minimal interference of CAAR killing efficiencies was observed at concentrations at or below 8 μg/mL. Concentrations around or below 8 μg/mL reflect ranges of ANCA concentrations ([ANCA]) observed in AAV patient sera samples (left panel, gray shaded box). Consistent with the levels of interference observed at ANCA concentration levels predicted in AAV samples, an AAV patient serum sample with a measured ANCA titer of 6 μg/mL was assessed in this system and was observed to reduce the maximum killing efficiencies by 40%, 30%, 20% and 0% for each of the four IgG+ CHO-S target cells, respectively. AAV patient serum, with an ANCA titer of 6 μg/mL, reduced the maximum killing efficiencies by 40%, 30%, 20% and 0% for each of the four IgG⁺ CHO-S target cells, respectively.

Table E4 summarizes potency interference data for all AAV subject sera samples tested and represents the maximum % inhibition of CAAR potency (killing) observed. Results shown are for an exemplary PR3 CAAR T construct (PR3_10_57). Most AAV sera samples did not reduce CAAR potency by >20%. Of note, any inhibition observed was able to be titrated out in all AAV sera samples tested, suggesting that reduced ANCA titers (as may be achieved, for example, by administration of cyclophosphamide) would limit or reduce any predicted inhibitory effect on CAAR potency in patients.

TABLE E4
Modest interference of CAAR killing potencies observed
with soluble ANCAs derived from AAV patients

| | Clinical | | | | | |
|---|---|---|---|---|---|---|
| AAV | ANCA | ELISA | Maximum Inhibition (%) of CAAR potency by ANCAs | | | |
| Donor | Value | Conc. | MAB684022 | MCPR3-3 | PR3-G2 | MCPR3-2 |
| ID | (RQ; (titer)) | (μg/ml) | (Epitope 2) | (Epitope 2) | (Epitope 4) | (Epitope 5) |
| 104940 | 26.3 (1:160) | 1.09 | 2 | 10 | 10 | 49 |
| 103945 | 22.0 (1:160) | 1.15 | 0.2 | 0.8 | 17 | 2 |
| 121491183 | N/A | 1.71 | 0.3 | 3 | 2 | 3 |
| 107177 | 38.4 (<1:20) | 1.91 | 0.8 | 0.6 | 2 | 2 |
| 104746 | 393.8 (1:160) | 2.12 | 1 | 2 | 48 | 10 |
| HMN182 | N/A | 3.15 | 3 | 2 | 9 | 4 |
| 104816 | 80.9 (1:40) | 3.48 | 0.9 | 0.7 | 2 | 4 |
| HMN342949 | N/A | 4.87 | 22 | 55 | 75 | 29 |
| 104803 | 35.6 (<1:20) | 6.07 | 1 | 7 | 0 | 18 |
| 104816 | 80.9 (1:40) | 6.27 | 1 | 0.8 | 0 | 0 |
| AAV10 | N/A | 8.67 | 4 | 33 | 13 | 29 |
| 104774 | >3,285.3 (>=1:1280) | 27.51 | 24 | 21 | 23 | 22 |

### Example 7: PR3-CAAR T Cell Targeting of Immunization-Elicited PR13-Reactive B Cell Populations In Vivo

Next, in vivo PR3-CAAR T cell targeting of immunization-elicited PR3-reactive B cell populations was examined. NOD mice were immunized with mutant human PR3-H44A protein (catalytically inactive). Immunization priming was performed on day 1 with subcutaneous injection of 100 μg PR3 protein in complete Freund's adjuvant (CFA). On day 5, mice were treated with a CAAR T pre-conditioning regimen of Cyclophosphamide (250 mg/kg) via intraperitoneal injection, followed by adoptive transfer of either mock or PR3_9_LS (PR3_9_55) CAAR T cells on day 6. On day 10, mice were ‘boosted’ with 40 μg PR3 protein in incomplete Freund's adjuvant (IFA) and CAAR T cell killing efficiencies and B cell depletion were assessed on day 17 of study.

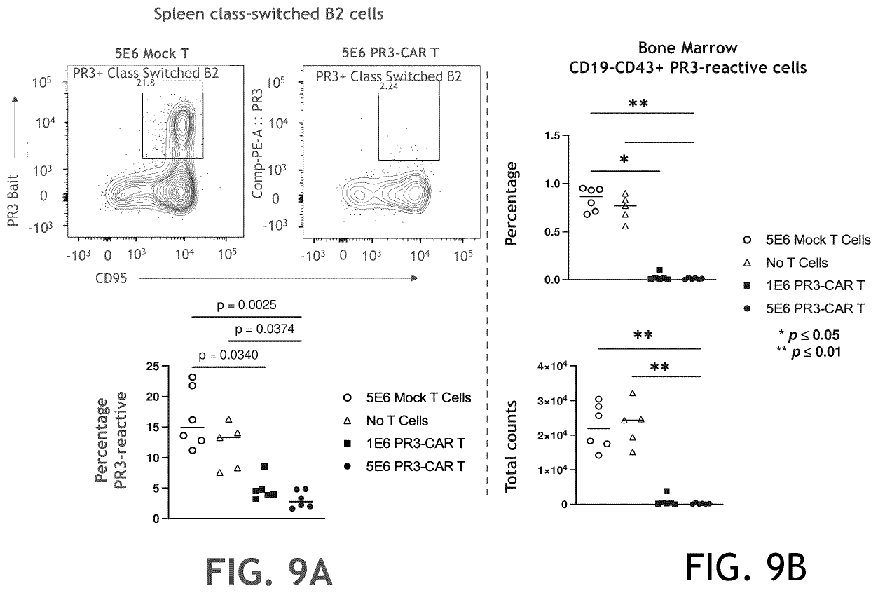

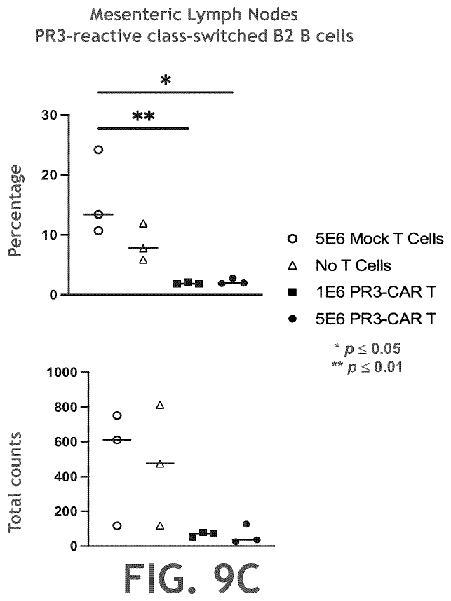

Representative flow cytometric plots of PR3-reactive class-switched B2 B cells in the spleen, demonstrate that, compared with PR3-immunized mice that received ‘Mock’ CAAR T cells, which show a robust and detectable population of PR3-reactive B cells, PR3-immunized mice that received PR3-CAAR T cells showed statistically significant depletion of PR3-reactive B cell populations FIG. 9A. Moreover, statistically significant depletion of additional PR3-reactive B cell populations was observed, both by frequency (%) and total numbers, in the bone marrow FIG. 91B and mesenteric lymph nodes (FIG. 9C).

Table E5 shows the reductions observed across all characterized PR3-reactive B cell populations in multiple tissue compartments (spleen, bone marrow (BM) & mesenteric lymph nodes (LN)). PR39_LS (PR3_9_55) was able to deplete multiple PR3-reactive B cell populations to a degree of >8000 in all tissue compartments assessed.

TABLE E5
In vivo PR3-CAAR T cell targeting of immunization-elicited PR3-reactive B cell populations
Anti-PR3 Expressing Population

| | | | | Extent of |
|---|---|---|---|---|
| | | | | depletion: |
| | | | Depletion | (5 × 10⁶ |
| | | | observed?; | CAARS |
| Organ | Population | Phenotype | p value | vs Mock) |
| Spleen | Class- | CD19 + B220 + IgD − IgM− | Yes; | 82% |
| | Switched B2 | | p = 0.003 | |
| | B1 B Cells | CD19 + CD43 + B220^{lo} | Yes; | 58% |
| | | | p = 0.005 | |
| | Unknown | CD19 − CD3 − CD138 − B220 − | Yes; | 86% |
| | Cells | CD43^{hi} | p = 0.03 | |
| | CD19+ | CD19^{mid}B220^{lo}CD138^{hi}CD20^{lo} | No | — |
| | Plasma Cells | | | |
| | CD19− | CD19^{lo}B220^{lo}CD138^{hi}CD20^{lo} | N/A | N/A |
| | Plasma Cells | | | |
| Bone | Unknown | CD19 − CD3 − CD138 − B220 − | Yes; | 99% |
| Marrow | Cells | CD43^{hi} | p = 0.004 | |
| | Plasma Cells | CD19^{lo}B220^{lo}CD138^{hi}CD20^{lo} | N/A | N/A |
| Mesenteric | Class- | CD19 + B220 + IgD − IgM− | Yes; | 87% |
| LNs | Switched B2 | | p = 0.01 | |

### Example 8: Minimal Off-Target Killing of IgG_{neg} Hybridomas by PR3-CAAR T Cells with G4S Linkers

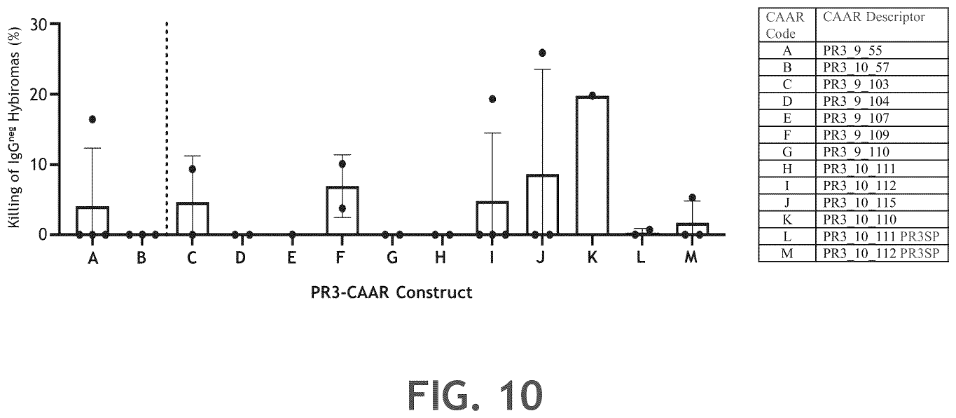

Killing efficiency of additional PR3-CAAR T cells was tested against the parental (IgG^{neg}) Spa2 hybridoma cell line, 11F8, to assess off-target killing by PR3-CAAR T cells. PR3-CAAR T cells were cultured only with IgG^{neg} hybridomas in a ratio of 4:1, and killing efficiencies against IgG^{neg}-control cells was assessed by flow cytometry following 24 hour culture. While some variability was observed (each data point represents CAAR T cells from an individual donor), most PR3-CAAR T cells demonstrated a mean off-target killing rate around 5% (FIG. 10). In particular, PR3_9_104, PR3_9_110, PR3_10_111, and PR3_10_111 PR3SP PR3-CAAR T cells showed off-target killing rates at or below 3%.

### Example 9: PR3-CAAR T Cell Activation Prior to or Following Exposure to αPR3-Expressing Cellular Targets

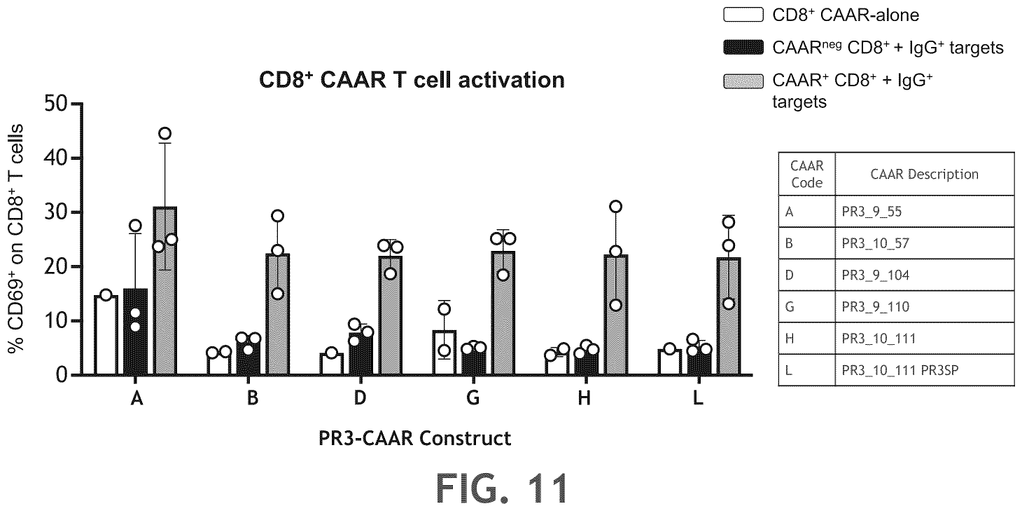

T cell activation was measured by flow cytometry assessment of CD69 levels on CD8+ PR3-CAAR T cells (FIG. 11). The PR3-CAAR T cells were made as described in Example 4A. In unactivated conditions (‘CAARs alone’), most primary human CD8+ PR3-CAAR T cells demonstrated a baseline activation of 2-5% CD69+, except for PR3_9_55 (Construct “A”), which exhibited elevated basal activation (˜15%) compared to other PR3-CAAR T cells (white bars). Following culture with IgG+ Spa2 hybridoma target cells for 24 hours, increased expression of CD69 was observed from all PR3-CAAR constructs, ranging from −15% to 45% of CAAR+ CD8+ T cells (gray bars). No T cell activation was observed on non-CAAR expressing (‘CARneg’) CD8+ T cells in culture (black bars), indicating that the activation is specific for PR3-CAARs in the presence of PR3-reactive targets. Similar results were observed for CD25 expression and for CD69 and CD25 levels on CD4+ PR3-CAAR T cells (data not shown).

### Example 10: In Vivo CAAR Efficacy in NSG Xenograft Model

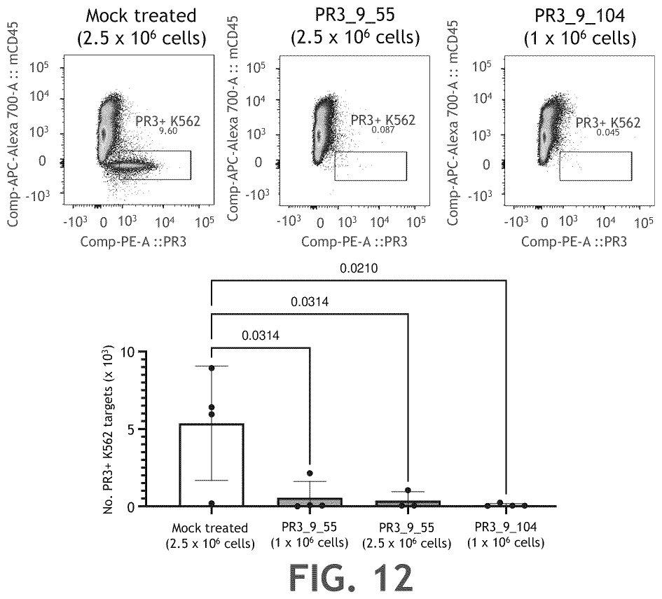

4-10 week old NOD-SCID-gamma chain −/− (NSG) mice were intravenously injected with 2-5×10⁶ K562 tumor cells, which express a PR3-reactive antibody. Human primary PR3-CAAR T cells were intravenously injected after the tumor cells were allowed to engraft for 13 days. For groups receiving PR3-CAAR T cells, 1×10⁶-2×10⁶ CAAR T-positive cells were infused. The number of T cells infused in the “Mock” group was the mean of the total T-cells that was injected in the CAAR T-cell groups. On day 7-14 after T-cell infusion, 3-5 mice from each group were sacrificed, and whole blood, lung tissue, lymph nodes and bone marrow were collected and assessed by flow cytometry for tumor levels. Compared to “Mock” treated animals, both dose levels (1×10⁶ and 2.5×10⁶) of PR3_9_55 and the 1×10⁶ dose of PR3_9_104, showed significant in vivo killing efficacy (p values <0.05) against PR3-reactive K562 target cells in the bone marrow (FIG. 12).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| # | SEQUENCE | ANNOTATION |
|---|---|---|
| 1 | IVGGHEAQPHSRPYMASLQMRGNPGSHFCGGTLIHPSF | Human PR3 |
| | VLTAAHCLRDIPQRLVNVVLGAHNVRTQEPTQQHFSVA | mature sequence |
| | QVFLNNYDAENKLNDVLLIQLSSPANLSASVATVQLPQ | (WT) or PR3_1 |
| | QDQPVPHGTQCLAMGWGRVGAHDPPAQVLQELNVTV | Amino acid |
| | VTFFCRPHNICTFVPRRKAGICFGDSGGPLICDGIIQGIDS | |
| | FVIWGCATRLFPDFFTRVALYVDWIRSTLR | |
| 2 | IVGGHEAQPHSRPYMASLQMRGNPGSHFCGGTLIHPSF | PR3_2 (S176A) |
| | VLTAAHCLRDIPQRLVNVVLGAHNVRTQEPTQQHFSVA | Amino acid |
| | QVFLNNYDAENKLNDVLLIQLSSPANLSASVATVQLPQ | |
| | QDQPVPHGTQCLAMGWGRVGAHDPPAQVLQELNVTV | |
| | VTFFCRPHNICTFVPRRKAGICFGDAGGPLICDGIIQGIDS | |
| | FVIWGCATRLFPDFFTRVALYVDWIRSTLR | |
| 3 | IVGGHEAQPHSRPYMASLQMRGNPGSHFCGGTLIHPSFVLTA | PR3_3 (C- |
| | AHCLRDIPQRLVNVVLGAHNVRTQEPTQQHFSVAQVFLNNY | terminal |
| | DAENKLNDVLLIQLSSPANLSASVATVQLPQQDQPVPHGTQC | addition) |
| | LAMGWGRVGAHDPPAQVLQELNVTVVTFFCRPHNICTFVPR | Amino acid |
| | RKAGICFGDAGGPLICDGIIQGIDSFVIWGCATRLFPDFFTRVA | |
| | LYVDWIRSTLRRVEAKGRP | |
| 4 | IVGGHEAQPHSRPYMASLQMRGNPGSHFCGGTLIHPSFVLTA | PR3_4 (D91N) |
| | AHCLRDIPQRLVNVVLGAHNVRTQEPTQQHFSVAQVFLNNY | Amino acid |
| | DAENKLNNVLLIQLSSPANLSASVATVQLPQQDQPVPHGTQC | |
| | LAMGWGRVGAHDPPAQVLQELNVTVVTFFCRPHNICTFVPR | |
| | RKAGICFGDSGGPLICDGIIQGIDSFVIWGCATRLFPDFFTRVA | |
| | LYVDWIRSTLR | |
| 5 | AEIVGGHEAQPHSRPYMASLQMRGNPGSHFCGGTLIHP | PR3 5 |
| | SFVLTAAHCLRDIPQRLVNVVLGAHNVRTQEPTQQHFS | (Unprocessed N- |
| | VAQVFLNNYDAENKLNDVLLIQLSSPANLSASVATVQL | terminus) |
| | PQQDQPVPHGTQCLAMGWGRVGAHDPPAQVLQELNV | Amino acid |
| | TVVTFFCRPHNICTFVPRRKAGICFGDSGGPLICDGIIQGI | |
| | DSFVIWGCATRLFPDFFTRVALYVDWIRSTLR | |
| 6 | VGGHEAQPHSRPYMASLQMRGNPGSHFCGGTLIHPSFV | PR3 6 (Ile |
| | LTAAHCLRDIPQRLVNVVLGAHNVRTQEPTQQHFSVAQ | Deletion) |
| | VFLNNYDAENKLNDVLLIQLSSPANLSASVATVQLPQQ | Amino acid |
| | DQPVPHGTQCLAMGWGRVGAHDPPAQVLQELNVTVV | |
| | TFFCRPHNICTFVPRRKAGICFGDSGGPLICDGIIQGIDSF | |
| | VIWGCATRLFPDFFTRVALYVDWIRSTLR | |
| 7 | IVGGHEAQPHSRPYMASLQMRGNPGSHFCGGTLIHPSF | PR3_7 (D175N) |
| | VLTAAHCLRDIPQRLVNVVLGAHNVRTQEPTQQHFSVA | Amino acid |
| | QVFLNNYDAENKLNDVLLIQLSSPANLSASVATVQLPQ | |
| | QDQPVPHGTQCLAMGWGRVGAHDPPAQVLQELNVTV | |
| | VTFFCRPHNICTFVPRRKAGICFGNSGGPLICDGIIQGIDS | |
| | FVIWGCATRLFPDFFTRVALYVDWIRSTLR | |
| 8 | IVGGHEAQPHSRPYMASLQMRGNPGSHFCGGTLIHPSFVLTA | PR3_8 (S176C) |
| | AHCLRDIPQRLVNVVLGAHNVRTQEPTQQHFSVAQVFLNNY | Amino acid |
| | DAENKLNDVLLIQLSSPANLSASVATVQLPQQDQPVPHGTQC | |
| | LAMGWGRVGAHDPPAQVLQELNVTVVTFFCRPHNICTFVPR | |
| | RKAGICFGDCGGPLICDGIIQGIDSFVIWGCATRLFPDFFTRVA | |
| | LYVDWIRSTLR | |
| 9 | IVGPHEAQPHSRPYMASLQMRGNPGSHFCGGTLIHPSFVLTA | PR3_9 (G4P) |
| | AHCLRDIPQRLVNVVLGAHNVRTQEPTQQHFSVAQVFLNNY | Amino acid |
| | DAENKLNDVLLIQLSSPANLSASVATVQLPQQDQPVPHGTQC | |
| | LAMGWGRVGAHDPPAQVLQELNVTVVTFFCRPHNICTFVPR | |
| | RKAGICFGDSGGPLICDGIIQGIDSFVIWGCATRLFPDFFTRVA | |
| | LYVDWIRSTLR | |
| 10 | IVGGHEAQPHSRPYMASLQMRGNPGSHFCGGTLIHPSFVLTA | PR3_10 (H44A) |
| | AACLRDIPQRLVNVVLGAHNVRTQEPTQQHFSVAQVFLNNY | Amino acid |
| | DAENKLNDVLLIQLSSPANLSASVATVQLPQQDQPVPHGTQC | |
| | LAMGWGRVGAHDPPAQVLQELNVTVVTFFCRPHNICTFVPR | |
| | RKAGICFGDSGGPLICDGIIQGIDSFVIWGCATRLFPDFFTRVA | |
| | LYVDWIRSTLR | |
| 11 | ATCGTTGGAGGACATGAAGCCCAGCCTCACAGCAGA | Human PR3 |
| | CCTTACATGGCCAGCCTGCAGATGAGAGGCAATCCC | mature sequence |
| | GGCTCTCACTTTTGTGGCGGCACCCTGATTCACCCCT | (WT) or PR3_1 |
| | CCTTCGTTCTGACCGCCGCTCACTGCCTGAGAGACAT | Nucleotide |
| | CCCTCAGAGACTGGTCAACGTGGTGCTGGGAGCCCA | |
| | CAACGTGCGGACACAAGAACCTACACAGCAGCACTT | |
| | CAGCGTGGCCCAGGTGTTCCTGAACAACTACGACGC | |
| | CGAGAACAAGCTGAACGACGTGCTGCTGATCCAGCT | |
| | GAGCAGCCCTGCTAATCTGTCTGCCTCTGTGGCCACT | |
| | GTGCAGCTGCCTCAGCAAGATCAGCCTGTGCCTCACG | |
| | GCACACAGTGTCTGGCTATGGGATGGGGAAGAGTGG | |
| | GCGCTCATGATCCTCCAGCTCAAGTGCTGCAAGAGCT | |
| | GAACGTGACCGTCGTGACCTTCTTCTGCCGGCCTCAC | |
| | AACATCTGCACCTTCGTGCCCAGAAGAAAGGCCGGC | |
| | ATCTGCTTCGGAGATTCTGGCGGCCCTCTGATCTGCG | |
| | ACGGAATCATCCAGGGCATCGACAGCTTCGTGATCTG | |
| | GGGCTGTGCCACCAGACTGTTCCCCGACTTCTTTACC | |
| | CGGGTGGCCCTCTACGTGGACTGGATCCGGTCTACAC | |
| | TGAGA | |
| 12 | ATCGTTGGAGGACATGAAGCCCAGCCTCACAGCAGA | PR3_2 S176A |
| | CCTTACATGGCCAGCCTGCAGATGAGAGGCAATCCC | Nucleotide |
| | GGCTCTCACTTTTGTGGCGGCACCCTGATTCACCCCT | |
| | CCTTCGTTCTGACCGCCGCTCACTGCCTGAGAGACAT | |
| | CCCTCAGAGACTGGTCAACGTGGTGCTGGGAGCCCA | |
| | CAACGTGCGGACACAAGAACCTACACAGCAGCACTT | |
| | CAGCGTGGCCCAGGTGTTCCTGAACAACTACGACGC | |
| | CGAGAACAAGCTGAACGACGTGCTGCTGATCCAGCT | |
| | GAGCAGCCCTGCTAATCTGTCTGCCTCTGTGGCCACT | |
| | GTGCAGCTGCCTCAGCAAGATCAGCCTGTGCCTCACG | |
| | GCACACAGTGTCTGGCTATGGGATGGGGAAGAGTGG | |
| | GCGCTCATGATCCTCCAGCTCAAGTGCTGCAAGAGCT | |
| | GAACGTGACCGTCGTGACCTTCTTCTGCCGGCCTCAC | |
| | AACATCTGCACCTTCGTGCCCAGAAGAAAGGCCGGC | |
| | ATCTGCTTTGGAGATGCTGGCGGCCCTCTGATCTGCG | |
| | ACGGAATCATCCAGGGCATCGACAGCTTCGTGATCTG | |
| | GGGCTGTGCCACCAGACTGTTCCCCGACTTCTTTACC | |
| | CGGGTGGCCCTCTACGTGGACTGGATCCGGTCTACAC | |
| | TGAGA | |
| 13 | ATCGTTGGAGGACATGAAGCCCAGCCTCACAGCAGACCTT | PR3_3 (C- |
| | ACATGGCCAGCCTGCAGATGAGAGGCAATCCCGGCTCTCA | terminal |
| | CTTTTGTGGCGGCACCCTGATTCACCCCTCCTTCGTTCTGA | addition) |
| | CCGCCGCTCACTGCCTGAGAGACATCCCTCAGAGACTGGT | Nucleotide |
| | CAACGTGGTGCTGGGAGCCCACAACGTGCGGACACAAGA | |
| | ACCTACACAGCAGCACTTCAGCGTGGCCCAGGTGTTCCTG | |
| | AACAACTACGACGCCGAGAACAAGCTGAACGACGTGCTG | |
| | CTGATCCAGCTGAGCAGCCCTGCTAATCTGTCTGCCTCTGT | |
| | GGCCACTGTGCAGCTGCCTCAGCAAGATCAGCCTGTGCCT | |
| | CACGGCACACAGTGTCTGGCTATGGGATGGGGAAGAGTG | |
| | GGCGCTCATGATCCTCCAGCTCAAGTGCTGCAAGAGCTGA | |
| | ACGTGACCGTCGTGACCTTCTTCTGCCGGCCTCACAACAT | |
| | CTGCACCTTCGTGCCCAGAAGAAAGGCCGGCATCTGCTTT | |
| | GGAGATGCTGGCGGCCCTCTGATCTGCGACGGAATCATCC | |
| | AGGGCATCGACAGCTTCGTGATCTGGGGCTGTGCCACCAG | |
| | ACTGTTCCCCGACTTCTTTACCCGCGTGGCCCTCTACGTGG | |
| | ACTGGATCAGATCTACCCTGCGGAGAGTGGAAGCCAAGG | |
| | GCAGACCT | |
| 14 | ATCGTTGGAGGACATGAAGCCCAGCCTCACAGCAGA | PR3_4 (D91N) |
| | CCTTACATGGCCAGCCTGCAGATGAGAGGCAATCCC | Nucleotide |
| | GGCTCTCACTTTTGTGGCGGCACCCTGATTCACCCCT | |
| | CCTTCGTTCTGACCGCCGCTCACTGCCTGAGAGACAT | |
| | CCCTCAGAGACTGGTCAACGTGGTGCTGGGAGCCCA | |
| | CAACGTGCGGACACAAGAACCTACACAGCAGCACTT | |
| | CAGCGTGGCCCAGGTGTTCCTGAACAACTACGACGC | |
| | CGAGAACAAGCTGAACAACGTGCTGCTGATCCAGCT | |
| | GAGCAGCCCTGCTAATCTGTCTGCCTCTGTGGCCACT | |
| | GTGCAGCTGCCTCAGCAAGATCAGCCTGTGCCTCACG | |
| | GCACACAGTGTCTGGCTATGGGATGGGGAAGAGTGG | |
| | GCGCTCATGATCCTCCAGCTCAAGTGCTGCAAGAGCT | |
| | GAACGTGACCGTCGTGACCTTCTTCTGCCGGCCTCAC | |
| | AACATCTGCACCTTCGTGCCCAGAAGAAAGGCCGGC | |
| | ATCTGCTTCGGAGATTCTGGCGGCCCTCTGATCTGCG | |
| | ACGGAATCATCCAGGGCATCGACAGCTTCGTGATCTG | |
| | GGGCTGTGCCACCAGACTGTTCCCCGACTTCTTTACC | |
| | CGGGTGGCCCTCTACGTGGACTGGATCCGGTCTACAC | |
| | TGAGA | |
| 15 | GCCGAGATCGTTGGAGGACATGAAGCCCAGCCTCACAGC | PR3_5 |
| | AGACCTTACATGGCCAGCCTGCAGATGAGAGGCAATCCCG | ((Unprocessed |
| | GCTCTCACTTTTGTGGCGGCACCCTGATTCACCCCTCCTTC | N-terminus) |
| | GTTCTGACCGCCGCTCACTGCCTGAGAGACATCCCTCAGA | Nucleotide |
| | GACTGGTCAACGTGGTGCTGGGAGCCCACAACGTGCGGA | |
| | CACAAGAACCTACACAGCAGCACTTCAGCGTGGCCCAGGT | |
| | GTTCCTGAACAACTACGACGCCGAGAACAAGCTGAACGA | |
| | CGTGCTGCTGATCCAGCTGAGCAGCCCTGCTAATCTGTCT | |
| | GCCTCTGTGGCCACTGTGCAGCTGCCTCAGCAAGATCAGC | |
| | CTGTGCCTCACGGCACACAGTGTCTGGCTATGGGATGGGG | |
| | AAGAGTGGGCGCTCATGATCCTCCAGCTCAAGTGCTGCAA | |
| | GAGCTGAACGTGACCGTCGTGACCTTCTTCTGCCGGCCTC | |
| | ACAACATCTGCACCTTCGTGCCCAGAAGAAAGGCCGGCAT | |
| | CTGCTTCGGAGATTCTGGCGGCCCTCTGATCTGCGACGGA | |
| | ATCATCCAGGGCATCGACAGCTTCGTGATCTGGGGCTGTG | |
| | CCACCAGACTGTTCCCCGACTTCTTTACCCGGGTGGCCCTC | |
| | TACGTGGACTGGATCCGGTCTACACTGAGA | |
| 16 | GTTGGAGGACATGAAGCCCAGCCTCACAGCAGACCT | PR3_6 (Ile |
| | TACATGGCCAGCCTGCAGATGAGAGGCAATCCCGGC | Deletion) |
| | TCTCACTTTTGTGGCGGCACCCTGATTCACCCCTCCTT | Nucleotide |
| | CGTTCTGACCGCCGCTCACTGCCTGAGAGACATCCCT | |
| | CAGAGACTGGTCAACGTGGTGCTGGGAGCCCACAAC | |
| | GTGCGGACACAAGAACCTACACAGCAGCACTTCAGC | |
| | GTGGCCCAGGTGTTCCTGAACAACTACGACGCCGAG | |
| | AACAAGCTGAACGACGTGCTGCTGATCCAGCTGAGC | |
| | AGCCCTGCTAATCTGTCTGCCTCTGTGGCCACTGTGC | |
| | AGCTGCCTCAGCAAGATCAGCCTGTGCCTCACGGCAC | |
| | ACAGTGTCTGGCTATGGGATGGGGAAGAGTGGGCGC | |
| | TCATGATCCTCCAGCTCAAGTGCTGCAAGAGCTGAAC | |
| | GTGACCGTCGTGACCTTCTTCTGCCGGCCTCACAACA | |
| | TCTGCACCTTCGTGCCCAGAAGAAAGGCCGGCATCTG | |
| | CTTCGGAGATTCTGGCGGCCCTCTGATCTGCGACGGA | |
| | ATCATCCAGGGCATCGACAGCTTCGTGATCTGGGGCT | |
| | GTGCCACCAGACTGTTCCCCGACTTCTTTACCCGGGT | |
| | GGCCCTCTACGTGGACTGGATCCGGTCTACACTGAGA | |
| 17 | ATCGTTGGAGGACATGAAGCCCAGCCTCACAGCAGACCTT | PR3_7 (D175N) |
| | ACATGGCCAGCCTGCAGATGAGAGGCAATCCCGGCTCTCA | Nucleotide |
| | CTTTTGTGGCGGCACCCTGATTCACCCCTCCTTCGTTCTGA | |
| | CCGCCGCTCACTGCCTGAGAGACATCCCTCAGAGACTGGT | |
| | CAACGTGGTGCTGGGAGCCCACAACGTGCGGACACAAGA | |
| | ACCTACACAGCAGCACTTCAGCGTGGCCCAGGTGTTCCTG | |
| | AACAACTACGACGCCGAGAACAAGCTGAACGACGTGCTG | |
| | CTGATCCAGCTGAGCAGCCCTGCTAATCTGTCTGCCTCTGT | |
| | GGCCACTGTGCAGCTGCCTCAGCAAGATCAGCCTGTGCCT | |
| | CACGGCACACAGTGTCTGGCTATGGGATGGGGAAGAGTG | |
| | GGCGCTCATGATCCTCCAGCTCAAGTGCTGCAAGAGCTGA | |
| | ACGTGACCGTCGTGACCTTCTTCTGCCGGCCTCACAACAT | |
| | CTGCACCTTCGTGCCCAGAAGAAAGGCCGGCATCTGCTTC | |
| | GGAAACTCTGGCGGCCCTCTGATCTGCGACGGAATCATCC | |
| | AGGGCATCGACAGCTTCGTGATCTGGGGCTGTGCCACCAG | |
| | ACTGTTCCCCGACTTCTTTACCCGGGTGGCCCTCTACGTGG | |
| | ACTGGATCCGGTCTACACTGAGA | |
| 18 | ATCGTTGGAGGACATGAAGCCCAGCCTCACAGCAGA | PR3_8 (S176C) |
| | CCTTACATGGCCAGCCTGCAGATGAGAGGCAATCCC | Nucleotide |
| | GGCTCTCACTTTTGTGGCGGCACCCTGATTCACCCCT | |
| | CCTTCGTTCTGACCGCCGCTCACTGCCTGAGAGACAT | |
| | CCCTCAGAGACTGGTCAACGTGGTGCTGGGAGCCCA | |
| | CAACGTGCGGACACAAGAACCTACACAGCAGCACTT | |
| | CAGCGTGGCCCAGGTGTTCCTGAACAACTACGACGC | |
| | CGAGAACAAGCTGAACGACGTGCTGCTGATCCAGCT | |
| | GAGCAGCCCTGCTAATCTGTCTGCCTCTGTGGCCACT | |
| | GTGCAGCTGCCTCAGCAAGATCAGCCTGTGCCTCACG | |
| | GCACACAGTGTCTGGCTATGGGATGGGGAAGAGTGG | |
| | GCGCTCATGATCCTCCAGCTCAAGTGCTGCAAGAGCT | |
| | GAACGTGACCGTCGTGACCTTCTTCTGCCGGCCTCAC | |
| | AACATCTGCACCTTCGTGCCCAGAAGAAAGGCCGGC | |
| | ATCTGCTTCGGAGATTGCGGCGGCCCTCTGATCTGCG | |
| | ACGGAATCATCCAGGGCATCGACAGCTTCGTGATCTG | |
| | GGGCTGTGCCACCAGACTGTTCCCCGACTTCTTTACC | |
| | CGGGTGGCCCTCTACGTGGACTGGATCCGGTCTACAC | |
| | TGAGA | |
| 19 | ATCGTTGGACCCCATGAAGCCCAGCCTCACAGCAGA | PR3_9 (G4P) |
| | CCTTACATGGCCAGCCTGCAGATGAGAGGCAATCCC | Nucleotide |
| | GGCTCTCACTTTTGTGGCGGCACCCTGATTCACCCCT | |
| | CCTTCGTTCTGACCGCCGCTCACTGCCTGAGAGACAT | |
| | CCCTCAGAGACTGGTCAACGTGGTGCTGGGAGCCCA | |
| | CAACGTGCGGACACAAGAACCTACACAGCAGCACTT | |
| | CAGCGTGGCCCAGGTGTTCCTGAACAACTACGACGC | |
| | CGAGAACAAGCTGAACGACGTGCTGCTGATCCAGCT | |
| | GAGCAGCCCTGCTAATCTGTCTGCCTCTGTGGCCACT | |
| | GTGCAGCTGCCTCAGCAAGATCAGCCTGTGCCTCACG | |
| | GCACACAGTGTCTGGCTATGGGATGGGGAAGAGTGG | |
| | GCGCTCATGATCCTCCAGCTCAAGTGCTGCAAGAGCT | |
| | GAACGTGACCGTCGTGACCTTCTTCTGCCGGCCTCAC | |
| | AACATCTGCACCTTCGTGCCCAGAAGAAAGGCCGGC | |
| | ATCTGCTTCGGAGATTCTGGCGGCCCTCTGATCTGCG | |
| | ACGGAATCATCCAGGGCATCGACAGCTTCGTGATCTG | |
| | GGGCTGTGCCACCAGACTGTTCCCCGACTTCTTTACC | |
| | CGGGTGGCCCTCTACGTGGACTGGATCCGGTCTACAC | |
| | TGAGA | |
| 20 | ATCGTTGGAGGACATGAAGCCCAGCCTCACAGCAGACCTT | PR3_10 (H44A) |
| | ACATGGCCAGCCTGCAGATGAGAGGCAATCCCGGCTCTCA | Nucleotide |
| | CTTTTGTGGCGGCACCCTGATTCACCCCTCCTTCGTTCTGA | |
| | CCGCCGCTGCCTGCCTGAGAGACATCCCTCAGAGACTGGT | |
| | CAACGTGGTGCTGGGAGCCCACAACGTGCGGACACAAGA | |
| | ACCTACACAGCAGCACTTCAGCGTGGCCCAGGTGTTCCTG | |
| | AACAACTACGACGCCGAGAACAAGCTGAACGACGTGCTG | |
| | CTGATCCAGCTGAGCAGCCCTGCTAATCTGTCTGCCTCTGT | |
| | GGCCACTGTGCAGCTGCCTCAGCAAGATCAGCCTGTGCCT | |
| | CACGGCACACAGTGTCTGGCTATGGGATGGGGAAGAGTG | |
| | GGCGCTCATGATCCTCCAGCTCAAGTGCTGCAAGAGCTGA | |
| | ACGTGACCGTCGTGACCTTCTTCTGCCGGCCTCACAACAT | |
| | CTGCACCTTCGTGCCCAGAAGAAAGGCCGGCATCTGCTTC | |
| | GGAGATTCTGGCGGCCCTCTGATCTGCGACGGAATCATCC | |
| | AGGGCATCGACAGCTTCGTGATCTGGGGCTGTGCCACCAG | |
| | ACTGTTCCCCGACTTCTTTACCCGGGTGGCCCTCTACGTGG | |
| | ACTGGATCCGGTCTACACTGAGA | |
| 21 | MPLLLLLPLLWAGALA | CD33 signal |
| | | peptide |
| | | Amino acid |
| 22 | ESKYGPPCPPCPM | IgG4 hinge spacer |
| | | (aa) |
| | | Short spacer (SS) |
| 23 | ESKYGPPCPPCP | IgG4 hinge spacer |
| | | (aa) |
| | | Short spacer (SS) |
| 24 | ESKYGPPCPPCPGQPREPQVYTLPPSQEEMTKNQVSLTCLVK | IgG4 Hinge-C_{H}3 |
| | GFYPSDIAVEWESNGQPENNYKTTPPVLDSDGSFFLYSRLTV | spacer (aa) |
| | DKSRWQEGNVFSCSVMHEALHNHYTQKSLSLSLGKM | Medium spacer |
| | | (MS) |
| 25 | ESKYGPPCPPCPAPPVAGPSVFLFPPKPKDTLMISRTPEVTCV | IgG4 hinge- |
| | VVDVSQEDPEVQFNWYVDGVEVHNAKTKPREEQFQSTYRV | IgG2/1gG4 C_{H}2- |
| | VSVLTVLHQDWLNGKEYKCKVSNKGLPSSIEKTISKAKGQP | IgG4 C_{H}3 spacer |
| | REPQVYTLPPSQEEMTKNQVSLTCLVKGFYPSDIAVEWESNG | (aa) |
| | QPENNYKTTPPVLDSDGSFFLYSRLTVDKSRWQEGNVFSCSV | Long spacer (LS) |
| | MHEALHNHYTQKSLSLSLGKM | |
| 26 | FWVLVVVGGVLACYSLLVTVAFIIFWV | CD28 |
| | | transmembrane |
| | | domain (aa) |
| 27 | KRGRKKLLYIFKQPFMRPVQTTQEEDGCSCRFPEEEEGGCEL | 4-1BB-derived |
| | | intracellular co- |
| | | signaling |
| | | sequence (aa) |
| 28 | RVKFSRSADAPAYQQGQNQLYNELNLGRREEYDVLDK | CD3-zeta derived |
| | RRGRDPEMGGKPRRKNPQEGLYNELQKDKMAEAYSEI | intracellular |
| | GMKGERRRGKGHDGLYQGLSTATKDTYDALHMQALP | signaling domain |
| | PR | (aa) |
| 29 | IVGGHEAQPHSRPYMASLQMRGNPGSHFCGGTLIHPSF | Human PR3 |
| | VLTAAHCLRDIPQRLVNVVLGAHNVRTQEPTQQHFSVA | mature sequence |
| | QVFLNNYDAENKLNDVLLIQLSSPANLSASVATVQLPQ | (WT) or PR3_1 |
| | QDQPVPHGTQCLAMGWGRVGAHDPPAQVLQELNVTV | SS CAAR |
| | VTFFCRPHNICTFVPRRKAGICFGDSGGPLICDGIIQGIDS | (PR3_1_29) |
| | FVIWGCATRLFPDFFTRVALYVDWIRSTLRESKYGPPCP | Amino acid |
| | PCPMFWVLVVVGGVLACYSLLVTVAFIIFWVKRGRKK | |
| | LLYIFKQPFMRPVQTTQEEDGCSCRFPEEEEGGCELR | |
| | VKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPP | |
| | R | |
| 30 | IVGGHEAQPHSRPYMASLQMRGNPGSHFCGGTLIHPSFVLTA | Human PR3 |
| | AHCLRDIPQRLVNVVLGAHNVRTQEPTQQHFSVAQVFLNNY | mature sequence |
| | DAENKLNDVLLIQLSSPANLSASVATVQLPQQDQPVPHGTQC | (WT) or PR3_1 |
| | LAMGWGRVGAHDPPAQVLQELNVTVVTFFCRPHNICTFVPR | MS CAAR |
| | RKAGICFGDSGGPLICDGIIQGIDSFVIWGCATRLFPDFFTRVA | (PR3_1_30) |
| | LYVDWIRSTLRESKYGPPCPPCPGQPREPQVYTLPPSQEEMT | Amino acid |
| | KNQVSLTCLVKGFYPSDIAVEWESNGQPENNYKTTPPVLDS | |
| | DGSFFLYSRLTVDKSRWQEGNVFSCSVMHEALHNHYTQKSL | |
| | SLSLGKMFWVLVVVGGVLACYSLLVTVAFIIFWVKRGRKKL | |
| | LYIFKQPFMRPVQTTQEEDGCSCRFPEEEEGGCELRVKFSRSA | |
| | DAPAYQQGQNQLYNELNLGRREEYDVLDKRRGRDPEMGGK | |
| | PRRKNPQEGLYNELQKDKMAEAYSEIGMKGERRRGKGHDG | |
| | LYQGLSTATKDTYDALHMQALPPR | |
| 31 | IVGGHEAQPHSRPYMASLQMRGNPGSHFCGGTLIHPSF | Human PR3 |
| | VLTAAHCLRDIPQRLVNVVLGAHNVRTQEPTQQHFSVA | mature sequence |
| | QVFLNNYDAENKLNDVLLIQLSSPANLSASVATVQLPQ | (WT) or PR3_1 |
| | QDQPVPHGTQCLAMGWGRVGAHDPPAQVLQELNVTV | LS CAAR |
| | VTFFCRPHNICTFVPRRKAGICFGDSGGPLICDGIIQGIDS | (PR3_1_31) |
| | FVIWGCATRLFPDFFTRVALYVDWIRSTLRESKYGPPCP | Amino acid |
| | PCPAPPVAGPSVFLFPPKPKDTLMISRTPEVTCVVVDVS | |
| | QEDPEVQFNWYVDGVEVHNAKTKPREEQFQSTYRVVS | |
| | VLTVLHQDWLNGKEYKCKVSNKGLPSSIEKTISKAKGQ | |
| | PREPQVYTLPPSQEEMTKNQVSLTCLVKGFYPSDIAVE | |
| | WESNGQPENNYKTTPPVLDSDGSFFLYSRLTVDKSRWQ | |
| | EGNVFSCSVMHEALHNHYTQKSLSLSLGKMFWVLVVV | |
| | GGVLACYSLLVTVAFIIFWVKRGRKKLLYIFKQPFMRP | |
| | VQTTQEEDGCSCRFPEEEEGGCELRVKFSRSADAPAYQ | |
| | QGQNQLYNELNLGRREEYDVLDKRRGRDPEMGGKPRR | |
| | KNPQEGLYNELQKDKMAEAYSEIGMKGERRRGKGHD | |
| | GLYQGLSTATKDTYDALHMQALPPR | |
| 32 | IVGGHEAQPHSRPYMASLQMRGNPGSHFCGGTLIHPSF | PR3_2 (S176A) |
| | VLTAAHCLRDIPQRLVNVVLGAHNVRTQEPTQQHFSVA | SS CAAR |
| | QVFLNNYDAENKLNDVLLIQLSSPANLSASVATVQLPQ | (PR3_2_32) |
| | QDQPVPHGTQCLAMGWGRVGAHDPPAQVLQELNVTV | Amino acid |
| | VTFFCRPHNICTFVPRRKAGICFGDAGGPLICDGIIQGIDS | |
| | FVIWGCATRLFPDFFTRVALYVDWIRSTLRESKYGPPCP | |
| | PCPMFWVLVVVGGVLACYSLLVTVAFIIFWVKRGRKK | |
| | LLYIFKQPFMRPVQTTQEEDGCSCRFPEEEEGGCELR | |
| | VKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPP | |
| | R | |
| 33 | IVGGHEAQPHSRPYMASLQMRGNPGSHFCGGTLIHPSFVLTA | PR3_2 (S176A) |
| | AHCLRDIPQRLVNVVLGAHNVRTQEPTQQHFSVAQVFLNNY | MS CAAR |
| | DAENKLNDVLLIQLSSPANLSASVATVQLPQQDQPVPHGTQC | (PR3_2_33) |
| | LAMGWGRVGAHDPPAQVLQELNVTVVTFFCRPHNICTFVPR | Amino Acid |
| | RKAGICFGDAGGPLICDGIIQGIDSFVIWGCATRLFPDFFTRVA | |
| | LYVDWIRSTLRESKYGPPCPPCPGQPREPQVYTLPPSQEEMT | |
| | KNQVSLTCLVKGFYPSDIAVEWESNGQPENNYKTTPPVLDS | |
| | DGSFFLYSRLTVDKSRWQEGNVFSCSVMHEALHNHYTQKSL | |
| | SLSLGKMFWVLVVVGGVLACYSLLVTVAFIIFWVKRGRKKL | |
| | LYIFKQPFMRPVQTTQEEDGCSCRFPEEEEGGCELRVKFSRSA | |
| | DAPAYQQGQNQLYNELNLGRREEYDVLDKRRGRDPEMGGK | |
| | PRRKNPQEGLYNELQKDKMAEAYSEIGMKGERRRGKGHDG | |
| | LYQGLSTATKDTYDALHMQALPPR | |
| 34 | IVGGHEAQPHSRPYMASLQMRGNPGSHFCGGTLIHPSFVLTA | PR3_2 (S176A) |
| | AHCLRDIPQRLVNVVLGAHNVRTQEPTQQHFSVAQVFLNNY | LS CAAR |
| | DAENKLNDVLLIQLSSPANLSASVATVQLPQQDQPVPHGTQC | (PR3_2_34) |
| | LAMGWGRVGAHDPPAQVLQELNVTVVTFFCRPHNICTFVPR | Amino Acid |
| | RKAGICFGDAGGPLICDGIIQGIDSFVIWGCATRLFPDFFTRVA | |
| | LYVDWIRSTLRESKYGPPCPPCPAPPVAGPSVFLFPPKPKDTL | |
| | MISRTPEVTCVVVDVSQEDPEVQFNWYVDGVEVHNAKTKP | |
| | REEQFQSTYRVVSVLTVLHQDWLNGKEYKCKVSNKGLPSSI | |
| | EKTISKAKGQPREPQVYTLPPSQEEMTKNQVSLTCLVKGFYP | |
| | SDIAVEWESNGQPENNYKTTPPVLDSDGSFFLYSRLTVDKSR | |
| | WQEGNVFSCSVMHEALHNHYTQKSLSLSLGKMFWVLVVVG | |
| | GVLACYSLLVTVAFIIFWVKRGRKKLLYIFKQPFMRPVQTTQ | |
| | EEDGCSCRFPEEEEGGCELRVKFSRSADAPAYQQGQNQLYN | |
| | ELNLGRREEYDVLDKRRGRDPEMGGKPRRKNPQEGLYNEL | |
| | QKDKMAEAYSEIGMKGERRRGKGHDGLYQGLSTATKDTYD | |
| | ALHMQALPPR | |
| 35 | IVGGHEAQPHSRPYMASLQMRGNPGSHFCGGTLIHPSFVLTA | PR3 3 (C- |
| | AHCLRDIPQRLVNVVLGAHNVRTQEPTQQHFSVAQVFLNNY | terminal |
| | DAENKLNDVLLIQLSSPANLSASVATVQLPQQDQPVPHGTQC | addition) SS |
| | LAMGWGRVGAHDPPAQVLQELNVTVVTFFCRPHNICTFVPR | CAAR |
| | RKAGICFGDAGGPLICDGIIQGIDSFVIWGCATRLFPDFFTRVA | (PR3_3_35) |
| | LYVDWIRSTLRRVEAKGRPESKYGPPCPPCPMFWVLVVVGG | Amino acid |
| | VLACYSLLVTVAFIIFWVKRGRKKLLYIFKQPFMRPVQTTQE | |
| | EDGCSCRFPEEEEGGCELRVKFSRSADAPAYQQGQNQLYNE | |
| | LNLGRREEYDVLDKRRGRDPEMGGKPRRKNPQEGLYNELQ | |
| | KDKMAEAYSEIGMKGERRRGKGHDGLYQGLSTATKDTYDA | |
| | LHMQALPPR | |
| 36 | IVGGHEAQPHSRPYMASLQMRGNPGSHFCGGTLIHPSFVLTA | PR3_3 (C- |
| | AHCLRDIPQRLVNVVLGAHNVRTQEPTQQHFSVAQVFLNNY | terminal |
| | DAENKLNDVLLIQLSSPANLSASVATVQLPQQDQPVPHGTQC | addition) MS |
| | LAMGWGRVGAHDPPAQVLQELNVTVVTFFCRPHNICTFVPR | CAAR |
| | RKAGICFGDAGGPLICDGIIQGIDSFVIWGCATRLFPDFFTRVA | (PR3_3_36) |
| | LYVDWIRSTLRRVEAKGRPESKYGPPCPPCPGQPREPQVYTL | Amino acid |
| | PPSQEEMTKNQVSLTCLVKGFYPSDIAVEWESNGQPENNYK | |
| | TTPPVLDSDGSFFLYSRLTVDKSRWQEGNVFSCSVMHEALH | |
| | NHYTQKSLSLSLGKMFWVLVVVGGVLACYSLLVTVAFIIFW | |
| | VKRGRKKLLYIFKQPFMRPVQTTQEEDGCCRFPEEEEGGCE | |
| | LRVKFSRSADAPAYQQGQNQLYNELNLGRREEYDVLDKRR | |
| | GRDPEMGGKPRRKNPQEGLYNELQKDKMAEAYSEIGMKGE | |
| | RRRGKGHDGLYQGLSTATKDTYDALHMQALPPR | |
| 37 | IVGGHEAQPHSRPYMASLQMRGNPGSHFCGGTLIHPSFVLTA | PR3_3 (C- |
| | AHCLRDIPQRLVNVVLGAHNVRTQEPTQQHFSVAQVFLNNY | terminal |
| | DAENKLNDVLLIQLSSPANLSASVATVQLPQQDQPVPHGTQC | addition) LS |
| | LAMGWGRVGAHDPPAQVLQELNVTVVTFFCRPHNICTFVPR | CAAR |
| | RKAGICFGDAGGPLICDGIIQGIDSFVIWGCATRLFPDFFTRVA | (PR3_3_37) |
| | LYVDWIRSTLRRVEAKGRPESKYGPPCPPCPAPPVAGPSVFLF | Amino acid |
| | PPKPKDTLMISRTPEVTCVVVDVSQEDPEVQFNWYVDGVEV | |
| | HNAKTKPREEQFQSTYRVVSVLTVLHQDWLNGKEYKCKVS | |
| | NKGLPSSIEKTISKAKGQPREPQVYTLPPSQEEMTKNQVSLTC | |
| | LVKGFYPSDIAVEWESNGQPENNYKTTPPVLDSDGSFFLYSR | |
| | LTVDKSRWQEGNVFSCSVMHEALHNHYTQKSLSLSLGKMF | |
| | WVLVVVGGVLACYSLLVTVAFIIFWVKRGRKKLLYIFKQPF | |
| | MRPVQTTQEEDGCSCRFPEEEEGGCELRVKFSRSADAPAYQ | |
| | QGQNQLYNELNLGRREEYDVLDKRRGRDPEMGGKPRRKNP | |
| | QEGLYNELQKDKMAEAYSEIGMKGERRRGKGHDGLYQGLS | |
| | TATKDTYDALHMQALPPR | |
| 38 | IVGGHEAQPHSRPYMASLQMRGNPGSHFCGGTLIHPSF | PR3_4 (D91N) |
| | VLTAAHCLRDIPQRLVNVVLGAHNVRTQEPTQQHFSVA | SS CAAR |
| | QVFLNNYDAENKLNNVLLIQLSSPANLSASVATVQLPQ | (PR3_4_38) |
| | QDQPVPHGTQCLAMGWGRVGAHDPPAQVLQELNVTV | Amino acid |
| | VTFFCRPHNICTFVPRRKAGICFGDSGGPLICDGIIQGIDS | |
| | FVIWGCATRLFPDFFTRVALYVDWIRSTLRESKYGPPCP | |
| | PCPMFWVLVVVGGVLACYSLLVTVAFIIFWVKRGRKK | |
| | LLYIFKQPFMRPVQTTQEEDGCSCRFPEEEEGGCELR | |
| | VKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPP | |
| | R | |
| 39 | IVGGHEAQPHSRPYMASLQMRGNPGSHFCGGTLIHPSFVLTA | PR3_4 (D91N) |
| | AHCLRDIPQRLVNVVLGAHNVRTQEPTQQHFSVAQVFLNNY | MS CAAR |
| | DAENKLNNVLLIQLSSPANLSASVATVQLPQQDQPVPHGTQC | (PR3_4_39) |
| | LAMGWGRVGAHDPPAQVLQELNVTVVTFFCRPHNICTFVPR | Amino acid |
| | RKAGICFGDSGGPLICDGIIQGIDSFVIWGCATRLFPDFFTRVA | |
| | LYVDWIRSTLRESKYGPPCPPCPGQPREPQVYTLPPSQEEMT | |
| | KNQVSLTCLVKGFYPSDIAVEWESNGQPENNYKTTPPVLDS | |
| | DGSFFLYSRLTVDKSRWQEGNVFSCSVMHEALHNHYTQKSL | |
| | SLSLGKMFWVLVVVGGVLACYSLLVTVAFIIFWVKRGRKKL | |
| | LYIFKQPFMRPVQTTQEEDGCSCRFPEEEEGGCELRVKFSRSA | |
| | DAPAYQQGQNQLYNELNLGRREEYDVLDKRRGRDPEMGGK | |
| | PRRKNPQEGLYNELQKDKMAEAYSEIGMKGERRRGKGHDG | |
| | LYQGLSTATKDTYDALHMQALPPR | |
| 40 | IVGGHEAQPHSRPYMASLQMRGNPGSHFCGGTLIHPSFVLTA | PR3_4 (D91N) |
| | AHCLRDIPQRLVNVVLGAHNVRTQEPTQQHFSVAQVFLNNY | LS CAAR |
| | DAENKLNNVLLIQLSSPANLSASVATVQLPQQDQPVPHGTQC | (PR3_4_40) |
| | LAMGWGRVGAHDPPAQVLQELNVTVVTFFCRPHNICTFVPR | Amino acid |
| | RKAGICFGDSGGPLICDGIIQGIDSFVIWGCATRLFPDFFTRVA | |
| | LYVDWIRSTLRESKYGPPCPPCPAPPVAGPSVFLFPPKPKDTL | |
| | MISRTPEVTCVVVDVSQEDPEVQFNWYVDGVEVHNAKTKP | |
| | REEQFQSTYRVVSVLTVLHQDWLNGKEYKCKVSNKGLPSSI | |
| | EKTISKAKGQPREPQVYTLPPSQEEMTKNQVSLTCLVKGFYP | |
| | SDIAVEWESNGQPENNYKTTPPVLDSDGSFFLYSRLTVDKSR | |
| | WQEGNVFSCSVMHEALHNHYTQKSLSLSLGKMFWVLVVVG | |
| | GVLACYSLLVTVAFIIFWVKRGRKKLLYIFKQPFMRPVQTTQ | |
| | EEDGCSCRFPEEEEGGCELRVKFSRSADAPAYQQGQNQLYN | |
| | ELNLGRREEYDVLDKRRGRDPEMGGKPRRKNPQEGLYNEL | |
| | QKDKMAEAYSEIGMKGERRRGKGHDGLYQGLSTATKDTYD | |
| | ALHMQALPPR | |
| 41 | AEIVGGHEAQPHSRPYMASLQMRGNPGSHFCGGTLIHP | PR3_5 |
| | SFVLTAAHCLRDIPQRLVNVVLGAHNVRTQEPTQQHFS | (Unprocessed N- |
| | VAQVFLNNYDAENKLNDVLLIQLSSPANLSASVATVQL | terminus) SS |
| | PQQDQPVPHGTQCLAMGWGRVGAHDPPAQVLQELNV | CAAR |
| | TVVTFFCRPHNICTFVPRRKAGICFGDSGGPLICDGIIQGI | (PR3_5_41) |
| | DSFVIWGCATRLFPDFFTRVALYVDWIRSTLRESKYGPP | Amino acid |
| | CPPCPMFWVLVVVGGVLACYSLLVTVAFIIFWVKRGRK | |
| | KLLYIFKQPFMRPVQTTQEEDGCSCRFPEEEEGGCELRV | |
| | KFSRSADAPAYQQGQNQLYNELNLGRREEYDVLDKRR | |
| | GRDPEMGGKPRRKNPQEGLYNELQKDKMAEAYSEIGM | |
| | KGERRRGKGHDGLYQGLSTATKDTYDALHMQALPPR | |
| 42 | AEIVGGHEAQPHSRPYMASLQMRGNPGSHFCGGTLIHP | PR3_5 |
| | SFVLTAAHCLRDIPQRLVNVVLGAHNVRTQEPTQQHFS | (Unprocessed N- |
| | VAQVFLNNYDAENKLNDVLLIQLSSPANLSASVATVQL | terminus) MS |
| | PQQDQPVPHGTQCLAMGWGRVGAHDPPAQVLQELNV | CAAR |
| | TVVTFFCRPHNICTFVPRRKAGICFGDSGGPLICDGIIQGI | (PR3_5_42) |
| | DSFVIWGCATRLFPDFFTRVALYVDWIRSTLRESKYGPP | Amino acid |
| | CPPCPGQPREPQVYTLPPSQEEMTKNQVSLTCLVKGFYP | |
| | SDIAVEWESNGQPENNYKTTPPVLDSDGSFFLYSRLTVD | |
| | KSRWQEGNVFSCSVMHEALHNHYTQKSLSLSLGKMFW | |
| | VLVVVGGVLACYSLLVTVAFIIFWVKRGRKKLLYIFKQ | |
| | PFMRPVQTTQEEDGCSCRFPEEEEGGCELRVKFSRSAD | |
| | APAYQQGQNQLYNELNLGRREEYDVLDKRRGRDPEM | |
| | GGKPRRKNPQEGLYNELQKDKMAEAYSEIGMKGERRR | |
| | GKGHDGLYQGLSTATKDTYDALHMQALPPR | |
| 43 | AEIVGGHEAQPHSRPYMASLQMRGNPGSHFCGGTLIHP | PR3_5 |
| | SFVLTAAHCLRDIPQRLVNVVLGAHNVRTQEPTQQHFS | (Unprocessed N- |
| | VAQVFLNNYDAENKLNDVLLIQLSSPANLSASVATVQL | terminus) LS |
| | PQQDQPVPHGTQCLAMGWGRVGAHDPPAQVLQELNV | CAAR |
| | TVVTFFCRPHNICTFVPRRKAGICFGDSGGPLICDGIIQGI | (PR3_5_43) |
| | DSFVIWGCATRLFPDFFTRVALYVDWIRSTLRESKYGPP | Amino acid |
| | CPPCPAPPVAGPSVFLFPPKPKDTLMISRTPEVTCVVVD | |
| | VSQEDPEVQFNWYVDGVEVHNAKTKPREEQFQSTYRV | |
| | VSVLTVLHQDWLNGKEYKCKVSNKGLPSSIEKTISKAK | |
| | GQPREPQVYTLPPSQEEMTKNQVSLTCLVKGFYPSDIA | |
| | VEWESNGQPENNYKTTPPVLDSDGSFFLYSRLTVDKSR | |
| | WQEGNVFSCSVMHEALHNHYTQKSLSLSLGKMFWVL | |
| | VVVGGVLACYSLLVTVAFIIFWVKRGRKKLLYIFKQPF | |
| | MRPVQTTQEEDGCSCRFPEEEEGGCELRVKFSRSADAP | |
| | AYQQGQNQLYNELNLGRREEYDVLDKRRGRDPEMGG | |
| | KPRRKNPQEGLYNELQKDKMAEAYSEIGMKGERRRGK | |
| | GHDGLYQGLSTATKDTYDALHMQALPPR | |
| 44 | VGGHEAQPHSRPYMASLQMRGNPGSHFCGGTLIHPSFV | PR3_6 (Ile |
| | LTAAHCLRDIPQRLVNVVLGAHNVRTQEPTQQHFSVAQ | Deletion) SS |
| | VFLNNYDAENKLNDVLLIQLSSPANLSASVATVQLPQQ | CAAR |
| | DQPVPHGTQCLAMGWGRVGAHDPPAQVLQELNVTVV | (PR3_6_44) |
| | TFFCRPHNICTFVPRRKAGICFGDSGGPLICDGIIQGIDSF | Amino acid |
| | VIWGCATRLFPDFFTRVALYVDWIRSTLRESKYGPPCPP | |
| | CPMFWVLVVVGGVLACYSLLVTVAFIIFWVKRGRKKL | |
| | LYIFKQPFMRPVQTTQEEDGCSCRFPEEEEGGCELR | |
| | VKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPP | |
| | R | |
| 45 | VGGHEAQPHSRPYMASLQMRGNPGSHFCGGTLIHPSFV | PR3_6 (Ile |
| | LTAAHCLRDIPQRLVNVVLGAHNVRTQEPTQQHFSVAQ | Deletion) MS |
| | VFLNNYDAENKLNDVLLIQLSSPANLSASVATVQLPQQ | CAAR |
| | DQPVPHGTQCLAMGWGRVGAHDPPAQVLQELNVTVV | (PR3_6_45) |
| | TFFCRPHNICTFVPRRKAGICFGDSGGPLICDGIIQGIDSF | Amino acid |
| | VIWGCATRLFPDFFTRVALYVDWIRSTLRESKYGPPCPP | |
| | CPGQPREPQVYTLPPSQEEMTKNQVSLTCLVKGFYPSDI | |
| | AVEWESNGQPENNYKTTPPVLDSDGSFFLYSRLTVDKS | |
| | RWQEGNVFSCSVMHEALHNHYTQKSLSLSLGKMFWV | |
| | LVVVGGVLACYSLLVTVAFIIFWVKRGRKKLLYIFKQPF | |
| | MRPVQTTQEEDGCSCRFPEEEEGGCELRVKFSRSADAP | |
| | AYQQGQNQLYNELNLGRREEYDVLDKRRGRDPEMGG | |
| | KPRRKNPQEGLYNELQKDKMAEAYSEIGMKGERRRGK | |
| | GHDGLYQGLSTATKDTYDALHMQALPPR | |
| 46 | VGGHEAQPHSRPYMASLQMRGNPGSHFCGGTLIHPSFV | PR3_6 (Ile |
| | LTAAHCLRDIPQRLVNVVLGAHNVRTQEPTQQHFSVAQ | Deletion) LS |
| | VFLNNYDAENKLNDVLLIQLSSPANLSASVATVQLPQQ | CAAR |
| | DQPVPHGTQCLAMGWGRVGAHDPPAQVLQELNVTVV | (PR3_6_46) |
| | TFFCRPHNICTFVPRRKAGICFGDSGGPLICDGIIQGIDSF | Amino acid |
| | VIWGCATRLFPDFFTRVALYVDWIRSTLRESKYGPPCPP | |
| | CPAPPVAGPSVFLFPPKPKDTLMISRTPEVTCVVVDVSQ | |
| | EDPEVQFNWYVDGVEVHNAKTKPREEQFQSTYRVVSV | |
| | LTVLHQDWLNGKEYKCKVSNKGLPSSIEKTISKAKGQP | |
| | REPQVYTLPPSQEEMTKNQVSLTCLVKGFYPSDIAVEW | |
| | ESNGQPENNYKTTPPVLDSDGSFFLYSRLTVDKSRWQE | |
| | GNVFSCSVMHEALHNHYTQKSLSLSLGKMFWVLVVVG | |
| | GVLACYSLLVTVAFIIFWVKRGRKKLLYIFKQPFMRPV | |
| | QTTQEEDGCSCRFPEEEEGGCELRVKFSRSADAPAYQQ | |
| | GQNQLYNELNLGRREEYDVLDKRRGRDPEMGGKPRRK | |
| | NPQEGLYNELQKDKMAEAYSEIGMKGERRRGKGHDGL | |
| | YQGLSTATKDTYDALHMQALPPR | |
| 47 | IVGGHEAQPHSRPYMASLQMRGNPGSHFCGGTLIHPSF | PR3_7 (D175N) |
| | VLTAAHCLRDIPQRLVNVVLGAHNVRTQEPTQQHFSVA | SS CAAR |
| | QVFLNNYDAENKLNDVLLIQLSSPANLSASVATVQLPQ | (PR3_7_47) |
| | QDQPVPHGTQCLAMGWGRVGAHDPPAQVLQELNVTV | Amino acid |
| | VTFFCRPHNICTFVPRRKAGICFGNSGGPLICDGIIQGIDS | |
| | FVIWGCATRLFPDFFTRVALYVDWIRSTLRESKYGPPCP | |
| | PCPMFWVLVVVGGVLACYSLLVTVAFIIFWVKRGRKK | |
| | LLYIFKQPFMRPVQTTQEEDGCSCRFPEEEEGGCELR | |
| | VKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPP | |
| | R | |
| 48 | IVGGHEAQPHSRPYMASLQMRGNPGSHFCGGTLIHPSF | PR3_7 (D175N) |
| | VLTAAHCLRDIPQRLVNVVLGAHNVRTQEPTQQHFSVA | MS CAAR |
| | QVFLNNYDAENKLNDVLLIQLSSPANLSASVATVQLPQ | (PR3_7_48) |
| | QDQPVPHGTQCLAMGWGRVGAHDPPAQVLQELNVTV | Amino acid |
| | VTFFCRPHNICTFVPRRKAGICFGNSGGPLICDGIIQGIDS | |
| | FVIWGCATRLFPDFFTRVALYVDWIRSTLRESKYGPPCP | |
| | PCPGQPREPQVYTLPPSQEEMTKNQVSLTCLVKGFYPS | |
| | DIAVEWESNGQPENNYKTTPPVLDSDGSFFLYSRLTVD | |
| | KSRWQEGNVFSCSVMHEALHNHYTQKSLSLSLGKMFW | |
| | VLVVVGGVLACYSLLVTVAFIIFWVKRGRKKLLYIFKQ | |
| | PFMRPVQTTQEEDGCSCRFPEEEEGGCELRVKFSRSAD | |
| | APAYQQGQNQLYNELNLGRREEYDVLDKRRGRDPEM | |
| | GGKPRRKNPQEGLYNELQKDKMAEAYSEIGMKGERRR | |
| | GKGHDGLYQGLSTATKDTYDALHMQALPPR | |
| 49 | IVGGHEAQPHSRPYMASLQMRGNPGSHFCGGTLIHPSF | PR3_7 (D175N) |
| | VLTAAHCLRDIPQRLVNVVLGAHNVRTQEPTQQHFSVA | LS CAAR |
| | QVFLNNYDAENKLNDVLLIQLSSPANLSASVATVQLPQ | (PR3_7_49) |
| | QDQPVPHGTQCLAMGWGRVGAHDPPAQVLQELNVTV | Amino acid |
| | VTFFCRPHNICTFVPRRKAGICFGNSGGPLICDGIIQGIDS | |
| | FVIWGCATRLFPDFFTRVALYVDWIRSTLRESKYGPPCP | |
| | PCPAPPVAGPSVFLFPPKPKDTLMISRTPEVTCVVVDVS | |
| | QEDPEVQFNWYVDGVEVHNAKTKPREEQFQSTYRVVS | |
| | VLTVLHQDWLNGKEYKCKVSNKGLPSSIEKTISKAKGQ | |
| | PREPQVYTLPPSQEEMTKNQVSLTCLVKGFYPSDIAVE | |
| | WESNGQPENNYKTTPPVLDSDGSFFLYSRLTVDKSRWQ | |
| | EGNVFSCSVMHEALHNHYTQKSLSLSLGKMFWVLVVV | |
| | GGVLACYSLLVTVAFIIFWVKRGRKKLLYIFKQPFMRP | |
| | VQTTQEEDGCSCRFPEEEEGGCELRVKFSRSADAPAYQ | |
| | QGQNQLYNELNLGRREEYDVLDKRRGRDPEMGGKPRR | |
| | KNPQEGLYNELQKDKMAEAYSEIGMKGERRRGKGHD | |
| | GLYQGLSTATKDTYDALHMQALPPR | |
| 50 | IVGGHEAQPHSRPYMASLQMRGNPGSHFCGGTLIHPSF | PR3_8 (S176C) |
| | VLTAAHCLRDIPQRLVNVVLGAHNVRTQEPTQQHFSVA | SS CAAR |
| | QVFLNNYDAENKLNDVLLIQLSSPANLSASVATVQLPQ | (PR3_8_50) |
| | QDQPVPHGTQCLAMGWGRVGAHDPPAQVLQELNVTV | Amino acid |
| | VTFFCRPHNICTFVPRRKAGICFGDCGGPLICDGIIQGIDS | |
| | FVIWGCATRLFPDFFTRVALYVDWIRSTLRESKYGPPCP | |
| | PCPMFWVLVVVGGVLACYSLLVTVAFIIFWVKRGRKK | |
| | LLYIFKQPFMRPVQTTQEEDGCSCRFPEEEEGGCELR | |
| | VKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPP | |
| | R | |
| 51 | IVGGHEAQPHSRPYMASLQMRGNPGSHFCGGTLIHPSFVLTA | PR3_8 (S176C) |
| | AHCLRDIPQRLVNVVLGAHNVRTQEPTQQHFSVAQVFLNNY | MS CAAR |
| | DAENKLNDVLLIQLSSPANLSASVATVQLPQQDQPVPHGTQC | (PR3_8_51) |
| | LAMGWGRVGAHDPPAQVLQELNVTVVTFFCRPHNICTFVPR | Amino acid |
| | RKAGICFGDCGGPLICDGIIQGIDSFVIWGCATRLFPDFFTRVA | |
| | LYVDWIRSTLRESKYGPPCPPCPGQPREPQVYTLPPSQEEMT | |
| | KNQVSLTCLVKGFYPSDIAVEWESNGQPENNYKTTPPVLDS | |
| | DGSFFLYSRLTVDKSRWQEGNVFSCSVMHEALHNHYTQKSL | |
| | SLSLGKMFWVLVVVGGVLACYSLLVTVAFIIFWVKRGRKKL | |
| | LYIFKQPFMRPVQTTQEEDGCCRFPEEEEGGCELRVKFSRSA | |
| | DAPAYQQGQNQLYNELNLGRREEYDVLDKRRGRDPEMGGK | |
| | PRRKNPQEGLYNELQKDKMAEAYSEIGMKGERRRGKGHDG | |
| | LYQGLSTATKDTYDALHMQALPPR | |
| 52 | IVGGHEAQPHSRPYMASLQMRGNPGSHFCGGTLIHPSFVLTA | PR3_8 (S176C) |
| | AHCLRDIPQRLVNVVLGAHNVRTQEPTQQHFSVAQVFLNNY | LS CAAR |
| | DAENKLNDVLLIQLSSPANLSASVATVQLPQQDQPVPHGTQC | (PR3_8_52) |
| | LAMGWGRVGAHDPPAQVLQELNVTVVTFFCRPHNICTFVPR | Amino acid |
| | RKAGICFGDCGGPLICDGIIQGIDSFVIWGCATRLFPDFFTRVA | |
| | LYVDWIRSTLRESKYGPPCPPCPAPPVAGPSVFLFPPKPKDTL | |
| | MISRTPEVTCVVVDVSQEDPEVQFNWYVDGVEVHNAKTKP | |
| | REEQFQSTYRVVSVLTVLHQDWLNGKEYKCKVSNKGLPSSI | |
| | EKTISKAKGQPREPQVYTLPPSQEEMTKNQVSLTCLVKGFYP | |
| | SDIAVEWESNGQPENNYKTTPPVLDSDGSFFLYSRLTVDKSR | |
| | WQEGNVFSCSVMHEALHNHYTQKSLSLSLGKMFWVLVVVG | |
| | GVLACYSLLVTVAFIIFWVKRGRKKLLYIFKQPFMRPVQTTQ | |
| | EEDGCSCRFPEEEEGGCELRVKFSRSADAPAYQQGQNQLYN | |
| | ELNLGRREEYDVLDKRRGRDPEMGGKPRRKNPQEGLYNEL | |
| | QKDKMAEAYSEIGMKGERRRGKGHDGLYQGLSTATKDTYD | |
| | ALHMQALPPR | |
| 53 | IVGPHEAQPHSRPYMASLQMRGNPGSHFCGGTLIHPSFV | PR3_9 (G4P) SS |
| | LTAAHCLRDIPQRLVNVVLGAHNVRTQEPTQQHFSVAQ | CAAR |
| | VFLNNYDAENKLNDVLLIQLSSPANLSASVATVQLPQQ | (PR3_9_53) |
| | DQPVPHGTQCLAMGWGRVGAHDPPAQVLQELNVTVV | Amino acid |
| | TFFCRPHNICTFVPRRKAGICFGDSGGPLICDGIIQGIDSF | |
| | VIWGCATRLFPDFFTRVALYVDWIRSTLRESKYGPPCPP | |
| | CPMFWVLVVVGGVLACYSLLVTVAFIIFWVKRGRKKL | |
| | LYIFKQPFMRPVQTTQEEDGCSCRFPEEEEGGCELR | |
| | VKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPP | |
| | R | |
| 54 | IVGPHEAQPHSRPYMASLQMRGNPGSHFCGGTLIHPSFVLTA | PR3_9 (G4P) |
| | AHCLRDIPQRLVNVVLGAHNVRTQEPTQQHFSVAQVFLNNY | MS CAAR |
| | DAENKLNDVLLIQLSSPANLSASVATVQLPQQDQPVPHGTQC | (PR3_9_54) |
| | LAMGWGRVGAHDPPAQVLQELNVTVVTFFCRPHNICTFVPR | Amino acid |
| | RKAGICFGDSGGPLICDGIIQGIDSFVIWGCATRLFPDFFTRVA | |
| | LYVDWIRSTLRESKYGPPCPPCPGQPREPQVYTLPPSQEEMT | |
| | KNQVSLTCLVKGFYPSDIAVEWESNGQPENNYKTTPPVLDS | |
| | DGSFFLYSRLTVDKSRWQEGNVFSCSVMHEALHNHYTQKSL | |
| | SLSLGKMFWVLVVVGGVLACYSLLVTVAFIIFWVKRGRKKL | |
| | LYIFKQPFMRPVQTTQEEDGCSCRFPEEEEGGCELRVKFSRSA | |
| | DAPAYQQGQNQLYNELNLGRREEYDVLDKRRGRDPEMGGK | |
| | PRRKNPQEGLYNELQKDKMAEAYSEIGMKGERRRGKGHDG | |
| | LYQGLSTATKDTYDALHMQALPPR | |
| 55 | IVGPHEAQPHSRPYMASLQMRGNPGSHFCGGTLIHPSFVLTA | PR3_9 (G4P) LS |
| | AHCLRDIPQRLVNVVLGAHNVRTQEPTQQHFSVAQVFLNNY | CAAR |
| | DAENKLNDVLLIQLSSPANLSASVATVQLPQQDQPVPHGTQC | (PR3_9_55) |
| | LAMGWGRVGAHDPPAQVLQELNVTVVTFFCRPHNICTFVPR | Amino acid |
| | RKAGICFGDSGGPLICDGIIQGIDSFVIWGCATRLFPDFFTRVA | |
| | LYVDWIRSTLRESKYGPPCPPCPAPPVAGPSVFLFPPKPKDTL | |
| | MISRTPEVTCVVVDVSQEDPEVQFNWYVDGVEVHNAKTKP | |
| | REEQFQSTYRVVSVLTVLHQDWLNGKEYKCKVSNKGLPSSI | |
| | EKTISKAKGQPREPQVYTLPPSQEEMTKNQVSLTCLVKGFYP | |
| | SDIAVEWESNGQPENNYKTTPPVLDSDGSFFLYSRLTVDKSR | |
| | WQEGNVFSCSVMHEALHNHYTQKSLSLSLGKMFWVLVVVG | |
| | GVLACYSLLVTVAFIIFWVKRGRKKLLYIFKQPFMRPVQTTQ | |
| | EEDGCSCRFPEEEEGGCELRVKFSRSADAPAYQQGQNQLYN | |
| | ELNLGRREEYDVLDKRRGRDPEMGGKPRRKNPQEGLYNEL | |
| | QKDKMAEAYSEIGMKGERRRGKGHDGLYQGLSTATKDTYD | |
| | ALHMQALPPR | |
| 56 | IVGGHEAQPHSRPYMASLQMRGNPGSHFCGGTLIHPSF | PR3_10 (H44A) |
| | VLTAAACLRDIPQRLVNVVLGAHNVRTQEPTQQHFSVA | SS CAAR |
| | QVFLNNYDAENKLNDVLLIQLSSPANLSASVATVQLPQ | (PR3_10_56) |
| | QDQPVPHGTQCLAMGWGRVGAHDPPAQVLQELNVTV | Amino acid |
| | VTFFCRPHNICTFVPRRKAGICFGDSGGPLICDGIIQGIDS | |
| | FVIWGCATRLFPDFFTRVALYVDWIRSTLRESKYGPPCP | |
| | PCPMFWVLVVVGGVLACYSLLVTVAFIIFWVKRGRKK | |
| | LLYIFKQPFMRPVQTTQEEDGCSCRFPEEEEGGCELR | |
| | VKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPP | |
| | R | |
| 57 | IVGGHEAQPHSRPYMASLQMRGNPGSHFCGGTLIHPSFVLTA | PR3_10 (H44A) |
| | AACLRDIPQRLVNVVLGAHNVRTQEPTQQHFSVAQVFLNNY | MS CAAR |
| | DAENKLNDVLLIQLSSPANLSASVATVQLPQQDQPVPHGTQC | (PR3_10_57) |
| | LAMGWGRVGAHDPPAQVLQELNVTVVTFFCRPHNICTFVPR | Amino acid |
| | RKAGICFGDSGGPLICDGIIQGIDSFVIWGCATRLFPDFFTRVA | |
| | LYVDWIRSTLRESKYGPPCPPCPGQPREPQVYTLPPSQEEMT | |
| | KNQVSLTCLVKGFYPSDIAVEWESNGQPENNYKTTPPVLDS | |
| | DGSFFLYSRLTVDKSRWQEGNVFSCSVMHEALHNHYTQKSL | |
| | SLSLGKMFWVLVVVGGVLACYSLLVTVAFIIFWVKRGRKKL | |
| | LYIFKQPFMRPVQTTQEEDGCSCRFPEEEEGGCELRVKFSRSA | |
| | DAPAYQQGQNQLYNELNLGRREEYDVLDKRRGRDPEMGGK | |
| | PRRKNPQEGLYNELQKDKMAEAYSEIGMKGERRRGKGHDG | |
| | LYQGLSTATKDTYDALHMQALPPR | |
| 58 | IVGGHEAQPHSRPYMASLQMRGNPGSHFCGGTLIHPSFVLTA | PR3_10 (H44A) |
| | AACLRDIPQRLVNVVLGAHNVRTQEPTQQHFSVAQVFLNNY | LS CAAR |
| | DAENKLNDVLLIQLSSPANLSASVATVQLPQQDQPVPHGTQC | (PR3_10_58) |
| | LAMGWGRVGAHDPPAQVLQELNVTVVTFFCRPHNICTFVPR | Amino acid |
| | RKAGICFGDSGGPLICDGIIQGIDSFVIWGCATRLFPDFFTRVA | |
| | LYVDWIRSTLRESKYGPPCPPCPAPPVAGPSVFLFPPKPKDTL | |
| | MISRTPEVTCVVVDVSQEDPEVQFNWYVDGVEVHNAKTKP | |
| | REEQFQSTYRVVSVLTVLHQDWLNGKEYKCKVSNKGLPSSI | |
| | EKTISKAKGQPREPQVYTLPPSQEEMTKNQVSLTCLVKGFYP | |
| | SDIAVEWESNGQPENNYKTTPPVLDSDGSFFLYSRLTVDKSR | |
| | WQEGNVFSCSVMHEALHNHYTQKSLSLSLGKMFWVLVVVG | |
| | GVLACYSLLVTVAFIIFWVKRGRKKLLYIFKQPFMRPVQTTQ | |
| | EEDGCSCRFPEEEEGGCELRVKFSRSADAPAYQQGQNQLYN | |
| | ELNLGRREEYDVLDKRRGRDPEMGGKPRRKNPQEGLYNEL | |
| | QKDKMAEAYSEIGMKGERRRGKGHDGLYQGLSTATKDTYD | |
| | ALHMQALPPR | |
| 59 | ATGCCTTTGTTGCTGCTGCTGCCTCTTCTGTGGGCTGG | Human PR3 |
| | CGCTCTGGCTATCGTTGGAGGACATGAAGCCCAGCCT | sequence (WT) |
| | CACAGCAGACCTTACATGGCCAGCCTGCAGATGAGA | or PR3_1 SS |
| | GGCAATCCCGGCTCTCACTTTTGTGGCGGCACCCTGA | CAAR |
| | TTCACCCCTCCTTCGTTCTGACCGCCGCTCACTGCCTG | (PR3_1_29) |
| | AGAGACATCCCTCAGAGACTGGTCAACGTGGTGCTG | Nucleotide |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTCGGAGATTCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCATGTTCTGGGTGCTCGTGGTCGTTGG | |
| | CGGAGTGCTGGCCTGTTACAGCCTGCTGGTTACCGTG | |
| | GCCTTCATCATCTTTTGGGTCAAGCGGGGCAGAAAGA | |
| | AGCTGCTCTACATCTTCAAGCAGCCCTTCATGCGGCC | |
| | CGTGCAGACCACACAAGAGGAAGATGGCTGCTCCTG | |
| | CAGATTCCCCGAGGAAGAAGAAGGCGGCTGCGAGCT | |
| | GAGAGTGAAGTTCAGCAGATCCGCCGACGCTCCAGC | |
| | CTATCAGCAGGGCCAAAACCAGCTGTACAACGAGCT | |
| | GAACCTGGGGAGAAGAGAAGAGTACGACGTGCTGGA | |
| | TAAGCGGAGAGGCAGAGATCCTGAAATGGGCGGCAA | |
| | GCCCAGACGGAAGAATCCTCAAGAGGGCCTGTATAA | |
| | TGAGCTGCAGAAAGACAAGATGGCCGAGGCCTACAG | |
| | CGAGATCGGAATGAAGGGCGAGCGCAGAAGAGGCA | |
| | AGGGACACGATGGACTGTACCAGGGCCTGAGCACCG | |
| | CCACCAAGGATACCTATGACGCACTGCACATGCAGG | |
| | CCCTGCCACCTAGA | |
| 60 | ATGCCTTTGTTGCTGCTGCTGCCTCTTCTGTGGGCTGG | Human PR3 |
| | CGCTCTGGCTATCGTTGGAGGACATGAAGCCCAGCCT | sequence (WT) |
| | CACAGCAGACCTTACATGGCCAGCCTGCAGATGAGA | or PR3_1 MS |
| | GGCAATCCCGGCTCTCACTTTTGTGGCGGCACCCTGA | CAAR |
| | TTCACCCCTCCTTCGTTCTGACCGCCGCTCACTGCCTG | (PR3_1_30) |
| | AGAGACATCCCTCAGAGACTGGTCAACGTGGTGCTG | Nucleotide |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTCGGAGATTCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCGGCCAGCCAAGAGAGCCCCAGGTTT | |
| | ACACACTGCCTCCAAGCCAAGAGGAAATGACCAAGA | |
| | ATCAGGTGTCCCTGACATGCCTGGTCAAGGGCTTCTA | |
| | CCCCTCCGATATCGCCGTGGAATGGGAGAGCAATGG | |
| | CCAGCCTGAGAACAACTACAAGACCACACCTCCTGT | |
| | GCTGGACAGCGACGGCAGTTTCTTCCTGTATAGTAGA | |
| | CTCACCGTGGATAAATCAAGATGGCAAGAGGGCAAC | |
| | GTGTTCAGCTGCAGCGTGATGCACGAGGCCCTGCAC | |
| | AACCACTACACCCAGAAAAGCCTGAGCCTGTCTCTG | |
| | GGCAAGATGTTCTGGGTGCTCGTGGTCGTTGGCGGAG | |
| | TGCTGGCCTGTTACAGCCTGCTGGTTACCGTGGCCTT | |
| | CATCATCTTTTGGGTCAAGCGGGGCAGAAAGAAGCT | |
| | GCTCTACATCTTCAAGCAGCCCTTCATGCGGCCCGTG | |
| | CAGACCACACAAGAGGAAGATGGCTGCTCCTGCAGA | |
| | TTCCCCGAGGAAGAAGAAGGCGGCTGCGAGCTGAGA | |
| | GTGAAGTTCAGCAGATCCGCCGACGCTCCAGCCTATC | |
| | AGCAGGGCCAAAACCAGCTGTACAACGAGCTGAACC | |
| | TGGGGAGAAGAGAAGAGTACGACGTGCTGGATAAGC | |
| | GGAGAGGCAGAGATCCTGAAATGGGCGGCAAGCCCA | |
| | GACGGAAGAATCCTCAAGAGGGCCTGTATAATGAGC | |
| | TGCAGAAAGACAAGATGGCCGAGGCCTACAGCGAGA | |
| | TCGGAATGAAGGGCGAGCGCAGAAGAGGCAAGGGA | |
| | CACGATGGACTGTACCAGGGCCTGAGCACCGCCACC | |
| | AAGGATACCTATGACGCACTGCACATGCAGGCCCTG | |
| | CCACCTAGA | |
| 61 | ATGCCTTTGTTGCTGCTGCTGCCTCTTCTGTGGGCTGG | Human PR3 |
| | CGCTCTGGCTATCGTTGGAGGACATGAAGCCCAGCCT | sequence (WT) |
| | CACAGCAGACCTTACATGGCCAGCCTGCAGATGAGA | or PR3_1 LS |
| | GGCAATCCCGGCTCTCACTTTTGTGGCGGCACCCTGA | CAAR |
| | TTCACCCCTCCTTCGTTCTGACCGCCGCTCACTGCCTG | (PR3_1_31) |
| | AGAGACATCCCTCAGAGACTGGTCAACGTGGTGCTG | Nucleotide |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTCGGAGATTCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCGCTCCTCCTGTTGCCGGACCTTCCGT | |
| | GTTCCTGTTTCCTCCAAAGCCTAAGGACACCCTGATG | |
| | ATCAGCAGGACCCCTGAAGTGACCTGCGTGGTGGTG | |
| | GATGTGTCCCAAGAGGATCCCGAGGTGCAGTTCAAC | |
| | TGGTATGTGGACGGCGTGGAAGTGCACAACGCCAAG | |
| | ACCAAGCCTAGAGAGGAACAGTTCCAGAGCACCTAC | |
| | AGAGTGGTGTCCGTGCTGACAGTGCTGCACCAGGATT | |
| | GGCTGAACGGCAAAGAGTACAAGTGCAAGGTGTCCA | |
| | ACAAGGGCCTGCCTAGCAGCATCGAGAAAACCATCT | |
| | CCAAGGCCAAGGGCCAGCCAAGAGAGCCCCAGGTTT | |
| | ACACACTGCCTCCAAGCCAAGAGGAAATGACCAAGA | |
| | ATCAGGTGTCCCTGACATGCCTGGTCAAGGGCTTCTA | |
| | CCCCTCCGATATCGCCGTGGAATGGGAGAGCAATGG | |
| | CCAGCCTGAGAACAACTACAAGACCACACCTCCTGT | |
| | GCTGGACAGCGACGGCAGTTTCTTCCTGTATAGTAGA | |
| | CTCACCGTGGATAAATCAAGATGGCAAGAGGGCAAC | |
| | GTGTTCAGCTGCAGCGTGATGCACGAGGCCCTGCAC | |
| | AACCACTACACCCAGAAAAGCCTGAGCCTGTCTCTG | |
| | GGCAAGATGTTCTGGGTGCTCGTGGTCGTTGGCGGAG | |
| | TGCTGGCCTGTTACAGCCTGCTGGTTACCGTGGCCTT | |
| | CATCATCTTTTGGGTCAAGCGGGGCAGAAAGAAGCT | |
| | GCTCTACATCTTCAAGCAGCCCTTCATGCGGCCCGTG | |
| | CAGACCACACAAGAGGAAGATGGCTGCTCCTGCAGA | |
| | TTCCCCGAGGAAGAAGAAGGCGGCTGCGAGCTGAGA | |
| | GTGAAGTTCAGCAGATCCGCCGACGCTCCAGCCTATC | |
| | AGCAGGGCCAAAACCAGCTGTACAACGAGCTGAACC | |
| | TGGGGAGAAGAGAAGAGTACGACGTGCTGGATAAGC | |
| | GGAGAGGCAGAGATCCTGAAATGGGCGGCAAGCCCA | |
| | GACGGAAGAATCCTCAAGAGGGCCTGTATAATGAGC | |
| | TGCAGAAAGACAAGATGGCCGAGGCCTACAGCGAGA | |
| | TCGGAATGAAGGGCGAGCGCAGAAGAGGCAAGGGA | |
| | CACGATGGACTGTACCAGGGCCTGAGCACCGCCACC | |
| | AAGGATACCTATGACGCACTGCACATGCAGGCCCTG | |
| | CCACCTAGA | |
| 62 | ATGCCTTTGTTGCTGCTGCTGCCTCTTCTGTGGGCTGG | PR3_2 (S176A) |
| | CGCTCTGGCTATCGTTGGAGGACATGAAGCCCAGCCT | SS CAAR |
| | CACAGCAGACCTTACATGGCCAGCCTGCAGATGAGA | (PR3_2_32) |
| | GGCAATCCCGGCTCTCACTTTTGTGGCGGCACCCTGA | Nucleotide |
| | TTCACCCCTCCTTCGTTCTGACCGCCGCTCACTGCCTG | |
| | AGAGACATCCCTCAGAGACTGGTCAACGTGGTGCTG | |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTTGGAGATGCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCATGTTCTGGGTGCTCGTGGTCGTTGG | |
| | CGGAGTGCTGGCCTGTTACAGCCTGCTGGTTACCGTG | |
| | GCCTTCATCATCTTTTGGGTCAAGCGGGGCAGAAAGA | |
| | AGCTGCTCTACATCTTCAAGCAGCCCTTCATGCGGCC | |
| | CGTGCAGACCACACAAGAGGAAGATGGCTGCTCCTG | |
| | CAGATTCCCCGAGGAAGAAGAAGGCGGCTGCGAGCT | |
| | GAGAGTGAAGTTCAGCAGATCCGCCGACGCTCCAGC | |
| | CTATCAGCAGGGCCAAAACCAGCTGTACAACGAGCT | |
| | GAACCTGGGGAGAAGAGAAGAGTACGACGTGCTGGA | |
| | TAAGCGGAGAGGCAGAGATCCTGAAATGGGCGGCAA | |
| | GCCCAGACGGAAGAATCCTCAAGAGGGCCTGTATAA | |
| | TGAGCTGCAGAAAGACAAGATGGCCGAGGCCTACAG | |
| | CGAGATCGGAATGAAGGGCGAGCGCAGAAGAGGCA | |
| | AGGGACACGATGGACTGTACCAGGGCCTGAGCACCG | |
| | CCACCAAGGATACCTATGACGCACTGCACATGCAGG | |
| | CCCTGCCACCTAGA | |
| 63 | ATGCCTTTGTTGCTGCTGCTGCCTCTTCTGTGGGCTGG | PR3_2 (S176A) |
| | CGCTCTGGCTATCGTTGGAGGACATGAAGCCCAGCCT | MS CAAR |
| | CACAGCAGACCTTACATGGCCAGCCTGCAGATGAGA | (PR3_2_33) |
| | GGCAATCCCGGCTCTCACTTTTGTGGCGGCACCCTGA | Nucleotide |
| | TTCACCCCTCCTTCGTTCTGACCGCCGCTCACTGCCTG | |
| | AGAGACATCCCTCAGAGACTGGTCAACGTGGTGCTG | |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTTGGAGATGCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCGGCCAGCCAAGAGAGCCCCAGGTTT | |
| | ACACACTGCCTCCAAGCCAAGAGGAAATGACCAAGA | |
| | ATCAGGTGTCCCTGACATGCCTGGTCAAGGGCTTCTA | |
| | CCCCTCCGATATCGCCGTGGAATGGGAGAGCAATGG | |
| | CCAGCCTGAGAACAACTACAAGACCACACCTCCTGT | |
| | GCTGGACAGCGACGGCAGTTTCTTCCTGTATAGTAGA | |
| | CTCACCGTGGATAAATCAAGATGGCAAGAGGGCAAC | |
| | GTGTTCAGCTGCAGCGTGATGCACGAGGCCCTGCAC | |
| | AACCACTACACCCAGAAAAGCCTGAGCCTGTCTCTG | |
| | GGCAAGATGTTCTGGGTGCTCGTGGTCGTTGGCGGAG | |
| | TGCTGGCCTGTTACAGCCTGCTGGTTACCGTGGCCTT | |
| | CATCATCTTTTGGGTCAAGCGGGGCAGAAAGAAGCT | |
| | GCTCTACATCTTCAAGCAGCCCTTCATGCGGCCCGTG | |
| | CAGACCACACAAGAGGAAGATGGCTGCTCCTGCAGA | |
| | TTCCCCGAGGAAGAAGAAGGCGGCTGCGAGCTGAGA | |
| | GTGAAGTTCAGCAGATCCGCCGACGCTCCAGCCTATC | |
| | AGCAGGGCCAAAACCAGCTGTACAACGAGCTGAACC | |
| | TGGGGAGAAGAGAAGAGTACGACGTGCTGGATAAGC | |
| | GGAGAGGCAGAGATCCTGAAATGGGCGGCAAGCCCA | |
| | GACGGAAGAATCCTCAAGAGGGCCTGTATAATGAGC | |
| | TGCAGAAAGACAAGATGGCCGAGGCCTACAGCGAGA | |
| | TCGGAATGAAGGGCGAGCGCAGAAGAGGCAAGGGA | |
| | CACGATGGACTGTACCAGGGCCTGAGCACCGCCACC | |
| | AAGGATACCTATGACGCACTGCACATGCAGGCCCTG | |
| | CCACCTAGA | |
| 64 | ATGCCTTTGTTGCTGCTGCTGCCTCTTCTGTGGGCTGG | PR3_2 (S176A) |
| | CGCTCTGGCTATCGTTGGAGGACATGAAGCCCAGCCT | LS CAAR |
| | CACAGCAGACCTTACATGGCCAGCCTGCAGATGAGA | (PR3_2_34) |
| | GGCAATCCCGGCTCTCACTTTTGTGGCGGCACCCTGA | Nucleotide |
| | TTCACCCCTCCTTCGTTCTGACCGCCGCTCACTGCCTG | |
| | AGAGACATCCCTCAGAGACTGGTCAACGTGGTGCTG | |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTTGGAGATGCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCGCTCCTCCTGTTGCCGGACCTTCCGT | |
| | GTTCCTGTTTCCTCCAAAGCCTAAGGACACCCTGATG | |
| | ATCAGCAGGACCCCTGAAGTGACCTGCGTGGTGGTG | |
| | GATGTGTCCCAAGAGGATCCCGAGGTGCAGTTCAAC | |
| | TGGTATGTGGACGGCGTGGAAGTGCACAACGCCAAG | |
| | ACCAAGCCTAGAGAGGAACAGTTCCAGAGCACCTAC | |
| | AGAGTGGTGTCCGTGCTGACAGTGCTGCACCAGGATT | |
| | GGCTGAACGGCAAAGAGTACAAGTGCAAGGTGTCCA | |
| | ACAAGGGCCTGCCTAGCAGCATCGAGAAAACCATCT | |
| | CCAAGGCCAAGGGCCAGCCAAGAGAGCCCCAGGTTT | |
| | ACACACTGCCTCCAAGCCAAGAGGAAATGACCAAGA | |
| | ATCAGGTGTCCCTGACATGCCTGGTCAAGGGCTTCTA | |
| | CCCCTCCGATATCGCCGTGGAATGGGAGAGCAATGG | |
| | CCAGCCTGAGAACAACTACAAGACCACACCTCCTGT | |
| | GCTGGACAGCGACGGCAGTTTCTTCCTGTATAGTAGA | |
| | CTCACCGTGGATAAATCAAGATGGCAAGAGGGCAAC | |
| | GTGTTCAGCTGCAGCGTGATGCACGAGGCCCTGCAC | |
| | AACCACTACACCCAGAAAAGCCTGAGCCTGTCTCTG | |
| | GGCAAGATGTTCTGGGTGCTCGTGGTCGTTGGCGGAG | |
| | TGCTGGCCTGTTACAGCCTGCTGGTTACCGTGGCCTT | |
| | CATCATCTTTTGGGTCAAGCGGGGCAGAAAGAAGCT | |
| | GCTCTACATCTTCAAGCAGCCCTTCATGCGGCCCGTG | |
| | CAGACCACACAAGAGGAAGATGGCTGCTCCTGCAGA | |
| | TTCCCCGAGGAAGAAGAAGGCGGCTGCGAGCTGAGA | |
| | GTGAAGTTCAGCAGATCCGCCGACGCTCCAGCCTATC | |
| | AGCAGGGCCAAAACCAGCTGTACAACGAGCTGAACC | |
| | TGGGGAGAAGAGAAGAGTACGACGTGCTGGATAAGC | |
| | GGAGAGGCAGAGATCCTGAAATGGGCGGCAAGCCCA | |
| | GACGGAAGAATCCTCAAGAGGGCCTGTATAATGAGC | |
| | TGCAGAAAGACAAGATGGCCGAGGCCTACAGCGAGA | |
| | TCGGAATGAAGGGCGAGCGCAGAAGAGGCAAGGGA | |
| | CACGATGGACTGTACCAGGGCCTGAGCACCGCCACC | |
| | AAGGATACCTATGACGCACTGCACATGCAGGCCCTG | |
| | CCACCTAGA | |
| 65 | ATGCCTTTGTTGCTGCTGCTGCCTCTTCTGTGGGCTGG | PR3_3 (C- |
| | CGCTCTGGCTATCGTTGGAGGACATGAAGCCCAGCCT | terminal |
| | CACAGCAGACCTTACATGGCCAGCCTGCAGATGAGA | addition) SS |
| | GGCAATCCCGGCTCTCACTTTTGTGGCGGCACCCTGA | CAAR |
| | TTCACCCCTCCTTCGTTCTGACCGCCGCTCACTGCCTG | (PR3_3_35) |
| | AGAGACATCCCTCAGAGACTGGTCAACGTGGTGCTG | Nucleotide |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTTGGAGATGCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGCGTGGCCCTCTACGTGGACTGGATCAG | |
| | ATCTACCCTGCGGAGAGTGGAAGCCAAGGGCAGACC | |
| | TGAGTCTAAATACGGACCGCCTTGTCCTCCTTGTCCC | |
| | ATGTTCTGGGTGCTCGTGGTCGTTGGCGGAGTGCTGG | |
| | CCTGTTACAGCCTGCTGGTTACCGTGGCCTTCATCAT | |
| | CTTTTGGGTCAAGCGGGGCAGAAAGAAGCTGCTCTA | |
| | CATCTTCAAGCAGCCCTTCATGCGGCCCGTGCAGACC | |
| | ACACAAGAGGAAGATGGCTGCTCCTGCAGATTCCCC | |
| | GAGGAAGAAGAAGGCGGCTGCGAGCTGAGAGTGAA | |
| | GTTCAGCAGATCCGCCGACGCTCCAGCCTATCAGCAG | |
| | GGCCAAAACCAGCTGTACAACGAGCTGAACCTGGGG | |
| | AGAAGAGAAGAGTACGACGTGCTGGATAAGCGGAG | |
| | AGGCAGAGATCCTGAAATGGGCGGCAAGCCCAGACG | |
| | GAAGAATCCTCAAGAGGGCCTGTATAATGAGCTGCA | |
| | GAAAGACAAGATGGCCGAGGCCTACAGCGAGATCGG | |
| | AATGAAGGGCGAGCGCAGAAGAGGCAAGGGACACG | |
| | ATGGACTGTACCAGGGCCTGAGCACCGCCACCAAGG | |
| | ATACCTATGACGCACTGCACATGCAGGCCCTGCCACC | |
| | TAGA | |
| 66 | ATGCCTTTGTTGCTGCTGCTGCCTCTTCTGTGGGCTGG | PR3_3 (C- |
| | CGCTCTGGCTATCGTTGGAGGACATGAAGCCCAGCCT | terminal |
| | CACAGCAGACCTTACATGGCCAGCCTGCAGATGAGA | addition) MS |
| | GGCAATCCCGGCTCTCACTTTTGTGGCGGCACCCTGA | CAAR |
| | TTCACCCCTCCTTCGTTCTGACCGCCGCTCACTGCCTG | (PR3_3_36) |
| | AGAGACATCCCTCAGAGACTGGTCAACGTGGTGCTG | Nucleotide |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTTGGAGATGCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGCGTGGCCCTCTACGTGGACTGGATCAG | |
| | ATCTACCCTGCGGAGAGTGGAAGCCAAGGGCAGACC | |
| | TGAGTCTAAATACGGACCGCCTTGTCCTCCTTGTCCC | |
| | GGCCAGCCAAGAGAGCCCCAGGTTTACACACTGCCT | |
| | CCAAGCCAAGAGGAAATGACCAAGAATCAGGTGTCC | |
| | CTGACATGCCTGGTCAAGGGCTTCTACCCCTCCGATA | |
| | TCGCCGTGGAATGGGAGAGCAATGGCCAGCCTGAGA | |
| | ACAACTACAAGACCACACCTCCTGTGCTGGACAGCG | |
| | ACGGCAGTTTCTTCCTGTATAGTAGACTCACCGTGGA | |
| | TAAATCAAGATGGCAAGAGGGCAACGTGTTCAGCTG | |
| | CAGCGTGATGCACGAGGCCCTGCACAACCACTACAC | |
| | CCAGAAAAGCCTGAGCCTGTCTCTGGGCAAGATGTTC | |
| | TGGGTGCTCGTGGTCGTTGGCGGAGTGCTGGCCTGTT | |
| | ACAGCCTGCTGGTTACCGTGGCCTTCATCATCTTTTG | |
| | GGTCAAGCGGGGCAGAAAGAAGCTGCTCTACATCTT | |
| | CAAGCAGCCCTTCATGCGGCCCGTGCAGACCACACA | |
| | AGAGGAAGATGGCTGCTCCTGCAGATTCCCCGAGGA | |
| | AGAAGAAGGCGGCTGCGAGCTGAGAGTGAAGTTCAG | |
| | CAGATCCGCCGACGCTCCAGCCTATCAGCAGGGCCA | |
| | AAACCAGCTGTACAACGAGCTGAACCTGGGGAGAAG | |
| | AGAAGAGTACGACGTGCTGGATAAGCGGAGAGGCAG | |
| | AGATCCTGAAATGGGCGGCAAGCCCAGACGGAAGAA | |
| | TCCTCAAGAGGGCCTGTATAATGAGCTGCAGAAAGA | |
| | CAAGATGGCCGAGGCCTACAGCGAGATCGGAATGAA | |
| | GGGCGAGCGCAGAAGAGGCAAGGGACACGATGGAC | |
| | TGTACCAGGGCCTGAGCACCGCCACCAAGGATACCT | |
| | ATGACGCACTGCACATGCAGGCCCTGCCACCTAGA | |
| 67 | ATGCCTTTGTTGCTGCTGCTGCCTCTTCTGTGGGCTGG | PR3_3 (C- |
| | CGCTCTGGCTATCGTTGGAGGACATGAAGCCCAGCCT | terminal |
| | CACAGCAGACCTTACATGGCCAGCCTGCAGATGAGA | addition) LS |
| | GGCAATCCCGGCTCTCACTTTTGTGGCGGCACCCTGA | CAAR |
| | TTCACCCCTCCTTCGTTCTGACCGCCGCTCACTGCCTG | (PR3_3_37) |
| | AGAGACATCCCTCAGAGACTGGTCAACGTGGTGCTG | Nucleotide |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTTGGAGATGCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGCGTGGCCCTCTACGTGGACTGGATCAG | |
| | ATCTACCCTGCGGAGAGTGGAAGCCAAGGGCAGACC | |
| | TGAGTCTAAATACGGACCGCCTTGTCCTCCTTGTCCC | |
| | GCTCCTCCTGTTGCCGGACCTTCCGTGTTCCTGTTTCC | |
| | TCCAAAGCCTAAGGACACCCTGATGATCAGCAGGAC | |
| | CCCTGAAGTGACCTGCGTGGTGGTGGATGTGTCCCAA | |
| | GAGGATCCCGAGGTGCAGTTCAACTGGTATGTGGAC | |
| | GGCGTGGAAGTGCACAACGCCAAGACCAAGCCTAGA | |
| | GAGGAACAGTTCCAGAGCACCTACAGAGTGGTGTCC | |
| | GTGCTGACAGTGCTGCACCAGGATTGGCTGAACGGC | |
| | AAAGAGTACAAGTGCAAGGTGTCCAACAAGGGCCTG | |
| | CCTAGCAGCATCGAGAAAACCATCTCCAAGGCCAAG | |
| | GGCCAGCCAAGAGAGCCCCAGGTTTACACACTGCCT | |
| | CCAAGCCAAGAGGAAATGACCAAGAATCAGGTGTCC | |
| | CTGACATGCCTGGTCAAGGGCTTCTACCCCTCCGATA | |
| | TCGCCGTGGAATGGGAGAGCAATGGCCAGCCTGAGA | |
| | ACAACTACAAGACCACACCTCCTGTGCTGGACAGCG | |
| | ACGGCAGTTTCTTCCTGTATAGTAGACTCACCGTGGA | |
| | TAAATCAAGATGGCAAGAGGGCAACGTGTTCAGCTG | |
| | CAGCGTGATGCACGAGGCCCTGCACAACCACTACAC | |
| | CCAGAAAAGCCTGAGCCTGTCTCTGGGCAAGATGTTC | |
| | TGGGTGCTCGTGGTCGTTGGCGGAGTGCTGGCCTGTT | |
| | ACAGCCTGCTGGTTACCGTGGCCTTCATCATCTTTTG | |
| | GGTCAAGCGGGGCAGAAAGAAGCTGCTCTACATCTT | |
| | CAAGCAGCCCTTCATGCGGCCCGTGCAGACCACACA | |
| | AGAGGAAGATGGCTGCTCCTGCAGATTCCCCGAGGA | |
| | AGAAGAAGGCGGCTGCGAGCTGAGAGTGAAGTTCAG | |
| | CAGATCCGCCGACGCTCCAGCCTATCAGCAGGGCCA | |
| | AAACCAGCTGTACAACGAGCTGAACCTGGGGAGAAG | |
| | AGAAGAGTACGACGTGCTGGATAAGCGGAGAGGCAG | |
| | AGATCCTGAAATGGGCGGCAAGCCCAGACGGAAGAA | |
| | TCCTCAAGAGGGCCTGTATAATGAGCTGCAGAAAGA | |
| | CAAGATGGCCGAGGCCTACAGCGAGATCGGAATGAA | |
| | GGGCGAGCGCAGAAGAGGCAAGGGACACGATGGAC | |
| | TGTACCAGGGCCTGAGCACCGCCACCAAGGATACCT | |
| | ATGACGCACTGCACATGCAGGCCCTGCCACCTAGA | |
| 68 | ATGCCTTTGTTGCTGCTGCTGCCTCTTCTGTGGGCTGG | PR3_4 (D91N) |
| | CGCTCTGGCTATCGTTGGAGGACATGAAGCCCAGCCT | SS CAAR |
| | CACAGCAGACCTTACATGGCCAGCCTGCAGATGAGA | (PR3_4_38) |
| | GGCAATCCCGGCTCTCACTTTTGTGGCGGCACCCTGA | Nucleotide |
| | TTCACCCCTCCTTCGTTCTGACCGCCGCTCACTGCCTG | |
| | AGAGACATCCCTCAGAGACTGGTCAACGTGGTGCTG | |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACAACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTCGGAGATTCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCATGTTCTGGGTGCTCGTGGTCGTTGG | |
| | CGGAGTGCTGGCCTGTTACAGCCTGCTGGTTACCGTG | |
| | GCCTTCATCATCTTTTGGGTCAAGCGGGGCAGAAAGA | |
| | AGCTGCTCTACATCTTCAAGCAGCCCTTCATGCGGCC | |
| | CGTGCAGACCACACAAGAGGAAGATGGCTGCTCCTG | |
| | CAGATTCCCCGAGGAAGAAGAAGGCGGCTGCGAGCT | |
| | GAGAGTGAAGTTCAGCAGATCCGCCGACGCTCCAGC | |
| | CTATCAGCAGGGCCAAAACCAGCTGTACAACGAGCT | |
| | GAACCTGGGGAGAAGAGAAGAGTACGACGTGCTGGA | |
| | TAAGCGGAGAGGCAGAGATCCTGAAATGGGCGGCAA | |
| | GCCCAGACGGAAGAATCCTCAAGAGGGCCTGTATAA | |
| | TGAGCTGCAGAAAGACAAGATGGCCGAGGCCTACAG | |
| | CGAGATCGGAATGAAGGGCGAGCGCAGAAGAGGCA | |
| | AGGGACACGATGGACTGTACCAGGGCCTGAGCACCG | |
| | CCACCAAGGATACCTATGACGCACTGCACATGCAGG | |
| | CCCTGCCACCTAGA | |
| 69 | ATGCCTTTGTTGCTGCTGCTGCCTCTTCTGTGGGCTGG | PR3_4 (D91N) |
| | CGCTCTGGCTATCGTTGGAGGACATGAAGCCCAGCCT | MS CAAR |
| | CACAGCAGACCTTACATGGCCAGCCTGCAGATGAGA | (PR3_4_39) |
| | GGCAATCCCGGCTCTCACTTTTGTGGCGGCACCCTGA | Nucleotide |
| | TTCACCCCTCCTTCGTTCTGACCGCCGCTCACTGCCTG | |
| | AGAGACATCCCTCAGAGACTGGTCAACGTGGTGCTG | |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACAACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTCGGAGATTCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCGGCCAGCCAAGAGAGCCCCAGGTTT | |
| | ACACACTGCCTCCAAGCCAAGAGGAAATGACCAAGA | |
| | ATCAGGTGTCCCTGACATGCCTGGTCAAGGGCTTCTA | |
| | CCCCTCCGATATCGCCGTGGAATGGGAGAGCAATGG | |
| | CCAGCCTGAGAACAACTACAAGACCACACCTCCTGT | |
| | GCTGGACAGCGACGGCAGTTTCTTCCTGTATAGTAGA | |
| | CTCACCGTGGATAAATCAAGATGGCAAGAGGGCAAC | |
| | GTGTTCAGCTGCAGCGTGATGCACGAGGCCCTGCAC | |
| | AACCACTACACCCAGAAAAGCCTGAGCCTGTCTCTG | |
| | GGCAAGATGTTCTGGGTGCTCGTGGTCGTTGGCGGAG | |
| | TGCTGGCCTGTTACAGCCTGCTGGTTACCGTGGCCTT | |
| | CATCATCTTTTGGGTCAAGCGGGGCAGAAAGAAGCT | |
| | GCTCTACATCTTCAAGCAGCCCTTCATGCGGCCCGTG | |
| | CAGACCACACAAGAGGAAGATGGCTGCTCCTGCAGA | |
| | TTCCCCGAGGAAGAAGAAGGCGGCTGCGAGCTGAGA | |
| | GTGAAGTTCAGCAGATCCGCCGACGCTCCAGCCTATC | |
| | AGCAGGGCCAAAACCAGCTGTACAACGAGCTGAACC | |
| | TGGGGAGAAGAGAAGAGTACGACGTGCTGGATAAGC | |
| | GGAGAGGCAGAGATCCTGAAATGGGCGGCAAGCCCA | |
| | GACGGAAGAATCCTCAAGAGGGCCTGTATAATGAGC | |
| | TGCAGAAAGACAAGATGGCCGAGGCCTACAGCGAGA | |
| | TCGGAATGAAGGGCGAGCGCAGAAGAGGCAAGGGA | |
| | CACGATGGACTGTACCAGGGCCTGAGCACCGCCACC | |
| | AAGGATACCTATGACGCACTGCACATGCAGGCCCTG | |
| | CCACCTAGA | |
| | ATGCCTTTGTTGCTGCTGCTGCCTCTTCTGTGGGCTGG | PR3_4 (D91N) |
| 70 | CGCTCTGGCTATCGTTGGAGGACATGAAGCCCAGCCT | LS CAAR |
| | CACAGCAGACCTTACATGGCCAGCCTGCAGATGAGA | (PR3_4_40) |
| | GGCAATCCCGGCTCTCACTTTTGTGGCGGCACCCTGA | Nucleotide |
| | TTCACCCCTCCTTCGTTCTGACCGCCGCTCACTGCCTG | |
| | AGAGACATCCCTCAGAGACTGGTCAACGTGGTGCTG | |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACAACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTCGGAGATTCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCGCTCCTCCTGTTGCCGGACCTTCCGT | |
| | GTTCCTGTTTCCTCCAAAGCCTAAGGACACCCTGATG | |
| | ATCAGCAGGACCCCTGAAGTGACCTGCGTGGTGGTG | |
| | GATGTGTCCCAAGAGGATCCCGAGGTGCAGTTCAAC | |
| | TGGTATGTGGACGGCGTGGAAGTGCACAACGCCAAG | |
| | ACCAAGCCTAGAGAGGAACAGTTCCAGAGCACCTAC | |
| | AGAGTGGTGTCCGTGCTGACAGTGCTGCACCAGGATT | |
| | GGCTGAACGGCAAAGAGTACAAGTGCAAGGTGTCCA | |
| | ACAAGGGCCTGCCTAGCAGCATCGAGAAAACCATCT | |
| | CCAAGGCCAAGGGCCAGCCAAGAGAGCCCCAGGTTT | |
| | ACACACTGCCTCCAAGCCAAGAGGAAATGACCAAGA | |
| | ATCAGGTGTCCCTGACATGCCTGGTCAAGGGCTTCTA | |
| | CCCCTCCGATATCGCCGTGGAATGGGAGAGCAATGG | |
| | CCAGCCTGAGAACAACTACAAGACCACACCTCCTGT | |
| | GCTGGACAGCGACGGCAGTTTCTTCCTGTATAGTAGA | |
| | CTCACCGTGGATAAATCAAGATGGCAAGAGGGCAAC | |
| | GTGTTCAGCTGCAGCGTGATGCACGAGGCCCTGCAC | |
| | AACCACTACACCCAGAAAAGCCTGAGCCTGTCTCTG | |
| | GGCAAGATGTTCTGGGTGCTCGTGGTCGTTGGCGGAG | |
| | TGCTGGCCTGTTACAGCCTGCTGGTTACCGTGGCCTT | |
| | CATCATCTTTTGGGTCAAGCGGGGCAGAAAGAAGCT | |
| | GCTCTACATCTTCAAGCAGCCCTTCATGCGGCCCGTG | |
| | CAGACCACACAAGAGGAAGATGGCTGCTCCTGCAGA | |
| | TTCCCCGAGGAAGAAGAAGGCGGCTGCGAGCTGAGA | |
| | GTGAAGTTCAGCAGATCCGCCGACGCTCCAGCCTATC | |
| | AGCAGGGCCAAAACCAGCTGTACAACGAGCTGAACC | |
| | TGGGGAGAAGAGAAGAGTACGACGTGCTGGATAAGC | |
| | GGAGAGGCAGAGATCCTGAAATGGGCGGCAAGCCCA | |
| | GACGGAAGAATCCTCAAGAGGGCCTGTATAATGAGC | |
| | TGCAGAAAGACAAGATGGCCGAGGCCTACAGCGAGA | |
| | TCGGAATGAAGGGCGAGCGCAGAAGAGGCAAGGGA | |
| | CACGATGGACTGTACCAGGGCCTGAGCACCGCCACC | |
| | AAGGATACCTATGACGCACTGCACATGCAGGCCCTG | |
| | CCACCTAGA | |
| 71 | ATGCCTTTGTTGCTGCTGCTGCCTCTTCTGTGGGCTGG | PR3_5 |
| | CGCTCTGGCTGCCGAGATCGTTGGAGGACATGAAGC | (Unprocessed N- |
| | CCAGCCTCACAGCAGACCTTACATGGCCAGCCTGCA | terminus) SS |
| | GATGAGAGGCAATCCCGGCTCTCACTTTTGTGGCGGC | CAAR |
| | ACCCTGATTCACCCCTCCTTCGTTCTGACCGCCGCTC | (PR3_5_41) |
| | ACTGCCTGAGAGACATCCCTCAGAGACTGGTCAACG | Nucleotide |
| | TGGTGCTGGGAGCCCACAACGTGCGGACACAAGAAC | |
| | CTACACAGCAGCACTTCAGCGTGGCCCAGGTGTTCCT | |
| | GAACAACTACGACGCCGAGAACAAGCTGAACGACGT | |
| | GCTGCTGATCCAGCTGAGCAGCCCTGCTAATCTGTCT | |
| | GCCTCTGTGGCCACTGTGCAGCTGCCTCAGCAAGATC | |
| | AGCCTGTGCCTCACGGCACACAGTGTCTGGCTATGGG | |
| | ATGGGGAAGAGTGGGCGCTCATGATCCTCCAGCTCA | |
| | AGTGCTGCAAGAGCTGAACGTGACCGTCGTGACCTTC | |
| | TTCTGCCGGCCTCACAACATCTGCACCTTCGTGCCCA | |
| | GAAGAAAGGCCGGCATCTGCTTCGGAGATTCTGGCG | |
| | GCCCTCTGATCTGCGACGGAATCATCCAGGGCATCGA | |
| | CAGCTTCGTGATCTGGGGCTGTGCCACCAGACTGTTC | |
| | CCCGACTTCTTTACCCGGGTGGCCCTCTACGTGGACT | |
| | GGATCCGGTCTACACTGAGAGAGTCTAAATACGGAC | |
| | CGCCTTGTCCTCCTTGTCCCATGTTCTGGGTGCTCGTG | |
| | GTCGTTGGCGGAGTGCTGGCCTGTTACAGCCTGCTGG | |
| | TTACCGTGGCCTTCATCATCTTTTGGGTCAAGCGGGG | |
| | CAGAAAGAAGCTGCTCTACATCTTCAAGCAGCCCTTC | |
| | ATGCGGCCCGTGCAGACCACACAAGAGGAAGATGGC | |
| | TGCTCCTGCAGATTCCCCGAGGAAGAAGAAGGCGGC | |
| | TGCGAGCTGAGAGTGAAGTTCAGCAGATCCGCCGAC | |
| | GCTCCAGCCTATCAGCAGGGCCAAAACCAGCTGTAC | |
| | AACGAGCTGAACCTGGGGAGAAGAGAAGAGTACGA | |
| | CGTGCTGGATAAGCGGAGAGGCAGAGATCCTGAAAT | |
| | GGGCGGCAAGCCCAGACGGAAGAATCCTCAAGAGGG | |
| | CCTGTATAATGAGCTGCAGAAAGACAAGATGGCCGA | |
| | GGCCTACAGCGAGATCGGAATGAAGGGCGAGCGCAG | |
| | AAGAGGCAAGGGACACGATGGACTGTACCAGGGCCT | |
| | GAGCACCGCCACCAAGGATACCTATGACGCACTGCA | |
| | CATGCAGGCCCTGCCACCTAGA | |
| 72 | ATGCCTTTGTTGCTGCTGCTGCCTCTTCTGTGGGCTGG | PR3_5 |
| | CGCTCTGGCTGCCGAGATCGTTGGAGGACATGAAGC | (Unprocessed N- |
| | CCAGCCTCACAGCAGACCTTACATGGCCAGCCTGCA | terminus) MS |
| | GATGAGAGGCAATCCCGGCTCTCACTTTTGTGGCGGC | CAAR |
| | ACCCTGATTCACCCCTCCTTCGTTCTGACCGCCGCTC | (PR3_5_42) |
| | ACTGCCTGAGAGACATCCCTCAGAGACTGGTCAACG | Nucleotide |
| | TGGTGCTGGGAGCCCACAACGTGCGGACACAAGAAC | |
| | CTACACAGCAGCACTTCAGCGTGGCCCAGGTGTTCCT | |
| | GAACAACTACGACGCCGAGAACAAGCTGAACGACGT | |
| | GCTGCTGATCCAGCTGAGCAGCCCTGCTAATCTGTCT | |
| | GCCTCTGTGGCCACTGTGCAGCTGCCTCAGCAAGATC | |
| | AGCCTGTGCCTCACGGCACACAGTGTCTGGCTATGGG | |
| | ATGGGGAAGAGTGGGCGCTCATGATCCTCCAGCTCA | |
| | AGTGCTGCAAGAGCTGAACGTGACCGTCGTGACCTTC | |
| | TTCTGCCGGCCTCACAACATCTGCACCTTCGTGCCCA | |
| | GAAGAAAGGCCGGCATCTGCTTCGGAGATTCTGGCG | |
| | GCCCTCTGATCTGCGACGGAATCATCCAGGGCATCGA | |
| | CAGCTTCGTGATCTGGGGCTGTGCCACCAGACTGTTC | |
| | CCCGACTTCTTTACCCGGGTGGCCCTCTACGTGGACT | |
| | GGATCCGGTCTACACTGAGAGAGTCTAAATACGGAC | |
| | CGCCTTGTCCTCCTTGTCCCGGCCAGCCAAGAGAGCC | |
| | CCAGGTTTACACACTGCCTCCAAGCCAAGAGGAAAT | |
| | GACCAAGAATCAGGTGTCCCTGACATGCCTGGTCAA | |
| | GGGCTTCTACCCCTCCGATATCGCCGTGGAATGGGAG | |
| | AGCAATGGCCAGCCTGAGAACAACTACAAGACCACA | |
| | CCTCCTGTGCTGGACAGCGACGGCAGTTTCTTCCTGT | |
| | ATAGTAGACTCACCGTGGATAAATCAAGATGGCAAG | |
| | AGGGCAACGTGTTCAGCTGCAGCGTGATGCACGAGG | |
| | CCCTGCACAACCACTACACCCAGAAAAGCCTGAGCC | |
| | TGTCTCTGGGCAAGATGTTCTGGGTGCTCGTGGTCGT | |
| | TGGCGGAGTGCTGGCCTGTTACAGCCTGCTGGTTACC | |
| | GTGGCCTTCATCATCTTTTGGGTCAAGCGGGGCAGAA | |
| | AGAAGCTGCTCTACATCTTCAAGCAGCCCTTCATGCG | |
| | GCCCGTGCAGACCACACAAGAGGAAGATGGCTGCTC | |
| | CTGCAGATTCCCCGAGGAAGAAGAAGGCGGCTGCGA | |
| | GCTGAGAGTGAAGTTCAGCAGATCCGCCGACGCTCC | |
| | AGCCTATCAGCAGGGCCAAAACCAGCTGTACAACGA | |
| | GCTGAACCTGGGGAGAAGAGAAGAGTACGACGTGCT | |
| | GGATAAGCGGAGAGGCAGAGATCCTGAAATGGGCGG | |
| | CAAGCCCAGACGGAAGAATCCTCAAGAGGGCCTGTA | |
| | TAATGAGCTGCAGAAAGACAAGATGGCCGAGGCCTA | |
| | CAGCGAGATCGGAATGAAGGGCGAGCGCAGAAGAG | |
| | GCAAGGGACACGATGGACTGTACCAGGGCCTGAGCA | |
| | CCGCCACCAAGGATACCTATGACGCACTGCACATGC | |
| | AGGCCCTGCCACCTAGA | |
| 73 | ATGCCTTTGTTGCTGCTGCTGCCTCTTCTGTGGGCTGG | PR3_5 |
| | CGCTCTGGCTGCCGAGATCGTTGGAGGACATGAAGC | (Unprocessed N- |
| | CCAGCCTCACAGCAGACCTTACATGGCCAGCCTGCA | terminus) LS |
| | GATGAGAGGCAATCCCGGCTCTCACTTTTGTGGCGGC | CAAR |
| | ACCCTGATTCACCCCTCCTTCGTTCTGACCGCCGCTC | (PR3_5_43) |
| | ACTGCCTGAGAGACATCCCTCAGAGACTGGTCAACG | Nucleotide |
| | TGGTGCTGGGAGCCCACAACGTGCGGACACAAGAAC | |
| | CTACACAGCAGCACTTCAGCGTGGCCCAGGTGTTCCT | |
| | GAACAACTACGACGCCGAGAACAAGCTGAACGACGT | |
| | GCTGCTGATCCAGCTGAGCAGCCCTGCTAATCTGTCT | |
| | GCCTCTGTGGCCACTGTGCAGCTGCCTCAGCAAGATC | |
| | AGCCTGTGCCTCACGGCACACAGTGTCTGGCTATGGG | |
| | ATGGGGAAGAGTGGGCGCTCATGATCCTCCAGCTCA | |
| | AGTGCTGCAAGAGCTGAACGTGACCGTCGTGACCTTC | |
| | TTCTGCCGGCCTCACAACATCTGCACCTTCGTGCCCA | |
| | GAAGAAAGGCCGGCATCTGCTTCGGAGATTCTGGCG | |
| | GCCCTCTGATCTGCGACGGAATCATCCAGGGCATCGA | |
| | CAGCTTCGTGATCTGGGGCTGTGCCACCAGACTGTTC | |
| | CCCGACTTCTTTACCCGGGTGGCCCTCTACGTGGACT | |
| | GGATCCGGTCTACACTGAGAGAGTCTAAATACGGAC | |
| | CGCCTTGTCCTCCTTGTCCCGCTCCTCCTGTTGCCGGA | |
| | CCTTCCGTGTTCCTGTTTCCTCCAAAGCCTAAGGACA | |
| | CCCTGATGATCAGCAGGACCCCTGAAGTGACCTGCGT | |
| | GGTGGTGGATGTGTCCCAAGAGGATCCCGAGGTGCA | |
| | GTTCAACTGGTATGTGGACGGCGTGGAAGTGCACAA | |
| | CGCCAAGACCAAGCCTAGAGAGGAACAGTTCCAGAG | |
| | CACCTACAGAGTGGTGTCCGTGCTGACAGTGCTGCAC | |
| | CAGGATTGGCTGAACGGCAAAGAGTACAAGTGCAAG | |
| | GTGTCCAACAAGGGCCTGCCTAGCAGCATCGAGAAA | |
| | ACCATCTCCAAGGCCAAGGGCCAGCCAAGAGAGCCC | |
| | CAGGTTTACACACTGCCTCCAAGCCAAGAGGAAATG | |
| | ACCAAGAATCAGGTGTCCCTGACATGCCTGGTCAAG | |
| | GGCTTCTACCCCTCCGATATCGCCGTGGAATGGGAGA | |
| | GCAATGGCCAGCCTGAGAACAACTACAAGACCACAC | |
| | CTCCTGTGCTGGACAGCGACGGCAGTTTCTTCCTGTA | |
| | TAGTAGACTCACCGTGGATAAATCAAGATGGCAAGA | |
| | GGGCAACGTGTTCAGCTGCAGCGTGATGCACGAGGC | |
| | CCTGCACAACCACTACACCCAGAAAAGCCTGAGCCT | |
| | GTCTCTGGGCAAGATGTTCTGGGTGCTCGTGGTCGTT | |
| | GGCGGAGTGCTGGCCTGTTACAGCCTGCTGGTTACCG | |
| | TGGCCTTCATCATCTTTTGGGTCAAGCGGGGCAGAAA | |
| | GAAGCTGCTCTACATCTTCAAGCAGCCCTTCATGCGG | |
| | CCCGTGCAGACCACACAAGAGGAAGATGGCTGCTCC | |
| | TGCAGATTCCCCGAGGAAGAAGAAGGCGGCTGCGAG | |
| | CTGAGAGTGAAGTTCAGCAGATCCGCCGACGCTCCA | |
| | GCCTATCAGCAGGGCCAAAACCAGCTGTACAACGAG | |
| | CTGAACCTGGGGAGAAGAGAAGAGTACGACGTGCTG | |
| | GATAAGCGGAGAGGCAGAGATCCTGAAATGGGCGGC | |
| | AAGCCCAGACGGAAGAATCCTCAAGAGGGCCTGTAT | |
| | AATGAGCTGCAGAAAGACAAGATGGCCGAGGCCTAC | |
| | AGCGAGATCGGAATGAAGGGCGAGCGCAGAAGAGG | |
| | CAAGGGACACGATGGACTGTACCAGGGCCTGAGCAC | |
| | CGCCACCAAGGATACCTATGACGCACTGCACATGCA | |
| | GGCCCTGCCACCTAGA | |
| 74 | ATGCCTTTGTTGCTGCTGCTGCCTCTTCTGTGGGCTGG | PR3_6 (Ile |
| | CGCTCTGGCTGTTGGAGGACATGAAGCCCAGCCTCAC | Deletion) SS |
| | AGCAGACCTTACATGGCCAGCCTGCAGATGAGAGGC | CAAR |
| | AATCCCGGCTCTCACTTTTGTGGCGGCACCCTGATTC | (PR3_6_44) |
| | ACCCCTCCTTCGTTCTGACCGCCGCTCACTGCCTGAG | Nucleotide |
| | AGACATCCCTCAGAGACTGGTCAACGTGGTGCTGGG | |
| | AGCCCACAACGTGCGGACACAAGAACCTACACAGCA | |
| | GCACTTCAGCGTGGCCCAGGTGTTCCTGAACAACTAC | |
| | GACGCCGAGAACAAGCTGAACGACGTGCTGCTGATC | |
| | CAGCTGAGCAGCCCTGCTAATCTGTCTGCCTCTGTGG | |
| | CCACTGTGCAGCTGCCTCAGCAAGATCAGCCTGTGCC | |
| | TCACGGCACACAGTGTCTGGCTATGGGATGGGGAAG | |
| | AGTGGGCGCTCATGATCCTCCAGCTCAAGTGCTGCAA | |
| | GAGCTGAACGTGACCGTCGTGACCTTCTTCTGCCGGC | |
| | CTCACAACATCTGCACCTTCGTGCCCAGAAGAAAGG | |
| | CCGGCATCTGCTTCGGAGATTCTGGCGGCCCTCTGAT | |
| | CTGCGACGGAATCATCCAGGGCATCGACAGCTTCGT | |
| | GATCTGGGGCTGTGCCACCAGACTGTTCCCCGACTTC | |
| | TTTACCCGGGTGGCCCTCTACGTGGACTGGATCCGGT | |
| | CTACACTGAGAGAGTCTAAATACGGACCGCCTTGTCC | |
| | TCCTTGTCCCATGTTCTGGGTGCTCGTGGTCGTTGGC | |
| | GGAGTGCTGGCCTGTTACAGCCTGCTGGTTACCGTGG | |
| | CCTTCATCATCTTTTGGGTCAAGCGGGGCAGAAAGAA | |
| | GCTGCTCTACATCTTCAAGCAGCCCTTCATGCGGCCC | |
| | GTGCAGACCACACAAGAGGAAGATGGCTGCTCCTGC | |
| | AGATTCCCCGAGGAAGAAGAAGGCGGCTGCGAGCTG | |
| | AGAGTGAAGTTCAGCAGATCCGCCGACGCTCCAGCC | |
| | TATCAGCAGGGCCAAAACCAGCTGTACAACGAGCTG | |
| | AACCTGGGGAGAAGAGAAGAGTACGACGTGCTGGAT | |
| | AAGCGGAGAGGCAGAGATCCTGAAATGGGCGGCAA | |
| | GCCCAGACGGAAGAATCCTCAAGAGGGCCTGTATAA | |
| | TGAGCTGCAGAAAGACAAGATGGCCGAGGCCTACAG | |
| | CGAGATCGGAATGAAGGGCGAGCGCAGAAGAGGCA | |
| | AGGGACACGATGGACTGTACCAGGGCCTGAGCACCG | |
| | CCACCAAGGATACCTATGACGCACTGCACATGCAGG | |
| | CCCTGCCACCTAGA | |
| 75 | ATGCCTTTGTTGCTGCTGCTGCCTCTTCTGTGGGCTGG | PR3_6 (Ile |
| | CGCTCTGGCTGTTGGAGGACATGAAGCCCAGCCTCAC | Deletion) MS |
| | AGCAGACCTTACATGGCCAGCCTGCAGATGAGAGGC | CAAR |
| | AATCCCGGCTCTCACTTTTGTGGCGGCACCCTGATTC | (PR3_6_45) |
| | ACCCCTCCTTCGTTCTGACCGCCGCTCACTGCCTGAG | Nucleotide |
| | AGACATCCCTCAGAGACTGGTCAACGTGGTGCTGGG | |
| | AGCCCACAACGTGCGGACACAAGAACCTACACAGCA | |
| | GCACTTCAGCGTGGCCCAGGTGTTCCTGAACAACTAC | |
| | GACGCCGAGAACAAGCTGAACGACGTGCTGCTGATC | |
| | CAGCTGAGCAGCCCTGCTAATCTGTCTGCCTCTGTGG | |
| | CCACTGTGCAGCTGCCTCAGCAAGATCAGCCTGTGCC | |
| | TCACGGCACACAGTGTCTGGCTATGGGATGGGGAAG | |
| | AGTGGGCGCTCATGATCCTCCAGCTCAAGTGCTGCAA | |
| | GAGCTGAACGTGACCGTCGTGACCTTCTTCTGCCGGC | |
| | CTCACAACATCTGCACCTTCGTGCCCAGAAGAAAGG | |
| | CCGGCATCTGCTTCGGAGATTCTGGCGGCCCTCTGAT | |
| | CTGCGACGGAATCATCCAGGGCATCGACAGCTTCGT | |
| | GATCTGGGGCTGTGCCACCAGACTGTTCCCCGACTTC | |
| | TTTACCCGGGTGGCCCTCTACGTGGACTGGATCCGGT | |
| | CTACACTGAGAGAGTCTAAATACGGACCGCCTTGTCC | |
| | TCCTTGTCCCGGCCAGCCAAGAGAGCCCCAGGTTTAC | |
| | ACACTGCCTCCAAGCCAAGAGGAAATGACCAAGAAT | |
| | CAGGTGTCCCTGACATGCCTGGTCAAGGGCTTCTACC | |
| | CCTCCGATATCGCCGTGGAATGGGAGAGCAATGGCC | |
| | AGCCTGAGAACAACTACAAGACCACACCTCCTGTGC | |
| | TGGACAGCGACGGCAGTTTCTTCCTGTATAGTAGACT | |
| | CACCGTGGATAAATCAAGATGGCAAGAGGGCAACGT | |
| | GTTCAGCTGCAGCGTGATGCACGAGGCCCTGCACAA | |
| | CCACTACACCCAGAAAAGCCTGAGCCTGTCTCTGGGC | |
| | AAGATGTTCTGGGTGCTCGTGGTCGTTGGCGGAGTGC | |
| | TGGCCTGTTACAGCCTGCTGGTTACCGTGGCCTTCAT | |
| | CATCTTTTGGGTCAAGCGGGGCAGAAAGAAGCTGCT | |
| | CTACATCTTCAAGCAGCCCTTCATGCGGCCCGTGCAG | |
| | ACCACACAAGAGGAAGATGGCTGCTCCTGCAGATTC | |
| | CCCGAGGAAGAAGAAGGCGGCTGCGAGCTGAGAGTG | |
| | AAGTTCAGCAGATCCGCCGACGCTCCAGCCTATCAGC | |
| | AGGGCCAAAACCAGCTGTACAACGAGCTGAACCTGG | |
| | GGAGAAGAGAAGAGTACGACGTGCTGGATAAGCGG | |
| | AGAGGCAGAGATCCTGAAATGGGCGGCAAGCCCAGA | |
| | CGGAAGAATCCTCAAGAGGGCCTGTATAATGAGCTG | |
| | CAGAAAGACAAGATGGCCGAGGCCTACAGCGAGATC | |
| | GGAATGAAGGGCGAGCGCAGAAGAGGCAAGGGACA | |
| | CGATGGACTGTACCAGGGCCTGAGCACCGCCACCAA | |
| | GGATACCTATGACGCACTGCACATGCAGGCCCTGCC | |
| | ACCTAGA | |
| 76 | ATGCCTTTGTTGCTGCTGCTGCCTCTTCTGTGGGCTGG | PR3_6 (Ile |
| | CGCTCTGGCTGTTGGAGGACATGAAGCCCAGCCTCAC | Deletion) LS |
| | AGCAGACCTTACATGGCCAGCCTGCAGATGAGAGGC | CAAR |
| | AATCCCGGCTCTCACTTTTGTGGCGGCACCCTGATTC | (PR3_6_46) |
| | ACCCCTCCTTCGTTCTGACCGCCGCTCACTGCCTGAG | Nucleotide |
| | AGACATCCCTCAGAGACTGGTCAACGTGGTGCTGGG | |
| | AGCCCACAACGTGCGGACACAAGAACCTACACAGCA | |
| | GCACTTCAGCGTGGCCCAGGTGTTCCTGAACAACTAC | |
| | GACGCCGAGAACAAGCTGAACGACGTGCTGCTGATC | |
| | CAGCTGAGCAGCCCTGCTAATCTGTCTGCCTCTGTGG | |
| | CCACTGTGCAGCTGCCTCAGCAAGATCAGCCTGTGCC | |
| | TCACGGCACACAGTGTCTGGCTATGGGATGGGGAAG | |
| | AGTGGGCGCTCATGATCCTCCAGCTCAAGTGCTGCAA | |
| | GAGCTGAACGTGACCGTCGTGACCTTCTTCTGCCGGC | |
| | CTCACAACATCTGCACCTTCGTGCCCAGAAGAAAGG | |
| | CCGGCATCTGCTTCGGAGATTCTGGCGGCCCTCTGAT | |
| | CTGCGACGGAATCATCCAGGGCATCGACAGCTTCGT | |
| | GATCTGGGGCTGTGCCACCAGACTGTTCCCCGACTTC | |
| | TTTACCCGGGTGGCCCTCTACGTGGACTGGATCCGGT | |
| | CTACACTGAGAGAGTCTAAATACGGACCGCCTTGTCC | |
| | TCCTTGTCCCGCTCCTCCTGTTGCCGGACCTTCCGTGT | |
| | TCCTGTTTCCTCCAAAGCCTAAGGACACCCTGATGAT | |
| | CAGCAGGACCCCTGAAGTGACCTGCGTGGTGGTGGA | |
| | TGTGTCCCAAGAGGATCCCGAGGTGCAGTTCAACTG | |
| | GTATGTGGACGGCGTGGAAGTGCACAACGCCAAGAC | |
| | CAAGCCTAGAGAGGAACAGTTCCAGAGCACCTACAG | |
| | AGTGGTGTCCGTGCTGACAGTGCTGCACCAGGATTGG | |
| | CTGAACGGCAAAGAGTACAAGTGCAAGGTGTCCAAC | |
| | AAGGGCCTGCCTAGCAGCATCGAGAAAACCATCTCC | |
| | AAGGCCAAGGGCCAGCCAAGAGAGCCCCAGGTTTAC | |
| | ACACTGCCTCCAAGCCAAGAGGAAATGACCAAGAAT | |
| | CAGGTGTCCCTGACATGCCTGGTCAAGGGCTTCTACC | |
| | CCTCCGATATCGCCGTGGAATGGGAGAGCAATGGCC | |
| | AGCCTGAGAACAACTACAAGACCACACCTCCTGTGC | |
| | TGGACAGCGACGGCAGTTTCTTCCTGTATAGTAGACT | |
| | CACCGTGGATAAATCAAGATGGCAAGAGGGCAACGT | |
| | GTTCAGCTGCAGCGTGATGCACGAGGCCCTGCACAA | |
| | CCACTACACCCAGAAAAGCCTGAGCCTGTCTCTGGGC | |
| | AAGATGTTCTGGGTGCTCGTGGTCGTTGGCGGAGTGC | |
| | TGGCCTGTTACAGCCTGCTGGTTACCGTGGCCTTCAT | |
| | CATCTTTTGGGTCAAGCGGGGCAGAAAGAAGCTGCT | |
| | CTACATCTTCAAGCAGCCCTTCATGCGGCCCGTGCAG | |
| | ACCACACAAGAGGAAGATGGCTGCTCCTGCAGATTC | |
| | CCCGAGGAAGAAGAAGGCGGCTGCGAGCTGAGAGTG | |
| | AAGTTCAGCAGATCCGCCGACGCTCCAGCCTATCAGC | |
| | AGGGCCAAAACCAGCTGTACAACGAGCTGAACCTGG | |
| | GGAGAAGAGAAGAGTACGACGTGCTGGATAAGCGG | |
| | AGAGGCAGAGATCCTGAAATGGGCGGCAAGCCCAGA | |
| | CGGAAGAATCCTCAAGAGGGCCTGTATAATGAGCTG | |
| | CAGAAAGACAAGATGGCCGAGGCCTACAGCGAGATC | |
| | GGAATGAAGGGCGAGCGCAGAAGAGGCAAGGGACA | |
| | CGATGGACTGTACCAGGGCCTGAGCACCGCCACCAA | |
| | GGATACCTATGACGCACTGCACATGCAGGCCCTGCC | |
| | ACCTAGA | |
| 77 | ATGCCTTTGTTGCTGCTGCTGCCTCTTCTGTGGGCTGG | PR3_7 (D175N) |
| | CGCTCTGGCTATCGTTGGAGGACATGAAGCCCAGCCT | SS CAAR |
| | CACAGCAGACCTTACATGGCCAGCCTGCAGATGAGA | (PR3_7_47) |
| | GGCAATCCCGGCTCTCACTTTTGTGGCGGCACCCTGA | Nucleotide |
| | TTCACCCCTCCTTCGTTCTGACCGCCGCTCACTGCCTG | |
| | AGAGACATCCCTCAGAGACTGGTCAACGTGGTGCTG | |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTCGGAAACTCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCATGTTCTGGGTGCTCGTGGTCGTTGG | |
| | CGGAGTGCTGGCCTGTTACAGCCTGCTGGTTACCGTG | |
| | GCCTTCATCATCTTTTGGGTCAAGCGGGGCAGAAAGA | |
| | AGCTGCTCTACATCTTCAAGCAGCCCTTCATGCGGCC | |
| | CGTGCAGACCACACAAGAGGAAGATGGCTGCTCCTG | |
| | CAGATTCCCCGAGGAAGAAGAAGGCGGCTGCGAGCT | |
| | GAGAGTGAAGTTCAGCAGATCCGCCGACGCTCCAGC | |
| | CTATCAGCAGGGCCAAAACCAGCTGTACAACGAGCT | |
| | GAACCTGGGGAGAAGAGAAGAGTACGACGTGCTGGA | |
| | TAAGCGGAGAGGCAGAGATCCTGAAATGGGCGGCAA | |
| | GCCCAGACGGAAGAATCCTCAAGAGGGCCTGTATAA | |
| | TGAGCTGCAGAAAGACAAGATGGCCGAGGCCTACAG | |
| | CGAGATCGGAATGAAGGGCGAGCGCAGAAGAGGCA | |
| | AGGGACACGATGGACTGTACCAGGGCCTGAGCACCG | |
| | CCACCAAGGATACCTATGACGCACTGCACATGCAGG | |
| | CCCTGCCACCTAGA | |
| 78 | ATGCCTTTGTTGCTGCTGCTGCCTCTTCTGTGGGCTGG | PR3_7 (D175N) |
| | CGCTCTGGCTATCGTTGGAGGACATGAAGCCCAGCCT | MS CAAR |
| | CACAGCAGACCTTACATGGCCAGCCTGCAGATGAGA | (PR3_7_48) |
| | GGCAATCCCGGCTCTCACTTTTGTGGCGGCACCCTGA | Nucleotide |
| | TTCACCCCTCCTTCGTTCTGACCGCCGCTCACTGCCTG | |
| | AGAGACATCCCTCAGAGACTGGTCAACGTGGTGCTG | |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTCGGAAACTCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCGGCCAGCCAAGAGAGCCCCAGGTTT | |
| | ACACACTGCCTCCAAGCCAAGAGGAAATGACCAAGA | |
| | ATCAGGTGTCCCTGACATGCCTGGTCAAGGGCTTCTA | |
| | CCCCTCCGATATCGCCGTGGAATGGGAGAGCAATGG | |
| | CCAGCCTGAGAACAACTACAAGACCACACCTCCTGT | |
| | GCTGGACAGCGACGGCAGTTTCTTCCTGTATAGTAGA | |
| | CTCACCGTGGATAAATCAAGATGGCAAGAGGGCAAC | |
| | GTGTTCAGCTGCAGCGTGATGCACGAGGCCCTGCAC | |
| | AACCACTACACCCAGAAAAGCCTGAGCCTGTCTCTG | |
| | GGCAAGATGTTCTGGGTGCTCGTGGTCGTTGGCGGAG | |
| | TGCTGGCCTGTTACAGCCTGCTGGTTACCGTGGCCTT | |
| | CATCATCTTTTGGGTCAAGCGGGGCAGAAAGAAGCT | |
| | GCTCTACATCTTCAAGCAGCCCTTCATGCGGCCCGTG | |
| | CAGACCACACAAGAGGAAGATGGCTGCTCCTGCAGA | |
| | TTCCCCGAGGAAGAAGAAGGCGGCTGCGAGCTGAGA | |
| | GTGAAGTTCAGCAGATCCGCCGACGCTCCAGCCTATC | |
| | AGCAGGGCCAAAACCAGCTGTACAACGAGCTGAACC | |
| | TGGGGAGAAGAGAAGAGTACGACGTGCTGGATAAGC | |
| | GGAGAGGCAGAGATCCTGAAATGGGCGGCAAGCCCA | |
| | GACGGAAGAATCCTCAAGAGGGCCTGTATAATGAGC | |
| | TGCAGAAAGACAAGATGGCCGAGGCCTACAGCGAGA | |
| | TCGGAATGAAGGGCGAGCGCAGAAGAGGCAAGGGA | |
| | CACGATGGACTGTACCAGGGCCTGAGCACCGCCACC | |
| | AAGGATACCTATGACGCACTGCACATGCAGGCCCTG | |
| | CCACCTAGA | |
| 79 | ATGCCTTTGTTGCTGCTGCTGCCTCTTCTGTGGGCTGG | PR3_7 (D175N) |
| | CGCTCTGGCTATCGTTGGAGGACATGAAGCCCAGCCT | LS CAAR |
| | CACAGCAGACCTTACATGGCCAGCCTGCAGATGAGA | (PR3_7_49) |
| | GGCAATCCCGGCTCTCACTTTTGTGGCGGCACCCTGA | Nucleotide |
| | TTCACCCCTCCTTCGTTCTGACCGCCGCTCACTGCCTG | |
| | AGAGACATCCCTCAGAGACTGGTCAACGTGGTGCTG | |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTCGGAAACTCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCGCTCCTCCTGTTGCCGGACCTTCCGT | |
| | GTTCCTGTTTCCTCCAAAGCCTAAGGACACCCTGATG | |
| | ATCAGCAGGACCCCTGAAGTGACCTGCGTGGTGGTG | |
| | GATGTGTCCCAAGAGGATCCCGAGGTGCAGTTCAAC | |
| | TGGTATGTGGACGGCGTGGAAGTGCACAACGCCAAG | |
| | ACCAAGCCTAGAGAGGAACAGTTCCAGAGCACCTAC | |
| | AGAGTGGTGTCCGTGCTGACAGTGCTGCACCAGGATT | |
| | GGCTGAACGGCAAAGAGTACAAGTGCAAGGTGTCCA | |
| | ACAAGGGCCTGCCTAGCAGCATCGAGAAAACCATCT | |
| | CCAAGGCCAAGGGCCAGCCAAGAGAGCCCCAGGTTT | |
| | ACACACTGCCTCCAAGCCAAGAGGAAATGACCAAGA | |
| | ATCAGGTGTCCCTGACATGCCTGGTCAAGGGCTTCTA | |
| | CCCCTCCGATATCGCCGTGGAATGGGAGAGCAATGG | |
| | CCAGCCTGAGAACAACTACAAGACCACACCTCCTGT | |
| | GCTGGACAGCGACGGCAGTTTCTTCCTGTATAGTAGA | |
| | CTCACCGTGGATAAATCAAGATGGCAAGAGGGCAAC | |
| | GTGTTCAGCTGCAGCGTGATGCACGAGGCCCTGCAC | |
| | AACCACTACACCCAGAAAAGCCTGAGCCTGTCTCTG | |
| | GGCAAGATGTTCTGGGTGCTCGTGGTCGTTGGCGGAG | |
| | TGCTGGCCTGTTACAGCCTGCTGGTTACCGTGGCCTT | |
| | CATCATCTTTTGGGTCAAGCGGGGCAGAAAGAAGCT | |
| | GCTCTACATCTTCAAGCAGCCCTTCATGCGGCCCGTG | |
| | CAGACCACACAAGAGGAAGATGGCTGCTCCTGCAGA | |
| | TTCCCCGAGGAAGAAGAAGGCGGCTGCGAGCTGAGA | |
| | GTGAAGTTCAGCAGATCCGCCGACGCTCCAGCCTATC | |
| | AGCAGGGCCAAAACCAGCTGTACAACGAGCTGAACC | |
| | TGGGGAGAAGAGAAGAGTACGACGTGCTGGATAAGC | |
| | GGAGAGGCAGAGATCCTGAAATGGGCGGCAAGCCCA | |
| | GACGGAAGAATCCTCAAGAGGGCCTGTATAATGAGC | |
| | TGCAGAAAGACAAGATGGCCGAGGCCTACAGCGAGA | |
| | TCGGAATGAAGGGCGAGCGCAGAAGAGGCAAGGGA | |
| | CACGATGGACTGTACCAGGGCCTGAGCACCGCCACC | |
| | AAGGATACCTATGACGCACTGCACATGCAGGCCCTG | |
| | CCACCTAGA | |
| 80 | ATGCCTTTGTTGCTGCTGCTGCCTCTTCTGTGGGCTGG | PR3_8 (S176C) |
| | CGCTCTGGCTATCGTTGGAGGACATGAAGCCCAGCCT | SS CAAR |
| | CACAGCAGACCTTACATGGCCAGCCTGCAGATGAGA | (PR3_8_50) |
| | GGCAATCCCGGCTCTCACTTTTGTGGCGGCACCCTGA | Nucleotide |
| | TTCACCCCTCCTTCGTTCTGACCGCCGCTCACTGCCTG | |
| | AGAGACATCCCTCAGAGACTGGTCAACGTGGTGCTG | |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTCGGAGATTGCGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCATGTTCTGGGTGCTCGTGGTCGTTGG | |
| | CGGAGTGCTGGCCTGTTACAGCCTGCTGGTTACCGTG | |
| | GCCTTCATCATCTTTTGGGTCAAGCGGGGCAGAAAGA | |
| | AGCTGCTCTACATCTTCAAGCAGCCCTTCATGCGGCC | |
| | CGTGCAGACCACACAAGAGGAAGATGGCTGCTCCTG | |
| | CAGATTCCCCGAGGAAGAAGAAGGCGGCTGCGAGCT | |
| | GAGAGTGAAGTTCAGCAGATCCGCCGACGCTCCAGC | |
| | CTATCAGCAGGGCCAAAACCAGCTGTACAACGAGCT | |
| | GAACCTGGGGAGAAGAGAAGAGTACGACGTGCTGGA | |
| | TAAGCGGAGAGGCAGAGATCCTGAAATGGGCGGCAA | |
| | GCCCAGACGGAAGAATCCTCAAGAGGGCCTGTATAA | |
| | TGAGCTGCAGAAAGACAAGATGGCCGAGGCCTACAG | |
| | CGAGATCGGAATGAAGGGCGAGCGCAGAAGAGGCA | |
| | AGGGACACGATGGACTGTACCAGGGCCTGAGCACCG | |
| | CCACCAAGGATACCTATGACGCACTGCACATGCAGG | |
| | CCCTGCCACCTAGA | |
| 81 | ATGCCTTTGTTGCTGCTGCTGCCTCTTCTGTGGGCTGG | PR3_8 (S176C) |
| | CGCTCTGGCTATCGTTGGAGGACATGAAGCCCAGCCT | MS CAAR |
| | CACAGCAGACCTTACATGGCCAGCCTGCAGATGAGA | (PR3_8_51) |
| | GGCAATCCCGGCTCTCACTTTTGTGGCGGCACCCTGA | Nucleotide |
| | TTCACCCCTCCTTCGTTCTGACCGCCGCTCACTGCCTG | |
| | AGAGACATCCCTCAGAGACTGGTCAACGTGGTGCTG | |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTCGGAGATTGCGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCGGCCAGCCAAGAGAGCCCCAGGTTT | |
| | ACACACTGCCTCCAAGCCAAGAGGAAATGACCAAGA | |
| | ATCAGGTGTCCCTGACATGCCTGGTCAAGGGCTTCTA | |
| | CCCCTCCGATATCGCCGTGGAATGGGAGAGCAATGG | |
| | CCAGCCTGAGAACAACTACAAGACCACACCTCCTGT | |
| | GCTGGACAGCGACGGCAGTTTCTTCCTGTATAGTAGA | |
| | CTCACCGTGGATAAATCAAGATGGCAAGAGGGCAAC | |
| | GTGTTCAGCTGCAGCGTGATGCACGAGGCCCTGCAC | |
| | AACCACTACACCCAGAAAAGCCTGAGCCTGTCTCTG | |
| | GGCAAGATGTTCTGGGTGCTCGTGGTCGTTGGCGGAG | |
| | TGCTGGCCTGTTACAGCCTGCTGGTTACCGTGGCCTT | |
| | CATCATCTTTTGGGTCAAGCGGGGCAGAAAGAAGCT | |
| | GCTCTACATCTTCAAGCAGCCCTTCATGCGGCCCGTG | |
| | CAGACCACACAAGAGGAAGATGGCTGCTCCTGCAGA | |
| | TTCCCCGAGGAAGAAGAAGGCGGCTGCGAGCTGAGA | |
| | GTGAAGTTCAGCAGATCCGCCGACGCTCCAGCCTATC | |
| | AGCAGGGCCAAAACCAGCTGTACAACGAGCTGAACC | |
| | TGGGGAGAAGAGAAGAGTACGACGTGCTGGATAAGC | |
| | GGAGAGGCAGAGATCCTGAAATGGGCGGCAAGCCCA | |
| | GACGGAAGAATCCTCAAGAGGGCCTGTATAATGAGC | |
| | TGCAGAAAGACAAGATGGCCGAGGCCTACAGCGAGA | |
| | TCGGAATGAAGGGCGAGCGCAGAAGAGGCAAGGGA | |
| | CACGATGGACTGTACCAGGGCCTGAGCACCGCCACC | |
| | AAGGATACCTATGACGCACTGCACATGCAGGCCCTG | |
| | CCACCTAGA | |
| 82 | ATGCCTTTGTTGCTGCTGCTGCCTCTTCTGTGGGCTGG | PR3_8 (S176C) |
| | CGCTCTGGCTATCGTTGGAGGACATGAAGCCCAGCCT | LS CAAR |
| | CACAGCAGACCTTACATGGCCAGCCTGCAGATGAGA | (PR3_8_52) |
| | GGCAATCCCGGCTCTCACTTTTGTGGCGGCACCCTGA | Nucleotide |
| | TTCACCCCTCCTTCGTTCTGACCGCCGCTCACTGCCTG | |
| | AGAGACATCCCTCAGAGACTGGTCAACGTGGTGCTG | |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTCGGAGATTGCGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCGCTCCTCCTGTTGCCGGACCTTCCGT | |
| | GTTCCTGTTTCCTCCAAAGCCTAAGGACACCCTGATG | |
| | ATCAGCAGGACCCCTGAAGTGACCTGCGTGGTGGTG | |
| | GATGTGTCCCAAGAGGATCCCGAGGTGCAGTTCAAC | |
| | TGGTATGTGGACGGCGTGGAAGTGCACAACGCCAAG | |
| | ACCAAGCCTAGAGAGGAACAGTTCCAGAGCACCTAC | |
| | AGAGTGGTGTCCGTGCTGACAGTGCTGCACCAGGATT | |
| | GGCTGAACGGCAAAGAGTACAAGTGCAAGGTGTCCA | |
| | ACAAGGGCCTGCCTAGCAGCATCGAGAAAACCATCT | |
| | CCAAGGCCAAGGGCCAGCCAAGAGAGCCCCAGGTTT | |
| | ACACACTGCCTCCAAGCCAAGAGGAAATGACCAAGA | |
| | ATCAGGTGTCCCTGACATGCCTGGTCAAGGGCTTCTA | |
| | CCCCTCCGATATCGCCGTGGAATGGGAGAGCAATGG | |
| | CCAGCCTGAGAACAACTACAAGACCACACCTCCTGT | |
| | GCTGGACAGCGACGGCAGTTTCTTCCTGTATAGTAGA | |
| | CTCACCGTGGATAAATCAAGATGGCAAGAGGGCAAC | |
| | GTGTTCAGCTGCAGCGTGATGCACGAGGCCCTGCAC | |
| | AACCACTACACCCAGAAAAGCCTGAGCCTGTCTCTG | |
| | GGCAAGATGTTCTGGGTGCTCGTGGTCGTTGGCGGAG | |
| | TGCTGGCCTGTTACAGCCTGCTGGTTACCGTGGCCTT | |
| | CATCATCTTTTGGGTCAAGCGGGGCAGAAAGAAGCT | |
| | GCTCTACATCTTCAAGCAGCCCTTCATGCGGCCCGTG | |
| | CAGACCACACAAGAGGAAGATGGCTGCTCCTGCAGA | |
| | TTCCCCGAGGAAGAAGAAGGCGGCTGCGAGCTGAGA | |
| | GTGAAGTTCAGCAGATCCGCCGACGCTCCAGCCTATC | |
| | AGCAGGGCCAAAACCAGCTGTACAACGAGCTGAACC | |
| | TGGGGAGAAGAGAAGAGTACGACGTGCTGGATAAGC | |
| | GGAGAGGCAGAGATCCTGAAATGGGCGGCAAGCCCA | |
| | GACGGAAGAATCCTCAAGAGGGCCTGTATAATGAGC | |
| | TGCAGAAAGACAAGATGGCCGAGGCCTACAGCGAGA | |
| | TCGGAATGAAGGGCGAGCGCAGAAGAGGCAAGGGA | |
| | CACGATGGACTGTACCAGGGCCTGAGCACCGCCACC | |
| | AAGGATACCTATGACGCACTGCACATGCAGGCCCTG | |
| | CCACCTAGA | |
| 83 | ATGCCTTTGTTGCTGCTGCTGCCTCTTCTGTGGGCTGG | PR3_9 (G4P) SS |
| | CGCTCTGGCTATCGTTGGACCCCATGAAGCCCAGCCT | CAAR |
| | CACAGCAGACCTTACATGGCCAGCCTGCAGATGAGA | (PR3_9_53) |
| | GGCAATCCCGGCTCTCACTTTTGTGGCGGCACCCTGA | Nucleotide |
| | TTCACCCCTCCTTCGTTCTGACCGCCGCTCACTGCCTG | |
| | AGAGACATCCCTCAGAGACTGGTCAACGTGGTGCTG | |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTCGGAGATTCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCATGTTCTGGGTGCTCGTGGTCGTTGG | |
| | CGGAGTGCTGGCCTGTTACAGCCTGCTGGTTACCGTG | |
| | GCCTTCATCATCTTTTGGGTCAAGCGGGGCAGAAAGA | |
| | AGCTGCTCTACATCTTCAAGCAGCCCTTCATGCGGCC | |
| | CGTGCAGACCACACAAGAGGAAGATGGCTGCTCCTG | |
| | CAGATTCCCCGAGGAAGAAGAAGGCGGCTGCGAGCT | |
| | GAGAGTGAAGTTCAGCAGATCCGCCGACGCTCCAGC | |
| | CTATCAGCAGGGCCAAAACCAGCTGTACAACGAGCT | |
| | GAACCTGGGGAGAAGAGAAGAGTACGACGTGCTGGA | |
| | TAAGCGGAGAGGCAGAGATCCTGAAATGGGCGGCAA | |
| | GCCCAGACGGAAGAATCCTCAAGAGGGCCTGTATAA | |
| | TGAGCTGCAGAAAGACAAGATGGCCGAGGCCTACAG | |
| | CGAGATCGGAATGAAGGGCGAGCGCAGAAGAGGCA | |
| | AGGGACACGATGGACTGTACCAGGGCCTGAGCACCG | |
| | CCACCAAGGATACCTATGACGCACTGCACATGCAGG | |
| | CCCTGCCACCTAGA | |
| 84 | ATGCCTTTGTTGCTGCTGCTGCCTCTTCTGTGGGCTGG | PR3_9 (G4P) |
| | CGCTCTGGCTATCGTTGGACCCCATGAAGCCCAGCCT | MS CAAR |
| | CACAGCAGACCTTACATGGCCAGCCTGCAGATGAGA | (PR3_9_54) |
| | GGCAATCCCGGCTCTCACTTTTGTGGCGGCACCCTGA | Nucleotide |
| | TTCACCCCTCCTTCGTTCTGACCGCCGCTCACTGCCTG | |
| | AGAGACATCCCTCAGAGACTGGTCAACGTGGTGCTG | |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTCGGAGATTCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCGGCCAGCCAAGAGAGCCCCAGGTTT | |
| | ACACACTGCCTCCAAGCCAAGAGGAAATGACCAAGA | |
| | ATCAGGTGTCCCTGACATGCCTGGTCAAGGGCTTCTA | |
| | CCCCTCCGATATCGCCGTGGAATGGGAGAGCAATGG | |
| | CCAGCCTGAGAACAACTACAAGACCACACCTCCTGT | |
| | GCTGGACAGCGACGGCAGTTTCTTCCTGTATAGTAGA | |
| | CTCACCGTGGATAAATCAAGATGGCAAGAGGGCAAC | |
| | GTGTTCAGCTGCAGCGTGATGCACGAGGCCCTGCAC | |
| | AACCACTACACCCAGAAAAGCCTGAGCCTGTCTCTG | |
| | GGCAAGATGTTCTGGGTGCTCGTGGTCGTTGGCGGAG | |
| | TGCTGGCCTGTTACAGCCTGCTGGTTACCGTGGCCTT | |
| | CATCATCTTTTGGGTCAAGCGGGGCAGAAAGAAGCT | |
| | GCTCTACATCTTCAAGCAGCCCTTCATGCGGCCCGTG | |
| | CAGACCACACAAGAGGAAGATGGCTGCTCCTGCAGA | |
| | TTCCCCGAGGAAGAAGAAGGCGGCTGCGAGCTGAGA | |
| | GTGAAGTTCAGCAGATCCGCCGACGCTCCAGCCTATC | |
| | AGCAGGGCCAAAACCAGCTGTACAACGAGCTGAACC | |
| | TGGGGAGAAGAGAAGAGTACGACGTGCTGGATAAGC | |
| | GGAGAGGCAGAGATCCTGAAATGGGCGGCAAGCCCA | |
| | GACGGAAGAATCCTCAAGAGGGCCTGTATAATGAGC | |
| | TGCAGAAAGACAAGATGGCCGAGGCCTACAGCGAGA | |
| | TCGGAATGAAGGGCGAGCGCAGAAGAGGCAAGGGA | |
| | CACGATGGACTGTACCAGGGCCTGAGCACCGCCACC | |
| | AAGGATACCTATGACGCACTGCACATGCAGGCCCTG | |
| | CCACCTAGA | |
| 85 | ATGCCTTTGTTGCTGCTGCTGCCTCTTCTGTGGGCTGG | PR3_9 (G4P) LS |
| | CGCTCTGGCTATCGTTGGACCCCATGAAGCCCAGCCT | CAAR |
| | CACAGCAGACCTTACATGGCCAGCCTGCAGATGAGA | (PR3_9_55) |
| | GGCAATCCCGGCTCTCACTTTTGTGGCGGCACCCTGA | Nucleotide |
| | TTCACCCCTCCTTCGTTCTGACCGCCGCTCACTGCCTG | |
| | AGAGACATCCCTCAGAGACTGGTCAACGTGGTGCTG | |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTCGGAGATTCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCGCTCCTCCTGTTGCCGGACCTTCCGT | |
| | GTTCCTGTTTCCTCCAAAGCCTAAGGACACCCTGATG | |
| | ATCAGCAGGACCCCTGAAGTGACCTGCGTGGTGGTG | |
| | GATGTGTCCCAAGAGGATCCCGAGGTGCAGTTCAAC | |
| | TGGTATGTGGACGGCGTGGAAGTGCACAACGCCAAG | |
| | ACCAAGCCTAGAGAGGAACAGTTCCAGAGCACCTAC | |
| | AGAGTGGTGTCCGTGCTGACAGTGCTGCACCAGGATT | |
| | GGCTGAACGGCAAAGAGTACAAGTGCAAGGTGTCCA | |
| | ACAAGGGCCTGCCTAGCAGCATCGAGAAAACCATCT | |
| | CCAAGGCCAAGGGCCAGCCAAGAGAGCCCCAGGTTT | |
| | ACACACTGCCTCCAAGCCAAGAGGAAATGACCAAGA | |
| | ATCAGGTGTCCCTGACATGCCTGGTCAAGGGCTTCTA | |
| | CCCCTCCGATATCGCCGTGGAATGGGAGAGCAATGG | |
| | CCAGCCTGAGAACAACTACAAGACCACACCTCCTGT | |
| | GCTGGACAGCGACGGCAGTTTCTTCCTGTATAGTAGA | |
| | CTCACCGTGGATAAATCAAGATGGCAAGAGGGCAAC | |
| | GTGTTCAGCTGCAGCGTGATGCACGAGGCCCTGCAC | |
| | AACCACTACACCCAGAAAAGCCTGAGCCTGTCTCTG | |
| | GGCAAGATGTTCTGGGTGCTCGTGGTCGTTGGCGGAG | |
| | TGCTGGCCTGTTACAGCCTGCTGGTTACCGTGGCCTT | |
| | CATCATCTTTTGGGTCAAGCGGGGCAGAAAGAAGCT | |
| | GCTCTACATCTTCAAGCAGCCCTTCATGCGGCCCGTG | |
| | CAGACCACACAAGAGGAAGATGGCTGCTCCTGCAGA | |
| | TTCCCCGAGGAAGAAGAAGGCGGCTGCGAGCTGAGA | |
| | GTGAAGTTCAGCAGATCCGCCGACGCTCCAGCCTATC | |
| | AGCAGGGCCAAAACCAGCTGTACAACGAGCTGAACC | |
| | TGGGGAGAAGAGAAGAGTACGACGTGCTGGATAAGC | |
| | GGAGAGGCAGAGATCCTGAAATGGGCGGCAAGCCCA | |
| | GACGGAAGAATCCTCAAGAGGGCCTGTATAATGAGC | |
| | TGCAGAAAGACAAGATGGCCGAGGCCTACAGCGAGA | |
| | TCGGAATGAAGGGCGAGCGCAGAAGAGGCAAGGGA | |
| | CACGATGGACTGTACCAGGGCCTGAGCACCGCCACC | |
| | AAGGATACCTATGACGCACTGCACATGCAGGCCCTG | |
| | CCACCTAGA | |
| 86 | ATGCCTTTGTTGCTGCTGCTGCCTCTTCTGTGGGCTGG | PR3_10 (H44A) |
| | CGCTCTGGCTATCGTTGGAGGACATGAAGCCCAGCCT | SS CAAR |
| | CACAGCAGACCTTACATGGCCAGCCTGCAGATGAGA | (PR3_10_56) |
| | GGCAATCCCGGCTCTCACTTTTGTGGCGGCACCCTGA | Nucleotide |
| | TTCACCCCTCCTTCGTTCTGACCGCCGCTGCCTGCCTG | |
| | AGAGACATCCCTCAGAGACTGGTCAACGTGGTGCTG | |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTCGGAGATTCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCATGTTCTGGGTGCTCGTGGTCGTTGG | |
| | CGGAGTGCTGGCCTGTTACAGCCTGCTGGTTACCGTG | |
| | GCCTTCATCATCTTTTGGGTCAAGCGGGGCAGAAAGA | |
| | AGCTGCTCTACATCTTCAAGCAGCCCTTCATGCGGCC | |
| | CGTGCAGACCACACAAGAGGAAGATGGCTGCTCCTG | |
| | CAGATTCCCCGAGGAAGAAGAAGGCGGCTGCGAGCT | |
| | GAGAGTGAAGTTCAGCAGATCCGCCGACGCTCCAGC | |
| | CTATCAGCAGGGCCAAAACCAGCTGTACAACGAGCT | |
| | GAACCTGGGGAGAAGAGAAGAGTACGACGTGCTGGA | |
| | TAAGCGGAGAGGCAGAGATCCTGAAATGGGCGGCAA | |
| | GCCCAGACGGAAGAATCCTCAAGAGGGCCTGTATAA | |
| | TGAGCTGCAGAAAGACAAGATGGCCGAGGCCTACAG | |
| | CGAGATCGGAATGAAGGGCGAGCGCAGAAGAGGCA | |
| | AGGGACACGATGGACTGTACCAGGGCCTGAGCACCG | |
| | CCACCAAGGATACCTATGACGCACTGCACATGCAGG | |
| | CCCTGCCACCTAGA | |
| 87 | ATGCCTTTGTTGCTGCTGCTGCCTCTTCTGTGGGCTGG | PR3_10 (H44A) |
| | CGCTCTGGCTATCGTTGGAGGACATGAAGCCCAGCCT | MS CAAR |
| | CACAGCAGACCTTACATGGCCAGCCTGCAGATGAGA | (PR3_10_57) |
| | GGCAATCCCGGCTCTCACTTTTGTGGCGGCACCCTGA | Nucleotide |
| | TTCACCCCTCCTTCGTTCTGACCGCCGCTGCCTGCCTG | |
| | AGAGACATCCCTCAGAGACTGGTCAACGTGGTGCTG | |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTCGGAGATTCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCGGCCAGCCAAGAGAGCCCCAGGTTT | |
| | ACACACTGCCTCCAAGCCAAGAGGAAATGACCAAGA | |
| | ATCAGGTGTCCCTGACATGCCTGGTCAAGGGCTTCTA | |
| | CCCCTCCGATATCGCCGTGGAATGGGAGAGCAATGG | |
| | CCAGCCTGAGAACAACTACAAGACCACACCTCCTGT | |
| | GCTGGACAGCGACGGCAGTTTCTTCCTGTATAGTAGA | |
| | CTCACCGTGGATAAATCAAGATGGCAAGAGGGCAAC | |
| | GTGTTCAGCTGCAGCGTGATGCACGAGGCCCTGCAC | |
| | AACCACTACACCCAGAAAAGCCTGAGCCTGTCTCTG | |
| | GGCAAGATGTTCTGGGTGCTCGTGGTCGTTGGCGGAG | |
| | TGCTGGCCTGTTACAGCCTGCTGGTTACCGTGGCCTT | |
| | CATCATCTTTTGGGTCAAGCGGGGCAGAAAGAAGCT | |
| | GCTCTACATCTTCAAGCAGCCCTTCATGCGGCCCGTG | |
| | CAGACCACACAAGAGGAAGATGGCTGCTCCTGCAGA | |
| | TTCCCCGAGGAAGAAGAAGGCGGCTGCGAGCTGAGA | |
| | GTGAAGTTCAGCAGATCCGCCGACGCTCCAGCCTATC | |
| | AGCAGGGCCAAAACCAGCTGTACAACGAGCTGAACC | |
| | TGGGGAGAAGAGAAGAGTACGACGTGCTGGATAAGC | |
| | GGAGAGGCAGAGATCCTGAAATGGGCGGCAAGCCCA | |
| | GACGGAAGAATCCTCAAGAGGGCCTGTATAATGAGC | |
| | TGCAGAAAGACAAGATGGCCGAGGCCTACAGCGAGA | |
| | TCGGAATGAAGGGCGAGCGCAGAAGAGGCAAGGGA | |
| | CACGATGGACTGTACCAGGGCCTGAGCACCGCCACC | |
| | AAGGATACCTATGACGCACTGCACATGCAGGCCCTG | |
| | CCACCTAGA | |
| 88 | ATGCCTTTGTTGCTGCTGCTGCCTCTTCTGTGGGCTGG | PR3_10 (H44A) |
| | CGCTCTGGCTATCGTTGGAGGACATGAAGCCCAGCCT | LS CAAR |
| | CACAGCAGACCTTACATGGCCAGCCTGCAGATGAGA | (PR3_10_58) |
| | GGCAATCCCGGCTCTCACTTTTGTGGCGGCACCCTGA | Nucleotide |
| | TTCACCCCTCCTTCGTTCTGACCGCCGCTGCCTGCCTG | |
| | AGAGACATCCCTCAGAGACTGGTCAACGTGGTGCTG | |
| | GGAGCCCACAACGTGCGGACACAAGAACCTACACAG | |
| | CAGCACTTCAGCGTGGCCCAGGTGTTCCTGAACAACT | |
| | ACGACGCCGAGAACAAGCTGAACGACGTGCTGCTGA | |
| | TCCAGCTGAGCAGCCCTGCTAATCTGTCTGCCTCTGT | |
| | GGCCACTGTGCAGCTGCCTCAGCAAGATCAGCCTGTG | |
| | CCTCACGGCACACAGTGTCTGGCTATGGGATGGGGA | |
| | AGAGTGGGCGCTCATGATCCTCCAGCTCAAGTGCTGC | |
| | AAGAGCTGAACGTGACCGTCGTGACCTTCTTCTGCCG | |
| | GCCTCACAACATCTGCACCTTCGTGCCCAGAAGAAA | |
| | GGCCGGCATCTGCTTCGGAGATTCTGGCGGCCCTCTG | |
| | ATCTGCGACGGAATCATCCAGGGCATCGACAGCTTC | |
| | GTGATCTGGGGCTGTGCCACCAGACTGTTCCCCGACT | |
| | TCTTTACCCGGGTGGCCCTCTACGTGGACTGGATCCG | |
| | GTCTACACTGAGAGAGTCTAAATACGGACCGCCTTGT | |
| | CCTCCTTGTCCCGCTCCTCCTGTTGCCGGACCTTCCGT | |
| | GTTCCTGTTTCCTCCAAAGCCTAAGGACACCCTGATG | |
| | ATCAGCAGGACCCCTGAAGTGACCTGCGTGGTGGTG | |
| | GATGTGTCCCAAGAGGATCCCGAGGTGCAGTTCAAC | |
| | TGGTATGTGGACGGCGTGGAAGTGCACAACGCCAAG | |
| | ACCAAGCCTAGAGAGGAACAGTTCCAGAGCACCTAC | |
| | AGAGTGGTGTCCGTGCTGACAGTGCTGCACCAGGATT | |
| | GGCTGAACGGCAAAGAGTACAAGTGCAAGGTGTCCA | |
| | ACAAGGGCCTGCCTAGCAGCATCGAGAAAACCATCT | |
| | CCAAGGCCAAGGGCCAGCCAAGAGAGCCCCAGGTTT | |
| | ACACACTGCCTCCAAGCCAAGAGGAAATGACCAAGA | |
| | ATCAGGTGTCCCTGACATGCCTGGTCAAGGGCTTCTA | |
| | CCCCTCCGATATCGCCGTGGAATGGGAGAGCAATGG | |
| | CCAGCCTGAGAACAACTACAAGACCACACCTCCTGT | |
| | GCTGGACAGCGACGGCAGTTTCTTCCTGTATAGTAGA | |
| | CTCACCGTGGATAAATCAAGATGGCAAGAGGGCAAC | |
| | GTGTTCAGCTGCAGCGTGATGCACGAGGCCCTGCAC | |
| | AACCACTACACCCAGAAAAGCCTGAGCCTGTCTCTG | |
| | GGCAAGATGTTCTGGGTGCTCGTGGTCGTTGGCGGAG | |
| | TGCTGGCCTGTTACAGCCTGCTGGTTACCGTGGCCTT | |
| | CATCATCTTTTGGGTCAAGCGGGGCAGAAAGAAGCT | |
| | GCTCTACATCTTCAAGCAGCCCTTCATGCGGCCCGTG | |
| | CAGACCACACAAGAGGAAGATGGCTGCTCCTGCAGA | |
| | TTCCCCGAGGAAGAAGAAGGCGGCTGCGAGCTGAGA | |
| | GTGAAGTTCAGCAGATCCGCCGACGCTCCAGCCTATC | |
| | AGCAGGGCCAAAACCAGCTGTACAACGAGCTGAACC | |
| | TGGGGAGAAGAGAAGAGTACGACGTGCTGGATAAGC | |
| | GGAGAGGCAGAGATCCTGAAATGGGCGGCAAGCCCA | |
| | GACGGAAGAATCCTCAAGAGGGCCTGTATAATGAGC | |
| | TGCAGAAAGACAAGATGGCCGAGGCCTACAGCGAGA | |
| | TCGGAATGAAGGGCGAGCGCAGAAGAGGCAAGGGA | |
| | CACGATGGACTGTACCAGGGCCTGAGCACCGCCACC | |
| | AAGGATACCTATGACGCACTGCACATGCAGGCCCTG | |
| | CCACCTAGA | |
| 89 | MAHRPPSPAL | ANCA1 epitope |
| 90 | AQPHSRPYMAS | ANCA2 epitope |
| 91 | PGSHFCGG | ANCA3 epitope |
| 92 | VVLGAHNVRTQ | ANCA4 epitope |
| 93 | FLNNYDAE | ANCA5 epitope |
| 94 | PVPHGTQC | ANCA6 epitope |
| 95 | CFGDSGGP | ANCA7 eptitope |
| 96 | ESKYGPPCPPCPGQPREPQVYTLPPSQEEMTKNQVSLTCLVK | IgG4 Hinge-C_{H}3 |
| | GFYPSDIAVEWESNGQPENNYKTTPPVLDSDGSFFLYSRLTV | spacer (aa) |
| | DKSRWQEGNVFSCSVMHEALHNHYTQKSLSLSLGK | Medium spacer |
| | | (MS) |
| 97 | ESKYGPPCPPCPAPPVAGPSVFLFPPKPKDTLMISRTPEVTCV | IgG4 hinge- |
| | VVDVSQEDPEVQFNWYVDGVEVHNAKTKPREEQFQSTYRV | IgG2/IgG4 C_{H}2- |
| | VSVLTVLHQDWLNGKEYKCKVSNKGLPSSIEKTISKAKGQP | IgG4 C_{H}3 spacer |
| | REPQVYTLPPSQEEMTKNQVSLTCLVKGFYPSDIAVEWESNG | (aa) |
| | QPENNYKTTPPVLDSDGSFFLYSRLTVDKSRWQEGNVFSCSV | Long spacer (LS) |
| | MHEALHNHYTQKSLSLSLGK | |
| 98 | IYIWAPLAGTCGVLLLSLVIT | CD8a |
| | | transmembrane |
| | | domain (aa) |
| 99 | GGGGS | G4s spacer (aa) |
| 100 | GGGGSGGGGSGGGGS | (G4S)3 spacer |
| | | (aa) |
| 101 | KPTTTPAPRPPTPAPTIASQPLSLRPEACRPAAGGAVHTRGLD | CD8a spacer (aa) |
| | FACD | |
| 102 | EAAAK | EAAAK spacer |
| | | (aa) |
| 103 | IVGPHEAQPHSRPYMASLQMRGNPGSHFCGGTLIHPSFVLTA | PR3_9 |
| | AHCLRDIPQRLVNVVLGAHNVRTQEPTQQHFSVAQVFLNNY | (G4P)/(G4S)3/ |
| | DAENKLNDVLLIQLSSPANLSASVATVQLPQQDQPVPHGTQC | CD8aTM CAAR |
| | LAMGWGRVGAHDPPAQVLQELNVTVVTFFCRPHNICTFVPR | (PR3_9_103) |
| | RKAGICFGDSGGPLICDGIIQGIDSFVIWGCATRLFPDFFTRVA | Amino acid |
| | LYVDWIRSTLRGGGGSGGGGSGGGGSIYIWAPLAGTCGVLL | |
| | LSLVITKRGRKKLLYIFKQPFMR | |
| | PVQTTQEEDGCSCRFPEEEEGGCELRVKFS | |
| | RSADAPAYQQGQNQLYNELNLGRREEYDVLDKRRGRDPEM | |
| | GGKPRRKNPQEGLYNELQKDKMAEAYSEIGMKGERRRGKG | |
| | HDGLYQGLSTATKDTYDALHMQALPPR | |
| 104 | IVGPHEAQPHSRPYMASLQMRGNPGSHFCGGTLIHPSFVLTA | PR3_9 |
| | AHCLRDIPQRLVNVVLGAHNVRTQEPTQQHFSVAQVFLNNY | (G4P)/(G4S)3/ |
| | DAENKLNDVLLIQLSSPANLSASVATVQLPQQDQPVPHGTQC | CD28TM CAAR |
| | LAMGWGRVGAHDPPAQVLQELNVTVVTFFCRPHNICTFVPR | (PR3_9_104) |
| | RKAGICFGDSGGPLICDGIIQGIDSFVIWGCATRLFPDFFTRVA | Amino acid |
| | LYVDWIRSTLRGGGGSGGGGSGGGGSFWVLVVVGGVLACY | |
| | SLLVTVAFIIFWVKRGRKKLLYIFKQPFMRPVQTTQEEDGCS | |
| | CRFPEEEEGGCELRVKFSRSADAPAYQQGQNQLYNELNLGR | |
| | REEYDVLDKRRGRDPEMGGKPRRKNPQEGLYNELQKDKMA | |
| | EAYSEIGMKGERRRGKGHDGLYQGLSTATKDTYDALHMQA | |
| | LPPR | |
| 105 | IVGPHEAQPHSRPYMASLQMRGNPGSHFCGGTLIHPSFVLTA | PR3_9 |
| | AHCLRDIPQRLVNVVLGAHNVRTQEPTQQHFSVAQVFLNNY | (G4P)/(G4S)/ |
| | DAENKLNDVLLIQLSSPANLSASVATVQLPQQDQPVPHGTQC | CD8aTM CAAR |
| | LAMGWGRVGAHDPPAQVLQELNVTVVTFFCRPHNICTFVPR | (PR3_9_105) |
| | RKAGICFGDSGGPLICDGIIQGIDSFVIWGCATRLFPDFFTRVA | Amino acid |
| | LYVDWIRSTLRGGGGSIYIWAPLAGTCGVLLLSLVITKRGRK | |
| | KLLYIFKQPFMRPVQTTQEEDGCSCRFPEEEEGGCELRVKFS | |
| | RSADAPAYQQ | |
| | GQNQLYNELNLGRREEYDVLDKRRGRDPEMGGKPRRKNPQ | |
| | EGLYNELQKDKMAEAYSEIGMKGERRRGKGHDGLYQGLST | |
| | ATKDTYDALHMQALPPR | |
| 106 | IVGPHEAQPHSRPYMASLQMRGNPGSHFCGGTLIHPSFVLTA | PR3_9 |
| | AHCLRDIPQRLVNVVLGAHNVRTQEPTQQHFSVAQVFLNNY | (G4P)/(G4S)/ |
| | DAENKLNDVLLIQLSSPANLSASVATVQLPQQDQPVPHGTQC | CD28TM CAAR |
| | LAMGWGRVGAHDPPAQVLQELNVTVVTFFCRPHNICTFVPR | (PR3_9 106) |
| | RKAGICFGDSGGPLICDGIIQGIDSFVIWGCATRLFPDFFTRVA | Amino acid |
| | LYVDWIRSTLRGGGGSFWVLVVVGGVLACYSLLVTVAFIIF | |
| | WVKRGRKKLLYIFKQPFMRPVQTTQEEDGCSCRFPEEEEGG | |
| | CELRVKFSRSADAPAYQQGQNQLYNELNLGRREEYDVLDK | |
| | RRGRDPEMGGKP | |
| | RRKNPQEGLYNELQKDKMAEAYSEIGMKGERRRGKGHDGL | |
| | YQGLSTATKDTYDALHMQALPPR | |
| 107 | IVGPHEAQPHSRPYMASLQMRGNPGSHFCGGTLIHPSFVLTA | PR3_9 |
| | AHCLRDIPQRLVNVVLGAHNVRTQEPTQQHFSVAQVFLNNY | (G4P)/CD8a/ |
| | DAENKLNDVLLIQLSSPANLSASVATVQLPQQDQPVPHGTQC | CD8aTM CAAR |
| | LAMGWGRVGAHDPPAQVLQELNVTVVTFFCRPHNICTFVPR | (PR3_9_107) |
| | RKAGICFGDSGGPLICDGIIQGIDSFVIWGCATRLFPDFFTRVA | Amino acid |
| | LYVDWIRSTLRKPTTTPAPRPPTPAPTIASQPLSLRPEACRPAA | |
| | GGAVHTRGLDFACDIYIWAPLAGTCGVLLLSLVITKRGRKKL | |
| | LYIFKQPFMRPVQTTQEEDGCSCRFPEEEEGGCELRVKFSRSA | |
| | DAPAYQQGQNQLYNELNLGRREEYDVLDKRRGRDPEMGGK | |
| | PRRKNPQEGLYNELQKDKMAEAYSEIGMKGERRRGKGHDG | |
| | LYQGLSTATKDTYDALHMQALPPR | |
| 108 | IVGPHEAQPHSRPYMASLQMRGNPGSHFCGGTLIHPSFVLTA | PR3_9 |
| | AHCLRDIPQRLVNVVLGAHNVRTQEPTQQHFSVAQVFLNNY | (G4P)/EAAAK/ |
| | DAENKLNDVLLIQLSSPANLSASVATVQLPQQDQPVPHGTQC | CD8aTM CAAR |
| | LAMGWGRVGAHDPPAQVLQELNVTVVTFFCRPHNICTFVPR | (PR3_9_108) |
| | RKAGICFGDSGGPLICDGIIQGIDSFVIWGCATRLFPDFFTRVA | Amino acid |
| | LYVDWIRSTLREAAAKIYIWAPLAGTCGVLLLSLVITKRGRK | |
| | KLLYIFKQPFMRPVQTTQEEDGCSCRFPEEEEGGCELRVKFS | |
| | RSADAPAYQQGQNQLYNELNLGRREEYDVLDKRRGRDPEM | |
| | GGKPRRKNPQ | |
| | EGLYNELQKDKMAEAYSEIGMKGERRRGKGHDGLYQGLST | |
| | ATKDTYDALHMQALPPR | |
| 109 | IVGPHEAQPHSRPYMASLQMRGNPGSHFCGGTLIHPSFVLTA | PR3_9 |
| | AHCLRDIPQRLVNVVLGAHNVRTQEPTQQHFSVAQVFLNNY | (G4P)/MS/CD8aTM |
| | DAENKLNDVLLIQLSSPANLSASVATVQLPQQDQPVPHGTQC | CAAR |
| | LAMGWGRVGAHDPPAQVLQELNVTVVTFFCRPHNICTFVPR | (PR3_9_109) |
| | RKAGICFGDSGGPLICDGIIQGIDSFVIWGCATRLFPDFFTRVA | Amino acid |
| | LYVDWIRSTLRESKYGPPCPPCPGQPREPQVYTLPPSQEEMT | |
| | KNQVSLTCLVKGFYPSDIAVEWESNGQPENNYKTTPPVLDS | |
| | DGSFFLYSRLT | |
| | VDKSRWQEGNVFSCSVMHEALHNHYTQKSLSLSLGKMIYIW | |
| | APLAGTCGVLLLSLVITKRGRKKLLYIFKQPFMRPVQTTQEE | |
| | DGCSCRFPEEEEGGCELRVKFSRSADAPAYQQGQNQLYNEL | |
| | NLGRRE | |
| | EYDVLDKRRGRDPEMGGKPRRKNPQEGLYNELQKDKMAEA | |
| | YSEIGMKGERRRGKGHDGLYQGLSTATKDTYDALHMQALP | |
| | PR | |
| 110 | IVGPHEAQPHSRPYMASLQMRGNPGSHFCGGTLIHPSFVLTA | PR3_9 |
| | AHCLRDIPQRLVNVVLGAHNVRTQEPTQQHFSVAQVFLNNY | (G4P)/SS/CD8aTM |
| | DAENKLNDVLLIQLSSPANLSASVATVQLPQQDQPVPHGTQC | CAAR |
| | LAMGWGRVGAHDPPAQVLQELNVTVVTFFCRPHNICTFVPR | (PR3_9_110) |
| | RKAGICFGDSGGPLICDGIIQGIDSFVIWGCATRLFPDFFTRVA | Amino acid |
| | LYVDWIRSTLRESKYGPPCPPCPMIYIWAPLAGT | |
| | CGVLLLSLVITKRGRKKLLYIFKQPFMRPVQTTQEEDGCSCR | |
| | FPEEEEGGCELRVKFSRSADAPAYQQGQNQLYNELNLGRRE | |
| | EYDVLDKRRGRDPEMGGKPRRKNPQEGLYNELQKDKMAEA | |
| | YSEIGMKGERRRGKGHDGLYQGLSTATKDTYDALHMQALP | |
| | PR | |
| 111 | IVGGHEAQPHSRPYMASLQMRGNPGSHFCGGTLIHPSFVLTA | PR3_10 |
| | AACLRDIPQRLVNVVLGAHNVRTQEPTQQHFSVAQVFLNNY | (H44A)/(G4S)3/ |
| | DAENKLNDVLLIQLSSPANLSASVATVQLPQQDQPVPHGTQC | CD8aTM CAAR |
| | LAMGWGRVGAHDPPAQVLQELNVTVVTFFCRPHNICTFVPR | (PR3_10_111) |
| | RKAGICFGDSGGPLICDGIIQGIDSFVIWGCATRLFPDFFTRVA | Amino acid |
| | LYVDWIRSTLRGGGGSGGGGSGGGGSIYIWAPLAGTCGVLL | |
| | LSLVITKRGRKKLLYIFKQPFMRPVQTTQEEDGCSCRFPEEEE | |
| | GGCELRVKFSRSADAPAYQQGQNQLYNELNLGRREEYDVL | |
| | DKRRGRDPEMGGKPRRKNPQEGLYNELQKDKMAEAYSEIG | |
| | MKGERRRGKGHDGLYQGLSTATKDTYDALHMQALPPR | |
| 112 | IVGGHEAQPHSRPYMASLQMRGNPGSHFCGGTLIHPSFVLTA | PR3_10 |
| | AACLRDIPQRLVNVVLGAHNVRTQEPTQQHFSVAQVFLNNY | (H44A)/(G4S)3/ |
| | DAENKLNDVLLIQLSSPANLSASVATVQLPQQDQPVPHGTQC | CD28TM CAAR |
| | LAMGWGRVGAHDPPAQVLQELNVTVVTFFCRPHNICTFVPR | (PR3_10_112) |
| | RKAGICFGDSGGPLICDGIIQGIDSFVIWGCATRLFPDFFTRVA | Amino acid |
| | LYVDWIRSTLRGGGGSGGGGSGGGGSFWVLVVVGGVLACY | |
| | SLLVTVAFIIFWVKRGRKKLLYIFKQPFMRPVQTTQEEDGCS | |
| | CRFPEEEEGGCELRVKFSRSADAPAYQQGQNQLYNELNLGR | |
| | REEYDVLDKRRGRDPEMGGKPRRKNPQEGLYNELQKDKMA | |
| | EAYSEIGMKGERRRGKGHDGLYQGLSTATKDTYDALHMQA | |
| | LPPR | |
| 113 | IVGGHEAQPHSRPYMASLQMRGNPGSHFCGGTLIHPSFVLTA | PR3_10 |
| | AACLRDIPQRLVNVVLGAHNVRTQEPTQQHFSVAQVFLNNY | (H44A)/(G4S)/ |
| | DAENKLNDVLLIQLSSPANLSASVATVQLPQQDQPVPHGTQC | CD8aTM CAAR |
| | LAMGWGRVGAHDPPAQVLQELNVTVVTFFCRPHNICTFVPR | (PR3_10_113) |
| | RKAGICFGDSGGPLICDGIIQGIDSFVIWGCATRLFPDFFTRVA | Amino acid |
| | LYVDWIRSTLRGGGGSIYIWAPLAGTCGVLLLSLVITKRGRK | |
| | KLLYIFKQPFMRPVQTTQEEDGCSCRFPEEEEGGCELRVKFS | |
| | RSADAPAYQQGQNQLYNELNLGRREEYDVLDKRRGRDPEM | |
| | GGKPRRKNPQEGLYNELQKDKMAEAYSEIGMKGERRRGKG | |
| | HDGLYQGLSTATKDTYDALHMQALPPR | |
| 114 | IVGGHEAQPHSRPYMASLQMRGNPGSHFCGGTLIHPSFVLTA | PR3_10 |
| | AACLRDIPQRLVNVVLGAHNVRTQEPTQQHFSVAQVFLNNY | (H44A)/(G4S)/ |
| | DAENKLNDVLLIQLSSPANLSASVATVQLPQQDQPVPHGTQC | 2CD8TM CAAR |
| | LAMGWGRVGAHDPPAQVLQELNVTVVTFFCRPHNICTFVPR | (PR3_10_114) |
| | RKAGICFGDSGGPLICDGIIQGIDSFVIWGCATRLFPDFFTRVA | Amino acid |
| | LYVDWIRSTLRGGGGSFWVLVVVGGVLACYSLLVTVAFIIF | |
| | WVKRGRKKLLYIFKQPFMRPVQTTQEEDGCSCRFPEEEEGG | |
| | CELRVKFSRSADAPAYQQGQNQLYNELNLGRREEYDVLDK | |
| | RRGRDPEMGGKPRRKNPQEGLYNELQKDKMAEAYSEIGMK | |
| | GERRRGKGHDGLYQGLSTATKDTYDALHMQALPPR | |
| 115 | IVGGHEAQPHSRPYMASLQMRGNPGSHFCGGTLIHPSFVLTA | PR3_10 |
| | AACLRDIPQRLVNVVLGAHNVRTQEPTQQHFSVAQVFLNNY | (H44A)/CD8a/ |
| | DAENKLNDVLLIQLSSPANLSASVATVQLPQQDQPVPHGTQC | CD8aTM CAAR |
| | LAMGWGRVGAHDPPAQVLQELNVTVVTFFCRPHNICTFVPR | (PR3_10_115) |
| | RKAGICFGDSGGPLICDGIIQGIDSFVIWGCATRLFPDFFTRVA | Amino acid |
| | LYVDWIRSTLRKPTTTPAPRPPTPAPTIASQPLSLRPEACRPAA | |
| | GGAVHTRGLDFACDIYIWAPLAGTCGVLLLSLVITKRGRKKL | |
| | LYIFKQPFMRPVQTTQEEDGCSCRFPEEEEGGCELRVKFSRSA | |
| | DAPAYQQGQNQLYNELNLGRREEYDVLDKRRGRDPEMGGK | |
| | PRRKNPQEGLYNELQKDKMAEAYSEIGMKGERRRGKGHDG | |
| | LYQGLSTATKDTYDALHMQALPPR | |
| 116 | IVGGHEAQPHSRPYMASLQMRGNPGSHFCGGTLIHPSFVLTA | PR3_10 |
| | AACLRDIPQRLVNVVLGAHNVRTQEPTQQHFSVAQVFLNNY | (H44A)/EAAAK/ |
| | DAENKLNDVLLIQLSSPANLSASVATVQLPQQDQPVPHGTQC | CD8aTM CAAR |
| | LAMGWGRVGAHDPPAQVLQELNVTVVTFFCRPHNICTFVPR | (PR3_10_116) |
| | RKAGICFGDSGGPLICDGIIQGIDSFVIWGCATRLFPDFFTRVA | Amino acid |
| | LYVDWIRSTLREAAAKIYIWAPLAGTCGVLLLSLVITKRGRK | |
| | KLLYIFKQPFMRPVQTTQEEDGCSCRFPEEEEGGCELRVKFS | |
| | RSADAPAYQQGQNQLYNELNLGRREEYDVLDKRRGRDPEM | |
| | GGKPRRKNPQEGLYNELQKDKMAEAYSEIGMKGERRRGKG | |
| | HDGLYQGLSTATKDTYDALHMQALPPR | |
| 117 | IVGGHEAQPHSRPYMASLQMRGNPGSHFCGGTLIHPSFVLTA | PR3_10 |
| | AACLRDIPQRLVNVVLGAHNVRTQEPTQQHFSVAQVFLNNY | (H44A)/MS/ |
| | DAENKLNDVLLIQLSSPANLSASVATVQLPQQDQPVPHGTQC | CD8aTM CAAR |
| | LAMGWGRVGAHDPPAQVLQELNVTVVTFFCRPHNICTFVPR | (PR3_10_117) |
| | RKAGICFGDSGGPLICDGIIQGIDSFVIWGCATRLFPDFFTRVA | Amino acid |
| | LYVDWIRSTLRESKYGPPCPPCPGQPREPQVYTLPPSQEEMT | |
| | KNQVSLTCLVKGFYPSDIAVEWESNGQPENNYKTTPPVLDS | |
| | DGSFFLYSRLTVDKSRWQEGNVFSCSVMHEALHNHYTQKSL | |
| | SLSLGKMIYIWAPLAGTCGVLLLSLVITKRGRKKLLYIFKQPF | |
| | MRPVQTTQEEDGCSCRFPEEEEGGCELRVKFSRSADAPAYQ | |
| | QGQNQLYNELNLGRREEYDVLDKRRGRDPEMGGKPRRKNP | |
| | QEGLYNELQKDKMAEAYSEIGMKGERRRGKGHDGLYQGLS | |
| | TATKDTYDALHMQALPPR | |
| 118 | IVGGHEAQPHSRPYMASLQMRGNPGSHFCGGTLIHPSFVLTA | PR3_10 |
| | AACLRDIPQRLVNVVLGAHNVRTQEPTQQHFSVAQVFLNNY | (H44A)/SS/ |
| | DAENKLNDVLLIQLSSPANLSASVATVQLPQQDQPVPHGTQC | CD8aTM CAAR |
| | LAMGWGRVGAHDPPAQVLQELNVTVVTFFCRPHNICTFVPR | (PR3_10_118) |
| | RKAGICFGDSGGPLICDGIIQGIDSFVIWGCATRLFPDFFTRVA | Amino acid |
| | LYVDWIRSTLRESKYGPPCPPCPMIYIWAPLAGTCGVLLLSL | |
| | VITKRGRKKLLYIFKQPFMRPVQTTQEEDGCSCRFPEEEEGG | |
| | CELRVKFSRSADAPAYQQGQNQLYNELNLGRREEYDVLDK | |
| | RRGRDPEMGGKPRRKNPQEGLYNELQKDKMAEAYSEIGMK | |
| | GERRRGKGHDGLYQGLSTATKDTYDALHMQALPPR | |
| 119 | AEIVGGHEAQPHSRPYMASLQMRGNPGSHFCGGTLIHPSFVL | PR3_10 |
| | TAAACLRDIPQRLVNVVLGAHNVRTQEPTQQHFSVAQVFLN | (H44A)/(G4S)3/ |
| | NYDAENKLNDVLLIQLSSPANLSASVATVQLPQQDQPVPHG | CD8aTM CAAR |
| | TQCLAMGWGRVGAHDPPAQVLQELNVTVVTFFCRPHNICTF | (PR3_10_111 |
| | VPRRKAGICFGDSGGPLICDGIIQGIDSFVIWGCATRLFPDFFT | PR3SP) |
| | RVALYVDWIRSTLRGGGGSGGGGSGGGGSIYIWAPLAGTCG | Amino acid |
| | VLLLSLVITKRGRKKLLYIFKQPFMRPVQTTQEEDGCSCRFPE | |
| | EEEGGCELRVKFSRSADAPAYQQGQNQLYNELNLGRREEYD | |
| | VLDKRRGRDPEMGGKPRRKNPQEGLYNELQKDKMAEAYSE | |
| | IGMKGERRRGKGHDGLYQGLSTATKDTYDALHMQALPPR | |
| 120 | AEIVGGHEAQPHSRPYMASLQMRGNPGSHFCGGTLIHPSFVL | PR3_10 |
| | TAAACLRDIPQRLVNVVLGAHNVRTQEPTQQHFSVAQVFLN | (H44A)/(G4S)3/ |
| | NYDAENKLNDVLLIQLSSPANLSASVATVQLPQQDQPVPHG | CD28TM CAAR |
| | TQCLAMGWGRVGAHDPPAQVLQELNVTVVTFFCRPHNICTF | (PR3_10_112 |
| | VPRRKAGICFGDSGGPLICDGIIQGIDSFVIWGCATRLFPDFFT | PR3SP) |
| | RVALYVDWIRSTLRGGGGSGGGGSGGGGSFWVLVVVGGVL | Amino acid |
| | ACYSLLVTVAFIIFWVKRGRKKLLYIFKQPFMRPVQTTQEED | |
| | GCSCRFPEEEEGGCELRVKFSRSADAPAYQQGQNQLYNELN | |
| | LGRREEYDVLDKRRGRDPEMGGKPRRKNPQEGLYNELQKD | |
| | KMAEAYSEIGMKGERRRGKGHDGLYQGLSTATKDTYDALH | |
| | MQALPPR | |
| 121 | AEIVGGHEAQPHSRPYMASLQMRGNPGSHFCGGTLIHPSFVL | PR3_10 |
| | TAAACLRDIPQRLVNVVLGAHNVRTQEPTQQHFSVAQVFLN | (H44A)/(G4S)/ |
| | NYDAENKLNDVLLIQLSSPANLSASVATVQLPQQDQPVPHG | CD8aTM CAAR |
| | TQCLAMGWGRVGAHDPPAQVLQELNVTVVTFFCRPHNICTF | (PR3_10_113 |
| | VPRRKAGICFGDSGGPLICDGIIQGIDSFVIWGCATRLFPDFFT | PR3SP) |
| | RVALYVDWIRSTLRGGGGSIYIWAPLAGTCGVLLLSLVITKR | Amino acid |
| | GRKKLLYIFKQPFMRPVQTTQEEDGCSCRFPEEEEGGCELRV | |
| | KFSRSADAPAYQQGQNQLYNELNLGRREEYDVLDKRRGRD | |
| | PEMGGKPRRKNPQEGLYNELQKDKMAEAYSEIGMKGERRR | |
| | GKGHDGLYQGLSTATKDTYDALHMQALPPR | |
| 122 | AEIVGGHEAQPHSRPYMASLQMRGNPGSHFCGGTLIHPSFVL | PR3_10 |
| | TAAACLRDIPQRLVNVVLGAHNVRTQEPTQQHFSVAQVFLN | (H44A)/(G4S)/ |
| | NYDAENKLNDVLLIQLSSPANLSASVATVQLPQQDQPVPHG | CD28TM CAAR |
| | TQCLAMGWGRVGAHDPPAQVLQELNVTVVTFFCRPHNICTF | (PR3_10_114 |
| | VPRRKAGICFGDSGGPLICDGIIQGIDSFVIWGCATRLFPDFFT | PR3SP) |
| | RVALYVDWIRSTLRGGGGSFWVLVVVGGVLACYSLLVTVA | Amino acid |
| | FIIFWVKRGRKKLLYIFKQPFMRPVQTTQEEDGCSCRFPEEEE | |
| | GGCELRVKFSRSADAPAYQQGQNQLYNELNLGRREEYDVL | |
| | DKRRGRDPEMGGKPRRKNPQEGLYNELQKDKMAEAYSEIG | |
| | MKGERRRGKGHDGLYQGLSTATKDTYDALHMQALPPR | |
| 123 | MAHRPPSPALASVLLALLLSGAARA | PR3 Signal |
| | | Peptide |
| | | Amino Acid |
| 124 | X₁PPX₂P, X1 is glycine, cysteine or arginine and X2 is | Hinge consensus |
| | cysteine or threonine | |
| 125 | EPKSCDKTHTCPPCP | Hinge |
| 126 | ERKCCVECPPCP | Hinge |
| 127 | ELKTPLGDTHTCPRCPEPKSCDTPPPCPRCPEPKSCDTPPPCPR | Hinge |
| | CPEPKSCDTPPPCPRCP | |
| 128 | ESKYGPPCPSCP | Hinge |
| 129 | YGPPCPPCP | Hinge |
| 130 | KYGPPCPPCP | Hinge |
| 131 | EVVVKYGPPCPPCP | Hinge |
| 132 | ASTKGPSVFPLAPCSRSTSESTAALGCLVKDYFPEPVTVSWN | Human IgG2 Fc |
| | SGALTSGVHTFPAVLQSSGLYSLSSVVTVPSSNFGTQTYTCN | (Uniprot P01859) |
| | VDHKPSNTKVDKTVERKCCVECPPCPAPPVAGPSVFLFPPKP | |
| | KDTLMISRTPEVTCVVVDVSHEDPEVQFNWYVDGVEVHNA | |
| | KTKPREEQFNSTFRVVSVLTVVHQDWLNGKEYKCKVSNKG | |
| | LPAPIEKTISKTKGQPREPQVYTLPPSREEMTKNQVSLTCLVK | |
| | GFYPSDISVEWESNGQPENNYKTTPPMLDSDGSFFLYSKLTV | |
| | DKSRWQQGNVFSCSVMHEALHNHYTQKSLSLSPGK | |
| 133 | ASTKGPSVFPLAPCSRSTSESTAALGCLVKDYFPEPVTVSWN | Human IgG4 Fc |
| | SGALTSGVHTFPAVLQSSGLYSLSSVVTVPSSSLGTKTYTCN | (Uniprot P01861) |
| | VDHKPSNTKVDKRVESKYGPPCPSCPAPEFLGGPSVFLFPPKP | |
| | KDTLMISRTPEVTCVVVDVSQEDPEVQFNWYVDGVEVHNA | |
| | KTKPREEQFNSTYRVVSVLTVLHQDWLNGKEYKCKVSNKG | |
| | LPSSIEKTISKAKGQPREPQVYTLPPSQEEMTKNQVSLTCLVK | |
| | GFYPSDIAVEWESNGQPENNYKTTPPVLDSDGSFFLYSRLTV | |
| | DKSRWQEGNVFSCSVMHEALHNHYTQKSLSLSLGK | |
| 134 | MFWVLVVVGGVLACYSLLVTVAFIIFWV | CD28 |
| | | transmembrane |
| | | domain (aa) |
| 135 | MIYIWAPLAGTCGVLLLSLVIT | CD8a |
| | | transmembrane |
| | | domain (aa) |
| 136 | RVKFSRSAEPPAYQQGQNQLYNELNLGRREEYDVLDKRRGR | CD3-zeta derived |
| | DPEMGGKPRRKNPQEGLYNELQKDKMAEAYSEIGMKGERR | intracellular |
| | RGKGHDGLYQGLSTATKDTYDALHMQALPPR | signaling domain |
| | | (aa) |
| 137 | RVKFSRSADAPAYKQGQNQLYNELNLGRREEYDVLDKRRG | CD3-zeta derived |
| | RDPEMGGKPRRKNPQEGLYNELQKDKMAEAYSEIGMKGER | intracellular |
| | RRGKGHDGLYQGLSTATKDTYDALHMQALPPR | signaling domain |
| | | (aa) |
| 138 | RSKRSRLLHSDYMNMTPRRPGPTRKHYQPYAPPRDFAAYRS | CD28 (amino |
| | | acids 180-220 of |
| | | P10747) |
| 139 | AGGAGTAAGAGGAGCAGGCTCCTGCACAGTGACTACATG | CD28 |
| | AACATGACTCCCCGCCGCCCCGGGCCCACCCGCAAGCATT | endodomain (nt) |
| | ACCAGCCCTATGCCCCACCACGCGACTTCGCAGCCTATCG | |
| | CTCC | |
| 140 | RSKRSRGGHSDYMNMTPRRPGPTRKHYQPYAPPRDFAAYRS | CD28 (LL to GG) |
| 141 | MLLLVTSLLLCELPHPAFLLIPRKVCNGIGIGEFKDSLSINATN | tEGFR |
| | IKHFKNCTSISGDLHILPVAFRGDSFTHTPPLDPQELDILKTVK | |
| | EITGFLLIQAWPENRTDLHAFENLEIIRGRTKQHGQFSLAVVS | |
| | LNITSLGLRSLKEISDGDVIISGNKNLCYANTINWKKLFGTSG | |
| | QKTKIISNRGENSCKATGQVCHALCSPEGCWGPEPRDCVSCR | |
| | NVSRGRECVDKCNLLEGEPREFVENSECIQCHPECLPQAMNI | |
| | TCTGRGPDNCIQCAHYIDGPHCVKTCPAGVMGENNTLVWK | |
| | YADAGHVCHLCHPNCTYGCTGPGLEGCPTNGPKIPSIATGM | |
| | VGALLLLLVVALGIGLFM | |
| 142 | RKVCNGIGIGEFKDSLSINATNIKHFKNCTSISGDLHILPVAFR | tEGFR |
| | GDSFTHTPPLDPQELDILKTVKEITGFLLIQAWPENRTDLHAF | |
| | ENLEIIRGRTKQHGQFSLAVVSLNITSLGLRSLKEISDGDVIIS | |
| | GNKNLCYANTINWKKLFGTSGQKTKIISNRGENSCKATGQV | |
| | CHALCSPEGCWGPEPRDCVSCRNVSRGRECVDKCNLLEGEP | |
| | REFVENSECIQCHPECLPQAMNITCTGRGPDNCIQCAHYIDGP | |
| | HCVKTCPAGVMGENNTLVWKYADAGHVCHLCHPNCTYGC | |
| | TGPGLEGCPTNGPKIPSIATGMVGALLLLLVVALGIGLFM | |
| 143 | MLLLVTSLLLCELPHPAFLLIPRKVCNGIGIGEFKDSLSINATN | truncated EGFR |
| | IKHFKNCTSISGDLHILPVAFRGDSFTHTPPLDPQELDILKTVK | (tEGFR) sequence |
| | EITGFLLIQAWPENRTDLHAFENLEIIRGRTKQHGQFSLAVVS | (aa) |
| | LNITSLGLRSLKEISDGDVIISGNKNLCYANTINWKKLFGTSG | |
| | QKTKIISNRGENSCKATGQVCHALCSPEGCWGPEPRDCVSCR | |
| | NVSRGRECVDKCNLLEGEPREFVENSECIQCHPECLPQAMNI | |
| | TCTGRGPDNCIQCAHYIDGPHCVKTCPAGVMGENNTLVWK | |
| | YADAGHVCHLCHPNCTYGCTGPGLEGCPTNGPKIPSIATGM | |
| | VGALLLLLVVALGIGLFM | |
| 144 | ATCGTTGGACCCCATGAAGCCCAGCCTCACAGCAGACCTT | PR3_9 |
| | ACATGGCCAGCCTGCAGATGAGAGGCAATCCCGGCTCTCA | (G4P)/(G4S)3/ |
| | CTTTTGTGGCGGCACCCTGATTCACCCCTCCTTCGTTCTGA | CD8aTM CAAR |
| | CCGCCGCTCACTGCCTGAGAGACATCCCTCAGAGACTGGT | (PR3_9_103) |
| | CAACGTGGTGCTGGGAGCCCACAACGTGCGGACACAAGA | Nucleotide |
| | ACCTACACAGCAGCACTTCAGCGTGGCCCAGGTGTTCCTG | |
| | AACAACTACGACGCCGAGAACAAGCTGAACGACGTGCTG | |
| | CTGATCCAGCTGAGCAGCCCTGCTAATCTGTCTGCCTCTGT | |
| | GGCCACTGTGCAGCTGCCTCAGCAAGATCAGCCTGTGCCT | |
| | CACGGCACACAGTGTCTGGCTATGGGATGGGGAAGAGTG | |
| | GGCGCTCATGATCCTCCAGCTCAAGTGCTGCAAGAGCTGA | |
| | ACGTGACCGTCGTGACCTTCTTCTGCCGGCCTCACAACAT | |
| | CTGCACCTTCGTGCCCAGAAGAAAGGCCGGCATCTGCTTC | |
| | GGAGATTCTGGCGGCCCTCTGATCTGCGACGGAATCATCC | |
| | AGGGCATCGACAGCTTCGTGATCTGGGGCTGTGCCACCAG | |
| | ACTGTTCCCCGACTTCTTTACCCGGGTGGCCCTCTACGTGG | |
| | ACTGGATCCGGTCTACACTGAGAGGCGGTGGCGGTTCTGG | |
| | TGGCGGTGGCTCCGGCGGTGGAGGTTCAATCTATATCTGG | |
| | GCACCTCTGGCCGGAACATGTGGCGTGCTGCTGCTGTCTC | |
| | TGGTCATCACCAAACGGGGCAGAAAGAAACTCCTGTATAT | |
| | ATTCAAACAACCATTTATGAGACCAGTACAAACTACTCAA | |
| | GAGGAAGATGGCTGTAGCTGCCGATTTCCAGAAGAAGAA | |
| | GAAGGAGGATGTGAACTGCGGGTGAAGTTCAGCAGAAGC | |
| | GCCGACGCCCCTGCCTACCAGCAGGGCCAGAATCAGCTGT | |
| | ACAACGAGCTGAACCTGGGGAGAAGAGAAGAGTACGACG | |
| | TGCTGGATAAGCGGAGAGGCAGAGATCCTGAAATGGGCG | |
| | GCAAGCCCAGACGGAAGAATCCTCAAGAGGGCCTGTATA | |
| | ATGAGCTGCAGAAAGACAAGATGGCCGAGGCCTACAGCG | |
| | AGATCGGAATGAAGGGCGAGCGCAGAAGAGGCAAGGGA | |
| | CACGATGGACTGTACCAGGGCCTGAGCACCGCCACCAAG | |
| | GATACCTATGACGCACTGCACATGCAGGCCCTGCCACCTA | |
| | GA | |
| 145 | ATCGTTGGACCCCATGAAGCCCAGCCTCACAGCAGACCTT | PR3_9 |
| | ACATGGCCAGCCTGCAGATGAGAGGCAATCCCGGCTCTCA | (G4P)/(G4S)3/ |
| | CTTTTGTGGCGGCACCCTGATTCACCCCTCCTTCGTTCTGA | CD28TM CAAR |
| | CCGCCGCTCACTGCCTGAGAGACATCCCTCAGAGACTGGT | (PR3_9_104) |
| | CAACGTGGTGCTGGGAGCCCACAACGTGCGGACACAAGA | Nucleotide |
| | ACCTACACAGCAGCACTTCAGCGTGGCCCAGGTGTTCCTG | |
| | AACAACTACGACGCCGAGAACAAGCTGAACGACGTGCTG | |
| | CTGATCCAGCTGAGCAGCCCTGCTAATCTGTCTGCCTCTGT | |
| | GGCCACTGTGCAGCTGCCTCAGCAAGATCAGCCTGTGCCT | |
| | CACGGCACACAGTGTCTGGCTATGGGATGGGGAAGAGTG | |
| | GGCGCTCATGATCCTCCAGCTCAAGTGCTGCAAGAGCTGA | |
| | ACGTGACCGTCGTGACCTTCTTCTGCCGGCCTCACAACAT | |
| | CTGCACCTTCGTGCCCAGAAGAAAGGCCGGCATCTGCTTC | |
| | GGAGATTCTGGCGGCCCTCTGATCTGCGACGGAATCATCC | |
| | AGGGCATCGACAGCTTCGTGATCTGGGGCTGTGCCACCAG | |
| | ACTGTTCCCCGACTTCTTTACCCGGGTGGCCCTCTACGTGG | |
| | ACTGGATCCGGTCTACACTGAGAGGCGGTGGCGGTTCTGG | |
| | TGGCGGTGGCTCCGGCGGTGGAGGTTCATTCTGGGTGCTC | |
| | GTGGTCGTTGGCGGAGTGCTGGCCTGTTACAGCCTGCTGG | |
| | TTACCGTGGCCTTCATCATCTTTTGGGTCAAGCGGGGCAG | |
| | AAAGAAGCTGCTCTACATCTTCAAGCAGCCCTTCATGCGG | |
| | CCCGTGCAGACCACACAAGAGGAAGATGGCTGCTCCTGC | |
| | AGATTCCCCGAGGAAGAAGAAGGCGGCTGCGAGCTGAGA | |
| | GTGAAGTTCAGCAGATCCGCCGACGCTCCAGCCTATCAGC | |
| | AGGGCCAAAACCAGCTGTACAACGAGCTGAACCTGGGGA | |
| | GAAGAGAAGAGTACGACGTGCTGGATAAGCGGAGAGGCA | |
| | GAGATCCTGAAATGGGCGGCAAGCCCAGACGGAAGAATC | |
| | CTCAAGAGGGCCTGTATAATGAGCTGCAGAAAGACAAGA | |
| | TGGCCGAGGCCTACAGCGAGATCGGAATGAAGGGCGAGC | |
| | GCAGAAGAGGCAAGGGACACGATGGACTGTACCAGGGCC | |
| | TGAGCACCGCCACCAAGGATACCTATGACGCACTGCACAT | |
| | GCAGGCCCTGCCACCTAGA | |
| 146 | ATCGTTGGACCCCATGAAGCCCAGCCTCACAGCAGACCTT | PR3_9 |
| | ACATGGCCAGCCTGCAGATGAGAGGCAATCCCGGCTCTCA | (G4P)/(G4S)/ |
| | CTTTTGTGGCGGCACCCTGATTCACCCCTCCTTCGTTCTGA | CD8aTM CAAR |
| | CCGCCGCTCACTGCCTGAGAGACATCCCTCAGAGACTGGT | (PR3_9_105) |
| | CAACGTGGTGCTGGGAGCCCACAACGTGCGGACACAAGA | Nucleotide |
| | ACCTACACAGCAGCACTTCAGCGTGGCCCAGGTGTTCCTG | |
| | AACAACTACGACGCCGAGAACAAGCTGAACGACGTGCTG | |
| | CTGATCCAGCTGAGCAGCCCTGCTAATCTGTCTGCCTCTGT | |
| | GGCCACTGTGCAGCTGCCTCAGCAAGATCAGCCTGTGCCT | |
| | CACGGCACACAGTGTCTGGCTATGGGATGGGGAAGAGTG | |
| | GGCGCTCATGATCCTCCAGCTCAAGTGCTGCAAGAGCTGA | |
| | ACGTGACCGTCGTGACCTTCTTCTGCCGGCCTCACAACAT | |
| | CTGCACCTTCGTGCCCAGAAGAAAGGCCGGCATCTGCTTC | |
| | GGAGATTCTGGCGGCCCTCTGATCTGCGACGGAATCATCC | |
| | AGGGCATCGACAGCTTCGTGATCTGGGGCTGTGCCACCAG | |
| | ACTGTTCCCCGACTTCTTTACCCGGGTGGCCCTCTACGTGG | |
| | ACTGGATCCGGTCTACACTGAGAGGCGGTGGAGGTTCAAT | |
| | CTATATCTGGGCACCTCTGGCCGGAACATGTGGCGTGCTG | |
| | CTGCTGTCTCTGGTCATCACCAAACGGGGCAGAAAGAAAC | |
| | TCCTGTATATATTCAAACAACCATTTATGAGACCAGTACA | |
| | AACTACTCAAGAGGAAGATGGCTGTAGCTGCCGATTTCCA | |
| | GAAGAAGAAGAAGGAGGATGTGAACTGCGGGTGAAGTTC | |
| | AGCAGAAGCGCCGACGCCCCTGCCTACCAGCAGGGCCAG | |
| | AATCAGCTGTACAACGAGCTGAACCTGGGGAGAAGAGAA | |
| | GAGTACGACGTGCTGGATAAGCGGAGAGGCAGAGATCCT | |
| | GAAATGGGCGGCAAGCCCAGACGGAAGAATCCTCAAGAG | |
| | GGCCTGTATAATGAGCTGCAGAAAGACAAGATGGCCGAG | |
| | GCCTACAGCGAGATCGGAATGAAGGGCGAGCGCAGAAGA | |
| | GGCAAGGGACACGATGGACTGTACCAGGGCCTGAGCACC | |
| | GCCACCAAGGATACCTATGACGCACTGCACATGCAGGCCC | |
| | TGCCACCTAGA | |
| 147 | ATCGTTGGACCCCATGAAGCCCAGCCTCACAGCAGACCTT | PR3_9 |
| | ACATGGCCAGCCTGCAGATGAGAGGCAATCCCGGCTCTCA | (G4P)/(G4S)/ |
| | CTTTTGTGGCGGCACCCTGATTCACCCCTCCTTCGTTCTGA | CD28TM CAAR |
| | CCGCCGCTCACTGCCTGAGAGACATCCCTCAGAGACTGGT | (PR3_9_106) |
| | CAACGTGGTGCTGGGAGCCCACAACGTGCGGACACAAGA | Nucleotide |
| | ACCTACACAGCAGCACTTCAGCGTGGCCCAGGTGTTCCTG | |
| | AACAACTACGACGCCGAGAACAAGCTGAACGACGTGCTG | |
| | CTGATCCAGCTGAGCAGCCCTGCTAATCTGTCTGCCTCTGT | |
| | GGCCACTGTGCAGCTGCCTCAGCAAGATCAGCCTGTGCCT | |
| | CACGGCACACAGTGTCTGGCTATGGGATGGGGAAGAGTG | |
| | GGCGCTCATGATCCTCCAGCTCAAGTGCTGCAAGAGCTGA | |
| | ACGTGACCGTCGTGACCTTCTTCTGCCGGCCTCACAACAT | |
| | CTGCACCTTCGTGCCCAGAAGAAAGGCCGGCATCTGCTTC | |
| | GGAGATTCTGGCGGCCCTCTGATCTGCGACGGAATCATCC | |
| | AGGGCATCGACAGCTTCGTGATCTGGGGCTGTGCCACCAG | |
| | ACTGTTCCCCGACTTCTTTACCCGGGTGGCCCTCTACGTGG | |
| | ACTGGATCCGGTCTACACTGAGAGGCGGTGGAGGTTCATT | |
| | CTGGGTGCTCGTGGTCGTTGGCGGAGTGCTGGCCTGTTAC | |
| | AGCCTGCTGGTTACCGTGGCCTTCATCATCTTTTGGGTCAA | |
| | GCGGGGCAGAAAGAAGCTGCTCTACATCTTCAAGCAGCCC | |
| | TTCATGCGGCCCGTGCAGACCACACAAGAGGAAGATGGC | |
| | TGCTCCTGCAGATTCCCCGAGGAAGAAGAAGGCGGCTGC | |
| | GAGCTGAGAGTGAAGTTCAGCAGATCCGCCGACGCTCCA | |
| | GCCTATCAGCAGGGCCAAAACCAGCTGTACAACGAGCTG | |
| | AACCTGGGGAGAAGAGAAGAGTACGACGTGCTGGATAAG | |
| | CGGAGAGGCAGAGATCCTGAAATGGGCGGCAAGCCCAGA | |
| | CGGAAGAATCCTCAAGAGGGCCTGTATAATGAGCTGCAG | |
| | AAAGACAAGATGGCCGAGGCCTACAGCGAGATCGGAATG | |
| | AAGGGCGAGCGCAGAAGAGGCAAGGGACACGATGGACTG | |
| | TACCAGGGCCTGAGCACCGCCACCAAGGATACCTATGACG | |
| | CACTGCACATGCAGGCCCTGCCACCTAGA | |
| 148 | ATCGTTGGACCCCATGAAGCCCAGCCTCACAGCAGACCTT | PR3_9 |
| | ACATGGCCAGCCTGCAGATGAGAGGCAATCCCGGCTCTCA | (G4P)/CD8a/ |
| | CTTTTGTGGCGGCACCCTGATTCACCCCTCCTTCGTTCTGA | CD8aTM CAAR |
| | CCGCCGCTCACTGCCTGAGAGACATCCCTCAGAGACTGGT | (PR3_9_107) |
| | CAACGTGGTGCTGGGAGCCCACAACGTGCGGACACAAGA | Nucleotide |
| | ACCTACACAGCAGCACTTCAGCGTGGCCCAGGTGTTCCTG | |
| | AACAACTACGACGCCGAGAACAAGCTGAACGACGTGCTG | |
| | CTGATCCAGCTGAGCAGCCCTGCTAATCTGTCTGCCTCTGT | |
| | GGCCACTGTGCAGCTGCCTCAGCAAGATCAGCCTGTGCCT | |
| | CACGGCACACAGTGTCTGGCTATGGGATGGGGAAGAGTG | |
| | GGCGCTCATGATCCTCCAGCTCAAGTGCTGCAAGAGCTGA | |
| | ACGTGACCGTCGTGACCTTCTTCTGCCGGCCTCACAACAT | |
| | CTGCACCTTCGTGCCCAGAAGAAAGGCCGGCATCTGCTTC | |
| | GGAGATTCTGGCGGCCCTCTGATCTGCGACGGAATCATCC | |
| | AGGGCATCGACAGCTTCGTGATCTGGGGCTGTGCCACCAG | |
| | ACTGTTCCCCGACTTCTTTACCCGGGTGGCCCTCTACGTGG | |
| | ACTGGATCCGGTCTACACTGAGAAAGCCTACCACAACCCC | |
| | AGCACCTAGACCCCCTACACCAGCACCAACCATCGCCTCT | |
| | CAGCCACTGAGCCTGCGGCCCGAGGCCTGTAGGCCAGCA | |
| | GCAGGAGGAGCCGTGCACACCAGGGGCCTGGACTTTGCCT | |
| | GCGATATCTATATCTGGGCACCTCTGGCCGGAACATGTGG | |
| | CGTGCTGCTGCTGTCTCTGGTCATCACCAAGCGGGGCAGA | |
| | AAGAAGCTGCTCTACATCTTCAAGCAGCCCTTCATGCGGC | |
| | CCGTGCAGACCACACAAGAGGAAGATGGCTGCTCCTGCA | |
| | GATTCCCCGAGGAAGAAGAAGGCGGCTGCGAGCTGAGAG | |
| | TGAAGTTCAGCAGATCCGCCGACGCTCCAGCCTATCAGCA | |
| | GGGCCAAAACCAGCTGTACAACGAGCTGAACCTGGGGAG | |
| | AAGAGAAGAGTACGACGTGCTGGATAAGCGGAGAGGCAG | |
| | AGATCCTGAAATGGGCGGCAAGCCCAGACGGAAGAATCC | |
| | TCAAGAGGGCCTGTATAATGAGCTGCAGAAAGACAAGAT | |
| | GGCCGAGGCCTACAGCGAGATCGGAATGAAGGGCGAGCG | |
| | CAGAAGAGGCAAGGGACACGATGGACTGTACCAGGGCCT | |
| | GAGCACCGCCACCAAGGATACCTATGACGCACTGCACATG | |
| | CAGGCCCTGCCACCTAGA | |
| | ATCGTTGGACCCCATGAAGCCCAGCCTCACAGCAGACCTT | PR3_9 |
| 149 | ACATGGCCAGCCTGCAGATGAGAGGCAATCCCGGCTCTCA | (G4P)/EAAAK/ |
| | CTTTTGTGGCGGCACCCTGATTCACCCCTCCTTCGTTCTGA | CD8aTM CAAR |
| | CCGCCGCTCACTGCCTGAGAGACATCCCTCAGAGACTGGT | (PR3_9_108) |
| | CAACGTGGTGCTGGGAGCCCACAACGTGCGGACACAAGA | Nucleotide |
| | ACCTACACAGCAGCACTTCAGCGTGGCCCAGGTGTTCCTG | |
| | AACAACTACGACGCCGAGAACAAGCTGAACGACGTGCTG | |
| | CTGATCCAGCTGAGCAGCCCTGCTAATCTGTCTGCCTCTGT | |
| | GGCCACTGTGCAGCTGCCTCAGCAAGATCAGCCTGTGCCT | |
| | CACGGCACACAGTGTCTGGCTATGGGATGGGGAAGAGTG | |
| | GGCGCTCATGATCCTCCAGCTCAAGTGCTGCAAGAGCTGA | |
| | ACGTGACCGTCGTGACCTTCTTCTGCCGGCCTCACAACAT | |
| | CTGCACCTTCGTGCCCAGAAGAAAGGCCGGCATCTGCTTC | |
| | GGAGATTCTGGCGGCCCTCTGATCTGCGACGGAATCATCC | |
| | AGGGCATCGACAGCTTCGTGATCTGGGGCTGTGCCACCAG | |
| | ACTGTTCCCCGACTTCTTTACCCGGGTGGCCCTCTACGTGG | |
| | ACTGGATCCGGTCTACACTGAGAGAAGCCGCCGCTAAAAT | |
| | CTATATCTGGGCACCTCTGGCCGGAACATGTGGCGTGCTG | |
| | CTGCTGTCTCTGGTCATCACCAAACGGGGCAGAAAGAAAC | |
| | TCCTGTATATATTCAAACAACCATTTATGAGACCAGTACA | |
| | AACTACTCAAGAGGAAGATGGCTGTAGCTGCCGATTTCCA | |
| | GAAGAAGAAGAAGGAGGATGTGAACTGCGGGTGAAGTTC | |
| | AGCAGAAGCGCCGACGCCCCTGCCTACCAGCAGGGCCAG | |
| | AATCAGCTGTACAACGAGCTGAACCTGGGGAGAAGAGAA | |
| | GAGTACGACGTGCTGGATAAGCGGAGAGGCAGAGATCCT | |
| | GAAATGGGCGGCAAGCCCAGACGGAAGAATCCTCAAGAG | |
| | GGCCTGTATAATGAGCTGCAGAAAGACAAGATGGCCGAG | |
| | GCCTACAGCGAGATCGGAATGAAGGGCGAGCGCAGAAGA | |
| | GGCAAGGGACACGATGGACTGTACCAGGGCCTGAGCACC | |
| | GCCACCAAGGATACCTATGACGCACTGCACATGCAGGCCC | |
| | TGCCACCTAGA | |
| 150 | ATCGTTGGACCCCATGAAGCCCAGCCTCACAGCAGACCTT | PR3_9 |
| | ACATGGCCAGCCTGCAGATGAGAGGCAATCCCGGCTCTCA | (G4P)/MS/CD8aTM |
| | CTTTTGTGGCGGCACCCTGATTCACCCCTCCTTCGTTCTGA | CAAR |
| | CCGCCGCTCACTGCCTGAGAGACATCCCTCAGAGACTGGT | (PR3_9_109) |
| | CAACGTGGTGCTGGGAGCCCACAACGTGCGGACACAAGA | Nucleotide |
| | ACCTACACAGCAGCACTTCAGCGTGGCCCAGGTGTTCCTG | |
| | AACAACTACGACGCCGAGAACAAGCTGAACGACGTGCTG | |
| | CTGATCCAGCTGAGCAGCCCTGCTAATCTGTCTGCCTCTGT | |
| | GGCCACTGTGCAGCTGCCTCAGCAAGATCAGCCTGTGCCT | |
| | CACGGCACACAGTGTCTGGCTATGGGATGGGGAAGAGTG | |
| | GGCGCTCATGATCCTCCAGCTCAAGTGCTGCAAGAGCTGA | |
| | ACGTGACCGTCGTGACCTTCTTCTGCCGGCCTCACAACAT | |
| | CTGCACCTTCGTGCCCAGAAGAAAGGCCGGCATCTGCTTC | |
| | GGAGATTCTGGCGGCCCTCTGATCTGCGACGGAATCATCC | |
| | AGGGCATCGACAGCTTCGTGATCTGGGGCTGTGCCACCAG | |
| | ACTGTTCCCCGACTTCTTTACCCGGGTGGCCCTCTACGTGG | |
| | ACTGGATCCGGTCTACACTGAGAGAGTCTAAATACGGACC | |
| | GCCTTGTCCTCCTTGTCCCGGCCAGCCAAGAGAGCCCCAG | |
| | GTTTACACACTGCCTCCAAGCCAAGAGGAAATGACCAAG | |
| | AATCAGGTGTCCCTGACATGCCTGGTCAAGGGCTTCTACC | |
| | CCTCCGATATCGCCGTGGAATGGGAGAGCAATGGCCAGCC | |
| | TGAGAACAACTACAAGACCACACCTCCTGTGCTGGACAGC | |
| | GACGGCAGTTTCTTCCTGTATAGTAGACTCACCGTGGATA | |
| | AATCAAGATGGCAAGAGGGCAACGTGTTCAGCTGCAGCG | |
| | TGATGCACGAGGCCCTGCACAACCACTACACCCAGAAAA | |
| | GCCTGAGCCTGTCTCTGGGCAAGATGATCTATATCTGGGC | |
| | ACCTCTGGCCGGAACATGTGGCGTGCTGCTGCTGTCTCTG | |
| | GTCATCACCAAACGGGGCAGAAAGAAACTCCTGTATATAT | |
| | TCAAACAACCATTTATGAGACCAGTACAAACTACTCAAGA | |
| | GGAAGATGGCTGTAGCTGCCGATTTCCAGAAGAAGAAGA | |
| | AGGAGGATGTGAACTGCGGGTGAAGTTCAGCAGAAGCGC | |
| | CGACGCCCCTGCCTACCAGCAGGGCCAGAATCAGCTGTAC | |
| | AACGAGCTGAACCTGGGGAGAAGAGAAGAGTACGACGTG | |
| | CTGGATAAGCGGAGAGGCAGAGATCCTGAAATGGGCGGC | |
| | AAGCCCAGACGGAAGAATCCTCAAGAGGGCCTGTATAAT | |
| | GAGCTGCAGAAAGACAAGATGGCCGAGGCCTACAGCGAG | |
| | ATCGGAATGAAGGGCGAGCGCAGAAGAGGCAAGGGACAC | |
| | GATGGACTGTACCAGGGCCTGAGCACCGCCACCAAGGAT | |
| | ACCTATGACGCACTGCACATGCAGGCCCTGCCACCTAGA | |
| 151 | ATCGTTGGACCCCATGAAGCCCAGCCTCACAGCAGACCTT | PR3_9 |
| | ACATGGCCAGCCTGCAGATGAGAGGCAATCCCGGCTCTCA | (G4P)/SS/CD8aTM |
| | CTTTTGTGGCGGCACCCTGATTCACCCCTCCTTCGTTCTGA | CAAR |
| | CCGCCGCTCACTGCCTGAGAGACATCCCTCAGAGACTGGT | (PR3_9_110) |
| | CAACGTGGTGCTGGGAGCCCACAACGTGCGGACACAAGA | Nucleotide |
| | ACCTACACAGCAGCACTTCAGCGTGGCCCAGGTGTTCCTG | |
| | AACAACTACGACGCCGAGAACAAGCTGAACGACGTGCTG | |
| | CTGATCCAGCTGAGCAGCCCTGCTAATCTGTCTGCCTCTGT | |
| | GGCCACTGTGCAGCTGCCTCAGCAAGATCAGCCTGTGCCT | |
| | CACGGCACACAGTGTCTGGCTATGGGATGGGGAAGAGTG | |
| | GGCGCTCATGATCCTCCAGCTCAAGTGCTGCAAGAGCTGA | |
| | ACGTGACCGTCGTGACCTTCTTCTGCCGGCCTCACAACAT | |
| | CTGCACCTTCGTGCCCAGAAGAAAGGCCGGCATCTGCTTC | |
| | GGAGATTCTGGCGGCCCTCTGATCTGCGACGGAATCATCC | |
| | AGGGCATCGACAGCTTCGTGATCTGGGGCTGTGCCACCAG | |
| | ACTGTTCCCCGACTTCTTTACCCGGGTGGCCCTCTACGTGG | |
| | ACTGGATCCGGTCTACACTGAGAGAGTCTAAATACGGACC | |
| | GCCTTGTCCTCCTTGTCCCATGATCTATATCTGGGCACCTC | |
| | TGGCCGGAACATGTGGCGTGCTGCTGCTGTCTCTGGTCAT | |
| | CACCAAACGGGGCAGAAAGAAACTCCTGTATATATTCAA | |
| | ACAACCATTTATGAGACCAGTACAAACTACTCAAGAGGA | |
| | AGATGGCTGTAGCTGCCGATTTCCAGAAGAAGAAGAAGG | |
| | AGGATGTGAACTGCGGGTGAAGTTCAGCAGAAGCGCCGA | |
| | CGCCCCTGCCTACCAGCAGGGCCAGAATCAGCTGTACAAC | |
| | GAGCTGAACCTGGGGAGAAGAGAAGAGTACGACGTGCTG | |
| | GATAAGCGGAGAGGCAGAGATCCTGAAATGGGCGGCAAG | |
| | CCCAGACGGAAGAATCCTCAAGAGGGCCTGTATAATGAG | |
| | CTGCAGAAAGACAAGATGGCCGAGGCCTACAGCGAGATC | |
| | GGAATGAAGGGCGAGCGCAGAAGAGGCAAGGGACACGA | |
| | TGGACTGTACCAGGGCCTGAGCACCGCCACCAAGGATACC | |
| | TATGACGCACTGCACATGCAGGCCCTGCCACCTAGA | |
| 152 | ATCGTTGGAGGACATGAAGCCCAGCCTCACAGCAGACCTT | PR3_10 |
| | ACATGGCCAGCCTGCAGATGAGAGGCAATCCCGGCTCTCA | (H44A)/(G4S)3/ |
| | CTTTTGTGGCGGCACCCTGATTCACCCCTCCTTCGTTCTGA | CD8aTM CAAR |
| | CCGCCGCTGCCTGCCTGAGAGACATCCCTCAGAGACTGGT | (PR3_10_111) |
| | CAACGTGGTGCTGGGAGCCCACAACGTGCGGACACAAGA | Nucleotide |
| | ACCTACACAGCAGCACTTCAGCGTGGCCCAGGTGTTCCTG | |
| | AACAACTACGACGCCGAGAACAAGCTGAACGACGTGCTG | |
| | CTGATCCAGCTGAGCAGCCCTGCTAATCTGTCTGCCTCTGT | |
| | GGCCACTGTGCAGCTGCCTCAGCAAGATCAGCCTGTGCCT | |
| | CACGGCACACAGTGTCTGGCTATGGGATGGGGAAGAGTG | |
| | GGCGCTCATGATCCTCCAGCTCAAGTGCTGCAAGAGCTGA | |
| | ACGTGACCGTCGTGACCTTCTTCTGCCGGCCTCACAACAT | |
| | CTGCACCTTCGTGCCCAGAAGAAAGGCCGGCATCTGCTTC | |
| | GGAGATTCTGGCGGCCCTCTGATCTGCGACGGAATCATCC | |
| | AGGGCATCGACAGCTTCGTGATCTGGGGCTGTGCCACCAG | |
| | ACTGTTCCCCGACTTCTTTACCCGGGTGGCCCTCTACGTGG | |
| | ACTGGATCCGGTCTACACTGAGAGGCGGTGGCGGTTCTGG | |
| | TGGCGGTGGCTCCGGCGGTGGAGGTTCAATCTATATCTGG | |
| | GCACCTCTGGCCGGAACATGTGGCGTGCTGCTGCTGTCTC | |
| | TGGTCATCACCAAACGGGGCAGAAAGAAACTCCTGTATAT | |
| | ATTCAAACAACCATTTATGAGACCAGTACAAACTACTCAA | |
| | GAGGAAGATGGCTGTAGCTGCCGATTTCCAGAAGAAGAA | |
| | GAAGGAGGATGTGAACTGCGGGTGAAGTTCAGCAGAAGC | |
| | GCCGACGCCCCTGCCTACCAGCAGGGCCAGAATCAGCTGT | |
| | ACAACGAGCTGAACCTGGGGAGAAGAGAAGAGTACGACG | |
| | TGCTGGATAAGCGGAGAGGCAGAGATCCTGAAATGGGCG | |
| | GCAAGCCCAGACGGAAGAATCCTCAAGAGGGCCTGTATA | |
| | ATGAGCTGCAGAAAGACAAGATGGCCGAGGCCTACAGCG | |
| | AGATCGGAATGAAGGGCGAGCGCAGAAGAGGCAAGGGA | |
| | CACGATGGACTGTACCAGGGCCTGAGCACCGCCACCAAG | |
| | GATACCTATGACGCACTGCACATGCAGGCCCTGCCACCTA | |
| | GA | |
| 153 | ATCGTTGGAGGACATGAAGCCCAGCCTCACAGCAGACCTT | PR3_10 |
| | ACATGGCCAGCCTGCAGATGAGAGGCAATCCCGGCTCTCA | (H44A)/(G4S)3/ |
| | CTTTTGTGGCGGCACCCTGATTCACCCCTCCTTCGTTCTGA | CD28TM CAAR |
| | CCGCCGCTGCCTGCCTGAGAGACATCCCTCAGAGACTGGT | (PR3_10_112) |
| | CAACGTGGTGCTGGGAGCCCACAACGTGCGGACACAAGA | Nucleotide |
| | ACCTACACAGCAGCACTTCAGCGTGGCCCAGGTGTTCCTG | |
| | AACAACTACGACGCCGAGAACAAGCTGAACGACGTGCTG | |
| | CTGATCCAGCTGAGCAGCCCTGCTAATCTGTCTGCCTCTGT | |
| | GGCCACTGTGCAGCTGCCTCAGCAAGATCAGCCTGTGCCT | |
| | CACGGCACACAGTGTCTGGCTATGGGATGGGGAAGAGTG | |
| | GGCGCTCATGATCCTCCAGCTCAAGTGCTGCAAGAGCTGA | |
| | ACGTGACCGTCGTGACCTTCTTCTGCCGGCCTCACAACAT | |
| | CTGCACCTTCGTGCCCAGAAGAAAGGCCGGCATCTGCTTC | |
| | GGAGATTCTGGCGGCCCTCTGATCTGCGACGGAATCATCC | |
| | AGGGCATCGACAGCTTCGTGATCTGGGGCTGTGCCACCAG | |
| | ACTGTTCCCCGACTTCTTTACCCGGGTGGCCCTCTACGTGG | |
| | ACTGGATCCGGTCTACACTGAGAGGCGGTGGCGGTTCTGG | |
| | TGGCGGTGGCTCCGGCGGTGGAGGTTCATTCTGGGTGCTC | |
| | GTGGTCGTTGGCGGAGTGCTGGCCTGTTACAGCCTGCTGG | |
| | TTACCGTGGCCTTCATCATCTTTTGGGTCAAGCGGGGCAG | |
| | AAAGAAGCTGCTCTACATCTTCAAGCAGCCCTTCATGCGG | |
| | CCCGTGCAGACCACACAAGAGGAAGATGGCTGCTCCTGC | |
| | AGATTCCCCGAGGAAGAAGAAGGCGGCTGCGAGCTGAGA | |
| | GTGAAGTTCAGCAGATCCGCCGACGCTCCAGCCTATCAGC | |
| | AGGGCCAAAACCAGCTGTACAACGAGCTGAACCTGGGGA | |
| | GAAGAGAAGAGTACGACGTGCTGGATAAGCGGAGAGGCA | |
| | GAGATCCTGAAATGGGCGGCAAGCCCAGACGGAAGAATC | |
| | CTCAAGAGGGCCTGTATAATGAGCTGCAGAAAGACAAGA | |
| | TGGCCGAGGCCTACAGCGAGATCGGAATGAAGGGCGAGC | |
| | GCAGAAGAGGCAAGGGACACGATGGACTGTACCAGGGCC | |
| | TGAGCACCGCCACCAAGGATACCTATGACGCACTGCACAT | |
| | GCAGGCCCTGCCACCTAGA | |
| 154 | ATCGTTGGAGGACATGAAGCCCAGCCTCACAGCAGACCTT | PR3_10 |
| | ACATGGCCAGCCTGCAGATGAGAGGCAATCCCGGCTCTCA | (H44A)/(G4S)/ |
| | CTTTTGTGGCGGCACCCTGATTCACCCCTCCTTCGTTCTGA | CD8aTM CAAR |
| | CCGCCGCTGCCTGCCTGAGAGACATCCCTCAGAGACTGGT | (PR3_10_113) |
| | CAACGTGGTGCTGGGAGCCCACAACGTGCGGACACAAGA | Nucleotide |
| | ACCTACACAGCAGCACTTCAGCGTGGCCCAGGTGTTCCTG | |
| | AACAACTACGACGCCGAGAACAAGCTGAACGACGTGCTG | |
| | CTGATCCAGCTGAGCAGCCCTGCTAATCTGTCTGCCTCTGT | |
| | GGCCACTGTGCAGCTGCCTCAGCAAGATCAGCCTGTGCCT | |
| | CACGGCACACAGTGTCTGGCTATGGGATGGGGAAGAGTG | |
| | GGCGCTCATGATCCTCCAGCTCAAGTGCTGCAAGAGCTGA | |
| | ACGTGACCGTCGTGACCTTCTTCTGCCGGCCTCACAACAT | |
| | CTGCACCTTCGTGCCCAGAAGAAAGGCCGGCATCTGCTTC | |
| | GGAGATTCTGGCGGCCCTCTGATCTGCGACGGAATCATCC | |
| | AGGGCATCGACAGCTTCGTGATCTGGGGCTGTGCCACCAG | |
| | ACTGTTCCCCGACTTCTTTACCCGGGTGGCCCTCTACGTGG | |
| | ACTGGATCCGGTCTACACTGAGAGGCGGTGGAGGTTCAAT | |
| | CTATATCTGGGCACCTCTGGCCGGAACATGTGGCGTGCTG | |
| | CTGCTGTCTCTGGTCATCACCAAACGGGGCAGAAAGAAAC | |
| | TCCTGTATATATTCAAACAACCATTTATGAGACCAGTACA | |
| | AACTACTCAAGAGGAAGATGGCTGTAGCTGCCGATTTCCA | |
| | GAAGAAGAAGAAGGAGGATGTGAACTGCGGGTGAAGTTC | |
| | AGCAGAAGCGCCGACGCCCCTGCCTACCAGCAGGGCCAG | |
| | AATCAGCTGTACAACGAGCTGAACCTGGGGAGAAGAGAA | |
| | GAGTACGACGTGCTGGATAAGCGGAGAGGCAGAGATCCT | |
| | GAAATGGGCGGCAAGCCCAGACGGAAGAATCCTCAAGAG | |
| | GGCCTGTATAATGAGCTGCAGAAAGACAAGATGGCCGAG | |
| | GCCTACAGCGAGATCGGAATGAAGGGCGAGCGCAGAAGA | |
| | GGCAAGGGACACGATGGACTGTACCAGGGCCTGAGCACC | |
| | GCCACCAAGGATACCTATGACGCACTGCACATGCAGGCCC | |
| | TGCCACCTAGA | |
| 155 | ATCGTTGGAGGACATGAAGCCCAGCCTCACAGCAGACCTT | PR3_10 |
| | ACATGGCCAGCCTGCAGATGAGAGGCAATCCCGGCTCTCA | (H44A)/(G4S)/ |
| | CTTTTGTGGCGGCACCCTGATTCACCCCTCCTTCGTTCTGA | CD28TM CAAR |
| | CCGCCGCTGCCTGCCTGAGAGACATCCCTCAGAGACTGGT | (PR3_10_114) |
| | CAACGTGGTGCTGGGAGCCCACAACGTGCGGACACAAGA | Nucleotide |
| | ACCTACACAGCAGCACTTCAGCGTGGCCCAGGTGTTCCTG | |
| | AACAACTACGACGCCGAGAACAAGCTGAACGACGTGCTG | |
| | CTGATCCAGCTGAGCAGCCCTGCTAATCTGTCTGCCTCTGT | |
| | GGCCACTGTGCAGCTGCCTCAGCAAGATCAGCCTGTGCCT | |
| | CACGGCACACAGTGTCTGGCTATGGGATGGGGAAGAGTG | |
| | GGCGCTCATGATCCTCCAGCTCAAGTGCTGCAAGAGCTGA | |
| | ACGTGACCGTCGTGACCTTCTTCTGCCGGCCTCACAACAT | |
| | CTGCACCTTCGTGCCCAGAAGAAAGGCCGGCATCTGCTTC | |
| | GGAGATTCTGGCGGCCCTCTGATCTGCGACGGAATCATCC | |
| | AGGGCATCGACAGCTTCGTGATCTGGGGCTGTGCCACCAG | |
| | ACTGTTCCCCGACTTCTTTACCCGGGTGGCCCTCTACGTGG | |
| | ACTGGATCCGGTCTACACTGAGAGGCGGTGGAGGTTCATT | |
| | CTGGGTGCTCGTGGTCGTTGGCGGAGTGCTGGCCTGTTAC | |
| | AGCCTGCTGGTTACCGTGGCCTTCATCATCTTTTGGGTCAA | |
| | GCGGGGCAGAAAGAAGCTGCTCTACATCTTCAAGCAGCCC | |
| | TTCATGCGGCCCGTGCAGACCACACAAGAGGAAGATGGC | |
| | TGCTCCTGCAGATTCCCCGAGGAAGAAGAAGGCGGCTGC | |
| | GAGCTGAGAGTGAAGTTCAGCAGATCCGCCGACGCTCCA | |
| | GCCTATCAGCAGGGCCAAAACCAGCTGTACAACGAGCTG | |
| | AACCTGGGGAGAAGAGAAGAGTACGACGTGCTGGATAAG | |
| | CGGAGAGGCAGAGATCCTGAAATGGGCGGCAAGCCCAGA | |
| | CGGAAGAATCCTCAAGAGGGCCTGTATAATGAGCTGCAG | |
| | AAAGACAAGATGGCCGAGGCCTACAGCGAGATCGGAATG | |
| | AAGGGCGAGCGCAGAAGAGGCAAGGGACACGATGGACTG | |
| | TACCAGGGCCTGAGCACCGCCACCAAGGATACCTATGACG | |
| | CACTGCACATGCAGGCCCTGCCACCTAGA | |
| 156 | ATCGTTGGAGGACATGAAGCCCAGCCTCACAGCAGACCTT | PR3_10 |
| | ACATGGCCAGCCTGCAGATGAGAGGCAATCCCGGCTCTCA | (H44A)/CD8a/ |
| | CTTTTGTGGCGGCACCCTGATTCACCCCTCCTTCGTTCTGA | CD8aTM CAAR |
| | CCGCCGCTGCCTGCCTGAGAGACATCCCTCAGAGACTGGT | (PR3_10_115) |
| | CAACGTGGTGCTGGGAGCCCACAACGTGCGGACACAAGA | Nucleotide |
| | ACCTACACAGCAGCACTTCAGCGTGGCCCAGGTGTTCCTG | |
| | AACAACTACGACGCCGAGAACAAGCTGAACGACGTGCTG | |
| | CTGATCCAGCTGAGCAGCCCTGCTAATCTGTCTGCCTCTGT | |
| | GGCCACTGTGCAGCTGCCTCAGCAAGATCAGCCTGTGCCT | |
| | CACGGCACACAGTGTCTGGCTATGGGATGGGGAAGAGTG | |
| | GGCGCTCATGATCCTCCAGCTCAAGTGCTGCAAGAGCTGA | |
| | ACGTGACCGTCGTGACCTTCTTCTGCCGGCCTCACAACAT | |
| | CTGCACCTTCGTGCCCAGAAGAAAGGCCGGCATCTGCTTC | |
| | GGAGATTCTGGCGGCCCTCTGATCTGCGACGGAATCATCC | |
| | AGGGCATCGACAGCTTCGTGATCTGGGGCTGTGCCACCAG | |
| | ACTGTTCCCCGACTTCTTTACCCGGGTGGCCCTCTACGTGG | |
| | ACTGGATCCGGTCTACACTGAGAAAGCCTACCACAACCCC | |
| | AGCACCTAGACCCCCTACACCAGCACCAACCATCGCCTCT | |
| | CAGCCACTGAGCCTGCGGCCCGAGGCCTGTAGGCCAGCA | |
| | GCAGGAGGAGCCGTGCACACCAGGGGCCTGGACTTTGCCT | |
| | GCGATATCTATATCTGGGCACCTCTGGCCGGAACATGTGG | |
| | CGTGCTGCTGCTGTCTCTGGTCATCACCAAGCGGGGCAGA | |
| | AAGAAGCTGCTCTACATCTTCAAGCAGCCCTTCATGCGGC | |
| | CCGTGCAGACCACACAAGAGGAAGATGGCTGCTCCTGCA | |
| | GATTCCCCGAGGAAGAAGAAGGCGGCTGCGAGCTGAGAG | |
| | TGAAGTTCAGCAGATCCGCCGACGCTCCAGCCTATCAGCA | |
| | GGGCCAAAACCAGCTGTACAACGAGCTGAACCTGGGGAG | |
| | AAGAGAAGAGTACGACGTGCTGGATAAGCGGAGAGGCAG | |
| | AGATCCTGAAATGGGCGGCAAGCCCAGACGGAAGAATCC | |
| | TCAAGAGGGCCTGTATAATGAGCTGCAGAAAGACAAGAT | |
| | GGCCGAGGCCTACAGCGAGATCGGAATGAAGGGCGAGCG | |
| | CAGAAGAGGCAAGGGACACGATGGACTGTACCAGGGCCT | |
| | GAGCACCGCCACCAAGGATACCTATGACGCACTGCACATG | |
| | CAGGCCCTGCCACCTAGA | |
| 157 | ATCGTTGGAGGACATGAAGCCCAGCCTCACAGCAGACCTT | PR3_10 |
| | ACATGGCCAGCCTGCAGATGAGAGGCAATCCCGGCTCTCA | (H44A)/EAAAK/ |
| | CTTTTGTGGCGGCACCCTGATTCACCCCTCCTTCGTTCTGA | CD8aTM CAAR |
| | CCGCCGCTGCCTGCCTGAGAGACATCCCTCAGAGACTGGT | (PR3_10_116) |
| | CAACGTGGTGCTGGGAGCCCACAACGTGCGGACACAAGA | Nucleotide |
| | ACCTACACAGCAGCACTTCAGCGTGGCCCAGGTGTTCCTG | |
| | AACAACTACGACGCCGAGAACAAGCTGAACGACGTGCTG | |
| | CTGATCCAGCTGAGCAGCCCTGCTAATCTGTCTGCCTCTGT | |
| | GGCCACTGTGCAGCTGCCTCAGCAAGATCAGCCTGTGCCT | |
| | CACGGCACACAGTGTCTGGCTATGGGATGGGGAAGAGTG | |
| | GGCGCTCATGATCCTCCAGCTCAAGTGCTGCAAGAGCTGA | |
| | ACGTGACCGTCGTGACCTTCTTCTGCCGGCCTCACAACAT | |
| | CTGCACCTTCGTGCCCAGAAGAAAGGCCGGCATCTGCTTC | |
| | GGAGATTCTGGCGGCCCTCTGATCTGCGACGGAATCATCC | |
| | AGGGCATCGACAGCTTCGTGATCTGGGGCTGTGCCACCAG | |
| | ACTGTTCCCCGACTTCTTTACCCGGGTGGCCCTCTACGTGG | |
| | ACTGGATCCGGTCTACACTGAGAGAAGCCGCCGCTAAAAT | |
| | CTATATCTGGGCACCTCTGGCCGGAACATGTGGCGTGCTG | |
| | CTGCTGTCTCTGGTCATCACCAAACGGGGCAGAAAGAAAC | |
| | TCCTGTATATATTCAAACAACCATTTATGAGACCAGTACA | |
| | AACTACTCAAGAGGAAGATGGCTGTAGCTGCCGATTTCCA | |
| | GAAGAAGAAGAAGGAGGATGTGAACTGCGGGTGAAGTTC | |
| | AGCAGAAGCGCCGACGCCCCTGCCTACCAGCAGGGCCAG | |
| | AATCAGCTGTACAACGAGCTGAACCTGGGGAGAAGAGAA | |
| | GAGTACGACGTGCTGGATAAGCGGAGAGGCAGAGATCCT | |
| | GAAATGGGCGGCAAGCCCAGACGGAAGAATCCTCAAGAG | |
| | GGCCTGTATAATGAGCTGCAGAAAGACAAGATGGCCGAG | |
| | GCCTACAGCGAGATCGGAATGAAGGGCGAGCGCAGAAGA | |
| | GGCAAGGGACACGATGGACTGTACCAGGGCCTGAGCACC | |
| | GCCACCAAGGATACCTATGACGCACTGCACATGCAGGCCC | |
| | TGCCACCTAGA | |
| 158 | ATCGTTGGAGGACATGAAGCCCAGCCTCACAGCAGACCTT | PR3_10 |
| | ACATGGCCAGCCTGCAGATGAGAGGCAATCCCGGCTCTCA | (H44A)/MS/ |
| | CTTTTGTGGCGGCACCCTGATTCACCCCTCCTTCGTTCTGA | CD8aTM CAAR |
| | CCGCCGCTGCCTGCCTGAGAGACATCCCTCAGAGACTGGT | (PR3_10_117) |
| | CAACGTGGTGCTGGGAGCCCACAACGTGCGGACACAAGA | Nucleotide |
| | ACCTACACAGCAGCACTTCAGCGTGGCCCAGGTGTTCCTG | |
| | AACAACTACGACGCCGAGAACAAGCTGAACGACGTGCTG | |
| | CTGATCCAGCTGAGCAGCCCTGCTAATCTGTCTGCCTCTGT | |
| | GGCCACTGTGCAGCTGCCTCAGCAAGATCAGCCTGTGCCT | |
| | CACGGCACACAGTGTCTGGCTATGGGATGGGGAAGAGTG | |
| | GGCGCTCATGATCCTCCAGCTCAAGTGCTGCAAGAGCTGA | |
| | ACGTGACCGTCGTGACCTTCTTCTGCCGGCCTCACAACAT | |
| | CTGCACCTTCGTGCCCAGAAGAAAGGCCGGCATCTGCTTC | |
| | GGAGATTCTGGCGGCCCTCTGATCTGCGACGGAATCATCC | |
| | AGGGCATCGACAGCTTCGTGATCTGGGGCTGTGCCACCAG | |
| | ACTGTTCCCCGACTTCTTTACCCGGGTGGCCCTCTACGTGG | |
| | ACTGGATCCGGTCTACACTGAGAGAGTCTAAATACGGACC | |
| | GCCTTGTCCTCCTTGTCCCGGCCAGCCAAGAGAGCCCCAG | |
| | GTTTACACACTGCCTCCAAGCCAAGAGGAAATGACCAAG | |
| | AATCAGGTGTCCCTGACATGCCTGGTCAAGGGCTTCTACC | |
| | CCTCCGATATCGCCGTGGAATGGGAGAGCAATGGCCAGCC | |
| | TGAGAACAACTACAAGACCACACCTCCTGTGCTGGACAGC | |
| | GACGGCAGTTTCTTCCTGTATAGTAGACTCACCGTGGATA | |
| | AATCAAGATGGCAAGAGGGCAACGTGTTCAGCTGCAGCG | |
| | TGATGCACGAGGCCCTGCACAACCACTACACCCAGAAAA | |
| | GCCTGAGCCTGTCTCTGGGCAAGATGATCTATATCTGGGC | |
| | ACCTCTGGCCGGAACATGTGGCGTGCTGCTGCTGTCTCTG | |
| | GTCATCACCAAACGGGGCAGAAAGAAACTCCTGTATATAT | |
| | TCAAACAACCATTTATGAGACCAGTACAAACTACTCAAGA | |
| | GGAAGATGGCTGTAGCTGCCGATTTCCAGAAGAAGAAGA | |
| | AGGAGGATGTGAACTGCGGGTGAAGTTCAGCAGAAGCGC | |
| | CGACGCCCCTGCCTACCAGCAGGGCCAGAATCAGCTGTAC | |
| | AACGAGCTGAACCTGGGGAGAAGAGAAGAGTACGACGTG | |
| | CTGGATAAGCGGAGAGGCAGAGATCCTGAAATGGGCGGC | |
| | AAGCCCAGACGGAAGAATCCTCAAGAGGGCCTGTATAAT | |
| | GAGCTGCAGAAAGACAAGATGGCCGAGGCCTACAGCGAG | |
| | ATCGGAATGAAGGGCGAGCGCAGAAGAGGCAAGGGACAC | |
| | GATGGACTGTACCAGGGCCTGAGCACCGCCACCAAGGAT | |
| | ACCTATGACGCACTGCACATGCAGGCCCTGCCACCTAGA | |
| 159 | ATCGTTGGAGGACATGAAGCCCAGCCTCACAGCAGACCTT | PR3_10 |
| | ACATGGCCAGCCTGCAGATGAGAGGCAATCCCGGCTCTCA | (H44A)/SS/ |
| | CTTTTGTGGCGGCACCCTGATTCACCCCTCCTTCGTTCTGA | CD8aTM CAAR |
| | CCGCCGCTGCCTGCCTGAGAGACATCCCTCAGAGACTGGT | (PR3_10_118) |
| | CAACGTGGTGCTGGGAGCCCACAACGTGCGGACACAAGA | Nucleotide |
| | ACCTACACAGCAGCACTTCAGCGTGGCCCAGGTGTTCCTG | |
| | AACAACTACGACGCCGAGAACAAGCTGAACGACGTGCTG | |
| | CTGATCCAGCTGAGCAGCCCTGCTAATCTGTCTGCCTCTGT | |
| | GGCCACTGTGCAGCTGCCTCAGCAAGATCAGCCTGTGCCT | |
| | CACGGCACACAGTGTCTGGCTATGGGATGGGGAAGAGTG | |
| | GGCGCTCATGATCCTCCAGCTCAAGTGCTGCAAGAGCTGA | |
| | ACGTGACCGTCGTGACCTTCTTCTGCCGGCCTCACAACAT | |
| | CTGCACCTTCGTGCCCAGAAGAAAGGCCGGCATCTGCTTC | |
| | GGAGATTCTGGCGGCCCTCTGATCTGCGACGGAATCATCC | |
| | AGGGCATCGACAGCTTCGTGATCTGGGGCTGTGCCACCAG | |
| | ACTGTTCCCCGACTTCTTTACCCGGGTGGCCCTCTACGTGG | |
| | ACTGGATCCGGTCTACACTGAGAGAGTCTAAATACGGACC | |
| | GCCTTGTCCTCCTTGTCCCATGATCTATATCTGGGCACCTC | |
| | TGGCCGGAACATGTGGCGTGCTGCTGCTGTCTCTGGTCAT | |
| | CACCAAACGGGGCAGAAAGAAACTCCTGTATATATTCAA | |
| | ACAACCATTTATGAGACCAGTACAAACTACTCAAGAGGA | |
| | AGATGGCTGTAGCTGCCGATTTCCAGAAGAAGAAGAAGG | |
| | AGGATGTGAACTGCGGGTGAAGTTCAGCAGAAGCGCCGA | |
| | CGCCCCTGCCTACCAGCAGGGCCAGAATCAGCTGTACAAC | |
| | GAGCTGAACCTGGGGAGAAGAGAAGAGTACGACGTGCTG | |
| | GATAAGCGGAGAGGCAGAGATCCTGAAATGGGCGGCAAG | |
| | CCCAGACGGAAGAATCCTCAAGAGGGCCTGTATAATGAG | |
| | CTGCAGAAAGACAAGATGGCCGAGGCCTACAGCGAGATC | |
| | GGAATGAAGGGCGAGCGCAGAAGAGGCAAGGGACACGA | |
| | TGGACTGTACCAGGGCCTGAGCACCGCCACCAAGGATACC | |
| | TATGACGCACTGCACATGCAGGCCCTGCCACCTAGA | |

What is claimed:

1. A chimeric autoantibody receptor (CAAR) comprising:
(a) an extracellular proteinase 3 (PR3) antibody binding domain, wherein the PR3 antibody binding domain is a mutant PR3 protein that has reduced enzymatic activity compared with a wild type PR3 protein having the sequence set forth in SEQ ID NO:1;
(b) a transmembrane region; and
(c) an intracellular signaling region.

2. The CAAR of claim 1, further comprising a spacer between the extracellular PR3 antibody binding domain and the transmembrane domain.

3. The CAAR of claim 1 or 2, wherein the mutant PR3 protein comprises an amino acid substitution at a position selected from the group consisting of G4, H44, D91, D175 and S176 with reference to the numbering of positions in SEQ ID NO:1.

4. The CAAR of claim 3, wherein the amino acid substitution is selected from the group consisting of G4P, H44A, D91N, D175N, S176A and S176C.

5. The CAAR of claim 3, wherein the amino acid substitution is a conservative substitution of an amino acid substitution selected from the group consisting of G4P, H44A, D91N, D175N, S176A and S176C.

6. The CAAR of claim 1 or 2, wherein the mutant PR3 protein comprises an amino acid substitution that is in or near the catalytic triad, wherein the amino acid substitution is in an active site residue selected from the group consisting of H44, D91 and S176, with reference to numbering set forth in SEQ ID NO:1.

7. The CAAR of claim 6 wherein the amino acid substitution is selected from the group consisting of H44A, D91N, S176A and S176C.

8. The CAAR of any one of claims 1-7, wherein the mutant PR3 protein comprises an amino acid substitution at position H44, with reference to the numbering of positions in SEQ ID NO:1.

9. The CAAR of claim 1 or 2, wherein the mutant PR3 protein comprises an amino acid substitution at a position that interferes with the formation of the substrate binding pocket.

10. The CAAR of any one of claims 1-3 and 9, wherein the mutant PR3 protein comprises an amino acid substitution at position G4, with reference to the numbering of positions in SEQ ID NO:1.

11. The CAAR of any one of claims 3-10, wherein the mutant PR3 protein comprises 1, 2, 3, 4 or 5 amino acid substitutions compared to wild-type PR3 set forth in SEQ ID NO:1.

12. The CAAR of any one of claims 3-11, wherein the mutant PR3 protein comprises a single amino acid substitution compared to SEQ ID NO:1.

13. The CAAR of any one of claims 3-12, wherein the mutant PR3 protein comprises the amino acid substitution G4P.

14. A CAAR comprising:
(a) a PR3 antibody binding domain;
(b) a transmembrane region; and
(c) an intracellular signaling region,
wherein the PR3 antibody binding domain is a mutant PR3 protein comprising the amino acid substitution G4P.

15. The CAAR of any one of claims 1-5 and 7-14, wherein the PR3 antibody binding domain comprises a sequence of amino acids that exhibits at least 90% sequence identity to SEQ ID NO:9.

16. The CAAR of any one of claims 1-5 and 7-15, wherein the PR3 antibody binding domain comprises the sequence of amino acids set forth in SEQ ID NO:9.

17. The CAAR of any one of claims 1-5 and 7-16, wherein the amino acid sequence of the PR3 antibody binding domain consists of the sequence set forth in SEQ ID NO:9.

18. The CAAR of any one of claims 3-7, wherein the mutant PR3 protein comprises the amino acid substitution H44A.

19. A CAAR comprising:
(a) a PR3 antibody binding domain;
(b) a transmembrane region; and
(c) an intracellular signaling region,
wherein the PR3 antibody binding domain is a mutant PR3 protein comprising the amino acid substitution H44A.

20. The CAAR of any one of claims 1-6, 11, and 19, wherein the PR3 antibody binding domain comprises a sequence of amino acids that exhibits at least 90% sequence identity to SEQ ID NO:10.

21. The CAAR of any one of claims 1-6, 11, 19, and 20, wherein the PR3 antibody binding domain comprises the sequence of amino acids set forth in SEQ ID NO:10.

22. The CAAR of any one of claims 1-6, 11, or 19-21, wherein the amino acid sequence of the PR3 antibody binding domain consists of the sequence set forth in SEQ ID NO:10.

23. The CAAR of any one of claims 1-22, wherein the transmembrane region is or comprises a transmembrane domain from CD4, CD28, or CD8.

24. The CAAR of any one of claims 1-23, wherein the transmembrane region is or comprises a transmembrane domain from CD28, optionally a human CD28.

25. The CAAR of any one of claims 1-24, wherein the transmembrane region is or comprises SEQ ID NO: 26 or an amino acid sequence having at least at or about 90%, 91%, 92%, 93%, 94%, 95%, 96%, 97%, 98% or 99% sequence identity to SEQ ID NO:26.

26. The CAAR of any one of claims 1-25, wherein the transmembrane region is set forth in SEQ ID NO:26.

27. The CAAR of any one of claims 1-22, wherein the transmembrane region is or comprises a transmembrane domain from CD8a, optionally a human CD8a.

28. The CAAR of any one of claims 1-22 and 27, wherein the transmembrane region is or comprises SEQ ID NO: 98 or an amino acid sequence having at least at or about 90%, 91%, 92%, 93%, 94%, 95%, 96%, 97%, 98% or 99% sequence identity to SEQ ID NO:98.

29. The CAAR of any one of claims 1-22, 27, and 28, wherein the transmembrane region is set forth in SEQ ID NO:98.

30. The CAAR of any one of claims 1-29, wherein the intracellular signaling region comprises an intracellular signaling domain capable of inducing a primary activation signal in a T cell.

31. The CAAR of claim 30, wherein the intracellular signaling domain is a domain from a T cell receptor (TCR) component and/or comprises an immunoreceptor tyrosine-based activation motif (ITAM).

32. The CAAR of claim 30 or claim 31, wherein the intracellular signaling domain is a cytoplasmic signaling domain of a CD3-zeta (CD3ζ) chain, optionally a human CD3ζ chain.

33. The CAAR of any one of claims 30-32, wherein the intracellular signaling domain comprises the sequence set forth in SEQ ID NO:28, or an amino acid sequence having at least at or about 90%, 91%, 92%, 93%, 94%, 95%, 96%, 97%, 98% or 99% sequence identity to SEQ ID NO:28.

34. The CAAR of any one of claims 30-33, wherein the intracellular signaling domain consists of the sequence set forth in SEQ ID NO:28.

35. The CAAR of any one of claims 30-34, wherein the intracellular signaling region further comprises a costimulatory signaling region.

36. The CAAR of claim 35, wherein the costimulatory signaling region is between the transmembrane region and the intracellular signaling domain.

37. The CAAR of claim 35 or claim 36, wherein the costimulatory signaling region comprises an intracellular signaling domain of a T cell costimulatory molecule or a signaling portion thereof.

38. The CAAR of any one of claims 35-37, wherein the costimulatory signaling region comprises an intracellular signaling domain of CD28, 4-1BB, or ICOS.

39. The CAAR of any one of claims 35-38, wherein the costimulatory signaling region comprises an intracellular signaling domain of 4-1BB, optionally a human 4-1BB.

40. The CAAR of any one of claims 35-39, wherein the costimulatory signaling region comprises the sequence set forth in SEQ ID NO:27 or an amino acid sequence having at least at or about 90%, 91%, 92%, 93%, 94%, 95%, 96%, 97%, 98% or 99% sequence identity to SEQ ID NO:27.

41. The CAAR of any one of claims 35-40, wherein the costimulatory signaling region is set forth in SEQ ID NO:27.

42. The CAAR of any one of claims 2-41, wherein the spacer comprises at least a portion of an immunoglobulin or a variant thereof.

43. The CAAR of any one of claims 2-42, wherein the spacer comprises a hinge region of an immunoglobulin or a variant thereof.

44. The CAAR of claim 43, wherein the hinge region of an immunoglobulin is an IgG4 hinge region, optionally a human IgG4 hinge region, or a variant thereof.

45. The CAAR of any one of claims 42-44, wherein the spacer comprises a variant IgG4 hinge region comprising substitution of amino acids CPSC to CPPC compared to the wild-type IgG4 hinge region.

46. The CAAR of any one of claims 2-45, wherein the spacer is less than 15 amino acids in length.

47. The CAAR of any one of claims 2-45, wherein the spacer is between 12 and 15 amino acids in length.

48. The CAAR of any one of claims 2-47, wherein the spacer comprises the sequence set forth in SEQ ID NO: 22 or SEQ ID NO:23.

49. The CAAR of any one of claims 2-48, wherein the spacer is set forth in SEQ ID NO:22.

50. The CAAR of any one of claims 2-45 and 48, wherein the spacer is between 100 and 150 amino acids in length, optionally between 110 and 130 amino acids in length.

51. The CAAR of any one of claims 2-45, 48, and 50, wherein the spacer comprises a hinge region of an immunoglobulin and a CH3 region of an immunoglobulin.

52. The CAAR of claim 51, wherein the spacer comprises an IgG4 hinge region or a variant thereof and IgG4 CH3 region.

53. The CAAR of any one of claims 2-45, 48, and 50-52, wherein the spacer comprises the sequence set forth in SEQ ID NO: 24 or SEQ ID NO:96.

54. The CAAR of any one of claims 2-45, 48 and 50-53, wherein the spacer is set forth in SEQ ID NO:24.

55. The CAAR of any one of claims 2-45 and 48, wherein the spacer is between 200 and 250 amino acids in length, optionally between 220 and 240 amino acids in length.

56. The CAAR of any one of claims 2-45, 48 and 55 wherein the spacer comprises a hinge region of an immunoglobulin, a CH2 region of an immunoglobulin or a chimeric CH2 region of two different immunoglobulins, and a CH3 region of an immunoglobulin.

57. The CAAR of claim 56, wherein the spacer comprises IgG4 hinge region or a variant thereof, a chimeric CH2 region comprising a portion of an IgG4 CH2 and a portion of an IgG2 CH2 (IgG2/4 CH2 region), and an IgG4 CH3 region.

58. The CAAR of any one of claims 2-45, 48 and 55-57, wherein the spacer comprises the sequence set forth in SEQ ID NO: 25 or SEQ ID NO:97.

59. The CAAR of any one of claims 2-45, 48 and 55-58, wherein the spacer is set forth in SEQ ID NO:25.

60. The CAAR of any one of claims 2-41, 46 and 47, wherein the spacer comprises amino acids GGGGS (SEQ ID NO:99).

61. The CAAR of any one of claims 2-41, 46, 47 and 60, wherein the spacer is set forth in SEQ ID NO: 99.

62. The CAAR of any one of claims 2-41, 46, 47, and 60, wherein the spacer is set forth in SEQ ID NO: 100.

63. The CAAR of any one of claims 1-58, wherein the CAAR comprises a sequence of amino acids that exhibits at least 85% sequence identity to the sequence set forth in any of SEQ ID NOS: 29, 30, 31, 38, 39, 40, 53, 54, 55, 56, 57, 58, 104, 110, or 111 optionally wherein the CAAR comprises the sequence set forth in any one of SEQ ID NOS: 29, 30, 31, 38, 39, 40, 54, 55, 56, 57, 58, 59, 104, 110, or 111.

64. A CAAR comprising:
(a) a PR3 antibody binding domain comprising the sequence set forth in SEQ ID NO:9;
(b) a spacer comprising the sequence set forth in SEQ ID NO:25;
(c) a transmembrane region comprising the sequence set forth in SEQ ID NO:26;
(d) a costimulatory signaling region comprising the sequence set forth in SEQ ID NO:27; and
(e) an intracellular signaling region comprising the sequence set forth in SEQ ID NO:28.

65. A CAAR comprising a sequence of amino acids that exhibits at least 90% sequence identity to SEQ ID NO:55, wherein the sequence of amino acids comprises a G4P mutation with reference to the position numbering in SEQ ID NO:1.

66. The CAAR of claim 65, wherein the CAAR comprises the sequence of amino acids set forth in SEQ ID NO: 55.

67. A CAAR comprising:
(a) a PR3 antibody binding domain comprising the sequence set forth in SEQ ID NO:10;
(b) a spacer comprising the sequence set forth in SEQ ID NO:24;
(c) a transmembrane region comprising the sequence set forth in SEQ ID NO:26;
(d) a costimulatory signaling region comprising the sequence set forth in SEQ ID NO:27; and
(e) an intracellular signaling region comprising the sequence set forth in SEQ ID NO:28.

68. A CAAR comprising a sequence of amino acids that exhibits at least 90% sequence identity to SEQ ID NO:57, wherein the sequence of amino acids comprises a H44A mutation with reference to the numbering of positions in SEQ ID NO:1.

69. The CAAR of claim 68, wherein the CAAR comprises the sequence of amino acids set forth in SEQ ID NO: 57.

70. A CAAR comprising:
(a) a PR3 antibody binding domain comprising the sequence set forth in SEQ ID NO:9;
(b) a spacer comprising the sequence set forth in SEQ ID NO:100;
(c) a transmembrane region comprising the sequence set forth in SEQ ID NO:26;
(d) a costimulatory signaling region comprising the sequence set forth in SEQ ID NO:27; and
(e) an intracellular signaling region comprising the sequence set forth in SEQ ID NO:28.

71. A CAAR comprising a sequence of amino acids that exhibits at least 90% sequence identity to SEQ ID NO:104 wherein the sequence of amino acids comprises a G4P mutation with reference to the numbering of positions in SEQ ID NO:1.

72. The CAAR of claim 71, wherein the CAAR comprises the sequence of amino acids set forth in SEQ ID NO: 104.

73. A CAAR comprising:
(a) a PR3 antibody binding domain comprising the sequence set forth in SEQ ID NO:9;
(b) a spacer comprising the sequence set forth in SEQ ID NO:22;
(c) a transmembrane region comprising the sequence set forth in SEQ ID NO:98;
(d) a costimulatory signaling region comprising the sequence set forth in SEQ ID NO:27; and
(e) an intracellular signaling region comprising the sequence set forth in SEQ ID NO:28.

74. A CAAR comprising a sequence of amino acids that exhibits at least 90% sequence identity to SEQ ID NO:110 wherein the sequence of amino acids comprises a G4P mutation with reference to the numbering of positions in SEQ ID NO:1.

75. The CAAR of claim 74, wherein the CAAR comprises the sequence of amino acids set forth in SEQ ID NO: 110.

76. A CAAR comprising:
(a) a PR3 antibody binding domain comprising the sequence set forth in SEQ ID NO:10;
(b) a spacer comprising the sequence set forth in SEQ ID NO:100;
(c) a transmembrane region comprising the sequence set forth in SEQ ID NO:98;
(d) a costimulatory signaling region comprising the sequence set forth in SEQ ID NO:27; and
(e) an intracellular signaling region comprising the sequence set forth in SEQ ID NO:28.

77. A CAAR comprising a sequence of amino acids that exhibits at least 90% sequence identity to SEQ ID NO:111 or 119 wherein the sequence of amino acids comprises a H44A mutation with reference to the numbering of positions in in SEQ ID NO:1.

78. The CAAR of claim 77, wherein the CAAR comprises the sequence of amino acids set forth in SEQ ID NO: 111 or 119.

79. The CAAR of any one of claims 1-78, wherein the CAAR is able to be specifically bound by an anti-PR3 antineutrophil cytoplasmic antibody (PR3-ANCA).

80. The CAAR of any one of claims 1-79, wherein the PR3 antibody binding domain comprises at least 2, 3, 4, 5, 6 or 7 epitopes recognized by an anti-PR3 antineutrophil cytoplasmic antibody (PR3-ANCA).

81. The CAAR of claim 80, wherein the epitopes are selected from the group consisting of ANCA2 (AQPHSRPYMAS, SEQ ID NO:90), ANCA3 (PGSHFCGG, SEQ ID NO:91), ANCA4 (VVLGAHNVRTQ, SEQ ID NO:92), ANCA5 (FLNNYDAE, SEQ ID NO:93), ANCA6 (PVPHGTQC, SEQ ID NO: 94) and ANCA7 (CFGDSGGP, SEQ ID NO:95).

82. The CAAR of any one of claims 1-81, wherein the CAAR is specific for PR3-reactive B cells.

83. The CAAR of any one of claims 1-82, wherein Jurkat reporter cells which have Td-tomato knocked in at the endogenous Nur77 locus and that are transduced with the CAAR exhibit less than at or about 25%, at or about 20%, at or about 15%, at or about 10%, at or about 9%, at or about 8%, at or about 7%, at or about 5%, at or about 4%, at or about 3%, at or about 2% or at or about 1% tonic signalling, as measured by fluorescence of Td-Tomato by flow cytometry and determined as the total percentage of cells that express Td-Tomato in the transduced Jurkat reporter cells.

84. The CAAR of any one of claims 1-83, wherein cells transduced with the CAAR show antigen-specific activation after exposure to an anti-human PR3 antibody.

85. The CAAR of claim 83 or 84, wherein the CAAR comprises a sequence of amino acids that exhibits at least 85% sequence identity to the sequence set forth in any of SEQ ID NOS: 55, 57, 104, 110, and 111.

86. The CAAR of claim 85, wherein the CAAR comprises the sequence set forth in any one of SEQ ID NOS: 55, 57, 104, 110, and 111.

87. The CAAR of any one of claims 1-86, wherein the CAAR is able to kill at least 65% of an anti-PR3 antibody expressing cell target.

88. The CAAR of any one of claims 1-86, wherein the CAAR is able to kill at least 65% of each of at least three cell targets expressing different anti-PR3 antibodies, wherein each of the at least three different antibodies binds to a distinct PR3 epitope.

89. The CAAR of claim 87 or 88, wherein the CAAR comprises a sequence of amino acids that exhibits at least 85% sequence identity to the sequence set forth in any of SEQ ID NOS: 55, 57, 104, 110, and 111.

90. The CAAR of claim 89, wherein the CAAR comprises the sequence set forth in any one of SEQ ID NOS: 55, 57, 104, 110, and 111.

91. A polynucleotide comprising a nucleic acid encoding the CAAR of any one of claims 1-90.

92. The polynucleotide of claim 91, wherein the polynucleotide is optimized by splice site elimination.

93. The polynucleotide of claim 91 or claim 92, wherein the polynucleotide is codon-optimized for expression in a human cell.

94. The polynucleotide of any one of claims 91-93, wherein the polynucleotide comprises a nucleic acid sequence that exhibits at least 85% sequence identity to the sequence set forth in any of SEQ ID NOS: 59, 60, 61, 68, 69, 70, 83, 84, 85, 86, 87, 88, 144, 145, 146, 147, 148, 149, 150, 151, 152, 153, 154, 155, 156, 157, 158, or 159.

95. The polynucleotide of claim 94, wherein the polynucleotide comprises the sequence set forth in any one of SEQ ID NOS: 59, 60, 61, 68, 69, 70, 83, 84, 85, 86, 87, 88, 144, 145, 146, 147, 148, 149, 150, 151, 152, 153, 154, 155, 156, 157, 158, or 159.

96. The polynucleotide of any one of claims 91-93, wherein the polynucleotide comprises a nucleic acid sequence that exhibits at least 90% sequence identity to the sequence set forth in any of SEQ ID NOS: 59, 60, 68, 85, 86, 87, 88, 145, 151, or 152.

97. The polynucleotide of claim 96, wherein the polynucleotide comprises the sequence set forth in any one of SEQ ID NOS: 59, 60, 68, 85, 86, 87 or 88.

98. A vector, comprising the polynucleotide of any one of claims 91-97.

99. The vector of claim 98, wherein the vector is a viral vector.

100. The vector of claim 99, wherein the viral vector is a retroviral vector (e.g., lentiviral vector).

101. A cell comprising the CAAR of any one of claims 1-100.

102. A cell comprising the polynucleotide of any one of claims 91-97 or the vector of any one of claims 98-100.

103. The cell of claim 101 or claim 102, that is a lymphocyte.

104. The cell of any one of claims 101-103, wherein the cell is an NK cell or a T cell.

105. The cell of any one of claims 101-104, wherein the cell is a T cell and the T cell is a CD4+ T cell or a CD8+ T cell.

106. The cell of any one of claims 101-105, wherein the cell is a primary cell obtained from a subject.

107. The cell of claim 101 or claim 102, wherein the cell is an induced pluripotent stem cell.

108. The cell of any one of claims 101-105, wherein the cell has been differentiated from an induced pluripotent stem cell.

109. The cell of any one of claims 101-105, wherein the cell is an allogeneic cell.

110. The cell of any one of claims 101-105 and 109, wherein the cell is engineered to be hypoimmune.

111. The cell of any one of claims 101-110, wherein the cell exhibits cytotoxic activity against PR3-reactive B cells.

112. The cell of claim 111, wherein the cytotoxic activity is against a plurality of PR3-reactive B cells, wherein the plurality of PR3-reactive B cells are specific for two or more different PR3 antineutrophil cytoplasmic antibody (PR3-ANCA) epitopes.

113. The cell of claim 112, wherein the plurality of PR3-reactive B cells are specific for at least 2, 3, 4, 5, 6 or 7 different PR3 ANCA epitopes.

114. The cell of claim 112 or claim 113, wherein the plurality of PR3-reactive B cells are specific for at least 4 different PR3 ANCA eptitopes.

115. The cell of any one of claims 112-114, wherein the plurality of PR3-reactive B cells are specific for at least 5 different PR3 ANCA epitopes.

116. The cell of any one of claims 112-115, wherein the plurality of PR3-reactive B cells are specific for at least 6 different PR3 ANCA epitopes.

117. The cell of any one of claims 112-116, wherein the ANCA epitopes are selected from the group consisting of ANCA2 (AQPHSRPYMAS, SEQ ID NO:90), ANCA3 (PGSHFCGG, SEQ ID NO:91), ANCA4 (VVLGAHNVRTQ, SEQ ID NO:92), ANCA5 (FLNNYDAE, SEQ ID NO:93), ANCA6 (PVPHGTQC, SEQ ID NO: 94) and ANCA7 (CFGDSGGP, SEQ ID NO:95).

118. A composition comprising the cell of any one of claims 101-117.

119. The composition of claim 118, further comprising a pharmaceutically acceptable excipient.

120. The composition of claim 118 or claim 119, wherein the composition comprises CD4+ and CD8+ T cells.

121. The composition of claim 120, wherein the ratio of CD4+ to CD8+ T cells is from at or about 1:3 to 3:1, optionally at or about 1:2 to 2:1, optionally at or about 1:1.

122. The composition of any one of claims 118-121, wherein greater than at or about 90%, greater than at or about 95% or greater than at or about 98% of cells in the composition are CD3+ T cells.

123. The composition of any one of claims 118-122, wherein at least 40%, at least 50%, at least 60%, at least 70%, at least 80% or at least 90% of cells in the composition express the CAAR.

124. The composition of any one of claims 118-123, wherein, among a plurality of the cells in the composition expressing the CAAR, less than at or about 10%, at or about 9%, at or about 8%, at or about 7%, at or about 5%, at or about 4%, at or about 3%, at or about 2% or at or about 1% of the cells in the plurality exhibits tonic signaling and/or antigen independent activity or signaling.

125. A method of killing PR3-reactive B cells, the method comprising contacting a PR3-reactive B cell with the cell of any one of claims 101-117 or the composition of any one of claims 118-124.

126. The method of claim 125 that is performed in vitro or ex vivo.

127. The method of claim 125 that is performed in vivo in a subject.

128. The method of any one of claims 125-127, wherein the PR3-reactive B cell comprises a plurality of PR3-reactive B cells, wherein the plurality of PR3-reactive B cells are specific for two or more different PR3 antineutrophil cytoplasmic antibody (PR3-ANCA) epitopes.

129. The method of claim 128, wherein the plurality of PR3-reactive B cells are specific for 2, 3, 4, 5, 6 or 7 different PR3 ANCA epitopes.

130. The method of claim 128 or claim 129, wherein the plurality of PR3-reactive B cells are specific for at least 4 different PR3 ANCA eptitopes.

131. The method of any one of claims 128-130, wherein the plurality of PR3-reactive B cells are specific for at least 5 different PR3ANCA epitopes.

132. The method of any one of claims 128-131, wherein the plurality of PR3-reactive B cells are specific for at least 6 different PR3 ANCA epitopes.

133. The method of any one of claims 128-132, wherein the ANCA epitopes are selected from the group consisting of ANCA2 (AQPHSRPYMAS, SEQ ID NO:90), ANCA3 (PGSHFCGG, SEQ ID NO:91), ANCA4 (VVLGAHNVRTQ, SEQ ID NO:92), ANCA5 (FLNNYDAE, SEQ ID NO:93), ANCA6 (PVPHGTQC, SEQ ID NO: 94) and ANCA7 (CFGDSGGP, SEQ ID NO:95).

134. The method of any one of claims 125-133, wherein, among a plurality of PR3-reactive B cells, the method results in killing of greater than at or about 80%, greater than at or about 90%, greater than at or about 95%, PR3-reactive B cells, optionally wherein the PR3-reactive B cells are IgG+.

135. A method of treating a disease or disorder in a subject, the method comprising administering the cell of any one of claims 101-117 or the composition of any one of claims 118-124 to a subject in need of treatment thereof.

136. The method of claim 135, wherein the disease or disorder is an autoimmune condition.

137. The method of claim 135 and claim 136, wherein the disease or disorder is antineutrophil cytoplasmic antibody (ANCA)-associated vasculitis (AAV), optionally wherein the AAV is PR3-AAV.

138. The method of any one of claims 135-137, wherein the disease or disorder is selected from the group consisting of granulomatosis with polyangiitis (GPA), microscopic polyangiitis (MPA), or eosinophilic granulomatosis with polyangiitis (EGPA).

139. The method of claim 137 or 138, wherein the subject is positive for PR3-ANCA.

140. The method of any one of claims 137-139, wherein the method comprises testing the subject for PR3-ANCA.

141. The method of claim 140, wherein the method comprises verifying that the subject has tested positive for PR3-ANCA.

142. The method of any one of claims 137-141, wherein the subject has been treated with a prior therapy (e.g., one or more prior therapies) for AAV.

143. The method of claim 142, wherein the subject has failed to achieve remission or has relapsed following treatment with the prior therapy.

144. The method of claim 142 or 143, wherein the prior therapy comprises one or more of a glucocorticoid, cyclophosphamide (CYC), rituximab (RTX), plasma exchange, Avacopan, methotrexate (MTX), mycophenolate mofetil (MMF), azathioprine (AZA), leflunomide (LEF), belimumab, mepolizumab, and omalizumab.

145. The method of claim 144, wherein the glucocorticoid is one or more of prednisolone, methylprednisolone, prednisone, and dexamethasone.

146. The method of any one of claims 142-145, wherein the prior therapy comprises a remission induction therapy (e.g., a remission induction agent).

147. The method of claim 145, wherein the remission induction therapy comprises one or more of rituximab, cyclophosphamide, and a glucocorticoid.

148. The method of claim 147, wherein the glucocorticoid is one or more of prednisolone, methylprednisolone, prednisone, and dexamethasone.

149. The method of claim 148, wherein the glucocorticoid is prednisolone.

150. The method of any one of claims 142-149, wherein the prior therapy comprises a remission maintenance therapy.

151. The method of claim 150, wherein the remission maintenance therapy comprises one or more of rituximab, methotrexate, azathioprine, mycophenolate mofetil, leflunomide, mepolizumab, and omalizumab.

152. A method of treating a subject having PR3-ANCA vasculitis, the method comprising
(i) administering a remission induction therapy to the subject; and
(ii) administering the cell of any one of claims 101-117 or the composition of any one of claims 118-124 to the subject.

153. The method of claim 152, wherein the remission induction therapy comprises cyclophosphamide.

154. The method of claim 153, wherein cyclophosphamide is administered to the subject before the cell or composition is administered to the subject.

155. The method of claim 153, wherein a course of cyclophosphamide is administered to the subject at least 1 week, 2 weeks, 3 weeks, or 4 weeks before the cell or composition is administered to the subject.

156. The method of any one of claims 152-155, wherein the remission induction therapy further comprises a glucocorticoid.

157. The method of claim 156, wherein the glucocorticoid is prednisolone.

158. A method of treating a subject having PR3-ANCA vasculitis, the method comprising
(i) administering cyclophosphamide to the subject; and
(ii) administering the cell of any one of claims 101-117 or the composition of any one of claims 118-124 to the subject.

159. The method of claim 158, comprising administering cyclophosphamide to the subject before administering the cell of any of claims 101-117 or the composition of any of claims 118-124 to the subject.

160. The method of claim 159, wherein a course of cyclophosphamide is administered to the subject at least 1 week, 2 weeks, 3 weeks, or 4 weeks before the cell or composition is administered to the subject.

161. The method of claim 158 or 159, wherein the course of cyclophosphamide is administered according to a dosing schedule that has been determined to be sufficient to reduce ANCA titers in a patient having PR3-ANCA vasculitis.

162. The method of any one of claims 157-161, wherein cyclophosphamide is administered at an oral dose of 2 mg/kg/day for at least 1 week, 2 weeks, 3 weeks, or 4 weeks before the cell or composition is administered to the subject.

163. The method of any one of claims 157-161, wherein cyclophosphamide is administered at an IV dose of 15 mg/kg every 2 weeks for up to 3 doses prior to administering the cell or composition to the subject.

164. The method of any one of claims 157-163, wherein after the cell or composition is administered to the subject, the subject is not treated with cyclophosphamide.

165. The method of any one of claims 157-164, comprising administering a glucocorticoid to the subject before administering the cell or composition to the subject.

166. The method of any one of claims 157-165, comprising administering a glucocorticoid to the subject after administering the cell or composition to the subject.

167. A method of treating a subject having PR3-ANCA vasculitis, the method comprising
(i) administering to the subject a preconditioning therapy that has been shown to be effective to deplete ANCAs and subsequently; and
(ii) administering the cell of any of claims 101-117 or the composition of any of claims 118-124 to the subject.

168. The method of claim 167, wherein the preconditioning therapy comprises cyclophosphamide.
